(19) United States
(12) Patent Application Publication
(10) Pub. No.: US 2013/0338035 A1
(43) Pub. Date: Dec. 19, 2013

(54) MHC Genes and Risk of Graft Versus Host Disease

(71) Applicants: Ralf Dressel, Gottingen (DE); Anne Dickinson, Newcastle upon Tyne (GB); Bent Rolstadt, Oslo (NO); Lutz Walter, Gottingen (DE)

(72) Inventors: Ralf Dressel, Gottingen (DE); Anne Dickinson, Newcastle upon Tyne (GB); Bent Rolstadt, Oslo (NO); Lutz Walter, Gottingen (DE)

(73) Assignees: UNIVERSITY OF NEWCASTLE UPON TYNE, New Castle upon Tyne (GB); GEORG-AUSUST-UNIVERSITAT GOTTINGEN STIFTUNG OFFENTLICHEN RECHTS, UNIVERSITATSMEDIZIN, Gottingen (DE); DEUTSCHES PRIMATENZENTRUM GMBH, Gottingen (DE)

(21) Appl. No.: 13/993,964

(22) PCT Filed: Dec. 14, 2011
(86) PCT No.: PCT/EP11/72804

§ 371 Date: Aug. 28, 2013

(30) Foreign Application Priority Data
Dec. 14, 2010 (GB) 1021149.8

(51) Int. Cl. C12Q 1/68

(52) U.S. Cl. CPC C12Q 1/6883
USPC 506/9; 435/6.12; 435/7.92; 436/501; 435/7.1; 435/6.13

(57) ABSTRACT

The invention relates to the novel use of gene markers in a method of predicting the risk of or diagnosing a subject to develop graft versus host reaction (GvHR) or graft versus host disease (GvHD). In other aspects the invention also relates to methods of monitoring the efficacy of treatment of GvHR or GvHD, and methods of screening a candidate substance for the treatment of GvHR or GvHD.

### FIELD OF THE INVENTION

The major histocompatibility complex (MHC) is the most important genomic region that contributes to the risk of graft versus host disease (GVHD) after haematopoietic stem cell transplantation. Matching of MHC class I and II genes is essential for the success of transplantation. However, the MHC contains additional genes that also contribute to the risk of developing acute GVHD. The inventors identified rat and human MHC and NKC genes but also non-MHC and non-NKC genes that are regulated during graft versus host reaction (GVHR) in skin explant assays and could therefore serve as biomarkers for GVHD. Several of the respective human genes, including HLA-DMB, C2, AIF1, SPR1, UBD, and OLR1, are polymorphic. These candidates may therefore contribute to the genetic risk of GVHD in patients.

### BACKGROUND OF THE INVENTION

Haematopoietic stem cell transplantation (HSCT) is currently the only potentially curative treatment for many malignant and non-malignant haematological diseases. However, the overall survival rate after transplantation is still only 40% to 60% due to severe posttransplant complications, which include graft versus host disease (GVHD), relapse, and infection. Human leukocyte antigen (HLA) matching is essential to reduce the risk of graft rejection and GVHD. However, non-HLA genes also impact on transplant outcome and acute GVHD can be fatal even in patients receiving transplants from HLA-identical matched sibling donors (MSD). The cumulative incidence of grade 2 to 4 GVHD was 35% in a recent study evaluating 1960 MSD transplants. MSDs are currently available for about one third of the patients and, therefore, alternative donors are needed. HLA-matched unrelated donors (MUD) are more widely accepted than cord blood or mismatched related donors.

The level of HLA matching used for selection of MUDs has changed over time and usually includes now HLA-A, B, C, and DRB1 loci (8/8 match). In some studies matching has been extended to the HLA-DQB1 locus (10/10 match). A large recent study has compared MSD and 8/8 matched MUD transplants in a homogenous cohort of patients with chronic myeloid leukemia and found a 2.44 times higher risk of grade 2 to 4 acute GVHD in 8/8 matched MUD compared to MSD transplants (Arora M, et al. (2009) J Clin Oncol 27: 1644-1652). In another study, the incidence of grade 2 to 4 acute GVHD was still higher in 10/10 matched MUD compared to MSD transplants (Yakoub-Agha I, et al. (2006) J Clin Oncol 24: 5695-5702). The higher risk of GVHD after MUD compared to MSD transplants could be due to a higher degree of similarity in non-HLA genes for siblings, who share 50% of their genome with the respective recipient. However, also the HLA region itself could contribute to this difference since it harbors, in addition to the classical HLA class I and II genes, more than 200 other genes (Consortium T M S (1999) Nature 401: 921-923), many with immunological functions. In accordance with this hypothesis, mismatching of microsatellite markers in HLA class I, class II, and class III regions was associated with an increased risk of death in 10/10 matched MUD transplants. The HLA complex, as is the whole human genome, is organized into segments of closely linked genetic variants that are inherited as haplotypes on the same DNA strand. HLA haplotypes can be defined by HLA class I and II alleles and they are in strong linkage disequilibrium with defined genetic variants of non-class I/non-class II genes within the haplotype blocks within this region. Interestingly, HLA haplotype mismatching in 10/10 matched MUD transplants was associated with an increased risk of severe acute GVHD (Petersdorf E W, et al. (2007) PLoS Med 4: e8). This finding demonstrates that the HLA complex encodes in addition to HLA-A, B, C, DRB1, and DQB1 further unidentified genes that contribute significantly to the risk of developing acute GVHD. In case of disparity between donor and recipient alleles these genes may function as minor histocompatibility antigens. Alternatively, specific allelic variants may also increase the risk of GVHD, e.g., TNFA, a gene located within the class III region of the MHC encoding the pro-inflammatory cytokine tumor necrosis factor alpha (TNF-alpha). Several TNFA polymorphisms have been associated with an increased risk of GVHD and some of them are associated with increased TNF-alpha levels (Dickinson A M, et al. (2007) Expert Rev Mol Med 9: 1-19). The strong linkage disequilibrium within the HLA complex makes it very difficult to identify further non-class I/non-class II HLA genes involved in the pathophysiology of GVHD by genetic association studies alone.

HLA gene expression profiling may be an alternative strategy to identify HLA genes that are involved in the pathophysiology of GVHD. The inventors assumed that at least some non-class I/non-class II HLA genes that contribute to the risk of GVHD change their expression levels during disease progression. However, the genetic variation between clinical samples complicates the situation because allelic variation of gene expression could interfere with expression change in the pathophysiological process.

Accordingly, there is still a need for the identification of genes that contribute significantly to the risk of developing acute GVHD. These genes or gene markers may be used in the assessment of the risk to develop GVHD or GVHR, for the diagnosis of GVHD or GVHR, for monitoring treatment of GVHD or GVHR, and for screening for immunomodulating substances which may be useful in the treatment of GVHD or GVHR.

### SUMMARY OF THE INVENTION

In a first aspect, the invention relates to a method of predicting the risk of a subject to develop graft versus host reaction (GvHR) or graft versus host disease (GvHD), comprising
(a) determining the expression level of one or more prognostic RNA transcripts, or their corresponding cDNAs, or their expression products, in a sample obtained from said subject, wherein said transcript(s) or expression products is/are the transcript or expression product of one or more genes selected from the group consisting of:
   (i) Msr1, Pik3ap1, Pstpip1, Ctss, Pbx2, Grem1, Ly6g6e, Olr1, Spr1, Spic, Nfe2, Tnfaip8l2, and Ier3; or
   (ii) Msr1, Ctss, Pbx2, Grem1, Ly6g6e, Olr1, Spr1, Spic, and Nfe2; or
   (iii) Pik3ap1, Pstpip1, Tnfaip8l2, and Ier3;
(b) comparing the expression level of said one or more prognostic RNA transcript, or its corresponding cDNA, or its expression product with a corresponding baseline value;
   wherein
   (i) for every unit of increased expression of Olr1, Msr1, Pik3ap1, and/or Pstpip1; or the corresponding cDNA or expression product, said subject is expected to develop GvHR or GvHD; and
   (ii) for every unit of decreased expression of Ctss, Pbx2, Grem1, Ly6g6e, Spr1, Spic, Nfe2, Tnfaip8l2, and/or Ier3; or the corresponding cDNA or expression product, said subject is expected to develop GvHR or GvHD.

In a second aspect, the invention relates to a method of diagnosing graft versus host reaction (GvHR) or graft versus host disease (GvHD) in a subject following transplantation, comprising:
(a) determining the expression level of one or more prognostic RNA transcripts, or their corresponding cDNAs, or their expression products, in a sample obtained from said subject, wherein said transcript(s) or expression products is/are the transcript or expression product of one or more genes selected from the group consisting of:
   (i) Msr1, Pik3ap1, Pstpip1, Ctss, Pbx2, Grem1, Ly6g6e, Olr1, Spr1, Spic, Nfe2 Tnfaip8l2, and Ier3; or
   (ii) Msr1, Ctss, Pbx2, Grem1, Ly6g6e, Olr1, Spr1, Spic, and Nfe2; or
   (iii) Pik3ap1, Pstpip1, Tnfaip8l2, and Ier3;
(b) comparing the expression level of said one or more prognostic RNA transcript, or its corresponding cDNA, or its expression product with a corresponding baseline value;
   wherein
   (i) every unit of increased expression of Olr1, Msr1, Pik3ap1, and/or Pstpip1, or the corresponding cDNA or expression product, is indicative of GvHR or GvHD; and
   (ii) every unit of decreased expression of Ctss, Pbx2, Grem1, Ly6g6e, Spr1, Spic, Nfe2, Tnfaip8l2, and/or Ier3, or the corresponding cDNA or expression product, is indicative of GvHR or GvHD.

In a third aspect, the invention relates to a method of monitoring the efficacy of treatment of graft versus host reaction (GvHR) or graft versus host disease (GvHD) in a subject following transplantation, comprising:
(a) determining the expression level of one or more prognostic RNA transcripts, or their corresponding cDNAs, or their expression products, in a sample obtained from said subject at a first time point T1, and a later second time point T2, wherein said transcript(s) or expression products is/are the transcript or expression product of one or more genes selected from the group consisting of:
   (i) Msr1, Pik3ap1, Pstpip1, Ctss, Pbx2, Grem1, Ly6g6e, Olr1, Spr1, Spic, Nfe2, Tnfaip8l2, and Ier3; or
   (ii) Msr1, Ctss, Pbx2, Grem1, Ly6g6e, Olr1, Spr1, Spic, and Nfe2; or
   (iii) Pik3ap1, Pstpip1, Tnfaip8l2, and Ier3;
(b) comparing the expression level of said one or more prognostic RNA transcript, or its corresponding cDNA, or its expression product at time point T1 (Δ1) and time point T2 (Δ2) with a corresponding baseline value;
   wherein
   (i) a decline in units of an increased expression of Olr1, Msr1, Pik3ap1, and/or Pstpip1; or the corresponding cDNA or expression product at time point T2 in comparison with the increased expression of said at least one gene at the time point T1 (ΔΔ=Δ1−Δ2), is indicative of effective treatment of GvHR or GvHD; and
   (ii) a decline in units of a decreased expression of Ctss, Pbx2, Grem1, Ly6g6e, Spr1, Spic, Nfe2, Tnfaip8l2, and/or Ier3; or the corresponding cDNA or expression product at time point T2 in comparison with the decreased expression of said at least one gene at the time point T1(M=Δ1−Δ2), is indicative of effective treatment of GvHR or GvHD.

In a fourth aspect, the invention relates to a method of screening for a candidate substance for treatment of graft versus host reaction (GvHR) or graft versus host disease (GvHD), comprising:
(a) monitoring the efficacy of treatment by said candidate substance by using the method according to the third aspect in
   (i) a non-human animal model which suffers from GvHR or GvHD and to which the candidate substance has been administered, or
   (ii) in an ex vivo model, including but not limited to cell-based and/or tissue-based GvHR or HvHD assay such as the Skin Explant Assay, wherein said cells and/or tissue have been contacted with said candidate substance; and
(b) selecting a candidate substance which shows effective treatment of GvHR or GvHD.

In a final aspect, the invention pertains to a use of a kit in a method of predicting the risk of developing graft versus host reaction (GvHR) or graft versus host disease (GvHD) according to the first aspect, or in a method of diagnosing GvHR or GvHD according to the second aspect, or in a method of monitoring the efficacy of treatment of GvHR or GvHD according to the third aspect, wherein the kit comprises at least one isolated polynucleotide, wherein each isolated polynucleotide independently comprises
(i) at least 20 contiguous nucleotides of the nucleotide sequence selected from SEQ ID NO: 1, 3, 5, 7, 8, 10, 12, 14, 16, 18, 20, 22, and/or 24; or SEQ ID NO: 26-47, or
(ii) a nucleotide sequence having at least 90% identity to (i), or
(iii) the coding region of a gene comprising a nucleotide sequence according to (i) or (ii), or
(iv) a nucleotide sequence that can specifically hybridize, under conditions of high stringency, to a polynucleotide having a nucleotide sequence according to (i), (ii) or (iii); and
   wherein the kit comprises no more than 9000 isolated polynucleotides in total.

### DETAILED DESCRIPTION OF THE INVENTION

In an exploratory experiment, the inventors analyzed the expression of 169 genes with human homologues, including the respective MHC and NKC region genes, identified in the rat in human skin explant samples (c.f. example 2, and Table 9). These human skin explants were cultured for 1, 2, or 3 days resulting in GVHR of grades I, II, and III, respectively. Notably, 69% of all tested human genes were found to be regulated in at least one of these human samples as predicted by the results of the rat expression profiling experiments. 21%, i.e. 36 of the tested genes, were regulated in all 3 human skin explant samples in accordance with the rat model, but this regulation varied depending on the GVHR grade and the time course of the skin explant assay. Although the inventors only validated these genes firstly on 3 samples, the unexpectedly high concordance rate between the results of rat and human skin explant assays strongly suggests that the rat skin explant assay is an informative model for human GVHR and possibly GVHD.

Interestingly, for some of the genes that were found to be regulated in GVHR and GVHD in the rat, the human homologues are polymorphic and disease associations of gene polymorphisms have been described. These include HLA-DMB, C2, AIF1, SPR1, and possibly UBD. Therefore, these genes are especially interesting candidates of further non-class I/class II HLA genes that might confer an increased genetic risk of GVHD after HSCT depending on the genotype. In addition, the OLR1 gene in the NKC is polymorphic and polymorphisms of this gene have been associated with atherosclerosis, myocardial infarction, and Alzheimer's disease.

Several laboratory tests have been assessed for their ability to predict the risk of GVHD in patients. The skin explant assay has a predictive value of about 80% when cyclosporine alone is used for GVHD prophylaxis. A gene expression analysis of selected genes may help to further improve the predictive value of the assay. Pretransplant gene expression profiling of donor peripheral blood mononuclear cells (PBMC) has recently been shown to be a useful tool to predict the risk of GVHD. Post transplant differences in the gene expression profile of PBMC of patients with acute and chronic GVHD compared to non-GVHD samples have been described.

The inventors identified rat and human MHC and NKC genes but also non-MHC and non-NKC genes that are regulated during GVHR in skin explant assays and could therefore serve as biomarkers for GVHD. Several of the respective human genes, including HLA-DMB, C2, AIF1, SPR1, UBD, and OLR1, are polymorphic. These candidates may therefore contribute to the genetic risk of GVHD in patients.

The inventors observed a statistically significant and strong up or down regulation of 11 MHC, 6 NKC, and 168 genes encoded in other genomic regions, i.e. 4.9%, 14.0%, and 2.6% of the tested genes respectively. The regulation of 7 selected MHC and 3 NKC genes was confirmed by quantitative real-time PCR and in independent skin explant assays. In addition, similar regulations of most of the selected genes were observed in GVHD-affected skin lesions of transplanted rats and in human skin explant assays.

The inventors aimed to identify genes that are regulated during GVHR in the skin explant assay because these genes could be involved in the pathophysiology of GVHR and contribute to the genetic risk of GVHD. Special attention was given to genes encoded within the MHC region for the following reasons: Firstly, evidence has been presented that further risk genes for GVHD in addition to MHC class I and class II genes are present in this region. Secondly, those genes cannot easily be identified by genetic linkage analysis alone due to the strong linkage disequilibrium with MHC class I and class II genes so that expression profiling could be a worthwhile alternative approach. Thirdly, the inventors wanted to focus in this initial study on a fully characterized genomic region of special immunological importance rather than to follow a whole genome expression profiling approach. Importantly, 39% of the BN rat MHC genes (RT1ⁿ haplotype) annotated by Hurt and colleagues (Hurt P, et al. (2004) Genome Res 14: 631-639) were at the time point of array construction not represented in the Agilent database and therefore not represented on the Agilent whole rat genome array. In addition to the MHC region, genes of the NKC region were included because this region encodes Ly49 genes and their products can function as receptors for the numerous MHC class Ia and Ib gene products encoded in the MHC. A higher percentage of MHC genes and NKC genes than genes in other regions of the genome were found to be regulated in the allogeneic skin explants compared to skin samples co-cultured with syngeneic lymphocytes. Of the 25 MHC genes found to be significantly regulated (p<0.05), 5 are known to be involved in antigen processing and presentation. Besides two of three MHC class Ia genes in the BN strain (RT1-A1 and RT1-A2) that present peptides to cytotoxic T lymphocytes (CTL), the genes Tap1 and Psmb8, encoding a subunit of the antigen transporter and a subunit of the immunoproteasome (also known as LMP7), were found to be up-regulated. RT1-DMb encodes a homologue of HLA-DMB, a chaperone in the MHC class II presentation pathway. Furthermore, non-classical MHC class Ib genes (RT1-CE2, RT1-CE3, RT1-CE5, RT1-CE8, RT1-CE10, RT1-CE16, RT1-T24-4, RT-BM1) were up-regulated during GVHR in the skin explants. The function of the RT1-C/E/M class I genes is not well defined. It is known that they can become targets of CTL and function as ligands for activating or inhibitory NK receptors. RT1-C/E/M incompatibility has been shown to induce skin and pancreas graft rejection and to modulate the fate of MHC class IImismatched heart grafts. The RT1-T24-4 gene belongs to a family of genes that was originally identified as pseudogenes in the haplotype r21. In the RT1ⁿ haplotype all four family members are presumably functional. However, their actual function has not been experimentally demonstrated so far. The RT-BM1 (RT1-S3) gene is assumed to be orthologous to the mouse H2-T23gene, which encodes the Qa-1 molecule. This is a functional homologue of HLA-E, which presents leader peptides of MHC class I molecules to the inhibitory NK receptor CD94/NKG2A. Interestingly, its expression can vary substantially depending on the RT1 haplotype. It has to be noticed that no human/rat orthology can be established for the class I genes in the various class I clusters. Therefore, with respect to class I genes, the rat cannot serve as a model for the HLA complex. However, the non-class I genes are clearly orthologous.

In addition to Tap1, Psmb8, and RT1-DMb, 12 further non-class I MHC genes were found to be regulated in the rat skin explant assays, some of them also involved in the immune response, such as the complement component C2, while such a role is strongly assumed for other genes. The allograft inflammatory factor 1 (Aif1), was cloned from chronically rejecting rat cardiac allografts and it was also found in transplanted human hearts. Persistent expression of AIF-1 is associated with the development of a cardiac allograft vasculopathy. The expression of AIF-1 is mostly limited to the monocyte/macrophage lineage, and can be augmented by interferon (IFN)-γ. The specific function of the leukocyte specific transcript 1 (Lst1) gene is not known, although its strong expression in dendritic cells and functional data suggest an immunomodulatory role. The expression of human LST1, specifically of splice variants encoding soluble isoforms, was increased in rheumatoid arthritis-affected blood and synovium and was up-regulated in response to IFN-γ. The immediate early response 3 (Ier3) gene is stress-inducible and is involved in the regulation of cell death and oncogenesis. The protein (also known as IEX-1 or IEX-1L) functions in the protection of cells from Fas or TNF-α-induced apoptosis. However, it increases the rate of apoptosis in ultraviolet B irradiated keratinocytes. Distinct domains of the proteins were described to be responsible for pro and anti-apoptotic activities of the protein. The diubiquitin gene (UbM has been shown to be expressed in rat lymphoblasts, thymus, and testis. In the mouse it is expressed in dendritic cells and B cells, is inducible by IFN-γ, and can cause apoptosis. The protein (also known as FAT10) provides an ubiquitin-independent signal for proteasomal degradation. It has been suggested to participate in antigen processing, but its expression did not affect MHC class I expression or antigen presentation. In view of the reported roles of these genes in the immune response, a direct involvement in GVHD is conceivable.

For the other regulated MHC genes an involvement in immune functions has not been established so far. Spr1 (or Psors1c2) is the psoriasis susceptibility 1 candidate 2 gene and was found to be expressed in the thymus of rats. Its human homologue is expressed in normal and psoriasis skin and has been suggested to confer susceptibility to psoriasis. The function of the gene product is not known so far. G18 (Gpsm3) is an activator of G-protein signaling. Pbx2 encodes an ubiquitously expressed transcriptional activator. The Ly6g6e gene belongs to the lymphocyte antigen 6 (Ly-6) superfamily that encodes proteins attached to the cell surface by a glycosylphosphatidylinositol (GPI) anchor that is directly involved in signal transduction. Mouse Ly6g6e was found to be highly expressed at the leading edges of cells, on filopodia, which are normally involved in cell adhesion and migration. The mitochondrial ribosomal protein S18B (Mrsps18b) gene encodes a 28S subunit protein that belongs to the ribosomal protein S18P family. The functions of the HLA-B associated transcript 5 (Bat5) and Fij13158 (or RGD1303066) genes have not been characterized so far.

Many of the up-regulated MHC genes are inducible by IFN-γ, a type II cytokine that is primarily secreted by activated T and NK cells. Several studies have demonstrated an increased level of IFN-γ in the early phase of GVHD. Therefore, this cytokine might be highly important for the regulation of the expression of MHC genes during GVHR.

The inventors also included the NKC region in the expression profiling which harbors the Ly49 genes that encode NK receptors of the killer cell lectin-like receptor type and some of these have been shown to interact with both MHC class Ia and Ib molecules. In contrast to the MHC region, no reference sequence has been published for the NKC region of the rat. Therefore, 20 genes that were recently assigned to this region in the assembly RGSC v3.4 (Twigger et al. (2008) Nat. Genet. 40: 523-527) were not represented on the array. However, for most of them no function associated with the immune system has been reported. Interestingly, only Ly49 receptor genes which have an ITIM motif in their cytoplasmic region were up-regulated in the allogeneic skin explant assays. This includes also the LOC690045 gene which encodes an immunoreceptor similar to Ly49si1. It is not clear whether one of these gene products interacts with the MHC class Ib molecules that the inventors found to be up-regulated. Ly49 receptors are normally present mainly on NK cells and the skin explants harbored few leukocytes. However, skin resident lymphocytes can become activated in human skin explant assays. Although few NK cells infiltrating a tissue that normally does not contain these cells might cause a drastic relative change in the presence of Ly49 transcripts, the possibility should not be dismissed that other cells may express the receptors under pathological conditions. The role of NK cells for GVHR in skin explants needs to be further explored. In general NK cells are assumed to prevent GVHR, improve engraftment and to exert strong graft-versusleukemia effects without causing GVHD.

In the NKC region the inventors found one non-Ly49 gene to be regulated. The Olr1 gene encodes a receptor protein which belongs to the C-type lectin superfamily. The protein (also known as LOX-1) binds, internalizes and degrades oxidized low-density lipoprotein, which induces vascular endothelial cell activation and dysfunction, resulting in pro-inflammatory responses, pro-oxidative conditions and apoptosis. In addition, it acts as a receptor for extracellular heat shock protein 70 on dendritic cells. Binding and internalization of heat shock protein 70/peptide complexes channels peptides into the MHC class I presentation pathway. Thus, the protein is involved in antigen cross-presentation to naive T cells.

In addition to the MHC and NKC region genes, 168 further genes were significantly regulated in allogeneic skin explants. Many of them also have immunological functions and need to be analyzed in more detail in subsequent studies.

The results obtained in the MHC and NKC gene expression profiling experiment were confirmed in most tested cases by qRT-PCR on the skin explant samples. Some genes, e.g. Aif1 and Ly49i9, appeared to be up-regulated even in grade I GVHR. Olr1, in contrast, was up-regulated predominantly in grade II and III GVHR in all comparisons. Importantly, several of the MHC and NKC genes that were identified to be regulated in the skin explant assays, including Aif1, Lst1, and Olr1, were also regulated in the GVHD affected skin of transplanted animals. Thus, the skin explant assay can model GVHD not only histologically but also with respect to gene regulation. However, the up-regulation of the tested Ly49 genes (Ly49si1 and Ly49i9) that were observed in the skin explant was not clearly confirmed in the GVHD-affected skin of transplanted rats. Skin lesions from transplanted animals are likely to be more heterogeneous with respect to the dynamics of the pathophysiological process than skin explant samples, and this may contribute to the variation in results.

In conclusion, the MHC gene expression profiling approach in the rat skin explant assay identified a number of non-class I/class II genes that might contribute to the MHC-associated risk of GVHD following HSCT. These genes could be directly involved in the pathophysiology of GVHD or serve as molecular markers for GVHD and GVHR. The possibility should not be dismissed, however, that these marker genes could indicate that protective pathways are induced which modulate tissue damage during inflammation. Moreover, their human homologues may be useful for risk assessment, diagnosis, and as potential targets for therapy of GVHD in patients.

Accordingly, in a first aspect, the invention relates to a method of predicting the risk of a subject to develop graft versus host reaction (GvHR) or graft versus host disease (GvHD), comprising
(a) determining the expression level of one or more prognostic RNA transcripts, or their corresponding cDNAs, or their expression products, in a sample obtained from said subject, wherein said transcript(s) or expression products is/are the transcript or expression product of one or more genes selected from the group consisting of:
   (i) Msr1, Pik3ap1, Pstpip1, Ctss, Pbx2, Grem1, Ly6g6e, Olr1, Spr1, Spic, Nfe2, Tnfaip8l2, and Ier3; or
   (ii) Msr1, Ctss, Pbx2, Grem1, Ly6g6e, Olr1, Spr1, Spic, and Nfe2; or
   (iii) Pik3ap1, Pstpip1, Tnfaip8l2, and Ier3;
(b) comparing the expression level of said one or more prognostic RNA transcript, or its corresponding cDNA, or its expression product with a corresponding baseline value;
   wherein
   (i) for every unit of increased expression of Olr1, Msr1, Pik3ap1, and/or is Pstpip1; or the corresponding cDNA or expression product, said subject is expected to develop GvHR or GvHD; and
   (ii) for every unit of decreased expression of Ctss, Pbx2, Grem1, Ly6g6e, Spr1, Spic, Nfe2, Tnfaip8l2, and/or Ier3; or the corresponding cDNA or expression product, said subject is expected to develop GvHR or GvHD.

The term “predicting the risk of a subject” is used herein to refer to the prediction of the likelihood of a subject to develop graft versus host reaction (GvHR) or graft versus host disease (GvHD). The method of the invention may be used clinically in order to determine the best treatment modalities and regimen and/or to evaluate whether said patient is likely to respond favourably to a treatment, such as surgical intervention, as for example a transplantation, in particular with regard to dosage and/or drug combinations.

The terms “graft versus host reaction” and “graft versus host disease” may be used synonymously. Usually, 3 criteria must be met in order for GvHD to occur: (1) Administration of an immunocompetent graft, with viable and functional immune cells, (2) the recipient is immunologically disparate—histoincompatible, and (3) the recipient is immunocompromised and therefore cannot destroy or inactivate the transplanted cells. Following transplantation, T cells present in the graft, either as contaminants or intentionally introduced into the host, perceive host tissues as antigenically foreign and attack the tissues of the transplant recipient. GvHD occurs not only when there is a mismatch of a major MHC class I or II antigen but also in the context of disparities between minor histocompatibility antigens. GvHD is a common complication in recipients of bone marrow transplants from, e.g., HLA-identical siblings, who typically differ from each other in many polymorphic proteins encoded by genes unlinked to the MHC.

Clinically, GvHD is divided into acute and chronic forms. Acute and chronic GvHD appear to involve different immune cell subsets, different cytokine profiles, different host targets, and respond differently to treatment. For example, the acute form of GvHD is normally observed within the first 100 days post-transplant, and is a major challenge to transplants owing to associated morbidity and mortality. In contrast thereto, the chronic form of GvHD normally occurs after 100 days. The appearance of moderate to severe cases of chronic GvHD adversely influences long-term survival.

In order to determine the expression level of one or more prognostic RNA transcripts, or their corresponding cDNAs, or their expression products of one or more genes, a sample comprising cells from the subject and, thus, the prognostic RNA transcripts or their expression products is first derived from said subject.

The term “sample”, as used herein, refers to a sample comprising cells of the subject to be tested, which may be the graft or the host in question, which cells may be homogenized and disrupted in order to release and optionally isolate the prognostic RNA transcripts. Preferably, the sample is a biopsy sample, preferably a biopsy sample of the tissue to be transplanted or of the tissue wherein the transplant is grafted, or a sample of Peripheral Blood Mononuclear Cells (PBMC). A peripheral blood mononuclear cell (PBMC) is a blood cell having a round nucleus. In general, these cells are immune cells, such as lymphocytes (e.g., T cells, B cells, and NK cells), monocytes or macrophages. These cells are often extracted from whole blood using ficoll, a hydrophilic polysaccharide that separates layers of blood, with monocytes and lymphocytes forming a buffy coat containing said PBMCs under a layer of plasma. Alternatively, PBMC can be extracted from whole blood using a hypotonic lysis which will preferentially lyse red blood cells. This method results in neutrophils and other polymorphonuclear (PMN) cells which are important in innate immune defence being obtained. However any other suitable method may be used in order to isolate PBMC from the subject.

Said RNA transcripts may subsequently be used directly or processed into another form, such as cRNA, cDNA or PCR amplification products, which still represent the expressed genes in said sample of cells, i.e. the transcripts of these genes. RNA can be isolated according to any of a number of methods well known to those of skill in the art. For example, mRNA is isolated using oligo d(T) column chromatography or glass beads. For example, RNA extraction may be performed by using TRIZOL reagent (Invitrogen, Carlsbad, Calif., USA), as described in more detail in the examples.

Alternatively, a cDNA may be reverse transcribed from said prognostic RNA transcript, RNA transcribed from that cDNA, a DNA amplified from that cDNA, RNA transcribed from the amplified DNA, or the like. Total mRNA can be converted to cDNA and amplified by conventional procedures, for example, by reverse transcription in a per se known manner. A cDNA may be amplified by any of a variety of conventional amplification procedures, including PCR. Suitable PCR primers can be selected using any well-known methods. Further examples of primers are given in the Examples section below.

For example, the level of expression of a prognostic RNA transcript or their corresponding cDNA in a sample is determined by hybridizing said RNA transcript or corresponding cDNA to a detectable probe, e.g. by performing a microarray, such as a DNA microarray. Alternatively, the expression level may be determined by using quantitative PCR. Then, the mRNA copy number may be calculated from the amount of hybridization, which generally reflects the level of expression of the polynucleotide in the cells of the sample, normalized to the amount of total RNA (or cDNA) or to the expression level of one or more housekeeping genes.

Methods for detecting hybridization are well known in the art. For example, the prognostic RNA transcript or corresponding cDNA may be labelled with a fluorescent label and levels and patterns of fluorescence indicative of hybridization are measured, e.g. by fluorescence microscopy, preferably confocal fluorescence microscopy. In this detection method, an argon ion laser excites the fluorescent label, emissions are directed to a photomultiplier and the amount of emitted light detected and quantitated. The detected signals are considered to be proportional to the amount of probe/target hybridization complex at each position of the microarray. Further, the fluorescence microscope may be associated with a computer-driven scanner device to generate a quantitative two-dimensional image of hybridization intensity. The scanned image is examined to determine the abundance/expression level of each hybridized target transcript. Alternatively, a fluorescent imaging device, such as a microarray scanner, may be used.

Typically, array fluorescence intensities can be normalized to take into account variations in hybridization intensities when more than one array is used under similar test conditions. This may be achieved by using the intensities derived from internal normalization controls contained on each microarray, e.g. from housekeeping genes. Accordingly, “normalized” refers to the expression level of an RNA transcript relative to the expression level of the total RNA or relative to the expression level of a housekeeping gene. Housekeeping genes are genes that are constitutively transcribed at a relatively constant level across many or all known conditions, since the housekeeping gene's products are typically needed for maintenance of the cell. Examples of housekeeping genes include actin, GAPDH, and ubiquitin.

However, further methods for determining the amount of a polynucleotide are well known in the art and may include any suitable quantitative method. Examples for such further methods are, for example, quantitative PCR, such as real-time PCR, or reverse transcription PCR (RT-PCR), using primers specific for those polynucleotides. Methods for selecting suitable primers for detecting and quantitating the amplified product are known in the art and exemplified in the Examples section below.

Alternatively, the expression level may be determined by the expression product(s), i.e. by the polypeptides encoded by said genes. This may be accomplished using immunological methods involving the use of antibodies directed against said polypeptides, e.g. the expression level of the corresponding expression product(s) is determined by ELISA or immunohistochemistry.

In order to perform an ELISA the sample with an unknown amount of expression is product is immobilized on a solid support either non-specifically via adsorption to the surface of the solid support or specifically by a so called capture-antibody specific to the expression product. After the antigen is immobilized the detection antibody is added, forming a complex with the antigen. The detection antibody can itself be covalently linked to an enzyme, or can be detected by a secondary antibody linked to an enzyme. Between each step the plate is typically washed with a mild detergent solution to remove any proteins or antibodies that are not specifically bound. Detection occurs by adding an enzymatic substrate to produce a visible signal, which indicates the quantity of expression product in the sample. Immunohistochemistry refers to a method involving localizing the expression product in said cells of the sample using fluorescence labelled antibodies and determining the fluorescence intensity.

However, any suitable method may be used for determining the expression level of said expression product(s), such as by way of Western blotting, protein microarray, flow cytometry or surface plasmon resonance.

Thus, in a preferred embodiment, the expression level is determined by DNA microarray analysis or quantitative PCR and subsequent calculation of the mRNA copy number normalized to the amount of total RNA or to the expression level of one or more housekeeping genes. In another preferred embodiment the expression level of the corresponding expression product(s) is determined by ELISA, Western blotting, protein microarray or immunohistochemistry, flow cytometry or surface plasmon resonance.

The term “every unit of increased expression” and the term “every unit of decreased expression” as used herein refers to an expression level of one or more prognostic RNA transcripts, or their corresponding cDNAs, or their expression product(s) that has been found differentially expressed in subjects suffering or prone to suffer from GvHD or GvHR in comparison to healthy subjects. Thus, in case of “every unit of increased expression”, the higher the expression level of a gene which is predominantly expressed in the cells of a subject who suffers or is prone to suffer from GvHD or GvHR, the higher is the risk that the subject to be tested is expected to develop GvHD or GvHR. Likewise, in case of “every unit of decreased expression”, the lower the expression level of a gene which is predominantly expressed in healthy subjects but not in subjects suffering or prone to suffer from GvHD or GvHR, the higher is the risk that the subject to be tested develops GvHR or GvHD.

The determined expression level may be compared to a corresponding baseline value. As used herein, the term “corresponding baseline value” refers to the level of gene expression in normal cells or PBMCs, e.g. in a sample from a healthy subject or from a “pool” of samples derived from healthy subjects; or from a pool of one or more tissues from healthy subjects. Any of the above types of baseline values may be available in a database compiled from such values. Therefore, in a preferred embodiment, the baseline value may be the expression level of said at least one gene in at least one healthy subject.

An expression level of a gene may be considered as being increased if the log 2-fold change is at least 1, such as at least 1.1, or at least 1.2, preferably at least 1.25, such as at least 1.5 or at least 1.75, more preferably at least 2.0, such as at least 2.25 or at least 2.5, and most preferably at least 2.75 or even at least 3.0. Likewise, an expression level of a gene may be considered as being decreased if the log 2-fold change is at least −1, such as at least −1.1, or at least −1.2, preferably at least −1.25, such as at least −1.5 or at least −1.75, more preferably at least −2.0, such as at least −2.25 or at least −2.5, and most preferably at least −2.75 or even at least −3.0.

Alternatively, the term “increased” amount means herein an amount which is typically at least 120%, at least 130%, at least 140%, at least 150%, at least 175%, preferably at least 200%, at least 225%, at least 250%, at least 275%, more preferably at least 300%, at least 350%, or at least 400%, most preferably at least 500% of the baseline value.

Likewise, the term “decreased”, as meant herein, refers to an amount which is typically less than 90%, less than 85%, less than 80%, less than 75%, more preferably less than 70%, less than 65%, less than 60%, even more preferably less than 50%, less than 40%, or less than 30%, most preferably less than 25%, less than 20%, or even less than 10% of the baseline value.

The term “one or more” as used herein means that either one, two, three, four, five, six, seven, eight, nine, ten, eleven, twelve, or all thirteen expression level(s) of said genes is/are determined.

The term “corresponding”, as used herein, refers to the baseline value of the same gene as determined in the sample. The genes and their respective reference sequence is given in Table 10 below as well as in SEQ ID NOs 1-25.

The following combinations of biomarkers are contemplated to be particularly useful:

| Combination | Ctss | Pbx2 | Grem1 | Ly6g6e | Olr1 | Spr1 | Msr1 | Spic | Nfe2 | Tnfaip8l2 | Ier3 | Pik3ap1 | Pstpip1 |
|---|---|---|---|---|---|---|---|---|---|---|---|---|---|
| 1 | + | | | | | | | | | | | | |
| 2 | | + | | | | | | | | | | | |
| 3 | | | + | | | | | | | | | | |
| 4 | | | | + | | | | | | | | | |
| 5 | | | | | + | | | | | | | | |
| 6 | | | | | | | | | | | | | |
| 7 | | | | | | + | | | | | | | |
| 8 | | | | | | | + | | | | | | |
| 9 | | | | | | | | + | | | | | |
| 10 | | | | | | | | | + | | | | |
| 11 | | | | | | | | | | + | | | |
| 12 | | | | | | | | | | | + | | |
| 13 | | | | | | | | | | | | + | |
| 14 | | | | | | | | | | | | | + |
| 15 | + | + | | | | | | | | | | | |
| 16 | + | | + | | | | | | | | | | |
| 17 | + | | | + | | | | | | | | | |
| 18 | + | | | | + | | | | | | | | |
| 19 | + | | | | | | | | | | | | |
| 20 | + | | | | | + | | | | | | | |
| 21 | + | | | | | | + | | | | | | |
| 22 | + | | | | | | | + | | | | | |
| 23 | + | | | | | | | | + | | | | |
| 24 | + | | | | | | | | | + | | | |
| 25 | + | | | | | | | | | | + | | |
| 26 | + | | | | | | | | | | | + | |
| 27 | + | | | | | | | | | | | | + |
| 28 | | + | + | | | | | | | | | | |
| 29 | | + | | + | | | | | | | | | |
| 30 | | + | | | + | | | | | | | | |
| 31 | | + | | | | | | | | | | | |
| 32 | | + | | | | + | | | | | | | |
| 33 | | + | | | | | + | | | | | | |
| 34 | | + | | | | | | + | | | | | |
| 35 | | + | | | | | | | + | | | | |
| 36 | | + | | | | | | | | + | | | |
| 37 | | + | | | | | | | | | + | | |
| 38 | | + | | | | | | | | | | + | |
| 39 | | + | | | | | | | | | | | + |
| 40 | | + | | | | | | | | | | | |
| 41 | | + | | | | | | | | | | | |
| 42 | | + | | | | | | | | | | | |
| 43 | | | + | + | | | | | | | | | |
| 44 | | | + | | + | | | | | | | | |
| 45 | | | + | | | | | | | | | | |
| 46 | | | + | | | + | | | | | | | |
| 47 | | | + | | | | + | | | | | | |
| 48 | | | + | | | | | + | | | | | |
| 49 | | | + | | | | | | + | | | | |
| 50 | | | + | | | | | | | + | | | |
| 51 | | | + | | | | | | | | + | | |
| 52 | | | + | | | | | | | | | + | |
| 53 | | | + | | | | | | | | | | + |
| 54 | | | | + | + | | | | | | | | |
| 55 | | | | + | | | | | | | | | |
| 56 | | | | + | | + | | | | | | | |
| 57 | | | | + | | | + | | | | | | |
| 58 | | | | + | | | | + | | | | | |
| 59 | | | | + | | | | | + | | | | |
| 60 | | | | + | | | | | | + | | | |
| 61 | | | | + | | | | | | | + | | |
| 62 | | | | + | | | | | | | | + | |
| 63 | | | | + | | | | | | | | | + |
| 64 | | | | | + | | | | | | | | |
| 65 | | | | | + | + | | | | | | | |
| 66 | | | | | + | | + | | | | | | |
| 67 | | | | | + | | | + | | | | | |
| 68 | | | | | + | | | | + | | | | |
| 69 | | | | | + | | | | | + | | | |
| 70 | | | | | + | | | | | | + | | |
| 71 | | | | | + | | | | | | | + | |
| 72 | | | | | + | | | | | | | | + |
| 73 | | | | | | + | | | | | | | |
| 74 | | | | | | | + | | | | | | |
| 75 | | | | | | | | + | | | | | |
| 76 | | | | | | | | | + | | | | |
| 77 | | | | | | | | | | + | | | |
| 78 | | | | | | | | | | | + | | |
| 79 | | | | | | | | | | | | + | |
| 80 | | | | | | | | | | | | | + |
| 81 | | | | | | + | + | | | | | | |
| 82 | | | | | | + | | + | | | | | |
| 83 | | | | | | + | | | + | | | | |
| 84 | | | | | | + | | | | + | | | |
| 85 | | | | | | + | | | | | + | | |
| 86 | | | | | | + | | | | | | + | |
| 87 | | | | | | + | | | | | | | + |
| 88 | | | | | | | + | + | | | | | |
| 89 | | | | | | | + | | + | | | | |
| 90 | | | | | | | + | | | + | | | |
| 91 | | | | | | | + | | | | + | | |
| 92 | | | | | | | + | | | | | + | |
| 93 | | | | | | | + | | | | | | + |
| 94 | | | | | | | | + | + | | | | |
| 95 | | | | | | | | + | | + | | | |
| 96 | | | | | | | | + | | | + | | |
| 97 | | | | | | | | + | | | | + | |
| 98 | | | | | | | | + | | | | | + |
| 100 | | | | | | | | | + | + | | | |
| 101 | | | | | | | | | + | | + | | |
| 102 | | | | | | | | | + | | | + | |
| 103 | | | | | | | | | + | | | | + |
| 104 | | | | | | | | | | + | + | | |
| 105 | | | | | | | | | | + | | + | |
| 106 | | | | | | | | | | + | | | + |
| 107 | | | | | | | | | | | + | + | |
| 108 | | | | | | | | | | | + | | + |
| 109 | | | | | | | | | | | | + | + |
| 110 | + | + | + | | | | | | | | | | |
| 112 | + | + | | + | | | | | | | | | |
| 113 | + | + | | | + | | | | | | | | |
| 114 | + | + | | | | | | | | | | | |
| 115 | + | + | | | | + | | | | | | | |
| 116 | + | + | | | | | + | | | | | | |
| 117 | + | + | | | | | | + | | | | | |
| 118 | + | + | | | | | | | + | | | | |
| 119 | + | + | | | | | | | | + | | | |
| 120 | + | + | | | | | | | | | + | | |
| 121 | + | + | | | | | | | | | | + | |
| 122 | + | + | | | | | | | | | | | + |
| 123 | | + | + | + | | | | | | | | | |
| 124 | | + | + | | + | | | | | | | | |
| 125 | | + | + | | | | | | | | | | |
| 126 | | + | + | | | + | | | | | | | |
| 127 | | + | + | | | | + | | | | | | |
| 128 | | + | + | | | | | + | | | | | |
| 129 | | + | + | | | | | | + | | | | |
| 130 | | + | + | | | | | | | + | | | |
| 131 | | + | + | | | | | | | | + | | |
| 132 | | + | + | | | | | | | | | + | |
| 133 | | + | + | | | | | | | | | | + |
| 134 | | | + | + | + | | | | | | | | |
| 135 | | | + | + | | | | | | | | | |
| 136 | | | + | + | | + | | | | | | | |
| 137 | | | + | + | | | + | | | | | | |
| 138 | | | + | + | | | | + | | | | | |
| 139 | | | + | + | | | | | + | | | | |
| 140 | | | + | + | | | | | | + | | | |
| 141 | | | + | + | | | | | | | + | | |
| 142 | | | + | + | | | | | | | | + | |
| 143 | | | + | + | | | | | | | | | + |
| 144 | | | + | + | | | | | | | | | |
| 145 | | | | + | + | + | | | | | | | |
| 146 | | | | + | + | | + | | | | | | |
| 147 | | | | + | + | | | + | | | | | |
| 148 | | | | + | + | | | | + | | | | |
| 149 | | | | + | + | | | | | + | | | |
| 150 | | | | + | + | | | | | | + | | |
| 151 | | | | + | + | | | | | | | + | |
| 152 | | | | + | + | | | | | | | | + |
| 153 | | | | | + | + | | | | | | | |
| 154 | | | | | + | | + | | | | | | |
| 155 | | | | | + | | | + | | | | | |
| 156 | | | | | + | | | | + | | | | |
| 157 | | | | | + | | | | | + | | | |
| 158 | | | | | + | | | | | | + | | |
| 159 | | | | | + | | | | | | | + | |
| 160 | | | | | + | | | | | | | | + |
| 161 | | | | | | + | + | | | | | | |
| 162 | | | | | | + | | + | | | | | |
| 163 | | | | | | + | | | + | | | | |
| 164 | | | | | | + | | | | + | | | |
| 165 | | | | | | + | | | | | + | | |
| 166 | | | | | | + | | | | | | + | |
| 167 | | | | | | + | | | | | | | + |
| 168 | | | | | | + | + | + | | | | | |
| 169 | | | | | | + | + | | + | | | | |
| 170 | | | | | | + | + | | | + | | | |
| 171 | | | | | | + | + | | | | + | | |
| 172 | | | | | | + | + | | | | | + | |
| 173 | | | | | | + | + | | | | | | + |
| 174 | | | | | | | + | + | + | | | | |
| 175 | | | | | | | + | + | | + | | | |
| 176 | | | | | | | + | + | | | + | | |
| 177 | | | | | | | + | + | | | | + | |
| 178 | | | | | | | + | + | | | | | + |
| 179 | | | | | | | | + | + | + | | | |
| 180 | | | | | | | | + | + | | + | | |
| 181 | | | | | | | | + | + | | | + | |
| 182 | | | | | | | | + | + | | | | + |
| 183 | | | | | | | | | + | + | + | | |
| 184 | | | | | | | | | + | + | | + | |
| 185 | | | | | | | | | + | + | | | + |
| 186 | | | | | | | | | | + | + | + | |
| 187 | | | | | | | | | | + | + | | + |
| 188 | | | | | | | | | | | + | + | + |
| 189 | + | + | + | + | | | | | | | | | |
| 190 | + | + | + | | + | | | | | | | | |
| 191 | + | + | + | | | | | | | | | | |
| 192 | + | + | + | | | + | | | | | | | |
| 193 | + | + | + | | | | + | | | | | | |
| 194 | + | + | + | | | | | + | | | | | |
| 195 | + | + | + | | | | | | + | | | | |
| 196 | + | + | + | | | | | | | + | | | |
| 197 | + | + | + | | | | | | | | + | | |
| 198 | + | + | + | | | | | | | | | + | |
| 199 | + | + | + | | | | | | | | | | + |
| 200 | | + | + | + | + | | | | | | | | |
| 201 | | + | + | + | | | | | | | | | |
| 202 | | + | + | + | | + | | | | | | | |
| 203 | | + | + | + | | | + | | | | | | |
| 204 | | + | + | + | | | | + | | | | | |
| 205 | | + | + | + | | | | | + | | | | |
| 206 | | + | + | + | | | | | | + | | | |
| 207 | | + | + | + | | | | | | | + | | |
| 208 | | + | + | + | | | | | | | | + | |
| 209 | | + | + | + | | | | | | | | | + |
| 210 | | | + | + | + | | | | | | | | |
| 211 | | | + | + | + | + | | | | | | | |
| 212 | | | + | + | + | | + | | | | | | |
| 213 | | | + | + | + | | | + | | | | | |
| 214 | | | + | + | + | | | | + | | | | |
| 215 | | | + | + | + | | | | | + | | | |
| 216 | | | + | + | + | | | | | | + | | |
| 217 | | | + | + | + | | | | | | | + | |
| 218 | | | + | + | + | | | | | | | | + |
| 219 | | | | + | + | + | | | | | | | |
| 220 | | | | + | + | | + | | | | | | |
| 221 | | | | + | + | | | + | | | | | |
| 222 | | | | + | + | | | | + | | | | |
| 223 | | | | + | + | | | | | + | | | |
| 224 | | | | + | + | | | | | | + | | |
| 225 | | | | + | + | | | | | | | + | |
| 226 | | | | + | + | | | | | | | | + |
| 227 | | | | | + | + | + | | | | | | |
| 228 | | | | | + | + | | + | | | | | |
| 229 | | | | | + | + | | | + | | | | |
| 230 | | | | | + | + | | | | + | | | |
| 231 | | | | | + | + | | | | | + | | |
| 232 | | | | | + | + | | | | | | + | |
| 233 | | | | | + | + | | | | | | | + |
| 234 | | | | | | + | + | + | | | | | |
| 235 | | | | | | + | + | | + | | | | |
| 236 | | | | | | + | + | | | + | | | |
| 237 | | | | | | + | + | | | | + | | |
| 238 | | | | | | + | + | | | | | + | |
| 239 | | | | | | + | + | | | | | | + |
| 240 | | | | | | + | + | + | + | | | | |
| 241 | | | | | | + | + | + | | + | | | |
| 242 | | | | | | + | + | + | | | + | | |
| 243 | | | | | | + | + | + | | | | + | |
| 244 | | | | | | + | + | + | | | | | + |
| 245 | | | | | | | + | + | + | + | | | |
| 246 | | | | | | | + | + | + | | + | | |
| 247 | | | | | | | + | + | + | | | + | |
| 248 | | | | | | | + | + | + | | | | + |
| 249 | | | | | | | | + | + | + | + | | |
| 250 | | | | | | | | + | + | + | | + | |
| 251 | | | | | | | | + | + | + | | | + |
| 252 | | | | | | | | | + | + | + | + | |
| 253 | | | | | | | | | + | + | + | | + |
| 254 | | | | | | | | | | + | + | + | + |
| 255 | + | + | + | + | + | | | | | | | | |
| 256 | + | + | + | + | | | | | | | | | |
| 257 | + | + | + | + | | + | | | | | | | |
| 258 | + | + | + | + | | | + | | | | | | |
| 259 | + | + | + | + | | | | + | | | | | |
| 260 | + | + | + | + | | | | | + | | | | |
| 261 | + | + | + | + | | | | | | + | | | |
| 262 | + | + | + | + | | | | | | | + | | |
| 263 | + | + | + | + | | | | | | | | + | |
| 264 | + | + | + | + | | | | | | | | | + |
| 265 | | + | + | + | + | | | | | | | | |
| 266 | | + | + | + | + | + | | | | | | | |
| 267 | | + | + | + | + | | + | | | | | | |
| 268 | | + | + | + | + | | | + | | | | | |
| 269 | | + | + | + | + | | | | + | | | | |
| 270 | | + | + | + | + | | | | | + | | | |
| 271 | | + | + | + | + | | | | | | + | | |
| 272 | | + | + | + | + | | | | | | | + | |
| 273 | | + | + | + | + | | | | | | | | + |
| 274 | | | + | + | + | + | | | | | | | |
| 275 | | | + | + | + | | + | | | | | | |
| 276 | | | + | + | + | | | + | | | | | |
| 277 | | | + | + | + | | | | + | | | | |
| 278 | | | + | + | + | | | | | + | | | |
| 279 | | | + | + | + | | | | | | + | | |
| 280 | | | + | + | + | | | | | | | + | |
| 281 | | | + | + | + | | | | | | | | + |
| 282 | | | | + | + | + | + | | | | | | |
| 283 | | | | + | + | + | | + | | | | | |
| 284 | | | | + | + | + | | | + | | | | |
| 285 | | | | + | + | + | | | | + | | | |
| 286 | | | | + | + | + | | | | | + | | |
| 287 | | | | + | + | + | | | | | | + | |
| 288 | | | | + | + | + | | | | | | | + |
| 289 | | | | | + | + | + | + | | | | | |
| 290 | | | | | + | + | + | | + | | | | |
| 291 | | | | | + | + | + | | | + | | | |
| 292 | | | | | + | + | + | | | | + | | |
| 293 | | | | | + | + | + | | | | | + | |
| 294 | | | | | + | + | + | | | | | | + |
| 295 | | | | | | + | + | + | + | | | | |
| 296 | | | | | | + | + | + | | + | | | |
| 297 | | | | | | + | + | + | | | + | | |
| 298 | | | | | | + | + | + | | | | + | |
| 299 | | | | | | + | + | + | | | | | + |
| 300 | | | | | | + | + | + | | | | | |
| 301 | | | | | | + | + | + | | | | | |
| 302 | | | | | | + | + | + | | | | | |
| 303 | | | | | | + | + | + | + | + | | | |
| 304 | | | | | | + | + | + | + | | + | | |
| 305 | | | | | | + | + | + | + | | | + | |
| 306 | | | | | | + | + | + | + | | | | + |
| 307 | | | | | | + | + | + | + | | | | |
| 308 | | | | | | + | + | + | + | | | | |
| 309 | | | | | | + | + | + | + | | | | |
| 310 | | | | | | | + | + | + | + | + | | |
| 311 | | | | | | | + | + | + | + | | + | |
| 312 | | | | | | | + | + | + | + | | | + |
| 313 | | | | | | | | + | + | + | + | + | |
| 314 | | | | | | | | + | + | + | + | | + |
| 315 | | | | | | | | | + | + | + | + | + |
| 316 | + | + | + | + | + | | | | | | | | |
| 317 | + | + | + | + | + | + | | | | | | | |
| 318 | + | + | + | + | + | | + | | | | | | |
| 319 | + | + | + | + | + | | | + | | | | | |
| 320 | + | + | + | + | + | | | | + | | | | |
| 321 | + | + | + | + | + | | | | | + | | | |
| 322 | + | + | + | + | + | | | | | | + | | |
| 323 | + | + | + | + | + | | | | | | | + | |
| 324 | + | + | + | + | + | | | | | | | | + |
| 325 | | + | + | + | + | + | | | | | | | |
| 326 | | + | + | + | + | | + | | | | | | |
| 327 | | + | + | + | + | | | + | | | | | |
| 328 | | + | + | + | + | | | | + | | | | |
| 329 | | + | + | + | + | | | | | + | | | |
| 330 | | + | + | + | + | | | | | | + | | |
| 331 | | + | + | + | + | | | | | | | + | |
| 332 | | + | + | + | + | | | | | | | | + |
| 333 | | | + | + | + | + | + | | | | | | |
| 334 | | | + | + | + | + | | + | | | | | |
| 335 | | | + | + | + | + | | | + | | | | |
| 336 | | | + | + | + | + | | | | + | | | |
| 337 | | | + | + | + | + | | | | | + | | |
| 338 | | | + | + | + | + | | | | | | + | |
| 339 | | | + | + | + | + | | | | | | | + |
| 340 | | | | + | + | + | + | + | | | | | |
| 341 | | | | + | + | + | + | | + | | | | |
| 342 | | | | + | + | + | + | | | + | | | |
| 343 | | | | + | + | + | + | | | | + | | |
| 344 | | | | + | + | + | + | | | | | + | |
| 345 | | | | + | + | + | + | | | | | | + |
| 346 | | | | | + | + | + | + | + | | | | |
| 347 | | | | | + | + | + | + | | + | | | |
| 348 | | | | | + | + | + | + | | | + | | |
| 349 | | | | | + | + | + | + | | | | + | |
| 350 | | | | | + | + | + | + | | | | | + |
| 351 | | | | | | + | + | + | + | + | | | |
| 352 | | | | | | + | + | + | + | | + | | |
| 353 | | | | | | + | + | + | + | | | + | |
| 354 | | | | | | + | + | + | + | | | | + |
| 355 | | | | | | + | + | + | + | + | + | | |
| 356 | | | | | | + | + | + | + | + | | + | |
| 357 | | | | | | + | + | + | + | + | | | + |
| 358 | | | | | | | + | + | + | + | + | + | |
| 359 | | | | | | | + | + | + | + | + | | + |
| 360 | | | | | | | | + | + | + | + | + | + |
| 361 | | | | | | | | + | + | + | + | + | |
| 362 | + | + | + | + | + | + | | | | | | | |
| 363 | + | + | + | + | + | | + | | | | | | |
| 364 | + | + | + | + | + | | | + | | | | | |
| 365 | + | + | + | + | + | | | | + | | | | |
| 366 | + | + | + | + | + | | | | | + | | | |
| 367 | + | + | + | + | + | | | | | | + | | |
| 368 | + | + | + | + | + | | | | | | | + | |
| 369 | + | + | + | + | + | | | | | | | | + |
| 370 | | + | + | + | + | + | + | | | | | | |
| 371 | | + | + | + | + | + | | + | | | | | |
| 372 | | + | + | + | + | + | | | + | | | | |
| 373 | | + | + | + | + | + | | | | + | | | |
| 374 | | + | + | + | + | + | | | | | + | | |
| 375 | | + | + | + | + | + | | | | | | + | |
| 376 | | + | + | + | + | + | | | | | | | + |
| 377 | | | + | + | + | + | + | + | | | | | |
| 378 | | | + | + | + | + | + | | + | | | | |
| 379 | | | + | + | + | + | + | | | + | | | |
| 380 | | | + | + | + | + | + | | | | + | | |
| 381 | | | + | + | + | + | + | | | | | + | |
| 382 | | | + | + | + | + | + | | | | | | + |
| 383 | | | | + | + | + | + | + | + | | | | |
| 384 | | | | + | + | + | + | + | | + | | | |
| 385 | | | | + | + | + | + | + | | | + | | |
| 386 | | | | + | + | + | + | + | | | | + | |
| 387 | | | | + | + | + | + | + | | | | | + |
| 388 | | | | | + | + | + | + | + | + | | | |
| 389 | | | | | + | + | + | + | + | | + | | |
| 390 | | | | | + | + | + | + | + | | | + | |
| 391 | | | | | + | + | + | + | + | | | | + |
| 392 | | | | | | + | + | + | + | + | + | | |
| 393 | | | | | | + | + | + | + | + | | + | |
| 394 | | | | | | + | + | + | + | + | | | + |
| 395 | | | | | | + | + | + | + | + | + | + | |
| 396 | | | | | | + | + | + | + | + | + | | + |
| 397 | | | | | | | + | + | + | + | + | + | + |
| 398 | + | + | + | + | + | + | + | | | | | | |
| 399 | + | + | + | + | + | + | | + | | | | | |
| 400 | + | + | + | + | + | + | | | + | | | | |
| 401 | + | + | + | + | + | + | | | | + | | | |
| 402 | + | + | + | + | + | + | | | | | + | | |
| 403 | + | + | + | + | + | + | | | | | | + | |
| 404 | + | + | + | + | + | + | | | | | | | + |
| 405 | | + | + | + | + | + | + | + | | | | | |
| 406 | | + | + | + | + | + | + | | + | | | | |
| 407 | | + | + | + | + | + | + | | | + | | | |
| 408 | | + | + | + | + | + | + | | | | + | | |
| 409 | | + | + | + | + | + | + | | | | | + | |
| 410 | | + | + | + | + | + | + | | | | | | + |
| 411 | | | + | + | + | + | + | + | + | | | | |
| 412 | | | + | + | + | + | + | + | | + | | | |
| 413 | | | + | + | + | + | + | + | | | + | | |
| 414 | | | + | + | + | + | + | + | | | | + | |
| 415 | | | + | + | + | + | + | + | | | | | + |
| 416 | | | | + | + | + | + | + | + | + | | | |
| 417 | | | | + | + | + | + | + | + | | + | | |
| 418 | | | | + | + | + | + | + | + | | | + | |
| 419 | | | | + | + | + | + | + | + | | | | + |
| 420 | | | | | + | + | + | + | + | + | + | | |
| 421 | | | | | + | + | + | + | + | + | | + | |
| 422 | | | | | + | + | + | + | + | + | | | + |
| 423 | | | | | | + | + | + | + | + | + | + | |
| 424 | | | | | | + | + | + | + | + | + | | + |
| 425 | | | | | | + | + | + | + | + | + | + | + |
| 426 | + | + | + | + | + | + | + | + | | | | | |
| 427 | + | + | + | + | + | + | + | | + | | | | |
| 428 | + | + | + | + | + | + | + | | | + | | | |
| 429 | + | + | + | + | + | + | + | | | | + | | |
| 430 | + | + | + | + | + | + | + | | | | | + | |
| 431 | + | + | + | + | + | + | + | | | | | | + |
| 432 | | + | + | + | + | + | + | + | + | | | | |
| 433 | | + | + | + | + | + | + | + | | + | | | |
| 434 | | + | + | + | + | + | + | + | | | + | | |
| 435 | | + | + | + | + | + | + | + | | | | + | |
| 436 | | + | + | + | + | + | + | + | | | | | + |
| 437 | | | + | + | + | + | + | + | + | + | | | |
| 438 | | | + | + | + | + | + | + | + | | + | | |
| 439 | | | + | + | + | + | + | + | + | | | + | |
| 440 | | | + | + | + | + | + | + | + | | | | + |
| 441 | | | | + | + | + | + | + | + | + | + | | |
| 442 | | | | + | + | + | + | + | + | + | | + | |
| 443 | | | | + | + | + | + | + | + | + | | | + |
| 444 | | | | | + | + | + | + | + | + | + | + | |
| 445 | | | | | + | + | + | + | + | + | + | | + |
| 446 | | | | | | + | + | + | + | + | + | + | + |
| 447 | + | + | + | + | + | + | + | + | + | | | | |
| 448 | + | + | + | + | + | + | + | + | | + | | | |
| 449 | + | + | + | + | + | + | + | + | | | + | | |
| 450 | + | + | + | + | + | + | + | + | | | | + | |
| 451 | + | + | + | + | + | + | + | + | | | | | + |
| 452 | | + | + | + | + | + | + | + | + | + | | | |
| 453 | | + | + | + | + | + | + | + | + | | + | | |
| 454 | | + | + | + | + | + | + | + | + | | | + | |
| 455 | | + | + | + | + | + | + | + | + | | | | + |
| 456 | | | + | + | + | + | + | + | + | + | + | | |
| 457 | | | + | + | + | + | + | + | + | + | | + | |
| 458 | | | + | + | + | + | + | + | + | + | | | + |
| 459 | | | | + | + | + | + | + | + | + | + | + | |
| 460 | | | | + | + | + | + | + | + | + | + | | + |
| 461 | | | | | + | + | + | + | + | + | + | + | + |
| 462 | + | + | + | + | + | + | + | + | + | + | | | |
| 463 | + | + | + | + | + | + | + | + | + | | + | | |
| 464 | + | + | + | + | + | + | + | + | + | | | + | |
| 465 | + | + | + | + | + | + | + | + | + | | | | + |
| 466 | | + | + | + | + | + | + | + | + | + | + | | |
| 467 | | + | + | + | + | + | + | + | + | + | | + | |
| 468 | | + | + | + | + | + | + | + | + | + | | | + |
| 469 | | | + | + | + | + | + | + | + | + | + | + | |
| 470 | | | + | + | + | + | + | + | + | + | + | | + |
| 471 | | | | + | + | + | + | + | + | + | + | + | + |
| 472 | + | + | + | + | + | + | + | + | + | + | + | | |
| 473 | + | + | + | + | + | + | + | + | + | + | | + | |
| 474 | + | + | + | + | + | + | + | + | + | + | | | + |
| 475 | | + | + | + | + | + | + | + | + | + | + | + | |
| 476 | | + | + | + | + | + | + | + | + | + | + | | + |
| 477 | | | + | + | + | + | + | + | + | + | + | + | + |
| 478 | + | + | + | + | + | + | + | + | + | + | + | + | |
| 479 | + | + | + | + | + | + | + | + | + | + | + | | + |
| 480 | + | + | + | + | + | + | + | + | + | + | + | + | + |
| 481 | | | | | | | + | | | | | + | + |

In a preferred embodiment, the subject is a mammal, preferably a mouse, rat, guinea pig, cat, dog, sheep, horse, cow, pig, more preferably the subject is a human.

In another preferred embodiment, the method further comprises determining the prognostic transcript of one or more genes selected from the group of genes consisting of Ubd, C2, Lst1, Aif1, C1QTNF7, CEACAM4, MME, IGFBP5, TAP1, CTGF, ANP32A, HCLS1, HTRA1, LGALS7, PTGER2, PTPN7, TGM2, TREM2 and CARD11; or their corresponding cDNAs, or their expression products, wherein
(i) for every unit of increased expression of one or more of Ubd, C2, Aif1, CEACAM4, TAP1, PTGER2, PTPN7, TGM2, TREM2, HCLS1 and/or CARD11; or the corresponding cDNA or expression product, said patient is expected to develop GvHR or GvHD; and
(ii) for every unit of decreased expression of one or more of Lst1, C1QTNF7, MME, CTGF, ANP32A, HTRA1, LGALS7 and/or IGFBP5; or the corresponding cDNAs or expression product(s), said patient is expected to develop GvHR or GvHD.

Accordingly, any combination of genes Ctss, Pbx2, Grem1, Ly6g6e, Olr1, Spr1, Msr1, Spic, Nfe2, Tnfaip8l2, Ier3, Pik3ap1, and Pstpip1 may be combined with any combination of genes Ubd, C2, Lst1, Aif1, C1QTNF7, CEACAM4, MME, IGFBP5, TAP1, CTGF, ANP32A, HCLS1, HTRA1, LGALS7, PTGER2, PTPN7, TGM2, TREM2 and CARD11.

In a second aspect, the invention relates to a method of diagnosing graft versus host reaction (GvHR) or graft versus host disease (GvHD) in a subject following transplantation, comprising:
(a) determining the expression level of one or more prognostic RNA transcripts, or their corresponding cDNAs, or their expression products, in a sample obtained from said subject, wherein said transcript(s) or expression products is/are the transcript or expression product of one or more genes selected from the group consisting of:
   (i) Msr1, Pik3ap1, Pstpip1, Ctss, Pbx2, Grem1, Ly6g6e, Olr1, Spr1, Spic, Nfe2 Tnfaip8l2, and Ier3; or
   (ii) Msr1, Ctss, Pbx2, Grem1, Ly6g6e, Olr1, Spr1, Spic, and Nfe2; or
   (iii) Pik3ap1, Pstpip1, Tnfaip8l2, and Ier3;
(b) comparing the expression level of said one or more prognostic RNA transcript, or its corresponding cDNA, or its expression product with a corresponding baseline value;
   wherein
   (i) every unit of increased expression of Olr1, Msr1, Pik3ap1, and/or Pstpip1, or the corresponding cDNA or expression product, is indicative of GvHR or GvHD; and
   (ii) every unit of decreased expression of Ctss, Pbx2, Grem1, Ly6g6e, Spr1, Spic, Nfe2, Tnfaip8l2, and/or Ier3, or the corresponding cDNA or expression product, is indicative of GvHR or GvHD.

The preferred embodiments of the first aspect are also preferred embodiments of the second aspect, and the same definitions apply.

However, in one particularly preferred embodiment, the baseline value is the expression level of said at least one gene in said subject prior to said transplantation and/or in at least one healthy subject.

In a preferred embodiment of the second aspect, said method further comprises determining the prognostic transcript of one or more genes selected from the group of genes consisting of Ubd, C2, Lst1, Aif1, C1QTNF7, CEACAM4, MME, IGFBP5, TAP1, CTGF, ANP32A, HCLS1, HTRA1, LGALS7, PTGER2, PTPN7, TGM2, TREM2 and CARD11; or their corresponding cDNAs, or their expression products, wherein
(i) every unit of increased expression of Ubd, C2, Aif1, CEACAM4, TAP1, PTGER2, PTPN7, TGM2, TREM2, HCLS1 and/or CARD11; or the corresponding cDNA or expression product, is indicative of GvHR or GvHD; and
(ii) every unit of decreased expression of Lst1, C1QTNF7, MME, CTGF, ANP32A, HTRA1, LGALS7 and/or IGFBP5; or the corresponding cDNA or expression product, is indicative of GvHR or GvHD.

In a third aspect, the invention relates to a method of monitoring the efficacy of treatment of graft versus host reaction (GvHR) or graft versus host disease (GvHD) in a subject following transplantation, comprising:
(a) determining the expression level of one or more prognostic RNA transcripts, or their corresponding cDNAs, or their expression products, in a sample obtained from said subject at a first time point T1, and a later second time point T2, wherein said transcript(s) or expression products is/are the transcript or expression product of one or more genes selected from the group consisting of:
   (i) Msr1, Pik3ap1, Pstpip1, Ctss, Pbx2, Grem1, Ly6g6e, Olr1, Spr1, Spic, Nfe2, Tnfaip8l2, and Ier3; or
   (ii) Msr1, Ctss, Pbx2, Grem1, Ly6g6e, Olr1, Spr1, Spic, and Nfe2; or
   (iii) Pik3ap1, Pstpip1, Tnfaip8l2, and Ier3;
(b) comparing the expression level of said one or more prognostic RNA transcript, or its corresponding cDNA, or its expression product at time point T1 (Δ1) and time point T2 (Δ2) with a corresponding baseline value;
   wherein
   (i) a decline in units of an increased expression of Olr1, Msr1, Pik3ap1, and/or Pstpip1; or the corresponding cDNA or expression product at time point T2 in comparison with the increased expression of said at least one gene at the time point T1 (ΔΔ=Δ1−Δ2), is indicative of effective treatment of GvHR or GvHD; and
   (ii) a decline in units of a decreased expression of Ctss, Pbx2, Grem1, Ly6g6e, Spr1, Spic, Nfe2, Tnfaip8l2, and/or Ier3; or the corresponding cDNA or expression product at time point T2 in comparison with the decreased expression of said at least one gene at the time point T1(ΔΔ=Δ1−Δ2), is indicative of effective treatment of GvHR or GvHD.

The preferred embodiments of the first and second aspect are also preferred embodiments of the third aspect, and the same definitions apply.

In another preferred embodiment, the method of the third aspect further comprises determining the prognostic transcript of one or more genes selected from the group of genes consisting of Ubd, C2, Lst1, Aif1, C1QTNF7, CEACAM4, MME, IGFBP5, Tap1, Ctgf, ANP32A, HCLS1, HTRA1, LGALS7, PTGER2, PTPN7, TGM2, TREM2 and CARD11; or their corresponding cDNAs, or their expression products, wherein
(i) a decline in units of an increased expression of Ubd, C2, Aif1, CEACAM4, Tap1, PTGER2, PTPN7, TGM2, TREM2, HCLS1 and/or CARD11; or the corresponding cDNA or expression product at time point T2 in comparison with the increased expression of said at least one gene at the time point T1 (M=Δ1−Δ2), is indicative of effective treatment of GvHR or GvHD; and
(ii) a decline in units of a decreased expression of Lst1, C1QTNF7, MME, Ctgf, ANP32A, HTRA1, LGALS7 and/or IGFBP5; or the corresponding cDNA or expression product at time point T2 in comparison with the decreased expression of said at least one gene at the time point T1(ΔΔ=Δ1−Δ2), is indicative of effective treatment of GvHR or GvHD.

In a very important fourth aspect, the invention further relates to a method of screening for a candidate substance for treatment of graft versus host reaction (GvHR) or graft versus host disease (GvHD), comprising:
(a) monitoring the efficacy of treatment by said candidate substance by using the method according to the third aspect in
   (i) a non-human animal model which suffers from GvHR or GvHD and to which the candidate substance has been administered, or
   (ii) in an ex vivo model, including but not limited to cell-based and/or tissue-based GvHR or HvHD assay such as the Skin Explant Assay, wherein said cells and/or tissue have been contacted with said candidate substance; and
(b) selecting a candidate substance which shows effective treatment of GvHR or GvHD.

Preferably, the screening method is carried out in vitro, i.e. in an ex vivo model, with cultured cells or with tissue, and by applying high throughput procedures. One example of such an ex vivo model is the Skin Explant Assay. This unique, non-artificial, (human) in vitro assay technology allows the study of primary and secondary immune responses in the presence of immunomodulatory drugs or allogeneic stem cells, reducing the need for extensive animal testing. Incubation with, for example, human skin, allows skin damage to be assessed by histopathology. The skin is graded for histological damage using criteria similar to that used and observed in the clinical setting. Results correlate with systemic disease and have been shown to predict outcome. The Skin Explant Assay is further exemplified in the Examples section and in the references cited therein.

Candidate substances selected by the screening method according to the invention may be subsequently also tested in vivo.

Alternatively, the screening assay may be directly performed in vivo by using a non-human animal model which suffers from GvHR or GvHD. Suitable non-human animal models include rats, mice, guinea pigs, pigs, dogs, and cats. However, it has to be made sure that the scientific gain outweighs any animal suffering, and that the testings are carried out in accordance with national restrictions for animal testings.

A variety of types of putative candidate substances may be tested and identified as suitable. For example, one can utilize known properties of a target protein to devise agents to stimulate or inhibit its production or activity, as desired. That is, one can devise a means to inhibit the action of, or bind, block, remove or otherwise diminish the presence, activity and/or availability of, a protein whose upregulation is associated with GvHD or GvHR; or one can devise a means to stimulate the action of, or to potentiate or enhance the activity of or availability of, a protein whose down-regulation is associated with GvHD or GvHR.

For example, in the case of a cellular receptor, one could expose the receptor to an antagonist, a soluble form of the receptor or a “decoy” ligand binding site of a receptor (to compete for ligand) to inhibit it. Antibodies may be administered to a cell to bind and inactivate (or compete with), or to enhance the activity of, secreted protein products or expressed cell-surface products of genes of interest.

Another approach is to employ antisense oligonucleotides or nucleic acid constructs that inhibit expression of a gene whose down-regulation is desired, in a highly specific manner. Methods to select, test and optimize putative antisense sequences are routine. Nucleic acid constructs may be used to express an antisense molecule of interest, or antisense oligonucleotides as such may be administered to a cell. The oligonucleotides can be DNA or RNA or chimeric mixtures or derivatives or modified versions thereof, single-stranded or double-stranded. The oligonucleotides can be modified at the base moiety, sugar moiety, or phosphate backbone. The oligonucleotide may include other appending groups such as peptides, or agents facilitating transport across the cell membrane, hybridization-triggered cleavage agents, or intercalating agents. Multiple antisense constructs or oligonucleotides specific for different genes can be employed together. The sequences of the down-regulated genes described herein can be used to design the antisense molecules. The antisense sequences may range from about 6 to about 50 nucleotides, and may be as large as 100 or 200 nucleotides, or larger. They may correspond to full-length coding sequences and/or may be genomic sequences that comprise non-coding sequences.

Another approach is to use ribozymes that can specifically cleave nucleic acids encoding the overexpressed genes disclosed herein. Such methods are routine in the art and methods of making and using any of a variety of appropriate ribozymes are well known to the skilled worker. A ribozyme having specificity for an mRNA of interest can be designed based upon the nucleotide sequence of, e.g., the corresponding cDNA. Alternatively, the sequence of an overexpressed gene disclosed herein can be used to select a catalytic RNA having a specific ribonuclease activity from a pool of RNA molecules.

Another approach involves double stranded RNAs called small interfering RNAs. A siRNA is a double-stranded RNA molecule comprising self-complementary sense and antisense regions, wherein the antisense region comprises nucleotide sequence that is complementary to nucleotide sequence in a target nucleic acid molecule or a portion thereof, and the sense region has a nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary. The siRNA can be assembled from a single oligonucleotide, where the self-complementary sense and antisense regions of the siRNA are linked by means of a nucleic acid based or non-nucleic acid-based linker. The siRNA may be a polynucleotide having a hairpin secondary structure, i.e. having self-complementary sense and antisense regions. The siRNA may be a circular single-stranded polynucleotide having two or more loop structures and a stem comprising self-complementary sense and antisense regions, wherein the circular polynucleotide can be processed either in vivo or in vitro to generate an active siRNA molecule capable of mediating RNAi. In certain embodiments, the siRNA molecule comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. RNAi molecules may be used to inhibit gene expression, using conventional procedures.

Another approach is to use small molecules, or “compounds”, isolated from natural sources or developed synthetically, e.g., by combinatorial chemistry. In general, such molecules are identified from large libraries of natural products or synthetic (or semisynthetic) extracts or chemical libraries according to methods known in the art. Those skilled in the field of drug discovery and development will understand that the precise source of test extracts or compounds is not critical to the methods of the invention. Accordingly, virtually any number of chemical extracts or compounds can be used in the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, e.g., Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

Methods for introducing candidate substances into cells are conventional. For example, methods of gene transfer may be used, wherein antisense molecules, ribozymes, or siRNAs are introduced into a rectal carcinoma cell of interest, or nucleic acids that encode proteins which modulate (up-regulate or down-regulate) the production or activity of one or more of the genes disclosed herein. Methods of gene transfer are conventional, and include virus-mediated gene transfer, for example, with retroviruses, lentiviruses, and recombinant adenovirus vectors. Adeno-associated virus (AAV) may also be used. Improved efficiency is attained by the use of promoter enhancer elements in the DNA constructs. In addition to virus-mediated gene transfer, physical means well-known in the art can be used for direct gene transfer, including administration of plasmid DNA and particle-bombardment mediated gene transfer. Furthermore, electroporation or calcium phosphate transfection, both well-known means to transfer genes into cell in vitro, may also be used. Gene transfer may also be achieved by using “carrier mediated gene transfer”. Preferred carriers are targeted liposomes such as immunoliposomes, which can incorporate acylated monoclonal antibodies into the lipid bilayer, or polycations such as asialoglycoprotein/polylysine. Liposomes have been used to encapsulate and deliver a variety of materials to cells, including nucleic acids and viral particles. Preformed liposomes that contain synthetic cationic lipids form stable complexes with polyanionic DNA. Cationic liposomes, liposomes comprising some cationic lipid, that contained a membrane fusion-promoting lipid dioctadecyldimethyl-ammonium-bromide (DDAB) have efficiently transferred heterologous genes into eukaryotic cells and can mediate high level cellular expression of transgenes, or mRNA, by delivering them into a variety of cultured cell lines.

In still a final aspect, the invention describes the use of a kit in a method of predicting the risk of developing graft versus host reaction (GvHR) or graft versus host disease (GvHD) according to the first aspect, or in a method of diagnosing GvHR or GvHD according to the second aspect, or in a method of monitoring the efficacy of treatment of GvHR or GvHD according to the third aspect, wherein the kit comprises at least one isolated polynucleotide, wherein each isolated polynucleotide independently comprises
(i) at least 20 contiguous nucleotides of the nucleotide sequence selected from SEQ ID NO: 1, 3, 5, 7, 8, 10, 12, 14, 16, 18, 20, 22, and/or 24; or SEQ ID NO: 26-47, or
(ii) a nucleotide sequence having at least 90% identity to (i), or
(iii) the coding region of a gene comprising a nucleotide sequence according to (i) or (ii), or
(iv) a nucleotide sequence that can specifically hybridize, under conditions of high stringency, to a polynucleotide having a nucleotide sequence according to (i), (ii) or (iii); and
   wherein the kit comprises no more than 9000 isolated polynucleotides in total.

The isolated polynucleotide may have at least 90% identity to a polynucleotide cornprising a nucleotide sequence selected from the group of nucleotide sequences consisting of at least 20 contiguous nucleotides of the nucleotide sequence selected from SEQ ID NO: 1, 3, 5, 7, 8, 10, 12, 14, 16, 18, 20, 22, and/or 24; or SEQ ID NO: 26-47; more preferably to the CDS encoded therein. Preferably, said isolated polynucleotide, has a nucleotide sequence having at least 92%, at least 94%, at least 96%, at least 98%, or 99% nucleotide sequence identity to a polynucleotide comprising a nucleotide sequence selected from the group of nucleotide sequences consisting of at least 20 contiguous nucleotides of the nucleotide sequence selected from SEQ ID NO: 1, 3, 5, 7, 8, 10, 12, 14, 16, 18, 20, 22, and/or 24; or SEQ ID NO: 26-47; more preferably to the CDS encoded therein.

Generally, a nucleotide sequence has “at least x % identity” with another nucleotide sequence or any of the sequences given above if, when the sequence identity between those to aligned sequences is at least x %. Such an alignment can be performed using for example publicly available computer homology programs such as the “BLAST” program provided at the NCBI homepage at http://www.ncbi.nlm.nih.gov/blast/blast.cgi, using the default settings provided therein. Further methods of calculating sequence identity percentages of sets of nucleic acid sequences are known in the art.

Preferably, the isolated polynucleotides comprise at least 25, preferably at least 30, more preferably at least 35, even more preferably at least 40, most preferably 50, in particular 60 contiguous nucleotides.

In another preferred embodiment, the isolated polynucleotides are arranged in an array, in particular wherein the kit comprises no more than 8000, preferably no more than 7000, more preferably no more than 6000, even more preferably no more than 5000 or even no more than 4000, most preferably no more than 3000 or even no more than 2000, in particular no more than 1000 or even no more than 500 or no more than 100 isolated polynucleotides in total.

The isolated polynucleotides of the kit may be used as probes in a hybridization method, however, in a more preferred embodiment, the isolated polynucleotides are arranged in an array. The term “array”, as used herein, means an ordered arrangement of addressable, accessible, spatially discrete or identifiable, molecules disposed on a surface. Moreover, the array may be a microarray (sometimes referred to as a DNA “chip”). Microarrays allow for massively parallel gene expression analysis. Furthermore, the hybridization signal from each of the array elements is individually distinguishable. Arrays can comprise any number of sites that comprise probes, from about 5 to, in the case of a microarray, tens to hundreds of thousands or more. Microfluidic devises are also contemplated.

Any suitable, compatible surfaces can be used in conjunction with this array. The surface (usually a solid, preferably a suitable rigid or semi-rigid support) may be any organic or inorganic material or a combination thereof, including, merely by way of example, plastics such as polypropylene or polystyrene; ceramic; silicon; (fused) silica, quartz or glass, which can have the thickness of, for example, a glass microscope slide or a glass cover slip; paper, such as filter paper; diazotized cellulose; nitrocellulose filters; nylon membrane; or polyacrylamide gel pad. Substrates that are trans-parent to light are useful when the method of performing an assay involves optical detection. Suitable surfaces include membranes, filters, chips, slides, wafers, fibers, magnetic or nonmagnetic beads, gels, tubing, plates, polymers, microparticles, capillades, or the like. The surface can have a variety of surface forms, such as wells, trenches, pins, channels and pores, to which the isolated polynucleotides are bound. It can, for example, be a flat surface such as a square, rectangle, or circle; a curved surface; or a three dimensional surface such as a bead, particle, strand, precipitate, tube, sphere, etc.

Methods of making DNA arrays, including microarrays are conventional. For example, the probes may be synthesized directly on the surface; or preformed molecules, such as oligonucleotides or cDNAs, may be introduced onto (e.g., bound to, or otherwise immobilized on) the surface. Among suitable fabrication methods are photolithography, pipetting, drop-touch, piezoelectric printing (ink-jet), or the like.

Furthermore, the probes do not have to be directly bound to the substrate, but rather can be bound to the substrate through a linker group. The linker groups are typically about 6 to 50 atoms long to provide exposure to the attached nucleic acid probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the nucleic acid probe.

The kit may optionally further comprise, isolated polynucleotides that act as internal controls. The controls may be positive controls or negative controls, examples of which will be evident to the skilled worker. The determined amounts obtained by use of the kit should reflect accurately the amounts of control target polynucleotide added to the sample.

The kit may further comprise means for carrying out a method of the invention, means for reading hybridization results and instructions for performing a method, such as a diagnostic method. Hybridization results may be units of fluorescence. Other optional elements of the kit may include suitable buffers, media components, or the like; a computer or computer-readable medium for storing and/or evaluating the assay results; containers; or packaging materials. Reagents for performing suitable controls may also be included. The reagents of the kit may be in containers in which the reagents are stable, e.g., in lyophilized form or stabilized liquids. The reagents may also be in single use form, e.g., in single reaction form for diagnostic use. The following examples are meant to further illustrate, but not limit, the invention. The examples comprise technical features, and it will be appreciated that the invention relates also to combinations of the technical features presented in this exemplifying section.

### BRIEF DESCRIPTION OF THE FIGURES

FIG. 1. Induction of a GVHR in BN rat skin explants exposed to PVG lymphocytes. A summary of the histological GVHR grading of BN skin samples cultured in medium alone, together with syngeneic BN lymphocytes, and together with pre-stimulated allogeneic PVG lymphocytes (n=12 in each group) is given. The samples represented by closed circles were used for both gene expression profiling and qRT-PCR experiments, whereas the other samples were only used for gene expression profiling. The pair-wise comparison (U test) indicated a significant difference between skin explant cultures with BN and PVG lymphocytes.

FIG. 2. Expression profiling of BN skin explant samples exposed to allogeneic (PVG) lymphocytes in comparison to those exposed to syngeneic (BN) lymphocytes. (A) The log 2-fold changes in gene expression of significantly regulated MHC genes (p<0.05) are shown. (B) The log 2-fold changes in gene expression of significantly regulated NKC genes (p<0.05) are shown. (C) The log 2-fold changes in gene expression of 168 significantly (p<0.05) and strongly (log 2-fold change ≧1 or ≦−1) regulated non-MHC and non-NKC genes indicate the range of observed alterations in gene expression levels among the 6342 tested genes. In panels A and B, black bars indicate a strong change (log 2-fold change or ≧1 or ≦−1), dotted bars alterations below this amplitude, and white bars expression changes that were not detected at a significant level with all, but at least with 50% of the probes present on the array for that gene. When more than one probe indicated a significant change of gene expression the means and standard deviations of the log 2-fold changes are shown (see Tab. 5, 6, and 7 for further details).

FIG. 3. Verification of the regulation in gene expression observed in the microarray experiment by qRT-PCR. A subgroup of 8 samples used for the microarray experiment (see FIG. 1) was analyzed by qRT-PCR for the expression of 10 MHC and 3 NKC genes. The ΔΔct value was calculated, i.e. the Δct (Gapdh—gene of interest) of the allogeneic skin explant samples minus Δct (Gapdh—gene of interest) of the corresponding control sample. The control sample was either a parallel skin explant exposed to syngeneic lymphocytes as in the microarray experiment (syngeneic control, black bars) or a parallel skin explant sample cultured in medium only (medium control, white bars). The means of the ΔΔct values plus SEM are shown. A positive value indicates an up-regulation of gene expression in the allogeneic samples.

FIG. 4. Analysis of T cell infiltration in skin explants. (A) Analysis of Cd3z gene expression in the same samples as shown in FIG. 3. (B) Correlation of Cd3z and other gene expression levels (ΔΔct values for allogeneic skin explants minus syngeneic controls) in these samples. Pearson's correlation coefficients (r) and the p-values for the corresponding tests are given above the diagrams. In brackets Spearman's correlation coefficients (r) and the p-values for the corresponding tests are shown.

FIG. 5. Induction of a GVHR in a second series of BN (filled circles) and LEW.1N (open circles) rat skin explants. Skin explants were co-cultured with pre-stimulated allogeneic lymphocytes from rats with a minor (BN lymphocytes and LEW.1N skin), major (LEW.1A (RT1^{a}) or LEW.1AV1 (Rn^{av1}) lymphocytes and LEW.1N skin), or a minor and major histoincompatibility (PVG lymphocytes (RT1^{c}) and BN skin or LOU/C (RT1^{U}) lymphocytes and LEW.1N skin). A summary of the histological GVHR grading of skin samples cultured in medium alone, together with syngeneic BN or LEW.1N lymphocytes, and together with allogeneic lymphocytes is given.

FIG. 6. Verification of gene regulations observed in the microarray experiment by qRT-PCR in an independent set of 17 skin explant assays. Three samples were derived from skin explant assays with minor (upper panel), 5 with major (middle panel), and 9 with minor and major histoincompatibility (lower panel). The GVHR grading for these samples is shown in FIG. 5. The expression of 7 MHC and 3 NKC was analyzed by qRT-PCR. The ΔΔct value, i.e. Δct (Gapdh—gene of interest) of the allogeneic skin explant samples minus mean of Δct (Gapdh—gene of interest) of the corresponding control samples (BN or LEW.1N, respectively), was calculated. The control samples were either skin explant samples exposed to syngeneic lymphocytes (syngeneic control) or skin explant samples cultured in medium only without added lymphocytes (medium control) and their GVHR grading is also shown in FIG. 5. The means of the ΔΔct values plus SEM are shown. A positive value indicates an up-regulation of gene expression in the allogeneic samples.

FIG. 7. Analysis of MHC and NKC gene regulation in skin explants exposed to pre-stimulated allogeneic lymphocytes depending on GVHR grading (from left to right: grade I (white), grade II (light grey/pointed), grade III (dark grey/striped), grade IV (black)). The expression of 7 MHC and 3 NKC was analyzed by qRT-PCR. The relative changes of gene expression levels were calculated using a mathematical model for relative quantification of real-time PCR data which also takes into account variations of the amplification efficiencies of different primer pairs (Pfaffl M W (2001) Nucleic Acids Res 29: e45). The means plus SEM are shown. A value >1 indicates an up-regulation of gene expression in the allogeneic samples. The control samples were either skin explant samples exposed to syngeneic lymphocytes (syngeneic control, upper panel), skin explant samples cultured in medium only (medium control, mean panel), or freshly frozen healthy skin samples (healthy skin control, lower panel).

FIG. 8. Analysis of MHC and NKC gene regulation in GVHD skin lesions from transplanted animals. BN (RT1ⁿ) rats were transplanted with bone marrow of PVG (RT1^{c}) rats. Rats that developed acute GVHD were scarified and skin lesions with signs of GVHD were obtained for RNA preparation and histology. The expression of 7 MHC and 3 NKC was analyzed by qRT-PCR using the B2m gene as reference. The relative changes of gene expression levels were calculated (Pfaffl M W (2001) Nucleic Acids Res 29: e45). The means plus SEM are shown for skin lesion with grade I and grade II GVHD. A value >1 indicates an up-regulation of gene expression in the allogeneic samples. The control samples were freshly frozen skin samples from healthy BN rats (n=7).

### EXAMPLES

### Example 1

### Expression Profiling of GVHR in Rat Skin Explants

The inventors decided to analyze a rat model of GVHD making use of genetically well-defined inbred stains. Importantly, the non-class I/non-class II genes of human (HLA) and rat (RT1) MHCs are highly conserved. However, the size and organization of MHC class I encoding regions are considerably variable and the rat possesses a significant number of MHC class Ib genes for which no human homologues exist. At least some of these genes have already been proven to encode ligands for inhibitory or activating natural killer (NK) receptors (Naper C, et al. (1999) Eur J Immunol 29: 2046-2053; Naper C, et al. (2005) J Immunol 174: 2702-2711). In the rat, in contrast to human, NK receptors of the Ly49 killer cell lectin-like receptor type predominate over killer cell Ig-like receptor genes. Therefore, the inventors also included the natural killer complex (NKC) in the expression profiling which harbors the Ly49 genes and additional natural cytotoxicity receptor genes.

To reduce the complexity of the experimental approach, the inventors used an invitro-model of the graft versus host reaction (GVHR)—the skin explant assay. This assay has been shown to be a sensitive predictor of GVHD in patients (Sviland L, et al. (2001) Hum Immunol 62: 1277-1281). It was also used to study the pathophysiology of GVHR (Dickinson A M, et al. (2002) Nat Med 8: 410-414). Recently, the inventors developed a rat skin explant assay (Novota P, et al. (2008) Transplantation 85: 1809-1816). This standardized in-vitro-model allows for studying gene expression during GVHR in a setting that is not influenced by undefined genetic differences between tissue samples which is unavoidable in human studies. Presently, the inventors used this model to analyze the MHC and NKC gene expression profiles of GVHR.

For the rat skin explant assays, rats of the inbred strains LEW.1N (RT1ⁿ), LEW.1A (RT1^{a}), LEW.1AV1 (RT1^{av1}), LOU/C (RT1^{u}), and BUF (RT1^{b}) were bred in the central animal facility of the Medical Faculty of the University of Gottingen. Rats of the strains PVG/OlaHsd (RT1^{c}) and BN/RijHsd (RT1ⁿ) were purchased from Harlan Winkelmann (Borchen, Germany). Animals between 10 and 20 weeks of age were used for the experiments. For transplantation experiments, PVG rats of the RT7.2 allotype (allelic variant RT7^{b}), originally obtained from Harlan OLAC, UK), were bred at the animal facility of the University of Oslo and BN rats were purchased from Harlan.

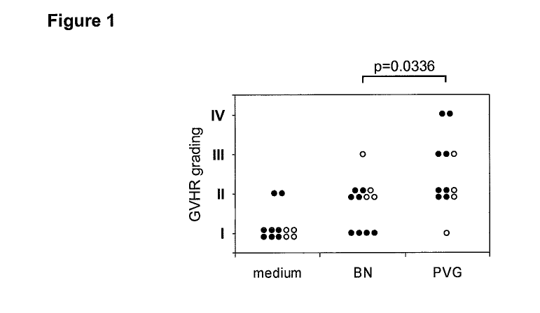

Rat skin explant assays were performed as previously described in detail (Novota P, Sviland L, Zinöcker S, Stocki P, Balavarca Y, et al. (2008) Correlation of Hsp70-1 and Hsp70-2 gene expression with the degree of graft-versus-host reaction in a rat skin explant model. Transplantation 85: 1809-1816). Briefly, mononuclear cells were obtained from rat spleens. Responder and irradiated (25 Gy) stimulator splenocytes were co-cultured in a MLR and the proliferation of responder lymphocytes was tested by [methyl-³H]-thymidine (Amersham, Braunschweig, Germany) incorporation. The stimulation index was calculated as described (Novota P, et al. (2008), supra). After 7 days 10⁶ responder lymphocytes were added to freshly obtained skin samples from the stimulator strain that were cultured in 200 μl NaHCO₃-buffered Dulbecco's modified Eagle's medium (DMEM; Biochrom) supplemented with 3% normal rat serum, 2 mM L-glutamine, 1 mM sodium pyruvate, and antibiotics in round-bottomed microtitre plates (Sarstedt, Nümbrecht, Germany). The skin samples were excised from the paws of rats after washing with 70% ethanol. The subcutaneous fat tissue was removed and the samples were trimmed to a size of approximately 1.5×1.5 mm. Skin samples cultured in medium only and samples co-cultured with lymphocytes from a “syngeneic MLR” were used as controls. After 3 days, the skin explants were washed with N-2-hydroxyethylpiperazine-N-2-ethanesulfonic acid (HEPES)-buffered DMEM and snap frozen in liquid nitrogen and stored at −80° C. for RNA preparation. Parallel samples were fixed in 10% neutral-buffered formalin, sectioned, and stained with hematoxylin and eosin (H&E). The histological evaluation of the skin explants was performed blind by an expert histopathologist (L.S.) based on the grading system described by Lerner (Lerner K G, et al. (1974) Transplant Proc 6: 367-371). To obtain skin explant samples for an expression profiling experiment, the inventors used BN rats (RT1ⁿ) as recipients and PVG rats (RT1^{c}) as donors. This combination is mismatched for minor and major histocompatibility antigens, which gives rise to GVHR grades I to IV (Novota P, et al. (2008), supra). PVG splenocytes were stimulated for 7 days in a mixed lymphocyte reaction (MLR) with irradiated BN splenocytes. Syngeneic co-cultures (BN plus irradiated BN splenocytes) were performed as control experiments. The stimulation index indicated a specific proliferation of PVG lymphocytes in response to irradiated BN lymphocytes in contrast to syngeneic cultures of BN lymphocytes (p<0.0001, U test; n=12 responder animals per strain, data not shown). After 7 days the PVG and BN lymphocytes were harvested, added to fresh BN skin samples from 12 individual animals and cultured for 3 further days. For further controls, additional BN skin samples from the same animals were cultured in medium only. On day 3 the skin samples were harvested and snap frozen for RNA preparation. Parallel samples were fixed and assayed for histological evidence of GVHR (FIG. 1). Co-culture of BN skin explants with pre-stimulated allogeneic PVG lymphocytes resulted in higher grade GVHR than co-culture with BN lymphocytes (p=0.0336; U test). As in a previous experimental series (Novota P, et al. (2008) Transplantation 85: 1809-1816), the syngeneic lymphocyte co-culture more frequently resulted in GVHR-like pathology of grade II or higher than culture of the skin explants in medium only.

RNA was prepared from the 24 BN skin explants exposed either to syngeneic (BN; n=12) or to allogeneic (PVG, n=12) lymphocytes and used for MHC gene expression profiling.

RNA extraction was carried out using TRIZOL reagent (Invitrogen, Carlsbad, Calif., USA) according to the manufacturer's recommendations. Afterwards, the RNA samples were treated with RQ1 RNase free DNase (Promega, Madison, Wis., USA) for 20 min at 37° C. in order to remove genomic DNA contaminations. The RNA was then purified as described previously (Novota P, et al. (2008) Transplantation 85: 1809-1816). Quantity and quality of extracted RNA were controlled by capillary electrophoresis

Microarray Experiment

For the expression profiling, a custom-designed oligo DNA microarray (Agilent) was designed. For this purpose the annotated sequence of the MHC of the BN strain was used (Hurt P, et al. (2004) Genome Res 14: 631-639). The 15K microarray covered 224 MHC genes by 649 oligonucleotide probes and 43 NKC genes by 101 probes. For 88 of these genes, i.e. 39.3%, the inventors had to design custom probes. A list of the MHC genes in the chromosomal order with all results obtained in the expression profiling experiment is given in the Table 5a.

These probes were spotted in triplicates. Further probes representing 6342 genes were added mainly to allow for data normalization. A two-color 12×2 paired swap design (Landgrebe J, et al. (2004) In Silico Biol 4: 461-470) using 24 arrays was applied, comparing RNA samples from 12 independent allogeneic and 12 independent syngeneic skin explant assays. Aliquots of total RNA (200 ng) were used as starting material. The “Low RNA Input linear Amplification Kit Plus, two color” (Agilent, 5188-5340) and the “RNA Spike-In Kit” (Agilent, 5188-5279) were used for cDNA synthesis and in-vitro transcription according to the manufacturer's recommendations. Quantity and dye incorporation rates of the amplified cRNAs were determined using the NanoDrop ND-1000 UV-VIS Spectrophotometer version 3.2.1 (NanoDrop Technologies, Wilmington, Del., USA). Afterwards, 300 ng aliquots of Cy3 and Cy5-labeled cRNAs from syngeneic and allogeneic skin explant assays, respectively, were mixed and hybridized to the microarrays. The hybridization was performed for 17 hours at 10 rpm and 65° C. After washing, Cy3 and Cy5 intensities were detected by two-color scanning using a DNA microarray scanner (Agilent, G2505B) at 5 micron resolution. Scanned image files were visually inspected for artifacts. The generated raw data were extracted using the Feature Extraction 9.1 software (Agilent).

The normalization of the raw microarray data was done with a non-linear loess regression (Yang Y H, et al. (2002) Nucleic Acids Res 30: e15). Differentially expressed genes were identified by an analysis of variance (ANOVA) mixed effects model (Landgrebe J, et al. (2004) In Silico Biol 4: 461-470) using SAS PROC MIXED. The resulting p-values were adjusted with the Benjamini-Hochberg method to control the false discovery rate (Benjamini Y, Hochberg Y (1995) J Roy Statist Soc Ser B 57: 289-300). The microarray data were generated conforming to the MIAME guidelines and have been deposited in NCBI's Gene Expression Omnibus (accessible through GEO series accession number GSE17928). For a general analysis of the gene expression data the PANTHER (Protein ANalysis THrough Evolutionary Relationships) system (Thomas P D, et al. (2003) Genome Res 13: 2129-2141) was used, which classifies genes by their functions (www.pantherdb.org/tools/genexAnalysis.jsp). The microarray data were mapped to PANTHER molecular function and biological process categories, as well as to biological pathways (Thomas P D, et al. (2006) Nucleic Acids Res 34: W645-650).

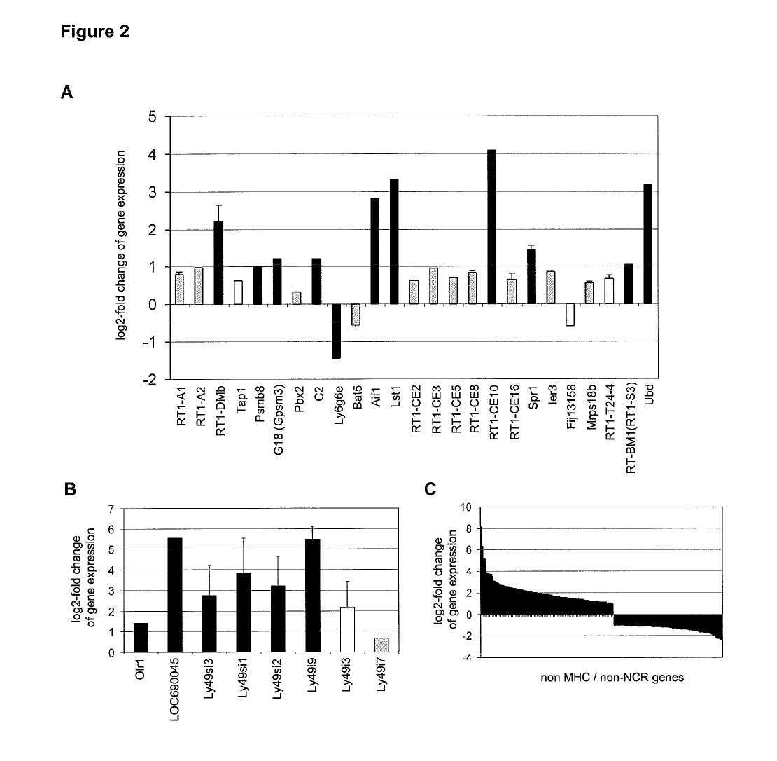

For 42 of the 224 MHC genes, a probe on the array indicated a significant regulation (p<0.05) in the allogeneic skin explant assays (n=12) compared to the syngeneic controls (n=12) (Tab. 5b). Eleven of these MHC genes showed on average at least a 2-fold up-regulation (log 2-fold change ≧1) or 50% reduction (log 2-fold change ≦−1) of mRNA levels (FIG. 2A, Tab. 5c). This amplitude of change is conventionally considered to be biologically relevant. Of these genes one was down-regulated (Ly6g6e) while 10 were up-regulated (FIG. 2A). Fourteen further MHC genes were regulated significantly (p<0.05) but with smaller amplitude (Tab. 5c). The regulation of 17 MHC genes appeared to be more doubtful because less than 50% of the probes for that gene indicated a significant regulation. Thus, the inventors considered 25 MHC genes to be significantly regulated in the expression profiling experiment (FIG. 2A). These included the classical class Ia genes RT1-A1 and RT1-A2, 8 non-classical class Ib genes (RT1-CE2, RT1-CE3, RT1-CE5, RT1-CE8, RT1-CE10, RT1-CE16, RT1-T24-4, RT-BM1) and 3 genes involved in antigen presentation (RT1-DMb, Tap1, Psmb8).

Furthermore, 43 genes of the NKC region, as a second important immune gene cluster, were represented on the array including all Ly49 genes in this region (Tab. 6a). For 8 of the 43 NKC genes represented on the array, a probe indicated a significant regulation (p<0.05) in the allogeneic skin explant assays compared to the syngeneic controls (Tab. 6b, 6c). In addition to the Olr1 gene, 6 Ly49 genes appeared to be up-regulated in the allogeneic skin explant assays (FIG. 2B). Not all probes for the Ly49i3 gene indicated a significant up-regulation. However, all significant results for this gene indicated a strong regulation (log 2-fold change >2). A statistically significant (p<0.05) but only moderate up-regulation (log 2-fold change <1) was detected for the Ly49i7gene.

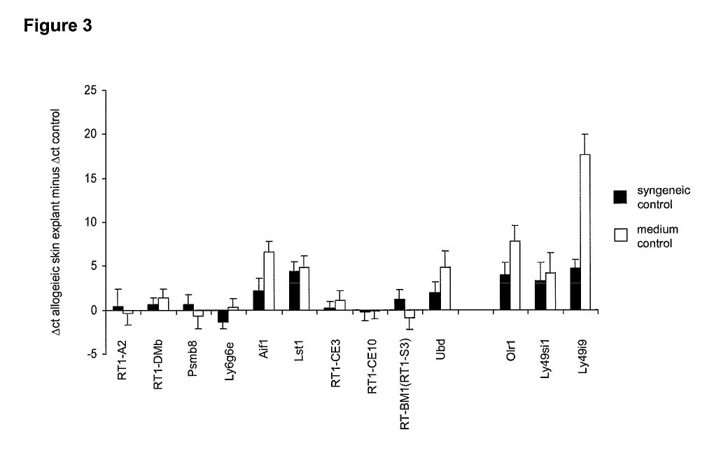

Probes for 6342 additional genes from all chromosomes were included mainly to allow for data normalization. For 168 of the non-MHC/non-NKC genes, a probe on the array indicated a significant (p<0.05) and strong (log 2-fold change ≧1 or ≦−1) regulation in the allogeneic skin explant assays compared to the syngeneic controls (FIG. 3C, Tab. 7). The 20 genes showing the strongest change in expression levels are shown in Table 1.

TABLE 1
The 20 most strongly regulated non-MHC/non-NKC genes in allogeneic skin explants
compared to syngeneic controls as revealed by the microarray experiment

| | log2-fold | adjusted | |
|---|---|---|---|
| gene | change | p-value | gene description |
| LOC685020 | 8.18 | 0.0100 | paired immunoglobin-like type 2 receptor alpha |
| Ptpns1l3 | 6.36 | 0.0100 | protein tyrosine phosphatase, non-receptor type substrate |
| | | | 1-like 3 |
| Fcgr3a | 5.24 | 0.0100 | Fc fragment of IgG, low affinity IIIa, receptor |
| Nat8 | 5.14 | 0.0100 | Rattus norvegicus endogenous retrovirus mRNA, partial |
| | | | sequence [AY212271] |
| Ccl9 | 4.16 | 0.0100 | chemokine (C-C motif) ligand 9 |
| XM_226926 | 3.92 | 0.0149 | Rattus norvegicus similar to protein tyrosine phosphatase, |
| | | | non-receptor type substrate; brain immunological-like with |
| | | | tyrosine-based motifs (LOC310212) |
| Hck | 3.87 | 0.0100 | hemopoietic cell kinase |
| Trem2 | 3.78 | 0.0100 | triggering receptor expressed on myeloid cells 2 |
| Ccl6 | 3.71 | 0.0100 | Rattus norvegicus chemokine (C-C motif) ligand 6 |
| Cd36 | 3.57 | 0.0100 | CD36 antigen |
| Igf1 | 3.23 | 0.0100 | insulin-like growth factor 1 |
| Ctss | 3.15 | 0.0100 | cathepsin S |
| Gzmc | 3.11 | 0.0373 | granzyme C |
| LOC100048479 | 2.97 | 0.0373 | one cut domain, family member 1 |
| Plscr1 | 2.83 | 0.0100 | phospholipid scramblase 1 |
| Nfe2 | 2.74 | 0.0149 | nuclear factor, erythroid derived 2 |
| Prg4 | 2.74 | 0.0149 | proteoglycan 4 |
| Spic | 2.68 | 0.0278 | Spi-C transcription factor |
| Fcgr2b | 2.62 | 0.0100 | Fc receptor, IgG, low affinity IIb |
| LOC498277 | 2.61 | 0.0100 | similar to Low affinity immunoglobulin gamma Fc region |
| | | | receptor III precursor |

All 20 genes were up-regulated and they included several genes with functions clearly associated with the immune response such as genes encoding chemokines (Ccl9, Ccl6), Fc receptors (Fcgr3a, Fcgr2b), the proteases cathepsin S (Ctss) and granzyme C (Gzmc), and the inflammatory triggering receptor on myeloid cells 2 (Trem2).

The percentage of significantly (p<0.05) and strongly (log 2-fold change ≧1 or ≦−1) up- or down-regulated genes was higher in the NKC region (14.0%) compared to MHC region (4.9%) and the genes encoded in other regions of the genome (2.6%). This difference was even more pronounced for up-regulated genes. 14.0% of the NKC, but only 4.5% of the MHC and 1.5% of the other genes were up-regulated (Tab. 2).

TABLE 2
Proportion of regulated genes as indicated
by the gene expression profiling experiment

| | analyzed | | | down- |
|---|---|---|---|---|
| region | genes | regulated¹ | up-regulated | regulated |
| MHC | 224 | 11 (4.9%) | 10 (4.5%) | 1 (0.4%) |
| NKC | 43 | 6 (14.0%) | 6 (14.0%) | 0 (0%) |
| others | 6342 | 168 (2.6%) | 93 (1.5%) | 75 (1.2%) |

¹Only those genes that were both significantly (p < 0.05) and strongly (log2-fold change ≧1 or ≦−1) regulated were taken into account for this comparison.

For a general analysis of the gene expression data the PANTHER system (Thomas P D, et al. (2003) Genome Res 13: 2129-2141) was used. With this tool the inventors found a significant up-regulation of genes taking part in “immunity and defence” (p<0.0001, binominal test). More specifically, genes involved in “T cell-mediated immunity” (p<0.0001), “NK cell-mediated immunity” (p<0.0001), “cytokine and chemokine-mediated signaling” (p=0.0032), and “B cell and antibody-mediated immunity” (p=0.0235) were up-regulated. Genes involved in “complement-mediated immunity” (p=0.0336) and “cell adhesion” (p=0.0003) were down-regulated (data not shown).

Validation of Rat Candidate Genes by Quantitative Real-Time PCR

To determine the reliability of the microarray results, the inventors analyzed the expression of 13 selected genes from the MHC and NKC regions by qRT-PCR experiments in 8 of the sample pairs that had been used for the microarrays (see FIG. 1). Specific primers for 10 MHC and 3 NKC genes were designed (Tab. 8). To generate external standard curves and to calculate the amplification efficiency of each primer pair, a pool of 20 random cDNAs was amplified in serial 10-fold dilutions (Pfaffl M W (2001) Nucleic Acids Res 29: e45). The amplification reactions were carried out as described previously (Novota P, et al. (2008) Transplantation 85: 1809-1816) using an ABI 7500 Real-Time PCR System. The data were analyzed with the ABI 7500 SDS software (Applied Biosystems). As internal control, mRNA expression of housekeeping genes Gapdh (Rn_Gapd_{—}1_SG QuantiTect Primer Assay QT00199633, Qiagen, Hilden, Germany) or B2m were monitored. To normalize variations in the RNA concentration in different samples, the ct values obtained in real-time PCR for the genes were corrected by the ct-value obtained for the housekeeping gene in the same sample (Δct=ct housekeeping gene−ct gene of interest). For direct comparison with microarray data, the relative changes of mRNA expression were calculated using the ΔΔct method (ΔΔct=Δct sample of interest−Δct control sample) (Livak K J, Schmittgen T D (2001) Methods 25: 402-408). For additional analyses, the relative changes of gene expression levels were calculated using a mathematical model for relative quantification of real-time PCR data which takes into account variations of the amplification efficiencies of different primer pairs (Pfaffl M W (2001), supra).

For 12 genes the regulation that was observed in the microarray experiment was confirmed by qRT-PCR as indicated by a regulation into the same direction when the allogeneic and syngeneic skin explant assays were compared using the ΔΔ cycle threshold (ct) method for relative quantification of gene expression (FIG. 3). Only one gene, RT1-CE10, was found to be strongly up-regulated in allogeneic skin explants in the microarray experiment but slightly down-regulated in qRT-PCR. In the qRT-PCR experiments, the inventors also included parallel skin explants that were cultured in medium only. Eight genes (RT1-DMb, Aif1, Lst1, RT1-CE3, Ubd, Olr1, Ly49si1, and Ly49i9) showed an up-regulation in the allogeneic skin explant assay also in this comparison (FIG. 3). Six of these genes (Aif1, Lst1, Ubd, Olr1, Ly49si1, and Ly49i9) were clearly found to be up-regulated in both comparisons.

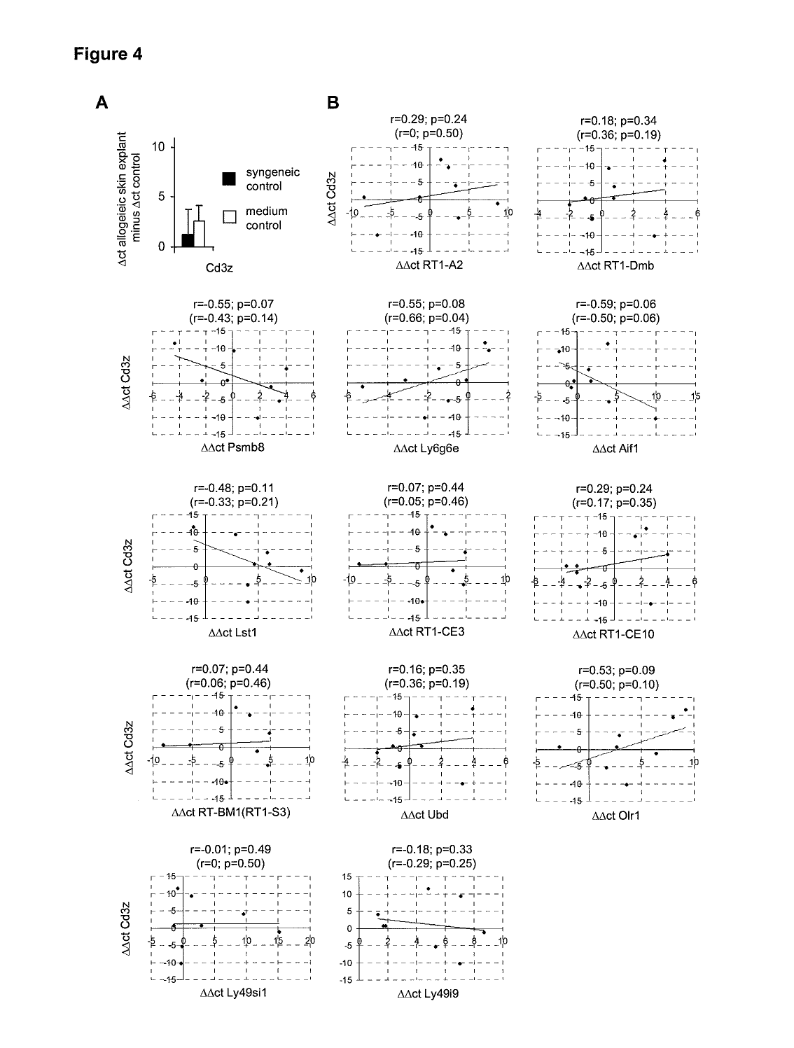

The up-regulation of genes in skin explants could be due to the change of gene expression in cells of the skin or due to infiltration of donor lymphocytes. Non-infiltrating or non-attaching donor lymphocytes were washed off before freezing of the skin explants and therefore would not contribute significantly to the results. Infiltrating lymphocytes were rarely seen in skin explants by histological analysis (data not shown). To further determine T cell infiltration at the RNA level, the inventors analyzed the expression of the CD3 zeta chain in qRT-PCR. Cd3z expression was found to be up-regulated in comparison to syngeneic controls and medium controls (FIG. 4A). The expression of most tested genes showed no correlation with Cd3z mRNA levels (FIG. 40). Only two of the genes analyzed in qRT-PCR (Ly6g6e and Olr1) showed a moderately positive correlation (r>0.50) with the Cd3z expression level (FIG. 4B). Importantly, Ly6g6e was down- and not up-regulated in allogeneic skin explants. The expression levels of three up-regulated genes (Psmb8, Aif1, and Lst1) were even negatively associated with Cd3z expression (FIG. 4B). Thus, of the tested genes only the increase of Olr1 expression may be formally explained by infiltrating T cells. However, Olr1 has not been described to be expressed in T cells. Therefore, infiltration of skin explants with T cells is unlikely to explain the observed gene expression changes.

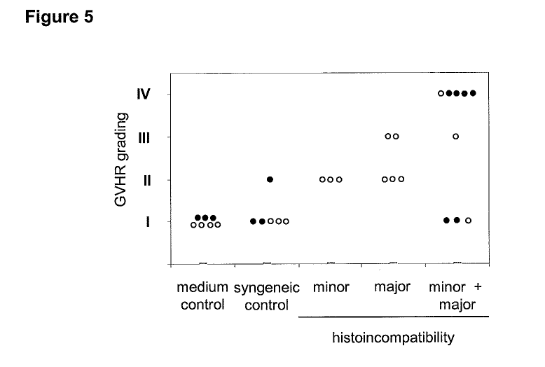

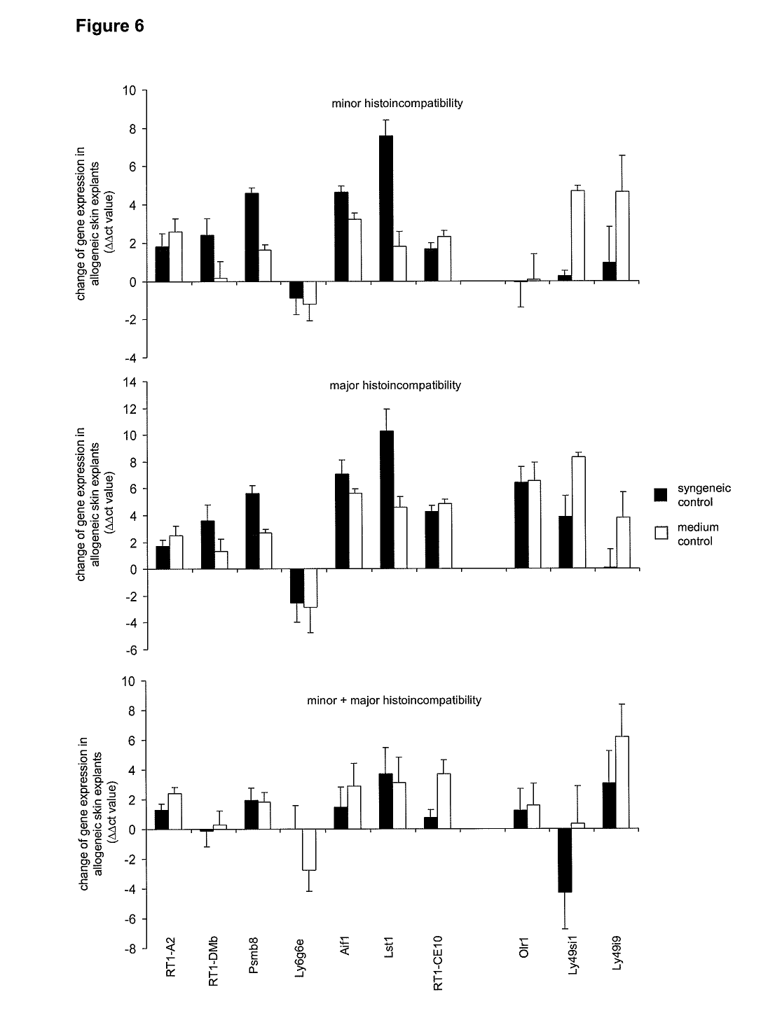

Next the inventors determined the expression of 10 selected genes in an independent set of skin explant assays. Skin explants derived from BN (RT1ⁿ) and LEW.1N (RT1ⁿ) rats were co-cultured with pre-stimulated allogeneic lymphocytes from rats with minor (BN lymphocytes and LEW.1N skin), major (LEW.1A (RT1^{a}) or LEW.1AV1 (RT1^{av1}) lymphocytes and LEW.1N skin), or minor and major histoincompatibility (PVG lymphocytes (RT1^{c}) and BN skin or LOU/C (RT1^{u}) lymphocytes and LEW.1N skin). Skin samples cultured with syngeneic lymphocytes (BN or LEW.1N) or cultured in medium only served as controls. The GVHR grading obtained in these experiments is shown in FIG. 5. The general regulation of the selected genes during GVHR was reproduced in this second experimental set when compared to skin explants exposed to syngeneic lymphocytes and also to samples cultured in medium only (FIG. 6). Aif1 and Lst1 were the most consistently up-regulated genes in skin explants with minor, major, and minor plus major histoincompatibility. The samples with minor plus major histoincompatibility showed the highest variation in gene regulation (FIG. 6). However, these samples were also most heterogeneous in the GVHR grading (FIG. 5). Therefore, the inventors analyzed the gene regulation dependent from the GVHR grading in samples from both experimental sets.

Regulation of Selected MHC and NKC Genes During GVHR

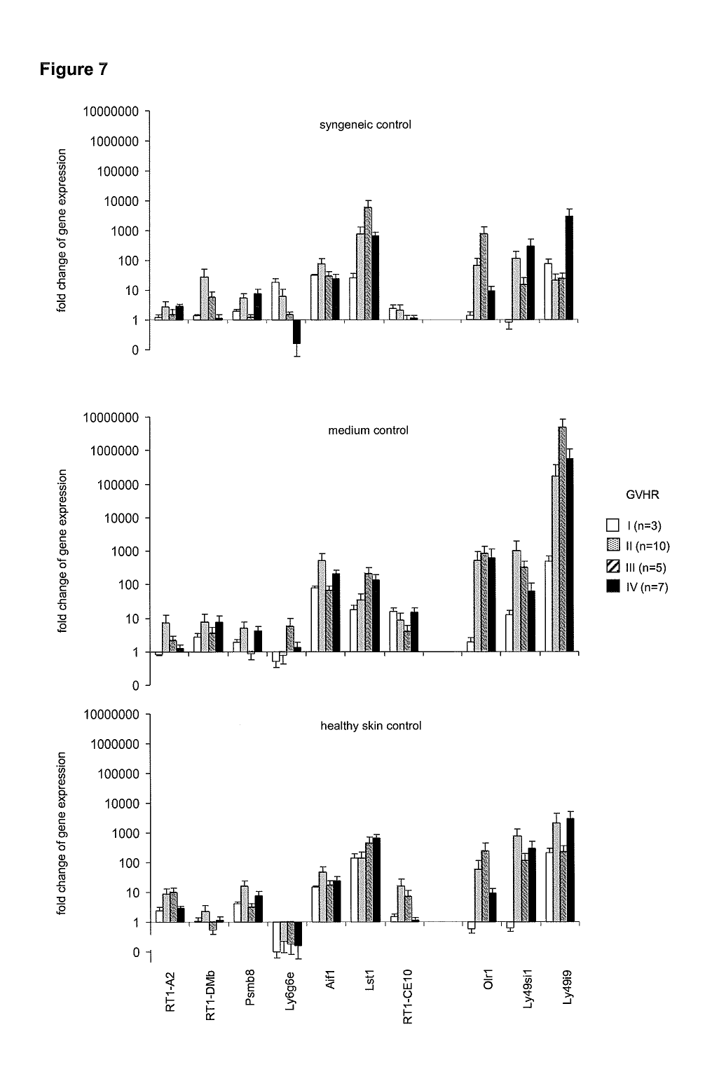

The expression of 7 MHC and 3 NKC genes was evaluated in the skin explant samples showing grade I, II, III or IV GVHR (FIG. 7). To provide an even more accurate comparison of the different genes in this evaluation of the data, the relative changes of gene expression levels were calculated using a mathematical model for relative quantification of real-time PCR data which takes into account variations in the amplification efficiencies of different primer pairs (Pfaffl M W (2001) Nucleic Acids Res 29: e45). When compared to skin explants exposed to syngeneic lymphocytes or to medium controls, the genes Aif1, Lst1, Olr1, and Ly49i9 were consistently up-regulated. Ly6g6e was down-regulated in some but not all comparisons. The expression of Aif1, Lst1 and Ly49i9 was found to be increased in all GVHR grades. The extremely high up-regulation of Ly49i9 encoding an NK receptor in comparison to medium controls might be explained by complete absence of NK cells in normal skin biopsies and infiltration of few NK cells during GVHR. When the gene expression was compared to freshly frozen healthy skin, the principal findings were confirmed. Interestingly, Olr1 was up-regulated mainly in grade II and III GVHR samples when compared to syngeneic control skin explants and healthy skin. Thus, this gene could be a marker of intermediate grade GVHR.

Regulation of Selected MHC and NKC Genes During GVHD

Next, the inventors wanted to know whether the genes found to be differentially expressed in GVHR in skin explant assays were also regulated in vivo in GVHD. For this purpose the inventors analyzed skin samples from BN rats that were transplanted with bone marrow from PVG rats and developed acute GVHD.

Transplantation experiments were approved by the Experimental Animal Board under the Ministry of Agriculture of Norway (ID 09.1514, 09.1515 and VIT 09.1512). Male PVG (RT7^{b}) rats served as bone marrow and lymph node donors. Mononuclear bone marrow cells were purified by density gradient centrifugation in Nycoprep 1.077A (Medinor ASA, Norway). The cells were depleted of T cells by magnetic separation using anti-CD5 (Ox19) and anti-αβ T cell receptor (R73) antibodies conjugated to pan-mouse IgG coated Dynabeads (Dynal Biotech ASA, Norway). This procedure reduced the CD3⁺ T cell content in the bone marrow from 3% to less than 0.3%. Male BN rats were used as recipients. They were irradiated (9 Gy) and subsequently received an i.v. injection of 30×10⁶ PVG.7b T cell-depleted bone marrow cells. 14 days post transplantation, 1.5×10⁶ lymph node cells were injected i.v. to evoke GVHD. The rats were regularly monitored for GVHD symptoms. Rats suffering from irreversible GVHD were sacrificed and skin samples were processed for RNA preparation and histology in parallel.

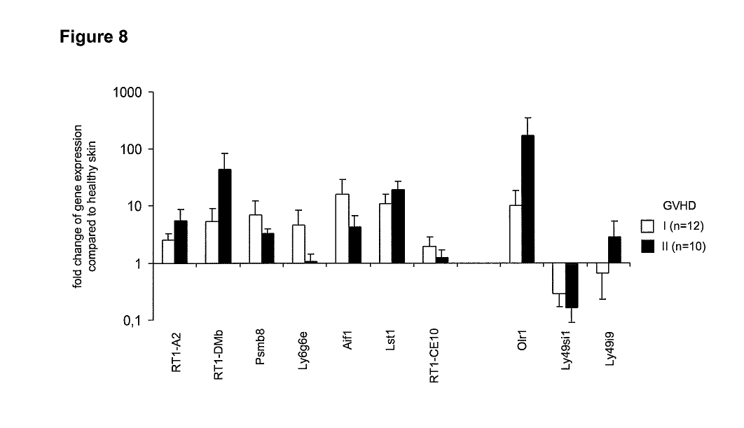

The analyzed skin samples showed in histology a grade I or grade II GVHD. The results of qRT-PCR for 7 MHC genes and 3 NKC genes are shown in FIG. 8. The strongest up-regulation in GVHD-affected skin was observed for RT1-DMb, Aif1, Lst1, and Olr1. Thus, most genes that were found to be regulated in GVHR in skin explants were also regulated in GVHD-affected skin. However, the Ly49si1 gene that was up-regulated consistently in allogeneic skin explants showing GVHR of grade II and above appeared to be down-regulated in GVHD. Compared to the skin explant samples, also the Ly49i9 gene was only moderately up-regulated in grade II GVHD samples from transplanted rats.

### Example 2

### Regulation of Selected MHC and NKC Genes During GVHR in Human Skin Explant Assays

Finally, the inventors explored the regulation of the identified genes during GVHR in human skin explant assays.

Validation of the rat candidate genes with human homologues was done by qRT-PCR on clinical samples of GvhD skin and normal skin samples. This was done by relative quantification using custom designed Taqman low density array (TLDA) cards (Applied Biosystems), each card contained 4 replicates of 95 unique genes and a control gene, 185. The qRT-PCR reactions were set up using Taqman x2 gene expression mastermix (Applied Biosystems), 50 ng RNA equivalent of cDNA and the total volume adjusted to 200 μl with nuclease free water (Quiagen). The TLDA cards were run on a 7900 qRT-PCR system (Applied Biosystems) using the TLDA block and analysed using the RQ manager 1.2 software (Applied Biosystems). To normalize variations in the RNA concentration and quality in different samples, the ct values obtained in real-time PCR for the genes were corrected by the ct-value obtained for the housekeeping gene in the same sample (Δct=ct housekeeping gene−ct gene of interest) then the relative changes in RNA expression were calculated using the ΔΔct method (ΔΔct=Δct sample of interest−Δct control sample) using the average Δct values of 5 normal skins as the control sample for each of the 9 GVHD skins.

At 1, 2 and 3 days of co-culture with alloreactive lymphocytes skin samples of one donor were taken and analyzed in comparison to parallel samples cultured in medium only. At day 1a GVHR of grade I was observed that increased to grade II at day 2 and grade III at day 3. The inventors determined the expression of 15 MHC and 1 NKC gene by qRT-PCR (Tab. 3).

TABLE 3
Regulation of MHC and NCR candidate genes in human skin explants

| | regulation in rat | regulation in human skin explant assay | | | |
|---|---|---|---|---|---|
| | skin explant assays | day 1 | day 2 | days | concordance |
| | (expression profiling) | (GVHR I) | (GVHR II) | (GVHR III) | rate |
| MHC region | | | | | |
| HLA-DMB | ↑¹ | — | ↑ | — | 1/3 |
| TAP1 | (↑) | ↑ | ↑ | ↑ | 3/3 |
| PSMB8 | ↑ | ↑ | ↑ | ↑ | 3/3 |
| G18 (GPSM3) | ↑ | n.d. | n.d. | n.d. | |
| PBX2 | (↑) | ↓ | n.d. | ↑ | 1/3 |
| C2 | ↑ | ↑ | ↑ | ↓ | 2/3 |
| LY6G6E | ↓ | n.d. | ↑ | n.d. | 0/3 |
| BAT5 | ↓ | — | — | — | 0/3 |
| AIF1 | ↑ | ↓ | ↑ | ↓ | 1/3 |
| LST1 | ↑ | — | ↑ | n.d. | 1/3 |
| SPR1 | ↑ | — | — | ↑ | 1/3 |
| (PSORS1C2) | | | | | |
| IER3 | ↑ | ↓ | ↑ | — | 1/3 |
| FLI13158 | (↓) | ↓ | ↓ | — | 2/3 |
| MRPS18B | (↑) | ↓ | ↓ | ↓ | 0/3 |
| UBD | ↑ | ↑ | ↑ | ↑ | 3/3 |
| NCR region | | | | | |
| OLR1 | ↑ | ↑ | ↑ | n.d. | 2/3 |

¹Explanation of symbols:
↑ up-regulated mRNA expression level (log2-fold change ≧1)
↓ down-regulated mRNA level (log2-fold change ≦−1)
— unchanged mRNA expression level (log2-fold change >−1 and <1)
(↑) significant (p < 0.05) but moderate up-regulation (log2-fold change <1) of mRNA expression level in the rat expression profiling experiment
(↓) significant (p < 0.05) but moderate down-regulation (log2-fold change >−1) of mRNA expression level
n.d. no mRNA detected

Of these 16 genes 12 (75%) were regulated at least in one skin explant sample in the way predicted by the results of the rat expression profiling experiments (Tab. 4). Three genes TAP1, PSMB8, and UBD were up-regulated in all 3 human skin explant samples. The genes C2, FLI13158, and OLR1 were regulated in 2 of the 3 samples as predicted by the rat experiments. In addition, the inventors determined the expression of 153 non-MHC/non-NCR genes that were identified to be regulated in rat skin explant assays. Also of these genes 105 (69%) were regulated in at least one of the human skin explant samples in accordance with the results obtained in the rat model (Tab. 4). These results suggest that the in vitro rat model of the skin explant assay gives evidences of gene expression changes that are very likely to occur also in human skin explant assays during GVHR.

TABLE 4
Proportion of concordantly regulated in MHC, NKC, and genes en-
coded in other regions in human skin explant assays
in comparison to rat skin explant assays

| | | | concordantly regulated | | | |
|---|---|---|---|---|---|---|
| | ana- | | in human skin explant | | | not con- |
| | lyzed | mRNA | assays in comparison | | | cordantly |
| | human | not | to rat skin explant assays | | | regulated |
| region | genes | detected | 3/3 | 2/3 | 1/3 | 0/3 |
| MHC | 15 | 1 (7%) | 3 | 2 (13%) | 6 (40%) | 3 (20%) |
| | | | (20%) | | | |
| NKC | 1 | 0 (0%) | 0 | 1 | 0 (0%) | 0 (0%) |
| | | | (0%) | (100%) | | |
| others | 153 | 18 | 33 | 31 (20%) | 41 (27%) | 30 (20%) |
| | | (12%) | (22%) | | | |

In a follow-up study, 24 genes have been identified in additional validation tests. The results are shown in Table 9. The probes used and the reference sequences are shown in Table 10. The additional validation tests confirmed the significant regulation of gene expression, i.e. up-regulation or down-regulation, preferably down-regulation for Ctss, Pbx2, Spr1, Spic, Nfe2, Tnfaip8l2, Ier3, and Lst1.

Statistical Analyses not Related to Microarray Experiments

Paired comparisons between experimental groups were performed using the non-parametric Mann-Whitney U test. Pearson's and Spearman's correlation coefficients were calculated to determine the correlation between mRNA expression levels of two genes. The statistical analyses were performed using WinSTAT® software.

### Example 3

### mRNA Expression Profiling in Human Clinical GVHD Biopsies

Further studies were undertaken to evaluate the expression markers also under clinical conditions. Therefore, new tests were performed using skin explant assay as well as mRNA expression profiling studies directly on clinical GVHD biopsies to validate the results from the previous skin explant studies. The clinical GVHD biopsies were taken from hematopoietic stem cell transplantation (HSCT) patients. These data are summarized in Table 11.

Experimental Skin Explants Assays Using Autologous HSCT Patients and Normal Controls

Peripheral blood mononuclear cells (PBMC) and skin samples were obtained from autologous HSCT patients following informed consent and approval from the North Tyneside Research Ethics Committee. Buffy coat from HLA mismatched normal blood donations were obtained from Newcastle National Blood Service with consent. Skin explant assays were performed as previously described [5,6], 1×10⁷ responder PBMC from healthy volunteers was cultured with an equal number of irradiated PBMC from autologous HSCT patients, in 10 ml complete medium (RPMI 1640 supplemented with antibiotics, 2 mM L-glutamine and 10% heat inactivated human AB serum) in a 25 cm² flask. Standard 4 mm punch skin biopsy specimens were obtained pretransplant from the auto HSCT patients and divided into 12 equal sized pieces. After 7 days of culture, the MLR primed lymphocytes were washed and resuspended in complete medium supplemented with 20% heat inactivated autologous (patient) serum and co-cultured in duplicate with patient skin at a cell concentration of 1×10⁶ cells/well in a volume of 200 μl/well in 96-well round-bottomed microtitre plates. In addition each skin sample was also cultured in duplicate in culture medium alone as a negative or medium only control. A time course experiment was set up to enable RNA expression analysis to be assessed early, (day 1) and late, (days 2 and 3) to monitor the interaction of sensitised T cells with recipient skin. Parallel control skins were incubated in medium only on days 1, 2 and 3 and used as the comparators. The skin samples were removed from the time series, duplicate control and MLR skin explant on days one, two or three, one sample was fixed in 10% buffered formalin, sectioned and stained with H&E and duplicate sample placed in RNAlater (Ambion) and stored at −80° C. prior to RNA extraction.

The histopathological evaluation of the skin explants for graft versus host reaction (GVHR) was performed independently by at least two assessors. Grade I histopathological damage in skin biopsies was regarded as background and was normally observed in the medium control. All biopsies presenting histopathological damage of grade II or above were regarded as GVHR positive.

Clinical Biopsies

Standard 4 mm punch biopsies or scrape biopsies were obtained from 10 patients at various time points post transplant at onset of acute GvHD together with normal skin skin controls (n=10). RNA was extracted from these biopsies as described below.

RNA Extraction and cDNA Production

RNA was extracted from the skin samples stored in RNA later using the Ambion mirVana miRNA Isolation Kit according to the manufacturer's recommendations and quantified using the NanoDrop ND-1000 spectrophotometer (Thermo Scientific). cDNA was generated by random hexamer priming, briefly equal quantities of RNA and 2× strength cDNA mix containing random hexamer primers (Pharmacia), dNTPs (Roche), reverse transcriptase (MMLVRT—Invitrogen) and an RNase inhibitor (Rnasin—Promega) were incubated at 37° C. for 2 hours with a further incubation at 65° C. for 10 minutes to denature the reverse transcriptase.

Validation of Candidate Genes by Quantitative Real-Time PCR

Validation of the candidate genes in the human skin explant assay and clinical biopsies was done by qRT-PCR. For this relative quantification with three custom designed Taqman low density array (TLDA) cards (Applied Biosystems) were used each card contained 4 replicates of 94 unique genes and two control genes, 18S and GAPDH, giving a total of 282 genes. The qRT-PCR reactions were set up using Taqman x2 gene expression mastermix (Applied Biosystems), 50 ng RNA equivalent of cDNA and the total volume adjusted to 200 μl with nuclease free water (Quiagen). The reaction mix was loaded onto the TLDA cards and the cards were run on a 7900 qRT-PCR system (Applied Biosystems) and analysed using the RQ manager 1.2 software (Applied Biosystems). The relative changes in RNA expression were calculated using the ΔΔct method, that is, ΔΔct=Δct sample of interest−Δct control sample, where the Δct is the ct of the control gene−the ct of the gene of interest.

Genes which showed a consistent change in expression between the medium only control skin and the MLR skin or in the clinical aGVHD skin compared to normal skin were investigated further using additional normal (n=10) and clinical aGVHD (n=10) skin samples. Real time PCR was carried out using individual TaqMan assays (Applied Biosystems) for the genes of interest and the control gene GAPDH (Applied Biosystems). The reactions were set up in triplicate using Taqman x2 gene expression mastermix, 10 to 20 ng RNA equivalent of cDNA and the manufacturer's recommended concentration of primer/probe mix. The reactions were run on a 7900 qRT-PCR system (Applied Biosystems) and analysed using the SDS 2.3 software, normalisation of expression was performed using GAPDH gene, expression results and ACT values were calculated as above.

Statistical Analysis

Comparisons between the experimental groups were carried out using the non-parametric Mann-Whitney U test using Graphpad prism 5 software (Graphpad Software inc.).

Table 5. Expression Profiling Results of MHC Genes

In Table 5a, results for all 224 MHC genes are shown in their chromosomal order (Hurt P, et al. (2004) Genome Res 14: 631-639). The expression profiling results of BN skin explant samples exposed to pre-stimulated allogeneic (PVG) lymphocytes in comparison to those exposed to syngeneic (BN) lymphocytes are given. The log 2-fold changes and the fold changes in gene expression are shown for every oligonucleotide probe used. The adjusted p-values are indicated. Significant change is defined by p<0.05 and strong change is defined by log 2-fold change ≧1 or ≦−1; i.e. fold change ≧2 or ≦0.5. In addition, the identification numbers of the probes on the arrays are given (probe ID) together with the information whether these probes were taken from the Agilent database or custom designed. Table 5b contains the same information for all MHC genes for which at least one probe indicated a significant alteration of gene expression. In Table 5c, the data for those genes are summarized that are considered to be regulated significantly because either at least a single probe indicated a significant (p<0.05) and strong (log 2-fold change ≧1 or ≦−1) regulation or at least 50% of the gene probes indicated a significant (p<0.05) regulation of gene expression.

TABLE 5a

| | | log2- | | | | | |
|---|---|---|---|---|---|---|---|
| Gene | | Fold | Fold | adj. P- | | | Probe |
| order | Gene Symbol | Change | Change | value | Gene Description | Probe ID | Design |
| 1 | 3930402F13Rik | 0.08 | 1.06 | 0.7687 | zinc finger and BTB domain containing 9 | A_43_P10072 | Agilent |
| | (Zbtb9) | | | | | | |
| 1 | 3930402F13Rik | 0.04 | 1.03 | 0.8180 | zinc finger and BTB domain containing 9 | A_43_P20769 | Agilent |
| | (Zbtb9) | | | | | | |
| 2 | Syngap1 | −0.17 | 0.89 | 0.1557 | synaptic Ras GTPase activating protein 1 homolog (rat) | A_44_P470444 | Agilent |
| 3 | Cuta | 0.21 | 1.16 | 0.4430 | cutA divalent cation tolerance homolog (E. coli) | A_42_P765298 | Agilent |
| 4 | Phf1 | 0.21 | 1.16 | 0.2688 | PHD finger protein 1 | A_44_P1057137 | Agilent |
| 5 | Kifc1 | −0.23 | 0.85 | 0.4351 | kinesin family member C1 | A_44_P1042372 | Agilent |
| 6 | AA926063 | −0.06 | 0.96 | 0.6022 | gene corresponding to rat EST acc. no. AA926063 | A_44_P128110 | Agilent |
| 7 | Daxx | 0.07 | 1.05 | 0.5495 | Fas death domain-associated protein | A_42_P622574 | Agilent |
| 8 | Znf297 | −0.21 | 0.86 | 0.1375 | zinc finger protein 297 | A_43_P18449 | Agilent |
| 8 | Znf297 | −0.12 | 0.92 | 0.8050 | zinc finger protein 297 | A_42_P486012 | Agilent |
| 8 | Znf297 | −0.06 | 0.96 | 0.7324 | zinc finger protein 297 | A_43_P20215 | Agilent |
| 8 | Znf297 | 0.12 | 1.09 | 0.4409 | zinc finger protein 297 | A_43_P20683 | Agilent |
| 9 | Tapbp | 0.31 | 1.24 | 0.3259 | TAP binding protein | A_42_P698972 | Agilent |
| 10 | Rab2l | −0.15 | 0.90 | 0.3580 | RAB2, member RAS oncogene family-like | A_44_P465986 | Agilent |
| 11 | Ke2 | −0.07 | 0.95 | 0.8616 | H2-K region expressed gene 2 | A_44_P498712 | Agilent |
| 12 | Bing4 (Wdr46) | −0.13 | 0.91 | 0.6702 | WD repeat domain 46 | A_44_P158675 | Agilent |
| 13 | B3galt4 | 0.01 | 1.01 | 0.9910 | UDP-Gal:betaGlcNAc beta 1,3-galactosyltransferase, | A_42_P692926 | Agilent |
| | | | | | polypeptide 4 | | |
| 14 | Rps18 | −0.32 | 0.80 | 0.1017 | ribosomal protein S18 | A_42_P582859 | Agilent |
| 15 | Sacm2l (Vps52) | 0.00 | 1.00 | 0.9771 | similar to vacuolar protein sorting 52 | A_43_P12732 | Agilent |
| 16 | RT1-A1 | 0.70 | 1.62 | 0.0149 | RT1 class I | CUST_1_PI202535318 | custom |
| 16 | RT1-A1 | 0.75 | 1.68 | 0.0100 | RT1 class I | CUST_2_PI202535318 | custom |
| 16 | RT1-A1 | 0.80 | 1.74 | 0.0149 | RT1 class I | CUST_3_PI202535318 | custom |
| 16 | RT1-A1 | 0.86 | 1.82 | 0.0100 | RT1 class I | CUST_4_PI202535318 | custom |
| 16 | RT1-A1 | 0.91 | 1.88 | 0.0100 | RT1 class I | CUST_5_PI202535318 | custom |
| 17 | RT1-A2 | 0.98 | 1.97 | 0.0100 | RT1 class I | A_44_P296155 | Agilent |
| 18 | RT1-A3 | 0.28 | 1.21 | 0.4444 | RT1 class I | A_44_P501234 | Agilent |
| 19 | Ring1 | −0.14 | 0.91 | 0.5739 | ring finger protein 1 | A_44_P100117 | Agilent |
| 20 | Hsd17b8 | 0.06 | 1.04 | 0.8435 | hydroxysteroid (17-beta) dehydrogenase 8 | A_43_P15081 | Agilent |
| 21 | Ke4 | −0.03 | 0.98 | 0.8962 | RT1 class I, locus Ke4 | CUST_1_PI195698117 | custom |
| 21 | Ke4 | −0.04 | 0.97 | 0.8617 | RT1 class I, locus Ke4 | CUST_2_PI195698117 | custom |
| 21 | Ke4 | −0.02 | 0.99 | 0.9361 | RT1 class I, locus Ke4 | CUST_3_PI195698117 | custom |
| 21 | Ke4 | 0.01 | 1.01 | 0.9700 | RT1 class I, locus Ke4 | CUST_4_PI195698117 | custom |
| 21 | Ke4 | −0.05 | 0.97 | 0.7835 | RT1 class I, locus Ke4 | CUST_5_PI195698117 | custom |
| 22 | Rxrb | −0.14 | 0.91 | 0.5922 | retinoid X receptor beta | A_52_P519689 | Agilent |
| 22 | Rxrb | −0.06 | 0.96 | 0.8238 | retinoid X receptor beta | CUST_11_PI207500742 | custom |
| 22 | Rxrb | −0.03 | 0.98 | 0.8934 | retinoid X receptor beta | CUST_12_PI207500742 | custom |
| 22 | Rxrb | −0.08 | 0.95 | 0.6808 | retinoid X receptor beta | CUST_13_PI207500742 | custom |
| 22 | Rxrb | −0.03 | 0.98 | 0.9004 | retinoid X receptor beta | CUST_14_PI207500742 | custom |
| 22 | Rxrb | 0.01 | 1.01 | 0.9575 | retinoid X receptor beta | CUST_15_PI207500742 | custom |
| 23 | Col11a2 | −0.45 | 0.73 | 0.0533 | procollagen, type XI, alpha 2 | A_44_2527024 | Agilent |
| 24 | RT1-Hb | 0.04 | 1.03 | 0.7868 | RT1 class II, H beta | A_44_P250763 | Agilent |
| 25 | RT1-Ha | 0.07 | 1.05 | 0.6681 | RT1 class II, H alpha | CUST_1_PI195698201 | custom |
| 25 | RT1-Ha | 0.02 | 1.01 | 0.8589 | RT1 class II, H alpha | CUST_2_PI195698201 | custom |
| 25 | RT1-Ha | −0.11 | 0.93 | 0.5819 | RT1 class II, H alpha | CUST_3_PI195698201 | custom |
| 25 | RT1-Ha | 0.00 | 1.00 | 0.9980 | RT1 class II, H alpha | CUST_4_PI195698201 | custom |
| 25 | RT1-Ha | −0.08 | 0.95 | 0.5019 | RT1 class II, H alpha | CUST_5_PI195698201 | custom |
| 26 | RT1-DOa | −0.06 | 0.96 | 0.7324 | RT1 class II, locus DOa | A_44_P344228 | Agilent |
| 27 | Brd2 | 0.46 | 1.38 | 0.0825 | bromodomain containing 2 | A_42_P558503 | Agilent |
| 28 | RT1-DMa | 0.87 | 1.83 | 0.0681 | histocompatibility 2, class II, locus DMa | A_42_P473314 | Agilent |
| 29 | RT1-DMb | 2.59 | 6.02 | 0.0100 | major histocompatibility complex, class II, DM beta | CUST_1_PI195698203 | custom |
| 29 | RT1-DMb | 2.77 | 6.82 | 0.0100 | major histocompatibility complex, class II, DM beta | CUST_2_PI195698203 | custom |
| 29 | RT1-DMb | 1.93 | 3.81 | 0.0149 | major histocompatibility complex, class II, DM beta | CUST_3_PI195698203 | custom |
| 29 | RT1-DMb | 1.87 | 3.66 | 0.0149 | major histocompatibility complex, class II, DM beta | CUST_4_PI195698203 | custom |
| 29 | RT1-DMb | 1.94 | 3.84 | 0.0100 | major histocompatibility complex, class II, DM beta | CUST_5_PI195698203 | custom |
| 30 | Psmb9 | 0.50 | 1.41 | 0.1412 | proteasome (prosome, macropain) subunit, beta type 9 | A_42_P759756 | Agilent |
| | | | | | (large multi-functional peptidase 2) | | |
| 31 | Tap1 | 0.53 | 1.44 | 0.1159 | transporter 1, ATP-binding cassette, sub-family B | A_43_P15763 | Agilent |
| | | | | | (MDR/TAP) | | |
| 31 | Tap1 | 0.63 | 1.55 | 0.0390 | transporter 1, ATP-binding cassette, sub-family B | A_44_P451916 | Agilent |
| | | | | | (MDR/TAP) | | |
| 32 | Psmb8 | 1.00 | 2.00 | 0.0336 | proteasome (prosome, macropain) subunit, beta type 8 | A_42_P761035 | Agilent |
| | | | | | (large multi-functional peptidase 7) | | |
| 33 | Tap2 | 0.44 | 1.36 | 0.1639 | transporter 2, ATP-binding cassette, sub-family B | A_42_P797381 | Agilent |
| | | | | | (MDR/TAP) | | |
| 34 | RT1-DOb | 0.39 | 1.31 | 0.0573 | RT1 class II, locus DOb | A_44_P294965 | Agilent |
| 35 | RT1-Bb | −0.29 | 0.82 | 0.6142 | RT1 class II, locus Bb | A_44_P552452 | Agilent |
| 36 | RT1-Ba | −0.14 | 0.91 | 0.8110 | RT1 class II, locus Ba | A_44_P128248 | Agilent |
| 36 | RT1-Ba | 0.18 | 1.13 | 0.2934 | RT1 class II, locus Ba | A_44_P194167 | Agilent |
| 36 | RT1-Ba | −0.09 | 0.94 | 0.8443 | RT1 class II, locus Ba | A_43_P14429 | Agilent |
| 37 | RT1-Db1 | 0.36 | 1.28 | 0.5390 | RT1 class II, D beta 1 | A_44_P130513 | Agilent |
| 38 | RT1-Db2 | 0.62 | 1.54 | 0.2275 | RT1 class II, D beta 2 | CUST_1_PI201011278 | custom |
| 38 | RT1-Db2 | 0.65 | 1.57 | 0.2267 | RT1 class II, D beta 2 | CUST_2_PI201011278 | custom |
| 38 | RT1-Db2 | 0.67 | 1.59 | 0.1732 | RT1 class II, D beta 2 | CUST_3_PI201011278 | custom |
| 38 | RT1-Db2 | 0.86 | 1.82 | 0.0856 | RT1 class II, D beta 2 | CUST_4_PI201011278 | custom |
| 38 | RT1-Db2 | 1.00 | 2.00 | 0.0755 | RT1 class II, D beta 2 | CUST_5_PI201011278 | custom |
| 39 | RT1-Da | 0.22 | 1.16 | 0.6541 | RT1 class II, D alpha | A_44_P991532 | Agilent |
| 40 | Btnl2 | −0.09 | 0.94 | 0.4427 | butyrophilin-like 2 (MHC class II associated) | A_23_P376686 | Agilent |
| 41 | Btnl3 | −0.10 | 0.93 | 0.2949 | butyrophilin-like 3 | A_42_P788302 | Agilent |
| 42 | Tesb | 0.18 | 1.13 | 0.1346 | testis specific basic protein | CUST_4_PI1956982050 | custom |
| 42 | Tesb | 0.14 | 1.10 | 0.5685 | testis specific basic protein | CUST_5_PI1956982050 | custom |
| 43 | Btnl4 | 0.75 | 1.68 | 0.1649 | butyrophilin-like 4 | CUST_44_PI2408728340 | custom |
| 43 | Btnl4 | 0.71 | 1.64 | 0.1853 | butyrophilin-like 4 | CUST_45_PI2408728340 | custom |
| 44 | Btnl5 | 0.20 | 1.15 | 0.4751 | butyrophilin-like 5 | CUST_7_PI207500742 | custom |
| 45 | Btnl6 | −0.08 | 0.95 | 0.3599 | butyrophilin-like 6 | CUST_1_PI201011255 | custom |
| 45 | Btnl6 | −0.29 | 0.82 | 0.0847 | butyrophilin-like 6 | CUST_2_PI201011255 | custom |
| 45 | Btnl6 | −0.01 | 0.99 | 0.9153 | butyrophilin-like 6 | CUST_3_PI201011255 | custom |
| 45 | Btnl6 | 0.00 | 1.00 | 0.9864 | butyrophilin-like 6 | CUST_4_PI201011255 | custom |
| 45 | Btnl6 | 0.06 | 1.04 | 0.5623 | butyrophilin-like 6 | CUST_5_PI201011255 | custom |
| 46 | Btnl7 | −0.12 | 0.92 | 0.2797 | butyrophilin-like 7 | A_44_P212575 | Agilent |
| 46 | Btnl7 | 0.08 | 1.06 | 0.7469 | butyrophilin-like 7 | CUST_1_PI201011264 | custom |
| 46 | Btnl7 | −0.04 | 0.97 | 0.7249 | butyrophilin-like 7 | CUST_2_PI201011264 | custom |
| 46 | Btnl7 | −0.07 | 0.95 | 0.5583 | butyrophilin-like 7 | CUST_3_PI201011264 | custom |
| 46 | Btnl7 | −0.08 | 0.95 | 0.5922 | butyrophilin-like 7 | CUST_4_PI201011264 | custom |
| 46 | Btnl7 | −0.28 | 0.82 | 0.0879 | butyrophilin-like 7 | CUST_5_PI201011264 | custom |
| 47 | Btnl8 | 0.09 | 1.06 | 0.4680 | butyrophilin-like 8 | A_44_P379412 | Agilent |
| 47 | Btnl8 | 0.26 | 1.20 | 0.2238 | butyrophilin-like 8 | CUST_6_PI207500742 | custom |
| 47 | Btnl8 | −0.16 | 0.90 | 0.2688 | butyrophilin-like 8 | CUST_8_PI207500742 | custom |
| 47 | Btnl8 | −0.03 | 0.98 | 0.8236 | butyrophilin-like 8 | CUST_9_PI207500742 | custom |
| 47 | Btnl8 | 0.12 | 1.09 | 0.3144 | butyrophilin-like 8 | CUST_10_PI207500742 | custom |
| 48 | Btnl9 | −0.03 | 0.98 | 0.8134 | butyrophilin-like 9 | A_32_P187951 | Agilent |
| 48 | Btnl9 | −0.11 | 0.93 | 0.3818 | butyrophilin-like 9 | A_23_P81280 | Agilent |
| 49 | C4-2 | −0.97 | 0.51 | 0.1521 | complement component 4, gene 2 | A_42_P494900 | Agilent |
| 50 | Notch4 | −0.90 | 0.54 | 0.1159 | Notch homolog 4 | A_42_P734094 | Agilent |
| 51 | G18 (Gpsm3) | 1.23 | 2.35 | 0.0315 | G18 protein | A_42_P569708 | Agilent |
| 52 | Pbx2 | 0.33 | 1.26 | 0.0466 | pre-B-cell leukemia transcription factor 2 | A_42_P592157 | Agilent |
| 53 | Ager | 0.10 | 1.07 | 0.4914 | advanced glycosylation end product-specific receptor | A_43_P15393 | Agilent |
| 54 | Rnf5 | 0.57 | 1.48 | 0.0315 | ring finger protein 5 | A_51_P204582 | Agilent |
| 54 | Rnf5 | 0.26 | 1.20 | 0.0674 | ring finger protein 5 | CUST_1_PI207500742 | custom |
| 54 | Rnf5 | 0.21 | 1.16 | 0.1445 | ring finger protein 5 | CUST_2_PI207500742 | custom |
| 54 | Rnf5 | 0.17 | 1.13 | 0.2905 | ring finger protein 5 | CUST_3_PI207500742 | custom |
| 54 | Rnf5 | 0.22 | 1.16 | 0.1626 | ring finger protein 5 | CUST_4_PI207500742 | custom |
| 54 | Rnf5 | 0.19 | 1.14 | 0.1707 | ring finger protein 5 | CUST_5_PI207500742 | custom |
| 55 | Agpat1 | 0.14 | 1.10 | 0.6400 | 1-acylglycerol-3-phosphate O-acyltransferase 1 | A_44_P419004 | Agilent |
| 56 | Ng3 | −0.18 | 0.88 | 0.3016 | NG3 protein | CUST_51_PI209196805 | custom |
| 56 | Ng3 | 0.07 | 1.05 | 0.6808 | NG3 protein | CUST_52_PI209196805 | custom |
| 56 | Ng3 | −0.12 | 0.92 | 0.3181 | NG3 protein | CUST_53_PI209196805 | custom |
| 56 | Ng3 | −0.29 | 0.82 | 0.2314 | NG3 protein | CUST_54_PI209196805 | custom |
| 56 | Ng3 | −0.07 | 0.95 | 0.8401 | NG3 protein | CUST_55_PI209196805 | custom |
| 57 | Ppt2 | −0.12 | 0.92 | 0.6876 | palmitoyl-protein thioesterase 2 | A_44_P343303 | Agilent |
| 58 | Ng5 | 0.18 | 1.13 | 0.2523 | NG5 protein | CUST_1_PI195698205 | custom |
| 58 | Ng5 | 0.06 | 1.04 | 0.6520 | NG5 protein | CUST_2_PI195698205 | custom |
| 58 | Ng5 | 0.22 | 1.16 | 0.1750 | NG5 protein | CUST_3_PI195698205 | custom |
| 58 | Ng5 | 0.18 | 1.13 | 0.1494 | NG5 protein | CUST_4_PI195698205 | custom |
| 58 | Ng5 | 0.13 | 1.09 | 0.4775 | NG5 protein | CUST_5_PI195698205 | custom |
| 59 | Fkbpl | −0.05 | 0.97 | 0.8864 | FK506 binding protein-like | A_44_P1048901 | Agilent |
| 60 | Crebl1 | 0.36 | 1.28 | 0.5778 | cAMP responsive element binding protein-like 1 | A_44_P292503 | Agilent |
| 61 | Tnx | 0.04 | 1.03 | 0.6633 | tenascin-X | CUST_2_PI2010111961 | custom |
| 61 | Tnx | 0.03 | 1.02 | 0.8015 | tenascin-X | CUST_3_PI2010111961 | custom |
| 62 | Cyp21a1 | −0.09 | 0.94 | 0.3540 | cytochrome P450, family 21, subfamily a, polypeptide 1 | A_44_P381937 | Agilent |
| 63 | C4-1 | −0.76 | 0.59 | 0.1375 | complement component 4, gene 1 | A_43_P21634 | Agilent |
| 64 | Stk19 | 0.28 | 1.21 | 0.2657 | serine/threonine kinase 19 | A_44_P491782 | Agilent |
| 65 | Dom3z | 0.27 | 1.21 | 0.3198 | DOM-3 homolog Z | A_44_P158709 | Agilent |
| 66 | Skiv2l | −0.24 | 0.85 | 0.3408 | superkiller viralicidic activity 2-like | A_44_P292558 | Agilent |
| 67 | Rdbp | −0.47 | 0.72 | 0.1367 | RD RNA-binding protein | A_44_P266879 | Agilent |
| 68 | Bf (CfB) | −0.71 | 0.61 | 0.3004 | complement factor B | A_44_P419064 | Agilent |
| 69 | C2 | 1.22 | 2.33 | 0.0325 | complement component 2 | A_44_P332606 | Agilent |
| 70 | Ng35 | −0.04 | 0.97 | 0.7095 | Ng35 protein | A_43_P17778 | Agilent |
| 71 | Bat8 (Ehmt2) | −0.23 | 0.85 | 0.5125 | euchromatic histone lysine N-methyltransferase 2 | A_44_P1057272 | Agilent |
| 72 | Ng22 (Slc44a4) | −0.20 | 0.87 | 0.3134 | solute carrier family 44, member 4 | A_43_P18443 | Agilent |
| 72 | Ng22 (Slc44a4) | −0.81 | 0.57 | 0.0598 | solute carrier family 44, member 4 | A_44_P1037285 | Agilent |
| 73 | Neu1 | 0.22 | 1.16 | 0.3690 | neuraminidase 1 | A_43_P12574 | Agilent |
| 74 | Hspa1b | 0.06 | 1.04 | 0.5939 | heat shock 70 kD protein 1B (mapped) | A_44_P532958 | Agilent |
| 75 | Hspa1a | 0.07 | 1.05 | 0.8419 | heat shock 70 kD protein 1A | A_44_P1042876 | Agilent |
| 76 | Hspa1l | −0.20 | 0.87 | 0.2682 | heat shock 70 kD protein 1-like (mapped) | A_42_P541025 | Agilent |
| 77 | Lsm2 | 0.08 | 1.06 | 0.8227 | LSM2 homolog, U6 small nuclear RNA associated | A_51_P314931 | Agilent |
| | | | | | [S. cerevisiae] | | |
| 77 | Lsm2 | 0.03 | 1.02 | 0.9332 | LSM2 homolog, U6 small nuclear RNA associated | CUST_6_PI209196805 | custom |
| | | | | | [S. cerevisiae] | | |
| 77 | Lsm2 | 0.12 | 1.09 | 0.7330 | LSM2 homolog, U6 small nuclear RNA associated | CUST_7_PI209196805 | custom |
| | | | | | [S. cerevisiae] | | |
| 77 | Lsm2 | −0.04 | 0.97 | 0.8943 | LSM2 homolog, U6 small nuclear RNA associated | CUST_8_PI209196805 | custom |
| | | | | | [S. cerevisiae] | | |
| 77 | Lsm2 | 0.13 | 1.09 | 0.6868 | LSM2 homolog, U6 small nuclear RNA associated | CUST_9_PI209196805 | custom |
| | | | | | [S. cerevisiae] | | |
| 77 | Lsm2 | 0.06 | 1.04 | 0.8429 | LSM2 homolog, U6 small nuclear RNA associated | CUST_10_PI209196805 | custom |
| | | | | | [S. cerevisiae] | | |
| 78 | G7e | −0.35 | 0.78 | 0.8265 | G7e pseudogen | CUST_1_PI2010111701 | custom |
| 78 | G7e | −0.21 | 0.86 | 0.6235 | G7e pseudogen | CUST_2_PI2010111701 | custom |
| 79 | Vars2 | 0.08 | 1.06 | 0.7163 | valyl-tRNA synthetase | A_42_P646976 | Agilent |
| 80 | G7c | −0.14 | 0.91 | 0.2006 | G7c protein | A_44_P325599 | Agilent |
| 80 | G7c | −0.03 | 0.98 | 0.9121 | G7c protein | CUST_26_PI209196805 | custom |
| 80 | G7c | −0.10 | 0.93 | 0.6167 | G7c protein | CUST_27_PI209196805 | custom |
| 80 | G7c | −0.05 | 0.97 | 0.8127 | G7c protein | CUST_28_PI209196805 | custom |
| 80 | G7c | −0.10 | 0.93 | 0.3349 | G7c protein | CUST_29_PI209196805 | custom |
| 80 | G7c | −0.07 | 0.95 | 0.6486 | G7c protein | CUST_30_PI209196805 | custom |
| 81 | Ng23 | 0.00 | 1.00 | 0.9979 | Ng23 protein | A_51_P233727 | Agilent |
| 82 | Msh5 | 0.01 | 1.01 | 0.9515 | mutS homolog 5 (E. coli) | A_43_P23342 | Agilent |
| 83 | Clic1 | 0.05 | 1.04 | 0.7886 | chloride intracellular channel 1 | A_44_P1028007 | Agilent |
| 84 | Ddah2 | 0.11 | 1.08 | 0.6564 | dimethylarginine dimethylaminohydrolase 2 | CUST_1_PI195698222 | custom |
| 84 | Ddah2 | 0.17 | 1.13 | 0.3684 | dimethylarginine dimethylaminohydrolase 2 | CUST_2_PI195698222 | custom |
| 84 | Ddah2 | 0.15 | 1.11 | 0.4977 | dimethylarginine dimethylaminohydrolase 2 | CUST_3_PI195698222 | custom |
| 84 | Ddah2 | 0.13 | 1.09 | 0.5078 | dimethylarginine dimethylaminohydrolase 2 | CUST_4_PI195698222 | custom |
| 84 | Ddah2 | 0.12 | 1.09 | 0.5019 | dimethylarginine dimethylaminohydrolase 2 | CUST_5_PI195698222 | custom |
| 85 | G6b | −0.01 | 0.99 | 0.8795 | G6b protein | A_44_P334847 | Agilent |
| 86 | Ly6g6c | 0.12 | 1.09 | 0.7567 | lymphocyte antigen 6 complex, locus G6C | CUST_1_PI195698232 | custom |
| 86 | Ly6g6c | 0.11 | 1.08 | 0.7656 | lymphocyte antigen 6 complex, locus G6C | CUST_2_PI195698232 | custom |
| 86 | Ly6g6c | 0.12 | 1.09 | 0.7537 | lymphocyte antigen 6 complex, locus G6C | CUST_3_PI195698232 | custom |
| 86 | Ly6g6c | 0.37 | 1.29 | 0.2006 | lymphocyte antigen 6 complex, locus G6C | CUST_4_PI195698232 | custom |
| 86 | Ly6g6c | 0.38 | 1.30 | 0.1845 | lymphocyte antigen 6 complex, locus G6C | CUST_5_PI195698232 | custom |
| 87 | Ly6g6d | 0.30 | 1.23 | 0.4948 | lymphocyte antigen 6 complex, locus G6D | CUST_1_PI195698244 | custom |
| 87 | Ly6g6d | 0.28 | 1.21 | 0.4856 | lymphocyte antigen 6 complex, locus G6D | CUST_2_PI195698244 | custom |
| 87 | Ly6g6d | 0.19 | 1.14 | 0.6310 | lymphocyte antigen 6 complex, locus G6D | CUST_3_PI195698244 | custom |
| 87 | Ly6g6d | 0.47 | 1.39 | 0.3675 | lymphocyte antigen 6 complex, locus G6D | CUST_4_PI195698244 | custom |
| 87 | Ly6g6d | 0.36 | 1.28 | 0.5300 | lymphocyte antigen 6 complex, locus G6D | CUST_5_PI195698244 | custom |
| 88 | Ly6g6e | −1.38 | 0.38 | 0.0416 | lymphocyte antigen 6 complex, locus G6E | CUST_1_PI195698246 | custom |
| 88 | Ly6g6e | −1.42 | 0.37 | 0.0523 | lymphocyte antigen 6 complex, locus G6E | CUST_2_PI195698246 | custom |
| 88 | Ly6g6e | −1.39 | 0.38 | 0.0623 | lymphocyte antigen 6 complex, locus G6E | CUST_3_PI195698246 | custom |
| 88 | Ly6g6e | −1.44 | 0.37 | 0.0416 | lymphocyte antigen 6 complex, locus G6E | CUST_4_PI195698246 | custom |
| 88 | Ly6g6e | −1.46 | 0.36 | 0.0433 | lymphocyte antigen 6 complex, locus G6E | CUST_5_PI195698246 | custom |
| 89 | G6f (Ly6g6f) | −0.15 | 0.90 | 0.2839 | lymphocyte antigen 6 complex, locus G6F | CUST_1_PI195701417 | custom |
| 89 | G6f (Ly6g6f) | 0.22 | 1.16 | 0.0965 | lymphocyte antigen 6 complex, locus G6F | CUST_2_PI195701417 | custom |
| 89 | G6f (Ly6g6f) | −0.02 | 0.99 | 0.8965 | lymphocyte antigen 6 complex, locus G6F | CUST_3_PI195701417 | custom |
| 89 | G6f (Ly6g6f) | 0.05 | 1.04 | 0.7887 | lymphocyte antigen 6 complex, locus G6F | CUST_4_PI195701417 | custom |
| 89 | G6f (Ly6g6f) | 0.41 | 1.33 | 0.0716 | lymphocyte antigen 6 complex, locus G6F | CUST_5_PI195701417 | custom |
| 90 | Bat5 | −0.60 | 0.66 | 0.0100 | HLA-B associated transcript 5 | CUST_1_PI195830595 | custom |
| 90 | Bat5 | −0.48 | 0.72 | 0.0100 | HLA-B associated transcript 5 | CUST_2_PI195830595 | custom |
| 90 | Bat5 | −0.54 | 0.69 | 0.0180 | HLA-B associated transcript 5 | CUST_3_PI195830595 | custom |
| 90 | Bat5 | −0.53 | 0.69 | 0.0229 | HLA-B associated transcript 5 | CUST_4_PI195830595 | custom |
| 90 | Bat5 | −0.58 | 0.67 | 0.0100 | HLA-B associated transcript 5 | CUST_5_PI195830595 | custom |
| 91 | Ly6g5c | −0.18 | 0.88 | 0.4183 | lymphocyte antigen 6 complex, locus G5C | A_44_P355842 | Agilent |
| 92 | Ly6g5b | 0.01 | 1.01 | 0.9526 | lymphocyte antigen 6 complex, locus G5B | A_44_P111744 | Agilent |
| 93 | Csnk2b | −0.40 | 0.76 | 0.4907 | casein kinase 2, beta subunit | A_44_P453337 | Agilent |
| 94 | Bat4 | −0.06 | 0.96 | 0.7985 | Bat4 gene | CUST_1_PI195941286 | custom |
| 94 | Bat4 | 0.02 | 1.01 | 0.9500 | Bat4 gene | CUST_2_PI195941286 | custom |
| 94 | Bat4 | 0.00 | 1.00 | 0.9979 | Bat4 gene | CUST_3_PI195941286 | custom |
| 94 | Bat4 | 0.02 | 1.01 | 0.9284 | Bat4 gene | CUST_4_PI195941286 | custom |
| 94 | Bat4 | 0.04 | 1.03 | 0.8698 | Bat4 gene | CUST_5_PI195941286 | custom |
| 95 | G4 | −0.12 | 0.92 | 0.6277 | G4 protein | A_44_P327945 | Agilent |
| 96 | Apom | −0.31 | 0.81 | 0.1188 | apolipoprotein M | A_43_P15453 | Agilent |
| 97 | Bat3 | −0.04 | 0.97 | 0.8843 | HLA-B-associated transcript 3 | A_42_P506345 | Agilent |
| 98 | Bat2 | −0.08 | 0.95 | 0.6799 | HLA-B associated transcript 2 | CUST_1_PI195941289 | custom |
| 98 | Bat2 | −0.02 | 0.99 | 0.9413 | HLA-B associated transcript 2 | CUST_2_PI195941289 | custom |
| 98 | Bat2 | −0.07 | 0.95 | 0.7889 | HLA-B associated transcript 2 | CUST_3_PI195941289 | custom |
| 98 | Bat2 | −0.11 | 0.93 | 0.5007 | HLA-B associated transcript 2 | CUST_5_PI195941289 | custom |
| 98 | Bat2 | 0.05 | 1.04 | 0.63 | HLA-B associated transcript 2 | CUST_4_PI195941289 | custom |
| 99 | E230034O05Rik | −0.06 | 0.96 | 0.4994 | E230034O05Rik gene | A_44_P255078 | Agilent |
| 100 | Aif1 | 2.83 | 7.11 | 0.0100 | allograft inflammatory factor 1 | A_44_P421534 | Agilent |
| 101 | Ncr3 | −0.20 | 0.87 | 0.4300 | natural cytotoxicity triggering receptor 3 | A_43_P22986 | Agilent |
| 102 | Lst1 | 3.32 | 9.99 | 0.0100 | leucocyte specific transcript 1 | A_43_P12274 | Agilent |
| 103 | Ltb | 1.15 | 2.22 | 0.0693 | lymphotoxin B | A_42_P550914 | Agilent |
| 104 | Tnf | 0.32 | 1.25 | 0.0924 | tumor necrosis factor | A_43_P11513 | Agilent |
| 105 | Lta | 1.10 | 2.14 | 0.0523 | lymphotoxin A | A_43_P15592 | Agilent |
| 106 | Nfkbil1 | −0.01 | 0.99 | 0.9859 | nuclear factor of kappa light polypeptide gene enhancer | CUST_1_PI195941300 | custom |
| | | | | | in B-cells inhibitor-like 1 | | |
| 106 | Nfkbil1 | 0.10 | 1.07 | 0.8117 | nuclear factor of kappa light polypeptide gene enhancer | CUST_2_PI195941300 | custom |
| | | | | | in B-cells inhibitor-like 1 | | |
| 106 | Nfkbil1 | 0.10 | 1.07 | 0.8007 | nuclear factor of kappa light polypeptide gene enhancer | CUST_3_PI195941300 | custom |
| | | | | | in B-cells inhibitor-like 1 | | |
| 106 | Nfkbil1 | 0.03 | 1.02 | 0.9472 | nuclear factor of kappa light polypeptide gene enhancer | CUST_4_PI195941300 | custom |
| | | | | | in B-cells inhibitor-like 1 | | |
| 106 | Nfkbil1 | 0.17 | 1.13 | 0.6007 | nuclear factor of kappa light polypeptide gene enhancer | CUST_5_PI195941300 | custom |
| | | | | | in B-cells inhibitor-like 1 | | |
| 107 | Atp6v1g2 | −0.15 | 0.90 | 0.2622 | ATPase, H+ transporting, V1 subunit G isoform 2 | A_44_P484719 | Agilent |
| 108 | Bat1a | −0.56 | 0.68 | 0.0769 | HLA-B-associated transcript 1A | A_42_P784188 | Agilent |
| 109 | RT1-CE1 | 0.45 | 1.37 | 0.0668 | RT1 class I, CE1 | A_44_P513029 | Agilent |
| 110 | RT1-CE2 | 0.64 | 1.56 | 0.0278 | RT1 class I, CE2 | A_44_P107372 | Agilent |
| 111 | RT1-CE3 | 0.96 | 1.95 | 0.0100 | RT1 class I, CE3 | A_44_P274061 | Agilent |
| 112 | RT1-CE4 | 0.43 | 1.35 | 0.1222 | RT1 class I, CE4 | A_44_P440514 | Agilent |
| 113 | RT1-CE5 | 0.70 | 1.62 | 0.0395 | RT1 class I, CE5 | A_44_P172850 | Agilent |
| 114 | RT1-CE6 | 0.18 | 1.13 | 0.6413 | RT1-CE6 gene | A_44_P547954 | Agilent |
| 115 | RT1-CE7 | 0.45 | 1.37 | 0.1503 | RT1 class I, CE7 | A_42_P511265 | Agilent |
| 116 | RT1-CE8 | 0.90 | 1.87 | 0.0278 | RT1 class I, CE8 | CUST_1_PI201011245 | custom |
| 116 | RT1-CE8 | 0.91 | 1.88 | 0.0100 | RT1 class I, CE8 | CUST_2_PI201011245 | custom |
| 116 | RT1-CE8 | 0.78 | 1.72 | 0.0229 | RT1 class I, CE8 | CUST_3_PI201011245 | custom |
| 116 | RT1-CE8 | 0.84 | 1.79 | 0.0100 | RT1 class I, CE8 | CUST_4_PI201011245 | custom |
| 116 | RT1-CE8 | 0.79 | 1.73 | 0.0149 | RT1 class I, CE8 | CUST_5_PI201011245 | custom |
| 117 | RT1-CE9 | 0.80 | 1.74 | 0.0315 | RT1 class I, CE9 | CUST_1_PI201011241 | custom |
| 117 | RT1-CE9 | 0.35 | 1.27 | 0.1745 | RT1 class I, CE9 | CUST_2_PI201011241 | custom |
| 117 | RT1-CE9 | 0.74 | 1.67 | 0.0539 | RT1 class I, CE9 | CUST_3_PI201011241 | custom |
| 117 | RT1-CE9 | 0.24 | 1.18 | 0.3698 | RT1 class I, CE9 | CUST_4_PI201011241 | custom |
| 117 | RT1-CE9 | 0.81 | 1.75 | 0.0373 | RT1 class I, CE9 | CUST_5_PI201011241 | custom |
| 118 | RT1-CE10 | 4.09 | 17.03 | 0.0100 | RT1 class I, CE10 | A_44_P389019 | Agilent |
| 119 | RT1-CE11 | 0.28 | 1.21 | 0.2867 | RT1 class I, CE11 | CUST_1_PI195941302 | custom |
| 119 | RT1-CE11 | 0.65 | 1.57 | 0.0315 | RT1 class I, CE11 | CUST_2_PI195941302 | custom |
| 119 | RT1-CE11 | 0.22 | 1.16 | 0.2638 | RT1 class I, CE11 | CUST_3_PI195941302 | custom |
| 119 | RT1-CE11 | 0.16 | 1.12 | 0.3957 | RT1 class I, CE11 | CUST_4_PI195941302 | custom |
| 119 | RT1-CE11 | 0.38 | 1.30 | 0.0980 | RT1 class I, CE11 | CUST_5_PI195941302 | custom |
| 120 | RT1-CE12 | 0.43 | 1.35 | 0.1710 | RT1 class I, CE12 | CUST_1_PI195941305 | custom |
| 120 | RT1-CE12 | −0.10 | 0.93 | 0.4503 | RT1 class I, CE12 | CUST_2_PI195941305 | custom |
| 120 | RT1-CE12 | 0.34 | 1.27 | 0.1043 | RT1 class I, CE12 | CUST_3_PI195941305 | custom |
| 120 | RT1-CE12 | 0.04 | 1.03 | 0.8574 | RT1 class I, CE12 | CUST_4_PI195941305 | custom |
| 120 | RT1-CE12 | 0.56 | 1.47 | 0.0310 | RT1 class I, CE12 | CUST_5_PI195941305 | custom |
| 121 | RT1-CE13 | −0.42 | 0.75 | 0.1923 | RT1 class I, CE13 | CUST_1_PI197795816 | custom |
| 121 | RT1-CE13 | −0.46 | 0.73 | 0.2116 | RT1 class I, CE13 | CUST_2_PI197795816 | custom |
| 121 | RT1-CE13 | 0.37 | 1.29 | 0.1077 | RT1 class I, CE13 | CUST_3_PI197795816 | custom |
| 121 | RT1-CE13 | 0.38 | 1.30 | 0.1263 | RT1 class I, CE13 | CUST_4_PI197795816 | custom |
| 121 | RT1-CE13 | 0.40 | 1.32 | 0.0752 | RT1 class I, CE13 | CUST_5_PI197795816 | custom |
| 122 | RT1-CE14 | 0.39 | 1.31 | 0.1076 | RT1 class I, CE14 | CUST_1_PI195941310 | custom |
| 122 | RT1-CE14 | 0.35 | 1.27 | 0.1471 | RT1 class I, CE14 | CUST_2_PI195941310 | custom |
| 122 | RT1-CE14 | 0.30 | 1.23 | 0.1626 | RT1 class I, CE14 | CUST_3_PI195941310 | custom |
| 122 | RT1-CE14 | 0.25 | 1.19 | 0.2529 | RT1 class I, CE14 | CUST_4_PI195941310 | custom |
| 122 | RT1-CE14 | 0.25 | 1.19 | 0.2735 | RT1 class I, CE14 | CUST_5_PI195941310 | custom |
| 123 | RT1-CE15 | 0.28 | 1.21 | 0.2085 | RT1 class I, CE15 | CUST_1_PI195941312 | custom |
| 123 | RT1-CE15 | 0.26 | 1.20 | 0.2210 | RT1 class I, CE15 | CUST_2_PI195941312 | custom |
| 123 | RT1-CE15 | 0.30 | 1.23 | 0.1395 | RT1 class I, CE15 | CUST_3_PI195941312 | custom |
| 123 | RT1-CE15 | 0.29 | 1.22 | 0.1795 | RT1 class I, CE15 | CUST_4_PI195941312 | custom |
| 123 | RT1-CE15 | 0.35 | 1.27 | 0.1157 | RT1 class I, CE15 | CUST_5_PI195941312 | custom |
| 124 | RT1-CE16 | 0.54 | 1.45 | 0.0325 | RT1 class I, CE16 (RT1 class Ib, locus Cl) | A_44_P867246 | Agilent |
| 124 | RT1-CE16 | 0.78 | 1.72 | 0.0206 | RT1 class I, CE16 (RT1 class Ib. locus Cl) | A_44_P554925 | Agilent |
| 125 | Pou5f1 | 0.02 | 1.01 | 0.8552 | POU domain, class 5, transcription factor 1 | CUST_1_PI195941317 | custom |
| 125 | Pou5f1 | −0.12 | 0.92 | 0.5977 | POU domain, class 5, transcription factor 1 | CUST_2_PI195941317 | custom |
| 125 | Pou5f1 | 0.07 | 1.05 | 0.7099 | POU domain, class 5, transcription factor 1 | CUST_3_PI195941317 | custom |
| 125 | Pou5f1 | 0.15 | 1.11 | 0.2432 | POU domain, class 5, transcription factor 1 | CUST_4_PI195941317 | custom |
| 125 | Pou5f1 | −0.07 | 0.95 | 0.5946 | POU domain, class 5, transcription factor 1 | CUST_5_PI195941317 | custom |
| 126 | Tcf19 | −0.19 | 0.88 | 0.5212 | transcription factor 19 | A_42_P591665 | Agilent |
| 127 | Hcr | −0.19 | 0.88 | 0.1202 | HCR (a-helix coiled-coil rod homolog) | A_52_P669964 | Agilent |
| 127 | Hcr | −0.26 | 0.84 | 0.2118 | HCR (a-helix coiled-coil rod homolog) | CUST_11_PI209196805 | custom |
| 127 | Hcr | −0.26 | 0.84 | 0.2030 | HCR (a-helix coiled-coil rod homolog) | CUST_12_PI209196805 | custom |
| 127 | Hcr | −0.25 | 0.84 | 0.2461 | HCR (a-helix coiled-coil rod homolog) | CUST_13_PI209196805 | custom |
| 127 | Hcr | −0.16 | 0.90 | 0.3650 | HCR (a-helix coiled-coil rod homolog) | CUST_14_PI209196805 | custom |
| 127 | Hcr | −0.15 | 0.90 | 0.5193 | HCR (a-helix coiled-coil rod homolog) | CUST_15_PI209196805 | custom |
| 128 | Spr1 | 1.26 | 2.39 | 0.0206 | psoriasis susceptibility 1 candidate 2 (human) | A_66_P100662 | Agilent |
| 128 | Spr1 | 1.39 | 2.62 | 0.0180 | psoriasis susceptibility 1 candidate 2 (human) | A_51_P212958 | Agilent |
| 128 | Spr1 | 1.36 | 2.57 | 0.0206 | psoriasis susceptibility 1 candidate 2 (human) | A_51_P212956 | Agilent |
| 128 | Spr1 | 1.50 | 2.83 | 0.0100 | psoriasis susceptibility 1 candidate 2 (human) | CUST_56_PI209196805 | custom |
| 128 | Spr1 | 1.52 | 2.87 | 0.0100 | psoriasis susceptibility 1 candidate 2 (human) | CUST_57_PI209196805 | custom |
| 128 | Spr1 | 1.51 | 2.85 | 0.0100 | psoriasis susceptibility 1 candidate 2 (human) | CUST_58_PI209196805 | custom |
| 128 | Spr1 | 1.50 | 2.83 | 0.0100 | psoriasis susceptibility 1 candidate 2 (human) | CUST_59_PI209196805 | custom |
| 128 | Spr1 | 1.58 | 2.99 | 0.0100 | psoriasis susceptibility 1 candidate 2 (human) | CUST_60_PI209196805 | custom |
| 129 | Cdsn | 0.37 | 1.29 | 0.2732 | corneodesmosin | CUST_1_PI201011238 | custom |
| 129 | Cdsn | 0.84 | 1.79 | 0.0100 | corneodesmosin | CUST_2_PI201011238 | custom |
| 129 | Cdsn | 0.38 | 1.30 | 0.2184 | corneodesmosin | CUST_3_PI201011238 | custom |
| 129 | Cdsn | 0.32 | 1.25 | 0.3754 | corneodesmosin | CUST_4_PI201011238 | custom |
| 129 | Cdsn | 0.40 | 1.32 | 0.1769 | corneodesmosin | CUST_5_PI201011238 | custom |
| 130 | Stg | 0.13 | 1.09 | 0.4327 | Stg protein | A_44_P161038 | Agilent |
| 130 | Stg | 0.06 | 1.04 | 0.8258 | Stg protein | A_43_P12304 | Agilent |
| 131 | CB741658 | −0.09 | 0.94 | 0.3912 | CB741658 gene | CUST_1_PI197795805 | custom |
| 131 | CB741658 | 0.05 | 1.04 | 0.5990 | CB741658 gene | CUST_2_PI197795805 | custom |
| 131 | CB741658 | −0.05 | 0.97 | 0.6299 | CB741658 gene | CUST_3_PI197795805 | custom |
| 131 | CB741658 | −0.03 | 0.98 | 0.7498 | CB741658 gene | CUST_4_PI197795805 | custom |
| 131 | CB741658 | −0.05 | 0.97 | 0.5704 | CB741658 gene | CUST_5_PI197795805 | custom |
| 132 | Dpcr1 | −0.11 | 0.93 | 0.3540 | diffuse panbronchiolitis critical region 1 (human) | A_66_P112041 | Agilent |
| 132 | Dpcr1 | −0.17 | 0.89 | 0.0877 | diffuse panbronchiolitis critical region 1 (human) | CUST_36_PI209196805 | custom |
| 132 | Dpcr1 | −0.10 | 0.93 | 0.4426 | diffuse panbronchiolitis critical region 1 (human) | CUST_37_PI209196805 | custom |
| 132 | Dpcr1 | −0.11 | 0.93 | 0.2585 | diffuse panbronchiolitis critical region 1 (human) | CUST_38_PI209196805 | custom |
| 132 | Dpcr1 | −0.14 | 0.91 | 0.2435 | diffuse panbronchiolitis critical region 1 (human) | CUST_39_PI209196805 | custom |
| 132 | Dpcr1 | −0.04 | 0.97 | 0.7474 | diffuse panbronchiolitis critical region 1 (human) | CUST_40_PI209196805 | custom |
| 133 | E030032D13Rik | −0.24 | 0.85 | 0.0701 | E030032D13Rik gene | A_44_P341977 | Agilent |
| 134 | Kiaa1885 | −0.08 | 0.95 | 0.5867 | KIAA1885 protein | A_44_P1007561 | Agilent |
| 135 | Gtf2h4 | −0.09 | 0.94 | 0.7537 | general transcription factor II H, polypeptide 4 | CUST_1_PI197795807 | custom |
| 135 | Gtf2h4 | −0.03 | 0.98 | 0.9410 | general transcription factor II H, polypeptide 4 | CUST_2_PI197795807 | custom |
| 135 | Gtf2h4 | −0.12 | 0.92 | 0.7090 | general transcription factor II H, polypeptide 4 | CUST_3_PI197795807 | custom |
| 135 | Gtf2h4 | −0.02 | 0.99 | 0.9552 | general transcription factor II H, polypeptide 4 | CUST_4_PI197795807 | custom |
| 135 | Gtf2h4 | −0.06 | 0.96 | 0.8501 | general transcription factor II H, polypeptide 4 | CUST_5_PI197795807 | custom |
| 136 | Ddr1 | −0.18 | 0.88 | 0.1499 | discoidin domain receptor family, member 1 | A_44_P515494 | Agilent |
| 137 | CB707485I | −0.01 | 0.99 | 0.9561 | gene corresponding to rat EST CB707485 | CUST_1_PI201011227 | custom |
| 137 | CB707485I | 0.08 | 1.06 | 0.5379 | gene corresponding to rat EST CB707485 | CUST_2_PI201011227 | custom |
| 137 | CB707485I | 0.04 | 1.03 | 0.7953 | gene corresponding to rat EST CB707485 | CUST_3_PI201011227 | custom |
| 137 | CB707485I | 0.07 | 1.05 | 0.5259 | gene corresponding to rat EST CB707485 | CUST_4_PI201011227 | custom |
| 137 | CB707485I | −0.08 | 0.95 | 0.7190 | gene corresponding to rat EST CB707485 | CUST_5_PI201011227 | custom |
| 138 | Ier3 | 0.87 | 1.83 | 0.0229 | immediate early response 3 | A_42_P515405 | Agilent |
| 139 | Flot1 | 0.04 | 1.03 | 0.8901 | flotillin 1 | A_44_P1023498 | Agilent |
| 140 | Tubb5 | 0.16 | 1.12 | 0.2875 | tubulin, beta 5 | A_44_P825566 | Agilent |
| 141 | Kiaa0170 (Mdc1) | 0.02 | 1.01 | 0.9108 | mediator of DNA damage checkpoint 1 | A_42_P627572 | Agilent |
| 142 | Nrm | −0.06 | 0.96 | 0.8031 | nurim (nuclear envelope membrane protein) | CUST_1_PI197795809 | custom |
| 142 | Nrm | −0.11 | 0.93 | 0.6622 | nurim (nuclear envelope membrane protein) | CUST_2_PI197795809 | custom |
| 142 | Nrm | −0.20 | 0.87 | 0.3384 | nurim (nuclear envelope membrane protein) | CUST_3_PI197795809 | custom |
| 142 | Nrm | −0.05 | 0.97 | 0.8551 | nurim (nuclear envelope membrane protein) | CUST_4_PI197795809 | custom |
| 142 | Nrm | 0.03 | 1.02 | 0.8504 | nurim (nuclear envelope membrane protein) | CUST_5_PI197795809 | custom |
| 143 | Kiaa1949 | 0.42 | 1.34 | 0.0481 | KIAA1949 protein | CUST_1_PI201011218 | custom |
| 143 | Kiaa1949 | 0.49 | 1.40 | 0.0457 | KIAA1949 protein | CUST_2_PI201011218 | custom |
| 143 | Kiaa1949 | 0.33 | 1.26 | 0.1378 | KIAA1949 protein | CUST_3_PI201011218 | custom |
| 143 | Kiaa1949 | 0.39 | 1.31 | 0.0993 | KIAA1949 protein | CUST_4_PI201011218 | custom |
| 143 | Kiaa1949 | 0.34 | 1.27 | 0.1184 | KIAA1949 protein | CUST_5_PI201011218 | custom |
| 144 | Ddx16 | 0.04 | 1.03 | 0.8954 | DEAH (Asp-Glu-Ala-His) box polypeptide 16 | A_44_P379461 | Agilent |
| 144 | Ddx16 | −0.27 | 0.83 | 0.0797 | DEAH (Asp-Glu-Ala-His) box polypeptide 16 | A_43_P20689 | Agilent |
| 145 | Mgc15854 | 0.12 | 1.09 | 0.5094 | hypothetical protein MGC15854 | A_42_P508754 | Agilent |
| | (RGD1302996) | | | | | | |
| 145 | Mgc15854 | 0.05 | 1.04 | 0.8290 | hypothetical protein MGC15854 | A_44_P1002280 | Agilent |
| | (RGD1302996) | | | | | | |
| 146 | Flj13158 | −0.25 | 0.84 | 0.0832 | hypothetical protein FLJ13158 | A_44_P278509 | Agilent |
| | (RGD1303066) | | | | | | |
| 146 | Flj13158 | −0.57 | 0.67 | 0.0378 | hypothetical protein FLJ13158 | A_44_P654250 | Agilent |
| | (RGD1303066) | | | | | | |
| 147 | Mrps18b | 0.52 | 1.43 | 0.0474 | mitochondrial ribosomal protein S18B | CUST_1_PI197795811 | custom |
| 147 | Mrps18b | 0.49 | 1.40 | 0.0378 | mitochondrial ribosomal protein S18B | CUST_2_PI197795811 | custom |
| 147 | Mrps18b | 0.57 | 1.48 | 0.0267 | mitochondrial ribosomal protein S18B | CUST_3_PI197795811 | custom |
| 147 | Mrps18b | 0.59 | 1.51 | 0.0365 | mitochondrial ribosomal protein S18B | CUST_4_PI197795811 | custom |
| 147 | Mrps18b | 0.62 | 1.54 | 0.0254 | mitochondrial ribosomal protein S18B | CUST_5_PI197795811 | custom |
| 148 | Ppp1r10 | 0.49 | 1.40 | 0.1582 | protein phosphatase 1, regulatory subunit 10 | A_42_P497323 | Agilent |
| 149 | Abcf1 | 0.46 | 1.38 | 0.0832 | ATP-binding cassette, sub-family F (GCN20), member 1 | CUST_46_PI209196805 | custom |
| 149 | Abcf1 | 0.44 | 1.36 | 0.1863 | ATP-binding cassette, sub-family F (GCN20), member 1 | CUST_47_PI209196805 | custom |
| 149 | Abcf1 | 0.34 | 1.27 | 0.2797 | ATP-binding cassette, sub-family F (GCN20), member 1 | CUST_48_PI209196805 | custom |
| 149 | Abcf1 | 0.30 | 1.23 | 0.3188 | ATP-binding cassette, sub-family F (GCN20), member 1 | CUST_49_PI209196805 | custom |
| 149 | Abcf1 | 0.34 | 1.27 | 0.2180 | ATP-binding cassette, sub-family F (GCN20), member 1 | CUST_50_PI209196805 | custom |
| 150 | Cat56 (Prr3) | −0.01 | 0.99 | 0.9791 | proline-rich polypeptide 3 | A_44_P299349 | Agilent |
| 151 | Gnl1 | 0.05 | 1.04 | 0.8944 | guanine nucleotide binding protein, related sequence 1 | A_65_P05751 | Agilent |
| 151 | Gnl1 | −0.04 | 0.97 | 0.6698 | guanine nucleotide binding protein, related sequence 1 | A_66_P118660 | Agilent |
| 151 | Gnl1 | 0.02 | 1.01 | 0.9496 | guanine nucleotide binding protein, related sequence 1 | A_51_P102809 | Agilent |
| 151 | Gnl1 | −0.15 | 0.90 | 0.5093 | guanine nucleotide binding protein, related sequence 1 | A_51_P102814 | Agilent |
| 151 | Gnl1 | 0.07 | 1.05 | 0.8093 | guanine nucleotide binding protein, related sequence 1 | A_52_P491766 | Agilent |
| 151 | Gnl1 | −0.24 | 0.85 | 0.2708 | guanine nucleotide binding protein, related sequence 1 | CUST_41_PI209196805 | custom |
| 151 | Gnl1 | −0.17 | 0.89 | 0.4205 | guanine nucleotide binding protein, related sequence 1 | CUST_42_PI209196805 | custom |
| 151 | Gnl1 | 0.03 | 1.02 | 0.9311 | guanine nucleotide binding protein, related sequence 1 | CUST_43_PI209196805 | custom |
| 151 | Gnl1 | 0.02 | 1.01 | 0.9448 | guanine nucleotide binding protein, related sequence 1 | CUST_44_PI209196805 | custom |
| 151 | Gnl1 | 0.03 | 1.02 | 0.8853 | guanine nucleotide binding protein, related sequence 1 | CUST_45_PI209196805 | custom |
| 152 | RT1-T24-1 | 0.25 | 1.19 | 0.2040 | RT1 class I, T24, gene 1 | A_44_P187530 | Agilent |
| 153 | RT1-T24-2 | −0.01 | 0.99 | 0.9531 | RT1 class I, T24, gene 2 | A_44_P215023 | Agilent |
| 154 | RT1-T24-3 | 0.31 | 1.24 | 0.1540 | RT1 class I, T24, gene 3 | CUST_1_PI201011214 | custom |
| 154 | RT1-T24-3 | 0.42 | 1.34 | 0.0336 | RT1 class I, T24, gene 3 | CUST_2_PI201011214 | custom |
| 154 | RT1-T24-3 | 0.27 | 1.21 | 0.1454 | RT1 class I, T24, gene 3 | CUST_3_PI201011214 | custom |
| 154 | RT1-T24-3 | 0.31 | 1.24 | 0.0847 | RT1 class I, T24, gene 3 | CUST_4_PI201011214 | custom |
| 154 | RT1-T24-3 | 0.08 | 1.06 | 0.6030 | RT1 class I, T24, gene 3 | CUST_5_PI201011214 | custom |
| 155 | RT1-T24-4 | 0.57 | 1.48 | 0.0345 | RT1 class I, T24, gene 4 | CUST_1_PI197795813 | custom |
| 155 | RT1-T24-4 | 0.76 | 1.69 | 0.0206 | RT1 class I, T24, gene 4 | CUST_2_PI197795813 | custom |
| 155 | RT1-T24-4 | 0.72 | 1.65 | 0.0206 | RT1 class I, T24, gene 4 | CUST_3_PI197795813 | custom |
| 155 | RT1-T24-4 | 0.39 | 1.31 | 0.0611 | RT1 class I, T24, gene 4 | CUST_4_PI197795813 | custom |
| 155 | RT1-T24-4 | 0.51 | 1.42 | 0.0939 | RT1 class I, T24, gene 4 | CUST_5_PI197795813 | custom |
| 156 | RT-BM1 (RT1- | 1.06 | 2.08 | 0.0416 | RT1 class I, RT-BM1 | A_44_P454420 | Agilent |
| | S3) | | | | | | |
| 157 | RT1-N3 | 0.20 | 1.15 | 0.3890 | RT1 class I, N3 | A_42_P521707 | Agilent |
| 158 | RT1-O1 | −0.03 | 0.98 | 0.8512 | RT1 class I, O1 | CUST_1_PI197795863 | custom |
| 158 | RT1-O1 | −0.05 | 0.97 | 0.6261 | RT1 class I, O1 | CUST_2_PI197795863 | custom |
| 158 | RT1-O1 | 0.08 | 1.06 | 0.3128 | RT1 class I, O1 | CUST_3_PI197795863 | custom |
| 158 | RT1-O1 | 0.01 | 1.01 | 0.8904 | RT1 class I, O1 | CUST_4_PI197795863 | custom |
| 158 | RT1-O1 | −0.15 | 0.90 | 0.3468 | RT1 class I, O1 | CUST_5_PI197795863 | custom |
| 159 | RT1-S2 | −0.31 | 0.81 | 0.2437 | RT1 class I, S2 | CUST_1_PI2010111700 | custom |
| 159 | RT1-S2 | −0.25 | 0.84 | 0.2765 | RT1 class I, S2 | CUST_5_PI2010111700 | custom |
| 160 | RT1-N2 | 0.06 | 1.04 | 0.7225 | RT1 class I, N2 | CUST_1_PI197795818 | custom |
| 160 | RT1-N2 | −0.02 | 0.99 | 0.9142 | RT1 class I, N2 | CUST_2_PI197795818 | custom |
| 160 | RT1-N2 | 0.09 | 1.06 | 0.6061 | RT1 class I, N2 | CUST_3_PI197795818 | custom |
| 160 | RT1-N2 | 0.01 | 1.01 | 0.9575 | RT1 class I, N2 | CUST_4_PI197795818 | custom |
| 160 | RT1-N2 | 0.01 | 1.01 | 0.9481 | RT1 class I, N2 | CUST_5_PI197795818 | custom |
| 160 | RT1-N2 | 0.02 | 1.01 | 0.9122 | RT1 class I, N2 | A_44_P379367 | Agilent |
| 161 | RT1-O2 | −0.38 | 0.77 | 0.2262 | RT1 class I, O2 | CUST_1_PI201011211 | custom |
| 161 | RT1-O2 | 0.57 | 1.48 | 0.0345 | RT1 class I, O2 | CUST_2_PI201011211 | custom |
| 161 | RT1-O2 | −0.09 | 0.94 | 0.6330 | RT1 class I, O2 | CUST_3_PI201011211 | custom |
| 161 | RT1-O2 | 0.55 | 1.46 | 0.0424 | RT1 class I, O2 | CUST_4_PI201011211 | custom |
| 161 | RT1-O2 | 0.22 | 1.16 | 0.3389 | RT1 class I, O2 | CUST_5_PI201011211 | custom |
| 162 | RT1-O3 | −0.30 | 0.81 | 0.2438 | RT1 class I, O3 | CUST_1_PI201011202 | custom |
| 162 | RT1-O3 | −0.13 | 0.91 | 0.5468 | RT1 class I, O3 | CUST_2_PI201011202 | custom |
| 162 | RT1-O3 | 0.50 | 1.41 | 0.0546 | RT1 class I, O3 | CUST_3_PI201011202 | custom |
| 162 | RT1-O3 | 0.50 | 1.41 | 0.0457 | RT1 class I, O3 | CUST_4_PI201011202 | custom |
| 162 | RT1-O3 | 0.23 | 1.17 | 0.2975 | RT1 class I, O3 | CUST_5_PI201011202 | custom |
| 163 | RT1-V1 | 0.10 | 1.07 | 0.5153 | RT1 class I, V1 | CUST_1_PI201011196 | custom |
| 163 | RT1-V1 | 0.05 | 1.04 | 0.6614 | RT1 class I, V1 | CUST_2_PI201011196 | custom |
| 163 | RT1-V1 | 0.03 | 1.02 | 0.8018 | RT1 class I, V1 | CUST_3_PI201011196 | custom |
| 163 | RT1-V1 | 0.03 | 1.02 | 0.7265 | RT1 class I, V1 | CUST_4_PI201011196 | custom |
| 163 | RT1-V1 | 0.11 | 1.08 | 0.3219 | RT1 class I, V1 | CUST_5_PI201011196 | custom |
| 164 | RT1-T18 | 0.12 | 1.09 | 0.5019 | histocompatibility 2, T region locus 18 | A_44_P358361 | Agilent |
| 164 | RT1-T18 | 0.67 | 1.59 | 0.0828 | histocompatibility 2, T region locus 18 | A_44_P358358 | Agilent |
| 165 | RT1-P1 | 0.42 | 1.34 | 0.1795 | RT1 class I, P1 | CUST_1_PI201011193 | custom |
| 165 | RT1-P1 | 0.43 | 1.35 | 0.1897 | RT1 class I, P1 | CUST_2_PI201011193 | custom |
| 165 | RT1-P1 | 0.33 | 1.26 | 0.3012 | RT1 class I, P1 | CUST_3_PI201011193 | custom |
| 165 | RT1-P1 | 0.38 | 1.30 | 0.2049 | RT1 class I, P1 | CUST_4_PI201011193 | custom |
| 165 | RT1-P1 | 0.31 | 1.24 | 0.2951 | RT1 class I, P1 | CUST_5_PI201011193 | custom |
| 166 | RT1-V2 | 0.02 | 1.01 | 0.8517 | RT1 class I, V2 | CUST_1_PI201011189 | custom |
| 166 | RT1-V2 | 0.07 | 1.05 | 0.3934 | RT1 class I, V2 | CUST_2_PI201011189 | custom |
| 166 | RT1-V2 | 0.01 | 1.01 | 0.9606 | RT1 class I, V2 | CUST_3_PI201011189 | custom |
| 166 | RT1-V2 | −0.01 | 0.99 | 0.9455 | RT1 class I, V2 | CUST_4_PI201011189 | custom |
| 166 | RT1-V2 | 0.06 | 1.04 | 0.6161 | RT1 class I, V2 | CUST_5_PI201011189 | custom |
| 167 | RT1-P2 | 0.14 | 1.10 | 0.2561 | RT1 class I, P2 | CUST_1_PI201011184 | custom |
| 167 | RT1-P2 | −0.01 | 0.99 | 0.9599 | RT1 class I, P2 | CUST_2_PI201011184 | custom |
| 167 | RT1-P2 | −0.03 | 0.98 | 0.7705 | RT1 class I, P2 | CUST_3_PI201011184 | custom |
| 167 | RT1-P2 | 0.01 | 1.01 | 0.9284 | RT1 class I, P2 | CUST_4_PI201011184 | custom |
| 167 | RT1-P2 | −0.03 | 0.98 | 0.8477 | RT1 class I, P2 | CUST_5_PI201011184 | custom |
| 168 | Flj22638 (Rpp21) | 0.10 | 1.07 | 0.6826 | ribonuclease P 21 subunit | A_44_P1017763 | Agilent |
| 168 | Flj22638 (Rpp21) | −0.02 | 0.99 | 0.8997 | ribonuclease P 21 subunit | A_44_P1017757 | Agilent |
| 169 | Trim39 | −0.32 | 0.80 | 0.1210 | tripartite motif-containing 39 | A_44_P245427 | Agilent |
| 170 | RT1-M10-1 | 0.02 | 1.01 | 0.8675 | RT1 class I, M10, gene 1 | CUST_1_PI201011161 | custom |
| 170 | RT1-M10-1 | −0.19 | 0.88 | 0.1707 | RT1 class I, M10, gene 1 | CUST_2_PI201011161 | custom |
| 170 | RT1-M10-1 | 0.03 | 1.02 | 0.7954 | RT1 class I, M10, gene 1 | CUST_3_PI201011161 | custom |
| 170 | RT1-M10-1 | −0.01 | 0.99 | 0.9161 | RT1 class I, M10, gene 1 | CUST_4_PI201011161 | custom |
| 170 | RT1-M10-1 | −0.04 | 0.97 | 0.6779 | RT1 class I, M10, gene 1 | CUST_5_PI201011161 | custom |
| 171 | RT1-M10-2 | −0.09 | 0.94 | 0.2987 | RT1 class I, M10, gene 2 | CUST_1_PI201011180 | custom |
| 171 | RT1-M10-2 | −0.06 | 0.96 | 0.6569 | RT1 class I, M10, gene 2 | CUST_2_PI201011180 | custom |
| 171 | RT1-M10-2 | −0.01 | 0.99 | 0.9375 | RT1 class I, M10, gene 2 | CUST_3_PI201011180 | custom |
| 171 | RT1-M10-2 | 0.03 | 1.02 | 0.7545 | RT1 class I, M10, gene 2 | CUST_4_PI201011180 | custom |
| 171 | RT1-M10-2 | −0.02 | 0.99 | 0.8053 | RT1 class I, M10, gene 2 | CUST_5_PI201011180 | custom |
| 172 | RT1-M1-1 | −0.01 | 0.99 | 0.9358 | RT1 class I, M1, gene 1 | CUST_1_PI201011178 | custom |
| 172 | RT1-M1-1 | −0.11 | 0.93 | 0.4445 | RT1 class I, M1, gene 1 | CUST_2_PI201011178 | custom |
| 172 | RT1-M1-1 | 0.54 | 1.45 | 0.0278 | RT1 class I, M1, gene 1 | CUST_3_PI201011178 | custom |
| 172 | RT1-M1-1 | −0.17 | 0.89 | 0.1632 | RT1 class I, M1, gene 1 | CUST_4_PI201011178 | custom |
| 172 | RT1-M1-1 | −0.05 | 0.97 | 0.7839 | RT1 class I, M1, gene 1 | CUST_5_PI201011178 | custom |
| 173 | RT1-M1-2 | −0.11 | 0.93 | 0.3479 | RT1 class I, M1, gene 2 | CUST_1_PI197795822 | custom |
| 173 | RT1-M1-2 | −0.22 | 0.86 | 0.1078 | RT1 class I, M1, gene 2 | CUST_2_PI197795822 | custom |
| 173 | RT1-M1-2 | −0.03 | 0.98 | 0.7000 | RT1 class I, M1, gene 2 | CUST_3_PI197795822 | custom |
| 173 | RT1-M1-2 | −0.02 | 0.99 | 0.8325 | RT1 class I, M1, gene 2 | CUST_4_PI197795822 | custom |
| 173 | RT1-M1-2 | 0.00 | 1.00 | 0.9910 | RT1 class I, M1, gene 2 | CUST_5_PI197795822 | custom |
| 174 | RT1-M1-3 | −0.10 | 0.93 | 0.2338 | RT1 class I, M1, gene 3 | CUST_1_PI201011175 | custom |
| 174 | RT1-M1-3 | −0.02 | 0.99 | 0.9164 | RT1 class I, M1, gene 3 | CUST_2_PI201011175 | custom |
| 174 | RT1-M1-3 | −0.01 | 0.99 | 0.9246 | RT1 class I, M1, gene 3 | CUST_3_PI201011175 | custom |
| 174 | RT1-M1-3 | 0.03 | 1.02 | 0.7901 | RT1 class I, M1, gene 3 | CUST_4_PI201011175 | custom |
| 174 | RT1-M1-3 | −0.09 | 0.94 | 0.2805 | RT1 class I, M1, gene 3 | CUST_5_PI201011175 | custom |
| 175 | RT1-M1-4 | −0.23 | 0.85 | 0.1261 | RT1 class I, M1, gene 4 | A_44_P213221 | Agilent |
| 176 | RT1-M1-5 | 0.04 | 1.03 | 0.7001 | RT1 class I, M1, gene 5 | A_44_P506413 | Agilent |
| 177 | RT1-M7 | −0.08 | 0.95 | 0.3109 | RT1 class I, M7 | CUST_1_PI201011173 | custom |
| 177 | RT1-M7 | −0.30 | 0.81 | 0.0433 | RT1 class I, M7 | CUST_2_PI201011173 | custom |
| 177 | RT1-M7 | 0.04 | 1.03 | 0.5727 | RT1 class I, M7 | CUST_3_PI201011173 | custom |
| 177 | RT1-M7 | −0.05 | 0.97 | 0.7154 | RT1 class I, M7 | CUST_4_PI201011173 | custom |
| 177 | RT1-M7 | −0.32 | 0.80 | 0.1162 | RT1 class I, M7 | CUST_5_PI201011173 | custom |
| 178 | RT1-M8 | −0.23 | 0.85 | 0.0654 | RT1 class I, M8 | CUST_1_PI201011170 | custom |
| 178 | RT1-M8 | −0.04 | 0.97 | 0.6168 | RT1 class I, M8 | CUST_2_PI201011170 | custom |
| 178 | RT1-M8 | −0.31 | 0.81 | 0.1655 | RT1 class I, M8 | CUST_3_PI201011170 | custom |
| 178 | RT1-M8 | −0.22 | 0.86 | 0.2766 | RT1 class I, M8 | CUST_5_PI201011170 | custom |
| 178 | RT1-M8 | 0.01 | 1.01 | 0.9933 | RT1 class I, M8 | CUST_4_PI201011170 | custom |
| 179 | RT1-M10-3 | −0.02 | 0.99 | 0.9071 | RT1 class I, M10, gene 3 | CUST_1_PI201011167 | custom |
| 179 | RT1-M10-3 | −0.27 | 0.83 | 0.0424 | RT1 class I, M10, gene 3 | CUST_2_PI201011167 | custom |
| 179 | RT1-M10-3 | −0.06 | 0.96 | 0.6730 | RT1 class I, M10, gene 3 | CUST_3_PI201011167 | custom |
| 179 | RT1-M10-3 | −0.04 | 0.97 | 0.6161 | RT1 class I, M10, gene 3 | CUST_4_PI201011167 | custom |
| 179 | RT1-M10-3 | −0.06 | 0.96 | 0.5878 | RT1 class I, M10, gene 3 | CUST_5_PI201011167 | custom |
| 180 | RT1-M10-4 | 0.08 | 1.06 | 0.4351 | RT1 class I, M10, gene 4 | CUST_1_PI197795820 | custom |
| 180 | RT1-M10-4 | 0.09 | 1.06 | 0.4057 | RT1 class I, M10, gene 4 | CUST_2_PI197795820 | custom |
| 180 | RT1-M10-4 | 0.26 | 1.20 | 0.3213 | RT1 class I, M10, gene 4 | CUST_3_PI197795820 | custom |
| 180 | RT1-M10-4 | −0.62 | 0.65 | 0.0539 | RT1 class I, M10, gene 4 | CUST_4_PI197795820 | custom |
| 180 | RT1-M10-4 | 0.07 | 1.05 | 0.6195 | RT1 class I, M10, gene 4 | CUST_5_PI197795820 | custom |
| 181 | Trim26 | −0.04 | 0.97 | 0.8676 | tripartite motif-containing 26 | CUST_1_PI197795824 | custom |
| 181 | Trim26 | −0.04 | 0.97 | 0.8113 | tripartite motif-containing 26 | CUST_2_PI197795824 | custom |
| 181 | Trim26 | −0.20 | 0.87 | 0.1379 | tripartite motif-containing 26 | CUST_3_PI197795824 | custom |
| 181 | Trim26 | 0.07 | 1.05 | 0.6779 | tripartite motif-containing 26 | CUST_4_PI197795824 | custom |
| 181 | Trim26 | −0.01 | 0.99 | 0.9756 | tripartite motif-containing 26 | CUST_5_PI197795824 | custom |
| 182 | Trim15 | −0.99 | 0.50 | 0.0539 | tripartite motif-containing 15 | CUST_1_PI201011159 | custom |
| 182 | Trim15 | −0.96 | 0.51 | 0.0722 | tripartite motif-containing 15 | CUST_2_PI201011159 | custom |
| 182 | Trim15 | −0.90 | 0.54 | 0.0858 | tripartite motif-containing 15 | CUST_3_PI201011159 | custom |
| 182 | Trim15 | −0.83 | 0.56 | 0.0654 | tripartite motif-containing 15 | CUST_4_PI201011159 | custom |
| 182 | Trim15 | −1.02 | 0.49 | 0.0603 | tripartite motif-containing 15 | CUST_5_PI201011159 | custom |
| 183 | Trim10 | −0.26 | 0.84 | 0.2418 | tripartite motif protein 10 | CUST_1_PI197795826 | custom |
| 183 | Trim10 | −0.18 | 0.88 | 0.5016 | tripartite motif protein 10 | CUST_2_PI197795826 | custom |
| 183 | Trim10 | −0.15 | 0.90 | 0.5471 | tripartite motif protein 10 | CUST_3_PI197795826 | custom |
| 183 | Trim10 | −0.26 | 0.84 | 0.2463 | tripartite motif protein 10 | CUST_4_PI197795826 | custom |
| 183 | Trim10 | −0.20 | 0.87 | 0.2290 | tripartite motif protein 10 | CUST_5_PI197795826 | custom |
| 184 | Trim40 | −0.22 | 0.86 | 0.0923 | tripartite motif-containing 40 | CUST_1_PI209196805 | custom |
| 184 | Trim40 | 0.00 | 1.00 | 0.9664 | tripartite motif-containing 40 | CUST_2_PI209196805 | custom |
| 184 | Trim40 | 0.08 | 1.06 | 0.5878 | tripartite motif-containing 40 | CUST_3_PI209196805 | custom |
| 184 | Trim40 | −0.06 | 0.96 | 0.6191 | tripartite motif-containing 40 | CUST_4_PI209196805 | custom |
| 184 | Trim40 | −0.08 | 0.95 | 0.4748 | tripartite motif-containing 40 | CUST_5_PI209196805 | custom |
| 185 | Trim31 | −0.04 | 0.97 | 0.8529 | tripartite motif-containing 31 | A_51_P490840 | Agilent |
| 185 | Trim31 | −0.04 | 0.97 | 0.7567 | tripartite motif-containing 31 | CUST_21_PI209196805 | custom |
| 185 | Trim31 | 0.00 | 1.00 | 0.9980 | tripartite motif-containing 31 | CUST_22_PI209196805 | custom |
| 185 | Trim31 | −0.06 | 0.96 | 0.5133 | tripartite motif-containing 31 | CUST_23_PI209196805 | custom |
| 185 | Trim31 | −0.16 | 0.90 | 0.1958 | tripartite motif-containing 31 | CUST_24_PI209196805 | custom |
| 185 | Trim31 | −0.04 | 0.97 | 0.7351 | tripartite motif-containing 31 | CUST_25_PI209196805 | custom |
| 186 | 1700031A10Rik | −0.14 | 0.91 | 0.4950 | gene corresponding to Riken clone 1700031A10 | A_52_P515192 | Agilent |
| 186 | 1700031A10Rik | −0.10 | 0.93 | 0.2703 | gene corresponding to Riken clone 1700031A10 | CUST_31_PI209196805 | custom |
| 186 | 1700031A10Rik | −0.13 | 0.91 | 0.3373 | gene corresponding to Riken clone 1700031A10 | CUST_32_PI209196805 | custom |
| 186 | 1700031A10Rik | −0.08 | 0.95 | 0.5598 | gene corresponding to Riken clone 1700031A10 | CUST_33_PI209196805 | custom |
| 186 | 1700031A10Rik | −0.02 | 0.99 | 0.9034 | gene corresponding to Riken clone 1700031A10 | CUST_34_PI209196805 | custom |
| 186 | 1700031A10Rik | −0.05 | 0.97 | 0.7178 | gene corresponding to Riken clone 1700031A10 | CUST_35_PI209196805 | custom |
| 187 | Rnf39 | 0.05 | 1.04 | 0.6605 | Ring finger protein Lirf | CUST_1_PI195698208 | custom |
| 187 | Rnf39 | 0.06 | 1.04 | 0.5793 | Ring finger protein Lirf | CUST_2_PI195698208 | custom |
| 187 | Rnf39 | −0.02 | 0.99 | 0.8552 | Ring finger protein Lirf | CUST_3_PI195698208 | custom |
| 187 | Rnf39 | −0.28 | 0.82 | 0.0579 | Ring finger protein Lirf | CUST_4_PI195698208 | custom |
| 187 | Rnf39 | −0.06 | 0.96 | 0.6264 | Ring finger protein Lirf | CUST_5_PI195698208 | custom |
| 188 | Ppp1r11 | 0.14 | 1.10 | 0.5417 | protein phosphatase 1, regulatory (inhibitor) subunit 11 | CUST_1_PI197795829 | custom |
| 188 | Ppp1r11 | 0.14 | 1.10 | 0.4917 | protein phosphatase 1, regulatory (inhibitor) subunit 11 | CUST_2_PI197795829 | custom |
| 188 | Ppp1r11 | 0.10 | 1.07 | 0.6213 | protein phosphatase 1, regulatory (inhibitor) subunit 11 | CUST_3_PI197795829 | custom |
| 188 | Ppp1r11 | 0.13 | 1.09 | 0.4615 | protein phosphatase 1, regulatory (inhibitor) subunit 11 | CUST_4_PI197795829 | custom |
| 188 | Ppp1r11 | 0.09 | 1.06 | 0.6711 | protein phosphatase 1, regulatory (inhibitor) subunit 11 | CUST_5_PI197795829 | custom |
| 189 | Znrd1 | 0.22 | 1.16 | 0.3879 | zinc ribbon domain containing, 1 | A_44_P404931 | Agilent |
| 190 | Tctex4 | −0.20 | 0.87 | 0.2497 | t-complex testis-expressed 4, rat homologue | CUST_1_PI201011154 | custom |
| 190 | Tctex4 | −0.10 | 0.93 | 0.6520 | t-complex testis-expressed 4, rat homologue | CUST_2_PI201011154 | custom |
| 190 | Tctex4 | −0.02 | 0.99 | 0.9728 | t-complex testis-expressed 4, rat homologue | CUST_3_PI201011154 | custom |
| 190 | Tctex4 | −0.14 | 0.91 | 0.7705 | t-complex testis-expressed 4, rat homologue | CUST_4_PI201011154 | custom |
| 190 | Tctex4 | −0.18 | 0.88 | 0.6959 | t-complex testis-expressed 4, rat homologue | CUST_5_PI201011154 | custom |
| 191 | RT1-M6-2 | 0.29 | 1.22 | 0.2232 | RT1 class I, M6, gene 2 | A_44_P309052 | Agilent |
| 192 | RT1-M6-1 | 0.25 | 1.19 | 0.1939 | RT1 class I, M6, gene 1 | CUST_1_PI197795831 | custom |
| 192 | RT1-M6-1 | 0.14 | 1.10 | 0.2419 | RT1 class I, M6, gene 1 | CUST_2_PI197795831 | custom |
| 192 | RT1-M6-1 | 0.09 | 1.06 | 0.5742 | RT1 class I, M6, gene 1 | CUST_3_PI197795831 | custom |
| 192 | RT1-M6-1 | 0.15 | 1.11 | 0.2707 | RT1 class I, M6, gene 1 | CUST_4_PI197795831 | custom |
| 192 | RT1-M6-1 | 0.13 | 1.09 | 0.5124 | RT1 class I, M6, gene 1 | CUST_5_PI197795831 | custom |
| 193 | RT1-M4 | −0.05 | 0.97 | 0.6379 | RT1 class I, M4 | A_44_P260445 | Agilent |
| 193 | RT1-M4 | −0.03 | 0.98 | 0.8888 | RT1 class I, M4 | CUST_1_PI201011151 | custom |
| 193 | RT1-M4 | 0.01 | 1.01 | 0.9694 | RT1 class I, M4 | CUST_2_PI201011151 | custom |
| 193 | RT1-M4 | 0.25 | 1.19 | 0.2536 | RT1 class I, M4 | CUST_3_PI201011151 | custom |
| 193 | RT1-M4 | −0.01 | 0.99 | 0.9413 | RT1 class I, M4 | CUST_4_PI201011151 | custom |
| 193 | RT1-M4 | −0.11 | 0.93 | 0.6425 | RT1 class I, M4 | CUST_5_PI201011151 | custom |
| 194 | RT1-M5 | −0.13 | 0.91 | 0.2545 | RT1 class Ib, locus M5 | CUST_1_PI197795834 | custom |
| 194 | RT1-M5 | −0.02 | 0.99 | 0.9122 | RT1 class Ib, locus M5 | CUST_2_PI197795834 | custom |
| 194 | RT1-M5 | −0.05 | 0.97 | 0.6483 | RT1 class Ib, locus M5 | CUST_3_PI197795834 | custom |
| 194 | RT1-M5 | 0.03 | 1.02 | 0.8395 | RT1 class Ib, locus M5 | CUST_4_PI197795834 | custom |
| 194 | RT1-M5 | −0.05 | 0.97 | 0.6199 | RT1 class Ib, locus M5 | CUST_5_PI197795834 | custom |
| 195 | Zfp57 | 0.13 | 1.09 | 0.6841 | zinc finger protein 57 | CUST_1_PI197795840 | custom |
| 195 | Zfp57 | −0.43 | 0.74 | 0.0681 | zinc finger protein 57 | CUST_2_PI197795840 | custom |
| 195 | Zfp57 | −0.40 | 0.76 | 0.0611 | zinc finger protein 57 | CUST_3_PI197795840 | custom |
| 195 | Zfp57 | −0.34 | 0.79 | 0.0401 | zinc finger protein 57 | CUST_4_PI197795840 | custom |
| 195 | Zfp57 | −0.29 | 0.82 | 0.0940 | zinc finger protein 57 | CUST_5_PI197795840 | custom |
| 196 | Mog | −0.26 | 0.84 | 0.1591 | myelin oligodendrocyte glycoprotein | A_43_P12283 | Agilent |
| 197 | Gabbr1 | −0.17 | 0.89 | 0.4183 | gamma-aminobutyric acid (GABA) B receptor 1 | A_43_P12481 | Agilent |
| 198 | 9430032L10Rik | 0.05 | 1.04 | 0.5965 | gene corresponding to Riken clone 9430032L10 | CUST_1_PI201011147 | custom |
| 198 | 9430032L10Rik | 0.02 | 1.01 | 0.8261 | gene corresponding to Riken clone 9430032L10 | CUST_2_PI201011147 | custom |
| 198 | 9430032L10Rik | 0.02 | 1.01 | 0.8425 | gene corresponding to Riken clone 9430032L10 | CUST_3_PI201011147 | custom |
| 198 | 9430032L10Rik | 0.04 | 1.03 | 0.8307 | gene corresponding to Riken clone 9430032L10 | CUST_4_PI201011147 | custom |
| 198 | 9430032L10Rik | −0.03 | 0.98 | 0.8685 | gene corresponding to Riken clone 9430032L10 | CUST_5_PI201011147 | custom |
| 199 | Or1 | −0.08 | 0.95 | 0.5471 | olfactory receptor 1750 (predicted) | A_52_P410245 | Agilent |
| 199 | Or1 | −0.02 | 0.99 | 0.8675 | olfactory receptor 1750 (predicted) | CUST_16_PI209196805 | custom |
| 199 | Or1 | −0.11 | 0.93 | 0.4597 | olfactory receptor 1750 (predicted) | CUST_17_PI209196805 | custom |
| 199 | Or1 | −0.02 | 0.99 | 0.9034 | olfactory receptor 1750 (predicted) | CUST_18_PI209196805 | custom |
| 199 | Or1 | −0.04 | 0.97 | 0.8090 | olfactory receptor 1750 (predicted) | CUST_19_PI209196805 | custom |
| 199 | Or1 | −0.05 | 0.97 | 0.7090 | olfactory receptor 1750 (predicted) | CUST_20_PI209196805 | custom |
| 200 | Or2 | −0.07 | 0.95 | 0.6299 | olfactory receptor 1749 (predicted) | CUST_1_PI197795848 | custom |
| 200 | Or2 | −0.08 | 0.95 | 0.3091 | olfactory receptor 1749 (predicted) | CUST_2_PI197795848 | custom |
| 200 | Or2 | 0.07 | 1.05 | 0.5007 | olfactory receptor 1749 (predicted) | CUST_3_PI197795848 | custom |
| 200 | Or2 | −0.05 | 0.97 | 0.6808 | olfactory receptor 1749 (predicted) | CUST_4_PI197795848 | custom |
| 200 | Or2 | −0.03 | 0.98 | 0.8117 | olfactory receptor 1749 (predicted) | CUST_5_PI197795848 | custom |
| 201 | Or3 | −0.11 | 0.93 | 0.4566 | olfactory receptor 1748 (predicted) | CUST_1_PI197795850 | custom |
| 201 | Or3 | −0.13 | 0.91 | 0.2329 | olfactory receptor 1748 (predicted) | CUST_2_PI197795850 | custom |
| 201 | Or3 | −0.17 | 0.89 | 0.1773 | olfactory receptor 1748 (predicted) | CUST_3_PI197795850 | custom |
| 201 | Or3 | −0.06 | 0.96 | 0.6310 | olfactory receptor 1748 (predicted) | CUST_4_PI197795850 | custom |
| 201 | Or3 | −0.27 | 0.83 | 0.1077 | olfactory receptor 1748 (predicted) | CUST_5_PI197795850 | custom |
| 202 | Or4 | −0.20 | 0.87 | 0.2322 | olfactory receptor 1747 (predicted) | CUST_1_PI201011143 | custom |
| 202 | Or4 | −0.01 | 0.99 | 0.9720 | olfactory receptor 1747 (predicted) | CUST_2_PI201011143 | custom |
| 202 | Or4 | −0.21 | 0.86 | 0.1923 | olfactory receptor 1747 (predicted) | CUST_3_PI201011143 | custom |
| 202 | Or4 | −0.18 | 0.88 | 0.2355 | olfactory receptor 1747 (predicted) | CUST_4_PI201011143 | custom |
| 202 | Or4 | −0.10 | 0.93 | 0.5972 | olfactory receptor 1747 (predicted) | CUST_5_PI201011143 | custom |
| 203 | Or5 | 0.10 | 1.07 | 0.4571 | olfactory receptor 1746 (predicted) | CUST_1_PI197795852 | custom |
| 203 | Or5 | 0.04 | 1.03 | 0.7809 | olfactory receptor 1746 (predicted) | CUST_2_PI197795852 | custom |
| 203 | Or5 | −0.03 | 0.98 | 0.8583 | olfactory receptor 1746 (predicted) | CUST_3_PI197795852 | custom |
| 203 | Or5 | 0.13 | 1.09 | 0.3836 | olfactory receptor 1746 (predicted) | CUST_4_PI197795852 | custom |
| 203 | Or5 | −0.02 | 0.99 | 0.9090 | olfactory receptor 1746 (predicted) | CUST_5_PI197795852 | custom |
| 204 | Ubd | 3.19 | 9.13 | 0.0345 | ubiquitin D | A_42_P602724 | Agilent |
| 205 | Or6 | 0.03 | 1.02 | 0.8855 | olfactory receptor 1745 (predicted) | CUST_1_PI201011139 | custom |
| 205 | Or6 | 0.14 | 1.10 | 0.2994 | olfactory receptor 1745 (predicted) | CUST_2_PI201011139 | custom |
| 205 | Or6 | −0.06 | 0.96 | 0.6676 | olfactory receptor 1745 (predicted) | CUST_3_PI201011139 | custom |
| 205 | Or6 | −0.09 | 0.94 | 0.8444 | olfactory receptor 1745 (predicted) | CUST_4_PI201011139 | custom |
| 205 | Or6 | −0.09 | 0.94 | 0.7413 | olfactory receptor 1745 (predicted) | CUST_5_PI201011139 | custom |
| 206 | Or7 | 0.14 | 1.10 | 0.2389 | olfactory receptor 1744 (predicted) | CUST_1_PI197795854 | custom |
| 206 | Or7 | 0.00 | 1.00 | 0.9829 | olfactory receptor 1744 (predicted) | CUST_2_PI197795854 | custom |
| 206 | Or7 | −0.09 | 0.94 | 0.6165 | olfactory receptor 1744 (predicted) | CUST_3_PI197795854 | custom |
| 206 | Or7 | 0.12 | 1.09 | 0.5423 | olfactory receptor 1744 (predicted) | CUST_4_PI197795854 | custom |
| 206 | Or7 | −0.07 | 0.95 | 0.4992 | olfactory receptor 1744 (predicted) | CUST_5_PI197795854 | custom |
| 207 | Or8 | 0.01 | 1.01 | 0.9701 | olfactory receptor 1743 (predicted) | CUST_1_PI197795856 | custom |
| 207 | Or8 | −0.10 | 0.93 | 0.5207 | olfactory receptor 1743 (predicted) | CUST_2_PI197795856 | custom |
| 207 | Or8 | −0.14 | 0.91 | 0.2325 | olfactory receptor 1743 (predicted) | CUST_3_PI197795856 | custom |
| 207 | Or8 | −0.10 | 0.93 | 0.4321 | olfactory receptor 1743 (predicted) | CUST_4_PI197795856 | custom |
| 207 | Or8 | −0.31 | 0.81 | 0.0310 | olfactory receptor 1743 (predicted) | CUST_5_PI197795856 | custom |
| 208 | Or9 | −0.02 | 0.99 | 0.8743 | olfactory receptor 1742 (predicted) | A_44_P365332 | Agilent |
| 208 | Or9 | 0.03 | 1.02 | 0.8131 | olfactory receptor 1742 (predicted) | CUST_1_PI197795876 | custom |
| 208 | Or9 | −0.19 | 0.88 | 0.1014 | olfactory receptor 1742 (predicted) | CUST_2_PI197795876 | custom |
| 208 | Or9 | −0.26 | 0.84 | 0.0623 | olfactory receptor 1742 (predicted) | CUST_3_PI197795876 | custom |
| 208 | Or9 | −0.04 | 0.97 | 0.6038 | olfactory receptor 1742 (predicted) | CUST_4_PI197795876 | custom |
| 208 | Or9 | −0.02 | 0.99 | 0.9014 | olfactory receptor 1742 (predicted) | CUST_5_PI197795876 | custom |
| 209 | RT1-M3-2 | −0.07 | 0.95 | 0.8384 | RT1 class Ib, locus M3 | CUST_1_PI201011135 | custom |
| 209 | RT1-M3-2 | −0.03 | 0.98 | 0.9327 | RT1 class Ib, locus M3 | CUST_2_PI201011135 | custom |
| 209 | RT1-M3-2 | −0.08 | 0.95 | 0.8425 | RT1 class Ib, locus M3 | CUST_3_PI201011135 | custom |
| 209 | RT1-M3-2 | −0.13 | 0.91 | 0.6541 | RT1 class Ib, locus M3 | CUST_4_PI201011135 | custom |
| 209 | RT1-M3-2 | −0.10 | 0.93 | 0.7667 | RT1 class Ib, locus M3 | CUST_5_PI201011135 | custom |
| 210 | Or10 | 0.07 | 1.05 | 0.6326 | olfactory receptor 1740 (predicted) | CUST_1_PI201011133 | custom |
| 210 | Or10 | −0.10 | 0.93 | 0.2049 | olfactory receptor 1740 (predicted) | CUST_2_PI201011133 | custom |
| 210 | Or10 | −0.09 | 0.94 | 0.5788 | olfactory receptor 1740 (predicted) | CUST_3_PI201011133 | custom |
| 210 | Or10 | −0.08 | 0.95 | 0.5345 | olfactory receptor 1740 (predicted) | CUST_4_PI201011133 | custom |
| 210 | Or10 | −0.10 | 0.93 | 0.2687 | olfactory receptor 1740 (predicted) | CUST_5_PI201011133 | custom |
| 211 | RT1-M3-1 | 0.24 | 1.18 | 0.4938 | RT1 class Ib, locus M3 | CUST_1_PI197795861 | custom |
| 211 | RT1-M3-1 | 0.27 | 1.21 | 0.5424 | RT1 class Ib, locus M3 | CUST_2_PI197795861 | custom |
| 211 | RT1-M3-1 | 0.25 | 1.19 | 0.5596 | RT1 class Ib, locus M3 | CUST_3_PI197795861 | custom |
| 211 | RT1-M3-1 | 0.03 | 1.02 | 0.9375 | RT1 class Ib, locus M3 | CUST_4_PI197795861 | custom |
| 211 | RT1-M3-1 | 0.14 | 1.10 | 0.7567 | RT1 class Ib, locus M3 | CUST_5_PI197795861 | custom |
| 212 | Or11 | 0.03 | 1.02 | 0.7761 | olfactory receptor 1739 (predicted) | A_44_P433163 | Agilent |
| 213 | Or12 | −0.07 | 0.95 | 0.6171 | olfactory receptor 1738 (predicted) | CUST_1_PI197795865 | custom |
| 213 | Or12 | −0.21 | 0.86 | 0.1188 | olfactory receptor 1738 (predicted) | CUST_2_PI197795865 | custom |
| 213 | Or12 | −0.19 | 0.88 | 0.1498 | olfactory receptor 1738 (predicted) | CUST_3_PI197795865 | custom |
| 213 | Or12 | 0.02 | 1.01 | 0.9088 | olfactory receptor 1738 (predicted) | CUST_4_PI197795865 | custom |
| 213 | Or12 | 0.06 | 1.04 | 0.6232 | olfactory receptor 1738 (predicted) | CUST_5_PI197795865 | custom |
| 214 | Or13 | −0.01 | 0.99 | 0.9278 | olfactory receptor 1737 (predicted) | CUST_1_PI197795867 | custom |
| 214 | Or13 | −0.38 | 0.77 | 0.0345 | olfactory receptor 1737 (predicted) | CUST_2_PI197795867 | custom |
| 214 | Or13 | −0.07 | 0.95 | 0.6831 | olfactory receptor 1737 (predicted) | CUST_3_PI197795867 | custom |
| 214 | Or13 | 0.06 | 1.04 | 0.6537 | olfactory receptor 1737 (predicted) | CUST_4_PI197795867 | custom |
| 214 | Or13 | 0.02 | 1.01 | 0.8695 | olfactory receptor 1737 (predicted) | CUST_5_PI197795867 | custom |
| 215 | Or14 | 0.04 | 1.03 | 0.6686 | olfactory receptor 1736 (predicted) | CUST_1_PI197795870 | custom |
| 215 | Or14 | 0.00 | 1.00 | 0.9849 | olfactory receptor 1736 (predicted) | CUST_2_PI197795870 | custom |
| 215 | Or14 | 0.01 | 1.01 | 0.9194 | olfactory receptor 1736 (predicted) | CUST_3_PI197795870 | custom |
| 215 | Or14 | −0.26 | 0.84 | 0.2740 | olfactory receptor 1736 (predicted) | CUST_4_PI197795870 | custom |
| 215 | Or14 | −0.14 | 0.91 | 0.1027 | olfactory receptor 1736 (predicted) | CUST_5_PI197795870 | custom |
| 216 | Or15 | −0.07 | 0.95 | 0.3931 | olfactory receptor 1735 (predicted) | CUST_1_PI197795872 | custom |
| 216 | Or15 | −0.14 | 0.91 | 0.2867 | olfactory receptor 1735 (predicted) | CUST_2_PI197795872 | custom |
| 216 | Or15 | 0.00 | 1.00 | 0.9952 | olfactory receptor 1735 (predicted) | CUST_3_PI197795872 | custom |
| 216 | Or15 | −0.08 | 0.95 | 0.6808 | olfactory receptor 1735 (predicted) | CUST_4_PI197795872 | custom |
| 216 | Or15 | −0.07 | 0.95 | 0.5993 | olfactory receptor 1735 (predicted) | CUST_5_PI197795872 | custom |
| 217 | Or27 | −0.04 | 0.97 | 0.8286 | olfactory receptor 1716 (predicted) | CUST_1_PI201011130 | custom |
| 217 | Or27 | −0.09 | 0.94 | 0.4929 | olfactory receptor 1716 (predicted) | CUST_2_PI201011130 | custom |
| 217 | Or27 | −0.01 | 0.99 | 0.9401 | olfactory receptor 1716 (predicted) | CUST_3_PI201011130 | custom |
| 217 | Or27 | −0.04 | 0.97 | 0.6989 | olfactory receptor 1716 (predicted) | CUST_4_PI201011130 | custom |
| 217 | Or27 | −0.07 | 0.95 | 0.6330 | olfactory receptor 1716 (predicted) | CUST_5_PI201011130 | custom |
| 218 | Or26 | −0.07 | 0.95 | 0.4471 | olfactory receptor 1718 (predicted) | A_44_P505752 | Agilent |
| 219 | Or28 | −0.05 | 0.97 | 0.5892 | olfactory receptor 1714 (predicted) | CUST_1_PI197795859 | custom |
| 219 | Or28 | −0.24 | 0.85 | 0.0490 | olfactory receptor 1714 (predicted) | CUST_2_PI197795859 | custom |
| 219 | Or28 | −0.01 | 0.99 | 0.9454 | olfactory receptor 1714 (predicted) | CUST_3_PI197795859 | custom |
| 219 | Or28 | −0.02 | 0.99 | 0.8444 | olfactory receptor 1714 (predicted) | CUST_4_PI197795859 | custom |
| 219 | Or28 | −0.03 | 0.98 | 0.8464 | olfactory receptor 1714 (predicted) | CUST_5_PI197795859 | custom |
| 220 | RT1-M3-3 | −0.12 | 0.92 | 0.3297 | RT1 class Ib, locus M3 | CUST_1_PI201011128 | custom |
| 220 | RT1-M3-3 | −0.06 | 0.96 | 0.6580 | RT1 class Ib, locus M3 | CUST_2_PI201011128 | custom |
| 220 | RT1-M3-3 | −0.08 | 0.95 | 0.3186 | RT1 class Ib, locus M3 | CUST_3_PI201011128 | custom |
| 220 | RT1-M3-3 | −0.12 | 0.92 | 0.3465 | RT1 class Ib, locus M3 | CUST_4_PI201011128 | custom |
| 220 | RT1-M3-3 | −0.18 | 0.88 | 0.3305 | RT1 class Ib, locus M3 | CUST_5_PI201011128 | custom |
| 222 | Or29 | −0.02 | 0.99 | 0.8250 | olfactory receptor 29 | A_44_P411999 | Agilent |
| 223 | RT1-M2 | 0.04 | 1.03 | 0.6219 | RT1 class Ib, locus M2 | A_44_P154023 | Agilent |
| 224 | Or30 | 0.03 | 1.02 | 0.7708 | olfactory receptor 1730 (predicted) | CUST_1_PI197795878 | custom |
| 224 | Or30 | −0.06 | 0.96 | 0.5708 | olfactory receptor 1730 (predicted) | CUST_2_PI197795878 | custom |
| 224 | Or30 | 0.00 | 1.00 | 0.9771 | olfactory receptor 1730 (predicted) | CUST_3_PI197795878 | custom |
| 224 | Or30 | −0.36 | 0.78 | 0.0940 | olfactory receptor 1730 (predicted) | CUST_4_PI197795878 | custom |
| 224 | Or30 | 0.05 | 1.04 | 0.7708 | olfactory receptor 1730 (predicted) | CUST_5_PI197795878 | custom |

TABLE 5b

| Gene | | log2-Fold | Fold | adj. P- | | | Probe |
|---|---|---|---|---|---|---|---|
| order | Gene Symbol | Change | Change | value | Gene Description | Probe ID | Design |
| 16 | RT1-A1 | 0.70 | 1.62 | 0.0149 | RT1 class I | CUST_1_PI202535318 | custom |
| 16 | RT1-A1 | 0.75 | 1.68 | 0.0100 | RT1 class I | CUST_2_PI202535318 | custom |
| 16 | RT1-A1 | 0.80 | 1.74 | 0.0149 | RT1 class I | CUST_3_PI202535318 | custom |
| 16 | RT1-A1 | 0.86 | 1.82 | 0.0100 | RT1 class I | CUST_4_PI202535318 | custom |
| 16 | RT1-A1 | 0.91 | 1.88 | 0.0100 | RT1 class I | CUST_5_PI202535318 | custom |
| 17 | RT1-A2 | 0.98 | 1.97 | 0.0100 | RT1 class I | A_44_P296155 | Agilent |
| 29 | RT1-DMb | 2.59 | 6.02 | 0.0100 | major histocompatibility complex, class II, DM beta | CUST_1_PI195698203 | custom |
| 29 | RT1-DMb | 2.77 | 6.82 | 0.0100 | major histocompatibility complex, class II, DM beta | CUST_2_PI195698203 | custom |
| 29 | RT1-DMb | 1.93 | 3.81 | 0.0149 | major histocompatibility complex, class II, DM beta | CUST_3_PI195698203 | custom |
| 29 | RT1-DMb | 1.87 | 3.66 | 0.0149 | major histocompatibility complex, class II, DM beta | CUST_4_PH95698203 | custom |
| 29 | RT1-DMb | 1.94 | 3.84 | 0.0100 | major histocompatibility complex, class II, DM beta | CUST_5_PI195698203 | custom |
| 31 | Tap1 | 0.53 | 1.44 | 0.1159 | transporter 1, ATP-binding cassette, sub-family B | A_43_P15763 | Agilent |
| | | | | | (MDR/TAP) | | |
| 31 | Tap1 | 0.63 | 1.55 | 0.0390 | transporter 1, ATP-binding cassette, sub-family B | A_44_P451916 | Agilent |
| | | | | | (MDR/TAP) | | |
| 32 | Psmb8 | 1.00 | 2.00 | 0.0336 | proteasome (prosome, macropain) subunit, beta type 8 | A_42_P761035 | Agilent |
| | | | | | (large multi-functional peptidase 7) | | |
| 51 | G18 (Gpsm3) | 1.23 | 2.35 | 0.0315 | G18 protein | A_42_P569708 | Agilent |
| 52 | Pbx2 | 0.33 | 1.26 | 0.0466 | pre-B-cell leukemia transcription factor 2 | A_42_P592157 | Agilent |
| 54 | Rnf5 | 0.57 | 1.48 | 0.0315 | ring finger protein 5 | A_51_P204582 | Agilent |
| 54 | Rnf5 | 0.26 | 1.20 | 0.0674 | ring finger protein 5 | CUST_1_PI207500742 | custom |
| 54 | Rnf5 | 0.21 | 1.16 | 0.1445 | ring finger protein 5 | CUST_2_PI207500742 | custom |
| 54 | Rnf5 | 0.17 | 1.13 | 0.2905 | ring finger protein 5 | CUST_3_PI207500742 | custom |
| 54 | Rnf5 | 0.22 | 1.16 | 0.1626 | ring finger protein 5 | CUST_4_PI207500742 | custom |
| 54 | Rnf5 | 0.19 | 1.14 | 0.1707 | ring finger protein 5 | CUST_5_PI207500742 | custom |
| 69 | C2 | 1.22 | 2.33 | 0.0325 | complement component 2 | A_44_P332606 | Agilent |
| 88 | Ly6g6e | −1.38 | 0.38 | 0.0416 | lymphocyte antigen 6 complex, locus G6E | CUST_1_PI195698246 | custom |
| 88 | Ly6g6e | −1.42 | 0.37 | 0.0523 | lymphocyte antigen 6 complex, locus G6E | CUST_2_PI195698246 | custom |
| 88 | Ly6g6e | −1.39 | 0.38 | 0.0623 | lymphocyte antigen 6 complex, locus G6E | CUST_3_PI195698246 | custom |
| 88 | Ly6g6e | −1.44 | 0.37 | 0.0416 | lymphocyte antigen 6 complex, locus G6E | CUST_4_PI195698246 | custom |
| 88 | Ly6g6e | −1.46 | 0.36 | 0.0433 | lymphocyte antigen 6 complex, locus G6E | CUST_5_PI195698246 | custom |
| 90 | Bat5 | −0.60 | 0.66 | 0.0100 | HLA-B associated transcript 5 | CUST_1_PI195830595 | custom |
| 90 | Bat5 | −0.48 | 0.72 | 0.0100 | HLA-B associated transcript 5 | CUST_2_PI195830595 | custom |
| 90 | Bat5 | −0.54 | 0.69 | 0.0180 | HLA-B associated transcript 5 | CUST_3_PI195830595 | custom |
| 90 | Bat5 | −0.53 | 0.69 | 0.0229 | HLA-B associated transcript 5 | CUST_4_PI195830595 | custom |
| 90 | Bat5 | −0.58 | 0.67 | 0.0100 | HLA-B associated transcript 5 | CUST_5_PI195830595 | custom |
| 100 | Aif1 | 2.83 | 7.11 | 0.0100 | allograft inflammatory factor 1 | A_44_P421534 | Agilent |
| 102 | Lst1 | 3.32 | 9.99 | 0.0100 | leucocyte specific transcript 1 | A_43_P12274 | Agilent |
| 110 | RT1-CE2 | 0.64 | 1.56 | 0.0278 | RT1 class I, CE2 | A_44_P107372 | Agilent |
| 111 | RT1-CE3 | 0.96 | 1.95 | 0.0100 | RT1 class I, CE3 | A_44_P274061 | Agilent |
| 113 | RT1-CE5 | 0.70 | 1.62 | 0.0395 | RT1 class I, CE5 | A_44_P172850 | Agilent |
| 116 | RT1-CE8 | 0.90 | 1.87 | 0.0278 | RT1 class I, CE8 | CUST_1_PI201011245 | custom |
| 116 | RT1-CE8 | 0.91 | 1.88 | 0.0100 | RT1 class I, CE8 | CUST_2_PI201011245 | custom |
| 116 | RT1-CE8 | 0.78 | 1.72 | 0.0229 | RT1 class I, CE8 | CUST_3_PI201011245 | custom |
| 116 | RT1-CE8 | 0.84 | 1.79 | 0.0100 | RT1 class I, CE8 | CUST_4_PI201011245 | custom |
| 116 | RT1-CE8 | 0.79 | 1.73 | 0.0149 | RT1 class I, CE8 | CUST_5_PI201011245 | custom |
| 117 | RT1-CE9 | 0.80 | 1.74 | 0.0315 | RT1 class I, CE9 | CUST_1_PI201011241 | custom |
| 117 | RT1-CE9 | 0.35 | 1.27 | 0.1745 | RT1 class I, CE9 | CUST_2_PI201011241 | custom |
| 117 | RT1-CE9 | 0.74 | 1.67 | 0.0539 | RT1 class I, CE9 | CUST_3_PI201011241 | custom |
| 117 | RT1-CE9 | 0.24 | 1.18 | 0.3698 | RT1 class I, CE9 | CUST_4_PI201011241 | custom |
| 117 | RT1-CE9 | 0.81 | 1.75 | 0.0373 | RT1 class I, CE9 | CUST_5_PI201011241 | custom |
| 118 | RT1-CE10 | 4.09 | 17.03 | 0.0100 | RT1 class I, CE10 | A_44_P389019 | Agilent |
| 119 | RT1-CE11 | 0.28 | 1.21 | 0.2867 | RT1 class I, CE11 | CUST_1_PI195941302 | custom |
| 119 | RT1-CE11 | 0.65 | 1.57 | 0.0315 | RT1 class I, CE11 | CUST_2_PI195941302 | custom |
| 119 | RT1-CE11 | 0.22 | 1.16 | 0.2638 | RT1 class I, CE11 | CUST_3_PI195941302 | custom |
| 119 | RT1-CE11 | 0.16 | 1.12 | 0.3957 | RT1 class I, CE11 | CUST_4_PI195941302 | custom |
| 119 | RT1-CE11 | 0.38 | 1.30 | 0.0980 | RT1 class I, CE11 | CUST_5_PI195941302 | custom |
| 120 | RT1-CE12 | 0.43 | 1.35 | 0.1710 | RT1 class I, CE12 | CUST_1_PI195941305 | custom |
| 120 | RT1-CE12 | −0.10 | 0.93 | 0.4503 | RT1 class I, CE12 | CUST_2_PI195941305 | custom |
| 120 | RT1-CE12 | 0.34 | 1.27 | 0.1043 | RT1 class I, CE12 | CUST_3_PI195941305 | custom |
| 120 | RT1-CE12 | 0.04 | 1.03 | 0.8574 | RT1 class I, CE12 | CUST_4_PH95941305 | custom |
| 120 | RT1-CE12 | 0.56 | 1.47 | 0.0310 | RT1 class I, CE12 | CUST_5_PI195941305 | custom |
| 124 | RT1-CE16 | 0.54 | 1.45 | 0.0325 | RT1 class I, CE16 (RT1 class Ib, locus Cl) | A_44_P867246 | Agilent |
| 124 | RT1-CE16 | 0.78 | 1.72 | 0.0206 | RT1 class I, CE16 (RT1 class Ib, locus Cl) | A_44_P554925 | Agilent |
| 128 | Spr1 | 1.26 | 2.39 | 0.0206 | psoriasis susceptibility 1 candidate 2 (human) | A_66_P100662 | Agilent |
| 128 | Spr1 | 1.39 | 2.62 | 0.0180 | psoriasis susceptibility 1 candidate 2 (human) | A_51_P212958 | Agilent |
| 128 | Spr1 | 1.36 | 2.57 | 0.0206 | psoriasis susceptibility 1 candidate 2 (human) | A_51_P212956 | Agilent |
| 128 | Spr1 | 1.50 | 2.83 | 0.0100 | psoriasis susceptibility 1 candidate 2 (human) | CUST_56_PI209196805 | custom |
| 128 | Spr1 | 1.52 | 2.87 | 0.0100 | psoriasis susceptibility 1 candidate 2 (human) | CUST_57_PI209196805 | custom |
| 128 | Spr1 | 1.51 | 2.85 | 0.0100 | psoriasis susceptibility 1 candidate 2 (human) | CUST_58_PI209196805 | custom |
| 128 | Spr1 | 1.50 | 2.83 | 0.0100 | psoriasis susceptibility 1 candidate 2 (human) | CUST_59_PI209196805 | custom |
| 128 | Spr1 | 1.58 | 2.99 | 0.0100 | psoriasis susceptibility 1 candidate 2 (human) | CUST_60_PI209196805 | custom |
| 129 | Cdsn | 0.37 | 1.29 | 0.2732 | corneodesmosin | CUST_1_PI201011238 | custom |
| 129 | Cdsn | 0.84 | 1.79 | 0.0100 | corneodesmosin | CUST_2_PI201011238 | custom |
| 129 | Cdsn | 0.38 | 1.30 | 0.2184 | corneodesmosin | CUST_3_PI201011238 | custom |
| 129 | Cdsn | 0.32 | 1.25 | 0.3754 | corneodesmosin | CUST_4_PI201011238 | custom |
| 129 | Cdsn | 0.40 | 1.32 | 0.1769 | corneodesmosin | CUST_5_PI201011238 | custom |
| 138 | Ier3 | 0.87 | 1.83 | 0.0229 | immediate early response 3 | A_42_P515405 | Agilent |
| 143 | Kiaa1949 | 0.42 | 1.34 | 0.0481 | KIAA1949 protein | CUST_1_PI201011218 | custom |
| 143 | Kiaa1949 | 0.49 | 1.40 | 0.0457 | KIAA1949 protein | CUST_2_PI201011218 | custom |
| 143 | Kiaa1949 | 0.33 | 1.26 | 0.1378 | KIAA1949 protein | CUST_3_PI201011218 | custom |
| 143 | Kiaa1949 | 0.39 | 1.31 | 0.0993 | KIAA1949 protein | CUST_4_PI201011218 | custom |
| 143 | Kiaa1949 | 0.34 | 1.27 | 0.1184 | KIAA1949 protein | CUST_5_PI201011218 | custom |
| 146 | Flj13158 | −0.25 | 0.84 | 0.0832 | hypothetical protein FLJ13158 | A_44_P278509 | Agilent |
| | (RGD1303066) | | | | | | |
| 146 | Flj13158 | −0.57 | 0.67 | 0.0378 | hypothetical protein FLJ13158 | A_44_P654250 | Agilent |
| | (RGD1303066) | | | | | | |
| 147 | Mrps18b | 0.52 | 1.43 | 0.0474 | mitochondrial ribosomal protein S18B | CUST_1_PI197795811 | custom |
| 147 | Mrps18b | 0.49 | 1.40 | 0.0378 | mitochondrial ribosomal protein S18B | CUST_2_PI197795811 | custom |
| 147 | Mrps18b | 0.57 | 1.48 | 0.0267 | mitochondrial ribosomal protein S18B | CUST_3_PI197795811 | custom |
| 147 | Mrps18b | 0.59 | 1.51 | 0.0365 | mitochondrial ribosomal protein S18B | CUST_4_PI197795811 | custom |
| 147 | Mrps18b | 0.62 | 1.54 | 0.0254 | mitochondrial ribosomal protein S18B | CUST_5_PI197795811 | custom |
| 154 | RT1-T24-3 | 0.31 | 1.24 | 0.1540 | RT1 class I, T24, gene 3 | CUST_1_PI201011214 | custom |
| 154 | RT1-T24-3 | 0.42 | 1.34 | 0.0336 | RT1 class I, T24, gene 3 | CUST_2_PI201011214 | custom |
| 154 | RT1-T24-3 | 0.27 | 1.21 | 0.1454 | RT1 class I, T24, gene 3 | CUST_3_PI201011214 | custom |
| 154 | RT1-T24-3 | 0.31 | 1.24 | 0.0847 | RT1 class I, T24, gene 3 | CUST_4_PI201011214 | custom |
| 154 | RT1-T24-3 | 0.08 | 1.06 | 0.6030 | RT1 class I, T24, gene 3 | CUST_5_PI201011214 | custom |
| 155 | RT1-T24-4 | 0.57 | 1.48 | 0.0345 | RT1 class I, T24, gene 4 | CUST_1_PI197795813 | custom |
| 155 | RT1-T24-4 | 0.76 | 1.69 | 0.0206 | RT1 class I, T24, gene 4 | CUST_2_PI197795813 | custom |
| 155 | RT1-T24-4 | 0.72 | 1.65 | 0.0206 | RT1 class I, T24, gene 4 | CUST_3_PI197795813 | custom |
| 155 | RT1-T24-4 | 0.39 | 1.31 | 0.0611 | RT1 class I, T24, gene 4 | CUST_4_PI197795813 | custom |
| 155 | RT1-T24-4 | 0.51 | 1.42 | 0.0939 | RT1 class I, T24, gene 4 | CUST_5_PI197795813 | custom |
| 156 | RT-BM1 (RT1- | 1.06 | 2.08 | 0.0416 | RT1 class I, RT-BM1 | A_44_P454420 | Agilent |
| | S3) | | | | | | |
| 161 | RT1-O2 | −0.38 | 0.77 | 0.2262 | RT1 class I, O2 | CUST_1_PI201011211 | custom |
| 161 | RT1-O2 | 0.57 | 1.48 | 0.0345 | RT1 class I, O2 | CUST_2_PI201011211 | custom |
| 161 | RT1-O2 | −0.09 | 0.94 | 0.6330 | RT1 class I, O2 | CUST_3_PI201011211 | custom |
| 161 | RT1-O2 | 0.55 | 1.46 | 0.0424 | RT1 class I, O2 | CUST_4_PI201011211 | custom |
| 161 | RT1-O2 | 0.22 | 1.16 | 0.3389 | RT1 class I, O2 | CUST_5_PI201011211 | custom |
| 162 | RT1-O3 | −0.30 | 0.81 | 0.2438 | RT1 class I, O3 | CUST_1_PI201011202 | custom |
| 162 | RT1-O3 | −0.13 | 0.91 | 0.5468 | RT1 class I, O3 | CUST_2_PI201011202 | custom |
| 162 | RT1-O3 | 0.50 | 1.41 | 0.0546 | RT1 class I, O3 | CUST_3_PI201011202 | custom |
| 162 | RT1-O3 | 0.50 | 1.41 | 0.0457 | RT1 class I, O3 | CUST_4_PI201011202 | custom |
| 162 | RT1-O3 | 0.23 | 1.17 | 0.2975 | RT1 class I, O3 | CUST_5_PI201011202 | custom |
| 172 | RT1-M1-1 | −0.01 | 0.99 | 0.9358 | RT1 class I, M1, gene 1 | CUST_1_PI201011178 | custom |
| 172 | RT1-M1-1 | −0.11 | 0.93 | 0.4445 | RT1 class I, M1, gene 1 | CUST_2_PI201011178 | custom |
| 172 | RT1-M1-1 | 0.54 | 1.45 | 0.0278 | RT1 class I, M1, gene 1 | CUST_3_PI201011178 | custom |
| 172 | RT1-M1-1 | −0.17 | 0.89 | 0.1632 | RT1 class I, M1, gene 1 | CUST_4_PI201011178 | custom |
| 172 | RT1-M1-1 | −0.05 | 0.97 | 0.7839 | RT1 class I, M1, gene 1 | CUST_5_PI201011178 | custom |
| 177 | RT1-M7 | −0.08 | 0.95 | 0.3109 | RT1 class I, M7 | CUST_1_PI201011173 | custom |
| 177 | RT1-M7 | −0.30 | 0.81 | 0.0433 | RT1 class I, M7 | CUST_2_PI201011173 | custom |
| 177 | RT1-M7 | 0.04 | 1.03 | 0.5727 | RT1 class I, M7 | CUST_3_PI201011173 | custom |
| 177 | RT1-M7 | −0.05 | 0.97 | 0.7154 | RT1 class I, M7 | CUST_4_PI201011173 | custom |
| 177 | RT1-M7 | −0.32 | 0.80 | 0.1162 | RT1 class I, M7 | CUST_5_PI201011173 | custom |
| 179 | RT1-M10-3 | −0.02 | 0.99 | 0.9071 | RT1 class I, M10, gene 3 | CUST_1_PI201011167 | custom |
| 179 | RT1-M10-3 | −0.27 | 0.83 | 0.0424 | RT1 class I, M10, gene 3 | CUST_2_PI201011167 | custom |
| 179 | RT1-M10-3 | −0.06 | 0.96 | 0.6730 | RT1 class I, M10, gene 3 | CUST_3_PI201011167 | custom |
| 179 | RT1-M10-3 | −0.04 | 0.97 | 0.6161 | RT1 class I, M10, gene 3 | CUST_4_PI201011167 | custom |
| 179 | RT1-M10-3 | −0.06 | 0.96 | 0.5878 | RT1 class I, M10, gene 3 | CUST_5_PI201011167 | custom |
| 195 | Zfp57 | 0.13 | 1.09 | 0.6841 | zinc finger protein 57 | CUST_1_PI197795840 | custom |
| 195 | Zfp57 | −0.43 | 0.74 | 0.0681 | zinc finger protein 57 | CUST_2_PI197795840 | custom |
| 195 | Zfp57 | −0.40 | 0.76 | 0.0611 | zinc finger protein 57 | CUST_3_PI197795840 | custom |
| 195 | Zfp57 | −0.34 | 0.79 | 0.0401 | zinc finger protein 57 | CUST_4_PI197795840 | custom |
| 195 | Zfp57 | −0.29 | 0.82 | 0.0940 | zinc finger protein 57 | CUST_5_PI197795840 | custom |
| 204 | Ubd | 3.19 | 9.13 | 0.0345 | ubiquitin D | A_42_P602724 | Agilent |
| 207 | Or8 | 0.01 | 1.01 | 0.9701 | olfactory receptor 1743 (predicted) | CUST_1_PI197795856 | custom |
| 207 | Or8 | −0.10 | 0.93 | 0.5207 | olfactory receptor 1743 (predicted) | CUST_2_PI197795856 | custom |
| 207 | Or8 | −0.14 | 0.91 | 0.2325 | olfactory receptor 1743 (predicted) | CUST_3_PI197795856 | custom |
| 207 | Or8 | −0.10 | 0.93 | 0.4321 | olfactory receptor 1743 (predicted) | CUST_4_PI197795856 | custom |
| 207 | Or8 | −0.31 | 0.81 | 0.0310 | olfactory receptor 1743 (predicted) | CUST_5_PI197795856 | custom |
| 214 | Or13 | −0.01 | 0.99 | 0.9278 | olfactory receptor 1737 (predicted) | CUST_1_PI197795867 | custom |
| 214 | Or13 | −0.38 | 0.77 | 0.0345 | olfactory receptor 1737 (predicted) | CUST_2_PI197795867 | custom |
| 214 | Or13 | −0.07 | 0.95 | 0.6831 | olfactory receptor 1737 (predicted) | CUST_3_PI197795867 | custom |
| 214 | Or13 | 0.06 | 1.04 | 0.6537 | olfactory receptor 1737 (predicted) | CUST_4_PI197795867 | custom |
| 214 | Or13 | 0.02 | 1.01 | 0.8695 | olfactory receptor 1737 (predicted) | CUST_5_PI197795867 | custom |
| 219 | Or28 | −0.05 | 0.97 | 0.5892 | olfactory receptor 1714 (predicted) | CUST_1_PI197795859 | custom |
| 219 | Or28 | −0.24 | 0.85 | 0.0490 | olfactory receptor 1714 (predicted) | CUST_2_PI197795859 | custom |
| 219 | Or28 | −0.01 | 0.99 | 0.9454 | olfactory receptor 1714 (predicted) | CUST_3_PI197795859 | custom |
| 219 | Or28 | −0.02 | 0.99 | 0.8444 | olfactory receptor 1714 (predicted) | CUST_4_PI197795859 | custom |
| 219 | Or28 | −0.03 | 0.98 | 0.8464 | olfactory receptor 1714 (predicted) | CUST_5_PI197795859 | custom |

TABLE 5c

| Gene | | log2-Fold | Fold | | | | Probe |
|---|---|---|---|---|---|---|---|
| order | Gene Symbol | Change | Change | adj. P-value | Gene Description | Probe ID | Design |
| 16 | RT1-A1 | 0.70 | 1.62 | 0.0149 | RT1 class I | CUST_1_PI202535318 | custom |
| 16 | RT1-A1 | 0.75 | 1.68 | 0.0100 | RT1 class I | CUST_2_PI202535318 | custom |
| 16 | RT1-A1 | 0.80 | 1.74 | 0.0149 | RT1 class I | CUST_3_PI202535318 | custom |
| 16 | RT1-A1 | 0.86 | 1.82 | 0.0100 | RT1 class I | CUST_4_PI202535318 | custom |
| 16 | RT1-A1 | 0.91 | 1.88 | 0.0100 | RT1 class I | CUST_5_PI202535318 | custom |
| 17 | RT1-A2 | 0.98 | 1.97 | 0.0100 | RT1 class I | A_44_P296155 | Agilent |
| 29 | RT1-DMb | 2.59 | 6.02 | 0.0100 | major histocompatibility complex, class II, DM beta | CUST_1_PI195698203 | custom |
| 29 | RT1-DMb | 2.77 | 6.82 | 0.0100 | major histocompatibility complex, class II, DM beta | CUST_2_PI195698203 | custom |
| 29 | RT1-DMb | 1.93 | 3.81 | 0.0149 | major histocompatibility complex, class II, DM beta | CUST_3_PI195698203 | custom |
| 29 | RT1-DMb | 1.87 | 3.66 | 0.0149 | major histocompatibility complex, class II, DM beta | CUST_4_PI195698203 | custom |
| 29 | RT1-DMb | 1.94 | 3.84 | 0.0100 | major histocompatibility complex, class II, DM beta | CUST_5_PI195698203 | custom |
| 31 | Tap1 | 0.63 | 1.55 | 0.0390 | transporter 1, ATP-binding cassette, sub-family B | A_44_P451916 | Agilent |
| | | | | | (MDR/TAP) | | |
| 32 | Psmb8 | 1.00 | 2.00 | 0.0336 | proteasome (prosome, macropain) subunit, | A_42_P761035 | Agilent |
| | | | | | beta type 8 (large multi-functional peptidase 7) | | |
| 51 | G18 (Gpsm3) | 1.23 | 2.35 | 0.0315 | G18 protein | A_42_P569708 | Agilent |
| 52 | Pbx2 | 0.33 | 1.26 | 0.0466 | pre-B-cell leukemia transcription factor 2 | A_42_P592157 | Agilent |
| 69 | C2 | 1.22 | 2.33 | 0.0325 | complement component 2 | A_44_P332606 | Agilent |
| 88 | Ly6g6e | −1.38 | 0.38 | 0.0416 | lymphocyte antigen 6 complex, locus G6E | CUST_1_PI195698246 | custom |
| 88 | Ly6g6e | −1.44 | 0.37 | 0.0416 | lymphocyte antigen 6 complex, locus G6E | CUST_4_PI195698246 | custom |
| 88 | Ly6g6e | −1.46 | 0.36 | 0.0433 | lymphocyte antigen 6 complex, locus G6E | CUST_5_PI195698246 | custom |
| 90 | Bat5 | −0.60 | 0.66 | 0.0100 | HLA-B associated transcript 5 | CUST_1_PI195830595 | custom |
| 90 | Bat5 | −0.48 | 0.72 | 0.0100 | HLA-B associated transcript 5 | CUST_2_PI195830595 | custom |
| 90 | Bat5 | −0.54 | 0.69 | 0.0180 | HLA-B associated transcript 5 | CUST_3_PI195830595 | custom |
| 90 | Bat5 | −0.53 | 0.69 | 0.0229 | HLA-B associated transcript 5 | CUST_4_PI195830595 | custom |
| 90 | Bat5 | −0.58 | 0.67 | 0.0100 | HLA-B associated transcript 5 | CUST_5_PI195830595 | custom |
| 100 | Aif1 | 2.83 | 7.11 | 0.0100 | allograft inflammatory factor 1 | A_44_P421534 | Agilent |
| 102 | Lst1 | 3.32 | 9.99 | 0.0100 | leucocyte specific transcript 1 | A_43_P12274 | Agilent |
| 110 | RT1-CE2 | 0.64 | 1.56 | 0.0278 | RT1 class I, CE2 | A_44_P107372 | Agilent |
| 111 | RT1-CE3 | 0.96 | 1.95 | 0.0100 | RT1 class I, CE3 | A_44_P274061 | Agilent |
| 113 | RT1-CE5 | 0.70 | 1.62 | 0.0395 | RT1 class I, CE5 | A_44_P172850 | Agilent |
| 116 | RT1-CE8 | 0.90 | 1.87 | 0.0278 | RT1 class I, CE8 | CUST_1_PI201011245 | custom |
| 116 | RT1-CE8 | 0.91 | 1.88 | 0.0100 | RT1 class I, CE8 | CUST_2_PI201011245 | custom |
| 116 | RT1-CE8 | 0.78 | 1.72 | 0.0229 | RT1 class I, CE8 | CUST_3_PI201011245 | custom |
| 116 | RT1-CE8 | 0.84 | 1.79 | 0.0100 | RT1 class I, CE8 | CUST_4_PI201011245 | custom |
| 116 | RT1-CE8 | 0.79 | 1.73 | 0.0149 | RT1 class I, CE8 | CUST_5_PI201011245 | custom |
| 118 | RT1-CE10 | 4.09 | 17.03 | 0.0100 | RT1 class I, CE10 | A_44_P389019 | Agilent |
| 124 | RT1-CE16 | 0.54 | 1.45 | 0.0325 | RT1 class I, CE16 (RT1 class Ib, locus Cl) | A_44_P867246 | Agilent |
| 124 | RT1-CE16 | 0.78 | 1.72 | 0.0206 | RT1 class I, CE16 (RT1 class Ib, locus Cl) | A_44_P554925 | Agilent |
| 128 | Spr1 | 1.26 | 2.39 | 0.0206 | psoriasis susceptibility 1 candidate 2 (human) | A_66_P100662 | Agilent |
| 128 | Spr1 | 1.39 | 2.62 | 0.0180 | psoriasis susceptibility 1 candidate 2 (human) | A_51_P212958 | Agilent |
| 128 | Spr1 | 1.36 | 2.57 | 0.0206 | psoriasis susceptibility 1 candidate 2 (human) | A_51_P212956 | Agilent |
| 128 | Spr1 | 1.50 | 2.83 | 0.0100 | psoriasis susceptibility 1 candidate 2 (human) | CUST_56_PI209196805 | custom |
| 128 | Spr1 | 1.52 | 2.87 | 0.0100 | psoriasis susceptibility 1 candidate 2 (human) | CUST_57_PI209196805 | custom |
| 128 | Spr1 | 1.51 | 2.85 | 0.0100 | psoriasis susceptibility 1 candidate 2 (human) | CUST_58_PI209196805 | custom |
| 128 | Spr1 | 1.50 | 2.83 | 0.0100 | psoriasis susceptibility 1 candidate 2 (human) | CUST_59_PI209196805 | custom |
| 128 | Spr1 | 1.58 | 2.99 | 0.0100 | psoriasis susceptibility 1 candidate 2 (human) | CUST_60_PI209196805 | custom |
| 138 | Ier3 | 0.87 | 1.83 | 0.0229 | immediate early response 3 | A_42_P515405 | Agilent |
| 146 | Flj13158 | −0.57 | 0.67 | 0.0378 | hypothetical protein FLJ13158 | A_44_P654250 | Agilent |
| | (RGD1303066) | | | | | | |
| 147 | Mrps18b | 0.52 | 1.43 | 0.0474 | mitochondrial ribosomal protein S18B | CUST_1_PI197795811 | custom |
| 147 | Mrps18b | 0.49 | 1.40 | 0.0378 | mitochondrial ribosomal protein S18B | CUST_2_PI197795811 | custom |
| 147 | Mrps18b | 0.57 | 1.48 | 0.0267 | mitochondrial ribosomal protein S18B | CUST_3_PI197795811 | custom |
| 147 | Mrps18b | 0.59 | 1.51 | 0.0365 | mitochondrial ribosomal protein S18B | CUST_4_PI197795811 | custom |
| 147 | Mrps18b | 0.62 | 1.54 | 0.0254 | mitochondrial ribosomal protein S18B | CUST_5_PI197795811 | custom |
| 155 | RT1-T24-4 | 0.57 | 1.48 | 0.0345 | RT1 class I, T24, gene 4 | CUST_1_PI197795813 | custom |
| 155 | RT1-T24-4 | 0.76 | 1.69 | 0.0206 | RT1 class I, T24, gene 4 | CUST_2_PI197795813 | custom |
| 155 | RT1-T24-4 | 0.72 | 1.65 | 0.0206 | RT1 class I, T24, gene 4 | CUST_3_PI197795813 | custom |
| 156 | RT-BM1 (RT1- | 1.06 | 2.08 | 0.0416 | RT1 class I, RT-BM1 | A_44_P454420 | Agilent |
| | S3) | | | | | | |
| 204 | Ubd | 3.19 | 9.13 | 0.0345 | ubiquitin D | A_42_P602724 | Agilent |

Table 6. Expression Profiling Results of NKC Genes

In Table 6a, results for all 43 NKC genes investigated are indicated in their chromosomal order (Klrg; Pzp to Csda). The expression profiling results of BN skin explant samples exposed to pre-stimulated allogeneic (PVG) lymphocytes in comparison to those exposed to syngeneic (BN) lymphocytes are given. The log 2-fold changes and the fold changes in gene expression are shown for every oligonucleotide probe used. The adjusted p-values are indicated. Significant change is defined by p<0.05 and strong change is defined by log 2-fold change ≧1 or ≦−1; i.e. fold change ≧2 or ≦0.5. In addition, the identification numbers of the probes on the arrays are given (probe ID) together with the information whether these probes were taken from the Agilent database or custom designed. Table 6b contains the information for all NKC genes for which at least one probe indicted a significant alteration of gene expression. In Table 6c, the data for those genes are summarized that are considered to be regulated significantly because either at least a single probe indicated a significant (p<0.05) and strong (log 2-fold change ≧1 or ≦−1) regulation or at least 50% of the probes indicated a significant (p<0.05) regulation of gene expression.

TABLE 6a

| Gene | | log2-Fold | Fold | adj. P- | | | Probe |
|---|---|---|---|---|---|---|---|
| order | Gene Symbol | Change | Change | value | Gene Description | Probe ID | Design |
| 1 | Klrg1 | −0.05 | 0.97 | 0.7388 | Rattus norvegicus killer cell lectin-like | A_43_P15509 | Agilent |
| | | | | | receptor subfamily G, member 1 (Klrg1) | | |
| 2 | Pzp | 0.20 | 1.15 | 0.1307 | pregnancy zone protein | A_44_P214900 | Agilent |
| 3 | RGD1565709 | nt | | | similar to ovostatin-2 | | |
| 4 | Klrb1a | 0.27 | 1.21 | 0.1808 | killer cell lectin-like receptor subfamily B | A_42_P598304 | Agilent |
| | | | | | member 1a | | |
| 5 | Klrb1b | −0.08 | 0.95 | 0.7253 | killer cell lectin-like receptor subfamily B | A_44_P210547 | Agilent |
| | | | | | member 1B | | |
| 6 | LOC500331 | −0.10 | 0.93 | 0.5986 | Rattus norvegicus similar to osteoclast | A_44_P311870 | Agilent |
| | | | | | inhibitory lectin (LOC500331) | | |
| 7 | RGD1562831 | −0.10 | 0.93 | 0.5986 | similar to osteoclast inhibitory lectin | A_44_P311870 | Agilent |
| 8 | LOC689757 | nt | | | similar to osteoclast inhibitory lectin | | |
| | (Clec2d3) | | | | | | |
| 9 | LOC689770 | nt | | | similar to osteoclast inhibitory lectin | | |
| | (Clr4, Clec2d4) | | | | | | |
| 10 | Clec2d (Ocil) | −0.33 | 0.80 | 0.3029 | C-type lectin domain family 2, | A_44_P137003 | Agilent |
| | | | | | member D (osteoclast inhibitory lectin) | | |
| 11 | Cle2dl1 | nt | | | C-type lectin domain family 2 member d-like 1 | | |
| 12 | LOC689800 | −0.02 | 0.99 | 0.9178 | similar to osteoclast inhibitory lectin | A_44_P391750 | Agilent |
| 13 | Klrb1f | nt | | | killer cell lectin-like receptor subfamily B | | |
| | | | | | member 1F | | |
| 14 | Clec2h | nt | | | C-type lectin domain family 2, member h | | |
| 15 | Clec2e | nt | | | C-type lectin domain family 2, member E | | |
| 16 | RGD1563148 | −0.02 | 0.99 | 0.9178 | similar to osteoclast inhibitory lectin | A_44_P391750 | Agilent |
| | (Clrb, Clec2d11) | | | | | | |
| 17 | Cd69 | 0.69 | 1.61 | 0.1845 | CD69 antigen | A_43_P16166 | Agilent |
| 18 | RGD1564770 | nt | | | similar to CD69 antigen | | |
| | | | | | (p60, early T-cell activation antigen) | | |
| 19 | Clec12b | nt | | | C-type lectin domain family 12, member B | | |
| 20 | Clec1b | 0.93 | 1.91 | 0.0500 | C-type lectin domain family 1, member b | A_44_P869774 | Agilent |
| 21 | Clec9a | nt | | | C-type lectin domain family 9, member a | | |
| 22 | Clec1a | nt | | | C-type lectin domain family 1, member a | | |
| 23 | Clec7a | nt | | | C-type lectin domain family 7, member a | | |
| 24 | Olr1 | 1.41 | 2.66 | 0.0390 | oxidized low density lipoprotein (lectin-like) | A_44_P377266 | Agilent |
| | | | | | receptor 1 | | |
| 25 | LOC689963 | nt | | | hypothetical protein LOC689963 | | |
| 26 | Gabarapl1 | 0.26 | 1.20 | 0.2049 | gamma-aminobutyric acid (GABA(A)) | CUST_6_PI209816013 | custom |
| | | | | | receptor-associated protein-like 1 | | |
| 26 | Gabarapl1 | 0.21 | 1.16 | 0.3468 | gamma-aminobutyric acid (GABA(A)) | CUST_7_PI209816013 | custom |
| | | | | | receptor-associated protein-like 1 | | |
| 26 | Gabarapl1 | 0.18 | 1.13 | 0.4143 | gamma-aminobutyric acid (GABA(A)) | CUST_8_PI209816013 | custom |
| | | | | | receptor-associated protein-like 1 | | |
| 26 | Gabarapl1 | 0.12 | 1.09 | 0.5487 | gamma-aminobutyric acid (GABA(A)) | CUST_9_PI209816013 | custom |
| | | | | | receptor-associated protein-like 1 | | |
| 26 | Gabarapl1 | 0.19 | 1.14 | 0.2042 | gamma-aminobutyric acid (GABA(A)) | CUST_10_PI209816013 | custom |
| | | | | | receptor-associated protein-like 1 | | |
| 27 | Klre1 | −0.17 | 0.89 | 0.2053 | killer cell lectin-like receptor family E member 1 | A_44_P536089 | Agilent |
| 27 | Klre1 | −0.12 | 0.92 | 0.6467 | killer cell lectin-like receptor family E member 1 | A_43_P16744 | Agilent |
| 28 | Klrd1 | 0.21 | 1.16 | 0.3290 | killer cell lectin-like receptor, subfamily D, | A_43_P11543 | Agilent |
| | | | | | member 1, CD94 | | |
| 29 | Klrk1 | −0.02 | 0.99 | 0.9332 | killer cell lectin-like receptor subfamily K, | A_43_P13194 | Agilent |
| | | | | | member 1, NKG2D | | |
| 30 | Klrc3 | −0.13 | 0.91 | 0.6020 | killer cell lectin-like receptor subfamily C member 3 | A_44_P255149 | Agilent |
| 31 | Klrc2 | 0.00 | 1.00 | 0.9796 | killer cell lectin-like receptor subfamily C, | A_43_P11997 | Agilent |
| | | | | | member 2 | | |
| 32 | Klrc1 | −0.13 | 0.91 | 0.6020 | killer cell lectin-like receptor subfamily C | A_44_P2551491 | Agilent |
| | | | | | member 1, NKG2A | | |
| 33 | Klri1 | −0.18 | 0.88 | 0.3321 | killer cell lectin-like receptor family I member 1 | CUST_46_PI209816013 | custom |
| 33 | Klri1 | −0.14 | 0.91 | 0.4180 | killer cell lectin-like receptor family I member 1 | CUST_47_PI209816013 | custom |
| 33 | Klri1 | 0.16 | 1.12 | 0.5780 | killer cell lectin-like receptor family I member 1 | CUST_48_PI209816013 | custom |
| 33 | Klri1 | −0.11 | 0.93 | 0.5043 | killer cell lectin-like receptor family I member 1 | CUST_49_PI209816013 | custom |
| 33 | Klri1 | −0.16 | 0.90 | 0.3331 | killer cell lectin-like receptor family I member 1 | CUST_50_PI209816013 | custom |
| 34 | Klri2 | −0.12 | 0.92 | 0.2434 | killer cell lectin-like receptor family I member 2 | A_44_P590906 | Agilent |
| 35 | Klrh1 | −0.08 | 0.95 | 0.6380 | killer cell lectin-like receptor subfamily H, member 1 | A_43_P13373 | Agilent |
| 36 | LOC690020 | nt | | | similar to killer cell lectin-like receptor, | | |
| | | | | | subfamily A, member 17 | | |
| 37 | LOC690045 | 5.56 | 47.18 | 0.0100 | similar to immunoreceptor Ly49si1 | A_43_P10690 | Agilent |
| 38 | Ly49si3 | 3.67 | 12.73 | 0.0180 | immunoreceptor Ly49si3 | CUST_21_PI209816013 | custom |
| 38 | Ly49si3 | 4.82 | 28.25 | 0.0100 | immunoreceptor Ly49si3 | CUST_22_PI209816013 | custom |
| 38 | Ly49si3 | 2.23 | 4.69 | 0.0310 | immunoreceptor Ly49si3 | CUST_23_PI209816013 | custom |
| 38 | Ly49si3 | 1.22 | 2.33 | 0.0411 | immunoreceptor Ly49si3 | CUST_24_PI209816013 | custom |
| 38 | Ly49si3 | 1.79 | 3.46 | 0.0365 | immunoreceptor Ly49si3 | CUST_25_PI209816013 | custom |
| 39 | RGD1561306 | nt | | | similar to immunoreceptor Ly49si3 | | |
| 40 | Ly49si1 | 1.82 | 3.53 | 0.0517 | immunoreceptor Ly49si1 | CUST_56_PI209816013 | custom |
| 40 | Ly49si1 | 2.71 | 6.54 | 0.0325 | immunoreceptor Ly49si1 | CUST_57_PI209816013 | custom |
| 40 | Ly49si1 | 2.18 | 4.53 | 0.0362 | immunoreceptor Ly49si1 | CUST_58_PI209816013 | custom |
| 40 | Ly49si1 | 5.79 | 55.33 | 0.0100 | immunoreceptor Ly49si1 | CUST_59_PI209816013 | custom |
| 40 | Ly49si1 | 4.67 | 25.46 | 0.0100 | immunoreceptor Ly49si1 | CUST_60_PI209816013 | custom |
| 41 | RGD1563110 | nt | | | similar to immunoreceptor Ly49si3 | | |
| 42 | Ly49si2 | 3.64 | 12.47 | 0.0180 | immunoreceptor Ly49si2 | CUST_36_PI209816013 | custom |
| 42 | Ly49si2 | 4.60 | 24.25 | 0.0100 | immunoreceptor Ly49si2 | CUST_37_PI209816013 | custom |
| 42 | Ly49si2 | 4.44 | 21.71 | 0.0100 | immunoreceptor Ly49si2 | CUST_38_PI209816013 | custom |
| 42 | Ly49si2 | 1.76 | 3.39 | 0.0310 | immunoreceptor Ly49si2 | CUST_39_PI209816013 | custom |
| 42 | Ly49si2 | 1.67 | 3.18 | 0.0373 | immunoreceptor Ly49si2 | CUST_40_PI209816013 | custom |
| 43 | LOC690097 | nt | | | similar to immunoreceptor Ly49si3 | | |
| 44 | LOC502907 | nt | | | similar to immunoreceptor Ly49si1 | | |
| 45 | Ly49i9 | 5.27 | 38.59 | 0.0100 | Ly49 inhibitory receptor 9 | CUST_66_PI209816013 | custom |
| 45 | Ly49i9 | 5.13 | 35.02 | 0.0100 | Ly49 inhibitory receptor 9 | CUST_67_PI209816013 | custom |
| 45 | Ly49i9 | 5.15 | 35.51 | 0.0100 | Ly49 inhibitory receptor 9 | CUST_68_PI209816013 | custom |
| 45 | Ly49i9 | 5.29 | 39.12 | 0.0100 | Ly49 inhibitory receptor 9 | CUST_69_PI209816013 | custom |
| 45 | Ly49i9 | 6.60 | 97.01 | 0.0100 | Ly49 inhibitory receptor 9 | CUST_70_PI209816013 | custom |
| 46 | Ly49s5 | 0.01 | 1.01 | 0.9723 | Ly49 stimulatory receptor 5 | CUST_41_PI209816013 | custom |
| 46 | Ly49s5 | −0.07 | 0.95 | 0.5774 | Ly49 stimulatory receptor 5 | CUST_42_PI209816013 | custom |
| 46 | Ly49s5 | −0.11 | 0.93 | 0.3468 | Ly49 stimulatory receptor 5 | CUST_43_PI209816013 | custom |
| 46 | Ly49s5 | −0.03 | 0.98 | 0.8268 | Ly49 stimulatory receptor 5 | CUST_44_PI209816013 | custom |
| 46 | Ly49s5 | −0.08 | 0.95 | 0.5682 | Ly49 stimulatory receptor 5 | CUST_45_PI209816013 | custom |
| 47 | Ly49i5 | −0.06 | 0.96 | 0.8065 | Ly49 inhibitory receptor 5 | CUST_76_PI209816013 | custom |
| 47 | Ly49i5 | 0.07 | 1.05 | 0.5957 | Ly49 inhibitory receptor 5 | CUST_77_PI209816013 | custom |
| 47 | Ly49i5 | −0.01 | 0.99 | 0.9703 | Ly49 inhibitory receptor 5 | CUST_78_PI209816013 | custom |
| 47 | Ly49i5 | 0.00 | 1.00 | 0.9905 | Ly49 inhibitory receptor 5 | CUST_79_PI209816013 | custom |
| 47 | Ly49i5 | 0.05 | 1.04 | 0.6808 | Ly49 inhibitory receptor 5 | CUST_80_PI209816013 | custom |
| 48 | Klra22 | −0.04 | 0.97 | 0.7497 | killer cell lectin-like receptor subfamily A, | A_44_P266817 | Agilent |
| | | | | | member 22 | | |
| 49 | Ly49s6 | 0.19 | 1.14 | 0.1938 | Ly49 stimulatory receptor 6 | CUST_26_PI209816013 | custom |
| 49 | Ly49s6 | −0.01 | 0.99 | 0.9561 | Ly49 stimulatory receptor 6 | CUST_27_PI209816013 | custom |
| 49 | Ly49s6 | 0.02 | 1.01 | 0.9047 | Ly49 stimulatory receptor 6 | CUST_28_PI209816013 | custom |
| 49 | Ly49s6 | −0.18 | 0.88 | 0.2611 | Ly49 stimulatory receptor 6 | CUST_29_PI209816013 | custom |
| 49 | Ly49s6 | −0.14 | 0.91 | 0.3529 | Ly49 stimulatory receptor 6 | CUST_30_PI209816013 | custom |
| 50 | Ly49s4 | 0.15 | 1.11 | 0.2267 | Ly49 stimulatory receptor 4 | CUST_61_PI209816013 | custom |
| 50 | Ly49s4 | 0.09 | 1.06 | 0.2799 | Ly49 stimulatory receptor 4 | CUST_62_PI209816013 | custom |
| 50 | Ly49s4 | 0.06 | 1.04 | 0.3468 | Ly49 stimulatory receptor 4 | CUST_63_PI209816013 | custom |
| 50 | Ly49s4 | −0.03 | 0.98 | 0.7923 | Ly49 stimulatory receptor 4 | CUST_64_PI209816013 | custom |
| 50 | Ly49s4 | −0.07 | 0.95 | 0.5814 | Ly49 stimulatory receptor 4 | CUST_65_PI209816013 | custom |
| 51 | Ly49s3 | 0.09 | 1.06 | 0.4744 | Ly-49 stimulatory receptor 3 | A_44_P111662 | Agilent |
| 52 | Ly49i4 | −0.01 | 0.99 | 0.9090 | Ly49 inhibitory receptor 4 | A_44_P250375 | Agilent |
| 53 | Ly49i3 | −0.14 | 0.91 | 0.3217 | Ly49 inhibitory receptor 3 | CUST_81_PI209816013 | custom |
| 53 | Ly49i3 | 0.14 | 1.10 | 0.1803 | Ly49 inhibitory receptor 3 | CUST_82_PI209816013 | custom |
| 53 | Ly49i3 | 1.30 | 2.46 | 0.0325 | Ly49 inhibitory receptor 3 | CUST_84_PI209816013 | custom |
| 53 | Ly49i3 | 3.06 | 8.34 | 0.0180 | Ly49 inhibitory receptor 3 | CUST_85_PI209816013 | custom |
| 53 | Ly49i3 | 0.01 | 1.01 | 0.9333 | Ly49 inhibitory receptor 3 | CUST_83_PI209816013 | custom |
| 54 | Ly49i2 | 0.03 | 1.02 | 0.8446 | Ly49 inhibitory receptor 2 | A_44_P360539 | Agilent |
| 55 | Ly49i6 | 0.05 | 1.04 | 0.7829 | Ly49 inhibitory receptor 6 | CUST_71_PI209816013 | custom |
| 55 | Ly49i6 | 0.18 | 1.13 | 0.1258 | Ly49 inhibitory receptor 6 | CUST_72_PI209816013 | custom |
| 55 | Ly49i6 | 0.14 | 1.10 | 0.4065 | Ly49 inhibitory receptor 6 | CUST_73_PI209816013 | custom |
| 55 | Ly49i6 | −0.07 | 0.95 | 0.6385 | Ly49 inhibitory receptor 6 | CUST_74_PI209816013 | custom |
| 55 | Ly49i6 | 0.05 | 1.04 | 0.7708 | Ly49 inhibitory receptor 6 | CUST_75_PI209816013 | custom |
| 56 | Ly49s8 | 0.01 | 1.01 | 0.9448 | Ly49 stimulatory receptor 8 | CUST_11_PI209816013 | custom |
| 56 | Ly49s8 | 0.69 | 1.61 | 0.0755 | Ly49 stimulatory receptor 8 | CUST_12_PI209816013 | custom |
| 56 | Ly49s8 | 1.12 | 2.17 | 0.1471 | Ly49 stimulatory receptor 8 | CUST_13_PI209816013 | custom |
| 56 | Ly49s8 | 0.55 | 1.46 | 0.0733 | Ly49 stimulatory receptor 8 | CUST_14_PI209816013 | custom |
| 56 | Ly49s8 | 0.66 | 1.58 | 0.1253 | Ly49 stimulatory receptor 8 | CUST_15_PI209816013 | custom |
| 57 | Ly49s7 | 0.12 | 1.09 | 0.3177 | Ly49 stimulatory receptor 7 | A_44_P118897 | Agilent |
| 58 | Klra5 | 1.27 | 2.41 | 0.0984 | killer cell lectin-like receptor, subfamily A, | CUST_1_PI209816013 | custom |
| | | | | | member 5 | | |
| 58 | Klra5 | 0.46 | 1.38 | 0.2146 | killer cell lectin-like receptor, subfamily A, | CUST_2_PI209816013 | custom |
| | | | | | member 5 | | |
| 58 | Klra5 | 1.00 | 2.00 | 0.0940 | killer cell lectin-like receptor, subfamily A, | CUST_3_PI209816013 | custom |
| | | | | | member 5 | | |
| 58 | Klra5 | 0.99 | 1.99 | 0.0844 | killer cell lectin-like receptor, subfamily A, | CUST_4_PI209816013 | custom |
| | | | | | member 5 | | |
| 58 | Klra5 | 0.90 | 1.87 | 0.0845 | killer cell lectin-like receptor, subfamily A, | CUST_5_PI209816013 | custom |
| | | | | | member 5 | | |
| 59 | Ly49i7 | 0.67 | 1.59 | 0.0395 | immunoreceptor Ly49i7 | A_44_P821875 | Agilent |
| 60 | Ly49i8 | 0.07 | 1.05 | 0.5972 | immunoreceptor Ly49i8 | A_44_P652293 | Agilent |
| 61 | LOC690303 | nt | | | similar to mago-nashi homolog | | |
| 62 | Styk1 | nt | | | serine/threonine/tyrosine kinase 1 | | |
| 63 | Csda | −0.50 | 0.71 | 0.09 | cold shock domain protein A | A_42_P631493 | Agilent |

TABLE 6b

| Gene | | log2-Fold | Fold | adj. P- | | | Probe |
|---|---|---|---|---|---|---|---|
| order | Gene Symbol | Change | Change | value | Gene Description | Probe ID | Design |
| 24 | Olr1 | 1.41 | 2.66 | 0.0390 | oxidized low density lipoprotein (lectin-like) receptor 1 | A_44_P377266 | Agilent |
| 37 | LOC690045 | 5.56 | 47.18 | 0.0100 | similar to immunoreceptor Ly49si1 | A_43_P10690 | Agilent |
| 38 | Ly49si3 | 3.67 | 12.73 | 0.0180 | immunoreceptor Ly49si3 | CUST_21_PI209816013 | custom |
| 38 | Ly49si3 | 4.82 | 28.25 | 0.0100 | immunoreceptor Ly49si3 | CUST_22_PI209816013 | custom |
| 38 | Ly49si3 | 2.23 | 4.69 | 0.0310 | immunoreceptor Ly49si3 | CUST_23_PI209816013 | custom |
| 38 | Ly49si3 | 1.22 | 2.33 | 0.0411 | immunoreceptor Ly49si3 | CUST_24_PI209816013 | custom |
| 38 | Ly49si3 | 1.79 | 3.46 | 0.0365 | immunoreceptor Ly49si3 | CUST_25_PI209816013 | custom |
| 40 | Ly49si1 | 1.82 | 3.53 | 0.0517 | immunoreceptor Ly49si1 | CUST_56_PI209816013 | custom |
| 40 | Ly49si1 | 2.71 | 6.54 | 0.0325 | immunoreceptor Ly49si1 | CUST_57_PI209816013 | custom |
| 40 | Ly49si1 | 2.18 | 4.53 | 0.0362 | immunoreceptor Ly49si1 | CUST_58_PI209816013 | custom |
| 40 | Ly49si1 | 5.79 | 55.33 | 0.0100 | immunoreceptor Ly49si1 | CUST_59_PI209816013 | custom |
| 40 | Ly49si1 | 4.67 | 25.46 | 0.0100 | immunoreceptor Ly49si1 | CUST_60_PI209816013 | custom |
| 42 | Ly49si2 | 3.64 | 12.47 | 0.0180 | immunoreceptor Ly49si2 | CUST_36_PI209816013 | custom |
| 42 | Ly49si2 | 4.60 | 24.25 | 0.0100 | immunoreceptor Ly49si2 | CUST_37_PI209816013 | custom |
| 42 | Ly49si2 | 4.44 | 21.71 | 0.0100 | immunoreceptor Ly49si2 | CUST_38_PI209816013 | custom |
| 42 | Ly49si2 | 1.76 | 3.39 | 0.0310 | immunoreceptor Ly49si2 | CUST_39_PI209816013 | custom |
| 42 | Ly49si2 | 1.67 | 3.18 | 0.0373 | immunoreceptor Ly49si2 | CUST_40_PI209816013 | custom |
| 45 | Ly49i9 | 5.27 | 38.59 | 0.0100 | Ly49 inhibitory receptor 9 | CUST_66_PI209816013 | custom |
| 45 | Ly49i9 | 5.13 | 35.02 | 0.0100 | Ly49 inhibitory receptor 9 | CUST_67_PI209816013 | custom |
| 45 | Ly49i9 | 5.15 | 35.51 | 0.0100 | Ly49 inhibitory receptor 9 | CUST_68_PI209816013 | custom |
| 45 | Ly49i9 | 5.29 | 39.12 | 0.0100 | Ly49 inhibitory receptor 9 | CUST_69_PI209816013 | custom |
| 45 | Ly49i9 | 6.60 | 97.01 | 0.0100 | Ly49 inhibitory receptor 9 | CUST_70_PI209816013 | custom |
| 53 | Ly49i3 | −0.14 | 0.91 | 0.3217 | Ly49 inhibitory receptor 3 | CUST_81_PI209816013 | custom |
| 53 | Ly49i3 | 0.14 | 1.10 | 0.1803 | Ly49 inhibitory receptor 3 | CUST_82_PI209816013 | custom |
| 53 | Ly49i3 | 1.30 | 2.46 | 0.0325 | Ly49 inhibitory receptor 3 | CUST_84_PI209816013 | custom |
| 53 | Ly49i3 | 3.06 | 8.34 | 0.0180 | Ly49 inhibitory receptor 3 | CUST_85_PI209816013 | custom |
| 53 | Ly49i3 | 0.01 | 1.01 | 0.9333 | Ly49 inhibitory receptor 3 | CUST_83_PI209816013 | custom |
| 59 | Ly49i7 | 0.67 | 1.59 | 0.0395 | immunoreceptor Ly49i7 | A_44_P821875 | Agilent |

TABLE 6c

| Gene | | log2-Fold | Fold | adj. P- | | | Probe |
|---|---|---|---|---|---|---|---|
| order | Gene Symbol | Change | Change | value | Gene Description | Probe ID | Design |
| 24 | Olr1 | 1.41 | 2.66 | 0.0390 | oxidized low density lipoprotein (lectin-like) receptor 1 | A_44_P377266 | Agilent |
| 37 | LOC690045 | 5.56 | 47.18 | 0.0100 | similar to immunoreceptor Ly49si1 | A_43_P10690 | Agilent |
| 38 | Ly49si3 | 3.67 | 12.73 | 0.0180 | immunoreceptor Ly49si3 | CUST_21_PI209816013 | custom |
| 38 | Ly49si3 | 4.82 | 28.25 | 0.0100 | immunoreceptor Ly49si3 | CUST_22_PI209816013 | custom |
| 38 | Ly49si3 | 2.23 | 4.69 | 0.0310 | immunoreceptor Ly49si3 | CUST_23_PI209816013 | custom |
| 38 | Ly49si3 | 1.22 | 2.33 | 0.0411 | immunoreceptor Ly49si3 | CUST_24_PI209816013 | custom |
| 38 | Ly49si3 | 1.79 | 3.46 | 0.0365 | immunoreceptor Ly49si3 | CUST_25_PI209816013 | custom |
| 40 | Ly49si1 | 2.71 | 6.54 | 0.0325 | immunoreceptor Ly49si1 | CUST_57_PI209816013 | custom |
| 40 | Ly49si1 | 2.18 | 4.53 | 0.0362 | immunoreceptor Ly49si1 | CUST_58_PI209816013 | custom |
| 40 | Ly49si1 | 5.79 | 55.33 | 0.0100 | immunoreceptor Ly49si1 | CUST_59_PI209816013 | custom |
| 40 | Ly49si1 | 4.67 | 25.46 | 0.0100 | immunoreceptor Ly49si1 | CUST_60_PI209816013 | custom |
| 42 | Ly49si2 | 3.64 | 12.47 | 0.0180 | immunoreceptor Ly49si2 | CUST_36_PI209816013 | custom |
| 42 | Ly49si2 | 4.60 | 24.25 | 0.0100 | immunoreceptor Ly49si2 | CUST_37_PI209816013 | custom |
| 42 | Ly49si2 | 4.44 | 21.71 | 0.0100 | immunoreceptor Ly49si2 | CUST_38_PI209816013 | custom |
| 42 | Ly49si2 | 1.76 | 3.39 | 0.0310 | immunoreceptor Ly49si2 | CUST_39_PI209816013 | custom |
| 42 | Ly49si2 | 1.67 | 3.18 | 0.0373 | immunoreceptor Ly49si2 | CUST_40_PI209816013 | custom |
| 45 | Ly49i9 | 5.27 | 38.59 | 0.0100 | Ly49 inhibitory receptor 9 | CUST_66_PI209816013 | custom |
| 45 | Ly49i9 | 5.13 | 35.02 | 0.0100 | Ly49 inhibitory receptor 9 | CUST_67_PI209816013 | custom |
| 45 | Ly49i9 | 5.15 | 35.51 | 0.0100 | Ly49 inhibitory receptor 9 | CUST_68_PI209816013 | custom |
| 45 | Ly49i9 | 5.29 | 39.12 | 0.0100 | Ly49 inhibitory receptor 9 | CUST_69_PI209816013 | custom |
| 45 | Ly49i9 | 6.60 | 97.01 | 0.0100 | Ly49 inhibitory receptor 9 | CUST_70_PI209816013 | custom |
| 53 | Ly49i3 | 1.30 | 2.46 | 0.0325 | Ly49 inhibitory receptor 3 | CUST_84_PI209816013 | custom |
| 53 | Ly49i3 | 3.06 | 8.34 | 0.0180 | Ly49 inhibitory receptor 3 | CUST_85_PI209816013 | custom |
| 59 | Ly49i7 | 0.67 | 1.59 | 0.0395 | immunoreceptor Ly49i7 | A_44_P821875 | Agilent |

TABLE 7
Regulated non-MHC non-NKC genes
The expression profiling results of non-MHC non-NKC genes are given for those genes that were both significantly (p < 0.05) and
strongly (log2-fold change ≧1 or ≦−1; i.e. fold change ≧2 or ≦0.5) regulated. The log2-fold changes and the fold changes in
gene expression are shown. The adjusted p-values are indicated. For 20 of these genes at least two different probes were present on
the array. In 8 cases (indicated by gene symbols in bold) the second probe indicated the same strong and significant regulation and in
7 further cases (indicated by gene symbols in italics) the second probe indicated a regulation with borderline amplitude or
significance. In 5 cases (indicated by gene symbols in blue font) the results of the two probes for a gene did not confirm each
other. Furthermore, the identification numbers of the probes on the arrays are given (probe ID) together with the information
whether these probes were taken from the Agilent database or custom designed.

| | | log2- | | | | |
|---|---|---|---|---|---|---|
| | | Fold | Fold | adj. P- | | |
| Genes | Gene Symbol | Change | Change | value | Gene Description | Probe ID |
| 106 | NCAM1 | −2.42 | 0.19 | 0.0180 | neural cell adhesion molecule 1 | A_43_P12573 |
| 118 | Pdzrn3 | −2.41 | 0.19 | 0.0100 | PDZ domain containing RING finger 3 | A_42_P481087 |
| 142 | Serpine1 | −2.26 | 0.21 | 0.0149 | serine (or cysteine) peptidase inhibitor, | A_42_P758220 |
| | | | | | clade E, member 1 | |
| 110 | Nfe2l3 | −2.24 | 0.21 | 0.0254 | nuclear factor, erythroid derived | A_44_P393978 |
| | | | | | 2, like 3 | |
| 46 | Drd5 | −2.19 | 0.22 | 0.0336 | dopamine receptor 5 | A_43_P15525 |
| 86 | Lmcd1 | −1.96 | 0.26 | 0.0206 | LIM and cysteine-rich domains 1 | A_42_P749591 |
| 146 | SNAP25 | −1.92 | 0.26 | 0.0416 | synaptosomal-associated protein | A_43_P12469 |
| | | | | | 25 | |
| 85 | Lgals7 | −1.87 | 0.27 | 0.0149 | lectin, galactose binding, soluble 7 | A_43_P12249 |
| 98 | Lox | −1.80 | 0.29 | 0.0229 | Rattus norvegicus lysyl oxidase | A_42_P585695 |
| | | | | | (Lox), mRNA [NM_017061] | |
| 59 | Grem1 | −1.78 | 0.29 | 0.0315 | gremlin 1 | A_42_P495820 |
| 29 | Cfi | −1.77 | 0.29 | 0.0362 | complement factor I | A_42_P693316 |
| 30 | Chl1 | −1.77 | 0.29 | 0.0100 | cell adhesion molecule with homology | A_44_P1029697 |
| | | | | | to L1CAM | |
| 124 | Postn | −1.73 | 0.30 | 0.0310 | periostin, osteoblast specific factor | A_44_P525235 |
| 132 | Ptprd | −1.68 | 0.31 | 0.0416 | protein tyrosine phosphatase, receptor | A_43_P10925 |
| | | | | | type, D | |
| 115 | Pcdh21 | −1.65 | 0.32 | 0.0325 | protocadherin 21 | A_42_P596050 |
| 137 | Rarres2 | −1.65 | 0.32 | 0.0100 | Rattus norvegicus retinoic acid | A_42_P628853 |
| | | | | | receptor responder (tazarotene | |
| | | | | | induced) 2 (Rarres2), mRNA | |
| | | | | | [NM_001013427] | |
| 19 | Ccl27 | −1.61 | 0.33 | 0.0373 | chemokine (C-C motif) ligand 27 | A_42_P683840 |
| 33 | COL12A1 | −1.57 | 0.34 | 0.0149 | collagen, type XII, alpha 1 | A_43_P15760 |
| 102 | Mme | −1.57 | 0.34 | 0.0278 | membrane metallo endopeptidase | A_43_P11484 |
| 42 | Cxcl12 | −1.56 | 0.34 | 0.0365 | chemokine (C—X—C motif) ligand 12 | A_43_P12144 |
| 152 | Tgfbi | −1.54 | 0.34 | 0.0100 | transforming growth factor, beta | A_44_P620106 |
| | | | | | induced | |
| 9 | Apoe | −1.52 | 0.35 | 0.0206 | apolipoprotein E | A_44_P171440 |
| 15 | C1s | −1.47 | 0.36 | 0.0149 | complement component 1, s sub- | A_43_P15364 |
| | | | | | component | |
| 34 | Col1a2 | −1.47 | 0.36 | 0.0149 | collagen, type I, alpha 2 | A_43_P12783 |
| 7 | Anp32a | −1.45 | 0.37 | 0.0278 | acidic (leucine-rich) nuclear phosphoprotein | A_43_P11613 |
| | | | | | 32 family, member A | |
| 36 | Col8a1 | −1.40 | 0.38 | 0.0229 | collagen, type VIII, alpha 1 | A_44_P140684 |
| 40 | Cthrc1 | −1.40 | 0.38 | 0.0490 | collagen triple helix repeat containing 1 | A_44_P144591 |
| 60 | Grin2c | −1.39 | 0.38 | 0.0373 | Rattus norvegicus glutamate receptor, | A_42_P738337 |
| | | | | | ionotropic, NMDA2C | |
| | | | | | (Grin2c), mRNA [NM_012575] | |
| 94 | LOC684607 | −1.39 | 0.38 | 0.0345 | similar to nuclear receptor binding | A_44_P191287 |
| | | | | | protein | |
| 73 | Igfbp5 | −1.36 | 0.39 | 0.0433 | Rattus norvegicus cDNA clone IMAGE: | A_44_P264240 |
| | | | | | 7110383 [BC087030] | |
| 119 | Perp | −1.36 | 0.39 | 0.0267 | PERP, TP53 apoptosis effector | A_42_P768883 |
| 127 | Prom2 | −1.35 | 0.39 | 0.0416 | prominin 2 | A_42_P530761 |
| 31 | Chn1 | −1.32 | 0.40 | 0.0100 | chimerin (chimaerin) 1 | A_43_P15576 |
| 87 | LOC100044927 | −1.30 | 0.41 | 0.0449 | similar to TNF-stimulated gene 6 | A_43_P16110 |
| | | | | | protein | |
| 35 | Col5a3 | −1.28 | 0.41 | 0.0395 | collagen, type V, alpha 3 | A_44_P197290 |
| 165 | Wisp1 | −1.27 | 0.41 | 0.0100 | WNT1 inducible signaling pathway | A_42_P816427 |
| | | | | | protein 1 | |
| 37 | Cpe | −1.25 | 0.42 | 0.0100 | carboxypeptidase E | A_42_P708169 |
| 42 | Cxcl12 | −1.24 | 0.42 | 0.0365 | chemokine (C—X—C motif) ligand 12 | A_44_P337351 |
| 108 | Nell2 | −1.23 | 0.43 | 0.0365 | Rattus norvegicus nel-like 2 homolog | A_43_P12500 |
| | | | | | (chicken) (Nell2), mRNA | |
| | | | | | [NM_031070] | |
| 151 | Tcfap2b | −1.21 | 0.43 | 0.0100 | transcription factor AP-2 beta | A_42_P463781 |
| 3 | Adcy2 | −1.20 | 0.44 | 0.0449 | adenylate cyclase 2 | A_43_P15311 |
| 32 | Clu | −1.20 | 0.44 | 0.0310 | clusterin | A_44_P311126 |
| 55 | Fst | −1.20 | 0.44 | 0.0298 | follistatin | A_44_P108588 |
| 56 | Fzd1 | −1.20 | 0.44 | 0.0149 | frizzled homolog 1 (Drosophila) | A_44_P170527 |
| 1 | Abcc1 | −1.18 | 0.44 | 0.0310 | ATP-binding cassette, sub-family C | A_44_P252417 |
| | | | | | (CFTR/MRP), member 1 | |
| 81 | Itpr3 | −1.18 | 0.44 | 0.0100 | inositol 1,4,5-triphosphate receptor 3 | A_42_P572461 |
| 126 | Prkcdbp | −1.18 | 0.44 | 0.0180 | protein kinase C, delta binding | A_42_P736812 |
| | | | | | protein | |
| 150 | Tacstd2 | −1.18 | 0.44 | 0.0206 | tumor-associated calcium signal | A_42_P468712 |
| | | | | | transducer 2 | |
| 10 | Asam | −1.15 | 0.45 | 0.0298 | adipocyte-specific adhesion molecule | A_44_P292495 |
| 44 | Dclk1 | −1.15 | 0.45 | 0.0267 | doublecortin-like kinase 1 | A_42_P787216 |
| 135 | Ptprz1 | −1.15 | 0.45 | 0.0100 | protein tyrosine phosphatase, receptor | A_42_P475885 |
| | | | | | type Z, polypeptide 1 | |
| 47 | EGR1 | −1.14 | 0.45 | 0.0149 | early growth response 1 | A_42_P623792 |
| 58 | Gpr98 | −1.14 | 0.45 | 0.0206 | G protein-coupled receptor 98 | A_42_P478080 |
| 84 | Lgals1 | −1.13 | 0.46 | 0.0229 | lectin, galactose binding, soluble 1 | A_42_P759159 |
| 111 | Nfib | −1.13 | 0.46 | 0.0533 | nuclear factor I/B | A_42_P752916 |
| 4 | Adcy8 | −1.12 | 0.46 | 0.0149 | adenylate cyclase 8 | A_42_P466362 |
| 27 | Cdh5_predicted | −1.12 | 0.46 | 0.0254 | PREDICTED: Rattus norvegicus | A_44_P121658 |
| | | | | | cadherin 5 (predicted) | |
| | | | | | (Cdh5_predicted), mRNA | |
| | | | | | [XM_226213] | |
| 44 | Dclk1 | −1.12 | 0.46 | 0.0278 | doublecortin-like kinase 1 | A_44_P172645 |
| 154 | Thbs4 | −1.12 | 0.46 | 0.0466 | thrombospondin 4 | A_44_P337311 |
| 69 | Htra1 | −1.11 | 0.46 | 0.0100 | HtrA serine peptidase 1 | A_43_P12648 |
| 133 | Ptprf | −1.10 | 0.47 | 0.0345 | protein tyrosine phosphatase, receptor | A_43_P11993 |
| | | | | | type, F | |
| 104 | Mtss1_predicted | −1.09 | 0.47 | 0.0325 | PREDICTED: Rattus norvegicus | A_44_P554679 |
| | | | | | metastasis suppressor 1 (predicted) | |
| | | | | | (Mtss1_predicted), mRNA | |
| | | | | | [XM_001064860] | |
| 144 | Serpinf1 | −1.09 | 0.47 | 0.0206 | serine (or cysteine) peptidase inhibitor, | A_42_P709525 |
| | | | | | clade F, member 1 | |
| 14 | C1qtnf7 | −1.08 | 0.47 | 0.0310 | C1q and tumor necrosis factor | A_44_P248172 |
| | | | | | related protein 7 | |
| 51 | Fam89a | −1.08 | 0.47 | 0.0481 | family with sequence similarity 89, | A_42_P619403 |
| | | | | | member A | |
| 143 | Serpine2 | −1.08 | 0.47 | 0.0481 | serine (or cysteine) peptidase inhibitor, | A_43_P15697 |
| | | | | | clade E, member 2 | |
| 61 | Gsn | −1.06 | 0.48 | 0.0267 | gelsolin | A_44_P1014163 |
| 101 | Med13l | −1.06 | 0.48 | 0.0229 | mediator complex subunit 13-like | A_44_P169863 |
| 113 | Ntrk2 | −1.06 | 0.48 | 0.0100 | neurotrophic tyrosine kinase, receptor, | A_42_P538400 |
| | | | | | type 2 | |
| 117 | Pdgfrb | −1.05 | 0.48 | 0.0206 | platelet derived growth factor receptor, | A_43_P15740 |
| | | | | | beta polypeptide | |
| 149 | Sulf1 | −1.05 | 0.48 | 0.0325 | sulfatase 1 | A_43_P13252 |
| 39 | Ctgf | −1.04 | 0.49 | 0.0466 | connective tissue growth factor | A_42_P484738 |
| 50 | Ercc5 | −1.04 | 0.49 | 0.0315 | excision repair cross- | A_44_P1019654 |
| | | | | | complementing rodent repair deficiency, | |
| | | | | | complementation group 5 | |
| 100 | Ltbp1 | −1.04 | 0.49 | 0.0180 | latent transforming growth factor | A_43_P14871 |
| | | | | | beta binding protein 1 | |
| 114 | Papss2 | −1.04 | 0.49 | 0.0395 | 3′-phosphoadenosine 5′- | A_42_P513050 |
| | | | | | phosphosulfate synthase 2 | |
| 111 | Nfib | −1.03 | 0.49 | 0.0457 | nuclear factor I/B | A_43_P15686 |
| 68 | Hrh3 | −1.02 | 0.49 | 0.0395 | histamine receptor H3 | A_43_P15338 |
| 23 | Ccnd1 | −1.01 | 0.50 | 0.0100 | cyclin D1 | A_44_P189299 |
| 94 | LOC684607 | −0.88 | 0.54 | 0.0634 | similar to nuclear receptor binding | A_44_P250983 |
| | | | | | protein | |
| 154 | Thbs4 | −0.87 | 0.55 | 0.0648 | thrombospondin 4 | A_43_P15768 |
| 151 | Tcfap2b | −0.85 | 0.55 | 0.0345 | transcription factor AP-2 beta | A_43_P18397 |
| 113 | Ntrk2 | −0.37 | 0.77 | 0.2827 | neurotrophic tyrosine kinase, receptor, | A_42_P631184 |
| | | | | | type 2 | |
| 101 | Med13l | −0.32 | 0.80 | 0.1024 | mediator complex subunit 13-like | A_44_P473186 |
| 166 | Wnt7a | −0.21 | 0.86 | 0.2198 | wingless-related MMTV integration | A_44_P623953 |
| | | | | | site 7A | |
| 114 | Papss2 | 0.02 | 1.01 | 0.9620 | 3′-phosphoadenosine 5′- | A_44_P119160 |
| | | | | | phosphosulfate synthase 2 | |
| 66 | Hmha1 | 0.22 | 1.16 | 0.1958 | histocompatibility (minor) HA-1 | A_43_P20339 |
| 168 | Zfp36 | 0.54 | 1.45 | 0.2006 | zinc finger protein 36 | A_44_P435596 |
| 18 | Ccl1 | 0.60 | 1.52 | 0.0791 | chemokine (C-C motif) ligand 1 | CUST_51_PI240872834 |
| 76 | Il1rn | 0.73 | 1.66 | 0.0100 | interleukin 1 receptor antagonist | A_43_P15503 |
| 163 | Tyrobp | 0.91 | 1.88 | 0.0229 | Tyro protein tyrosine kinase binding | A_44_P526676 |
| | | | | | protein | |
| 22 | Ccl9 | 1.00 | 2.00 | 0.0254 | chemokine (C-C motif) ligand 9 | A_43_P22206 |
| 153 | Tgm2 | 1.00 | 2.00 | 0.0254 | transglutaminase 2, C polypeptide | A_44_P1007347 |
| 121 | Plaur | 1.02 | 2.03 | 0.0229 | plasminogen activator, urokinase | A_44_P468141 |
| | | | | | receptor | |
| 128 | Pstpip1 | 1.07 | 2.10 | 0.0325 | proline-serine-threonine phosphatase- | A_44_P180717 |
| | | | | | interacting protein 1 | |
| 64 | Hcls1 | 1.09 | 2.13 | 0.0325 | hematopoietic cell specific Lyn | A_43_P21322 |
| | | | | | substrate 1 | |
| 160 | Treml1 | 1.10 | 2.14 | 0.0373 | triggering receptor expressed on | A_44_P798023 |
| | | | | | myeloid cells-like 1 | |
| 18 | Ccl1 | 1.12 | 2.17 | 0.0298 | chemokine (C-C motif) ligand 1 | CUST_52_PI240872834 |
| 77 | Il2rb | 1.13 | 2.19 | 0.0815 | interleukin 2 receptor, beta chain | A_44_P265709 |
| 26 | Cd8b1 | 1.16 | 2.23 | 0.0229 | CD8b molecule | A_42_P480723 |
| 79 | Itgax | 1.16 | 2.23 | 0.0457 | Rattus norvegicus integrin alpha X | A_42_P700646 |
| | | | | | (Itgax), mRNA [NM_031691] | |
| 20 | Ccl3 | 1.17 | 2.25 | 0.0762 | chemokine (C-C motif) ligand 3 | A_42_P714311 |
| 112 | Nfkbia | 1.18 | 2.27 | 0.0180 | nuclear factor of kappa light polypeptide | A_42_P544487 |
| | | | | | gene enhancer in B-cells | |
| | | | | | inhibitor, alpha | |
| 122 | Plk3 | 1.18 | 2.27 | 0.0315 | polo-like kinase 3 (Drosophila) | A_44_P135224 |
| 147 | Snx10 | 1.18 | 2.27 | 0.0345 | sorting nexin 10 | A_43_P16967 |
| 49 | Epha2 | 1.20 | 2.30 | 0.0278 | Eph receptor A2 | A_42_P569711 |
| 145 | Slfn2 | 1.20 | 2.30 | 0.0206 | schlafen 2 | A_44_P469113 |
| 120 | Pik3ap1 | 1.21 | 2.31 | 0.0278 | phosphoinositide-3-kinase adaptor | A_43_P21121 |
| | | | | | protein 1 | |
| 18 | Ccl1 | 1.23 | 2.35 | 0.0378 | chemokine (C-C motif) ligand 1 | CUST_53_PI240872834 |
| 166 | Wnt7a | 1.24 | 2.36 | 0.0336 | wingless-related MMTV integration | A_44_P135238 |
| | | | | | site 7A | |
| 78 | Itgam | 1.25 | 2.38 | 0.0424 | integrin alpha M | A_43_P15993 |
| 11 | AW141130 | 1.27 | 2.41 | 0.0254 | EST291162 Normalized rat brain, | A_44_P635423 |
| | | | | | Bento Soares Rattus sp. cDNA | |
| | | | | | clone RGIBD16 5′ end similar to | |
| | | | | | interleukin-3 receptor B subunit, | |
| | | | | | mRNA sequence [AW141130] | |
| 161 | Trib3 | 1.27 | 2.41 | 0.0278 | tribbles homolog 3 (Drosophila) | A_42_P543774 |
| 5 | Adipor2 | 1.31 | 2.48 | 0.0254 | adiponectin receptor 2 | A_44_P1013376 |
| 5 | Adipor2 | 1.31 | 2.48 | 0.0100 | adiponectin receptor 2 | A_42_P523357 |
| 82 | L37967 | 1.32 | 2.50 | 0.0345 | RATTCRAL Rattus norvegicus T- | A_43_P16248 |
| | | | | | cell receptor alpha-chain mRNA | |
| | | | | | [L37967] | |
| 134 | Ptprj | 1.37 | 2.58 | 0.0378 | protein tyrosine phosphatase, receptor | A_43_P15275 |
| | | | | | type, J | |
| 18 | Ccl1 | 1.38 | 2.60 | 0.0325 | chemokine (C-C motif) ligand 1 | CUST_54_PI240872834 |
| 2 | Adcy10 | 1.40 | 2.64 | 0.0325 | adenylate cyclase 10 | A_42_P460021 |
| 38 | Csf2 | 1.40 | 2.64 | 0.0401 | colony stimulating factor 2 (granulocyte- | A_43_P16294 |
| | | | | | macrophage) | |
| 71 | Ifitm1 | 1.43 | 2.69 | 0.0180 | interferon induced transmembrane | A_42_P676304 |
| | | | | | protein 1 | |
| 138 | RGD1561143 | 1.45 | 2.73 | 0.0310 | similar to cell surface receptor | A_44_P182601 |
| | | | | | FDFACT | |
| 92 | LOC681069 | 1.46 | 2.75 | 0.0401 | similar to paired immunoglobin- | A_44_P330565 |
| | | | | | like type 2 receptor beta | |
| 168 | Zfp36 | 1.47 | 2.77 | 0.0378 | zinc finger protein 36 | A_42_P648055 |
| 48 | Emb | 1.49 | 2.81 | 0.0315 | embigin | A_44_P304220 |
| 99 | Lpxn | 1.49 | 2.81 | 0.0278 | leupaxin | A_43_P23014 |
| 66 | Hmha1 | 1.51 | 2.85 | 0.0365 | histocompatibility (minor) HA-1 | A_44_P992516 |
| 141 | Rhoh | 1.52 | 2.87 | 0.0378 | ras homolog gene family, member H | A_43_P23152 |
| 18 | Ccl1 | 1.53 | 2.89 | 0.0390 | chemokine (C-C motif) ligand 1 | CUST_55_PI240872834 |
| 76 | Il1rn | 1.56 | 2.95 | 0.0100 | interleukin 1 receptor antagonist | A_44_P462661 |
| 6 | AF216218 | 1.57 | 2.97 | 0.0254 | AF216218 Rattus norvegicus orphanin | A_44_P442838 |
| | | | | | FQ receptor gene (OFQR), | |
| | | | | | complete cds, alternatively spliced | |
| | | | | | [AF216218] | |
| 67 | Hmox1 | 1.57 | 2.97 | 0.0100 | heme oxygenase (decycling) 1 | A_42_P652275 |
| 157 | Tnfsf13 | 1.57 | 2.97 | 0.0278 | tumor necrosis factor (ligand) superfamily, | A_42_P773636 |
| | | | | | member 13 | |
| 93 | LOC683463 | 1.58 | 2.99 | 0.0325 | similar to paired-Ig-like receptor B | A_42_P841620 |
| 70 | Ifi47 | 1.65 | 3.14 | 0.0310 | interferon gamma inducible protein | A_44_P174992 |
| | | | | | 47 | |
| 25 | Cd83 | 1.68 | 3.20 | 0.0481 | CD83 antigen | A_42_P767128 |
| 65 | Hk3 | 1.70 | 3.25 | 0.0206 | hexokinase 3 | A_44_P114207 |
| 45 | Dok3 | 1.72 | 3.29 | 0.0325 | docking protein 3 | A_42_P468452 |
| 28 | Ceacam10 | 1.74 | 3.34 | 0.0254 | CEA-related cell adhesion molecule | A_43_P13426 |
| | | | | | 10 | |
| 83 | Lcp2 | 1.78 | 3.43 | 0.0310 | lymphocyte cytosolic protein 2 | A_42_P671389 |
| 116 | Pcsk1 | 1.80 | 3.48 | 0.0395 | proprotein convertase subtilisin/ | A_42_P570848 |
| | | | | | kexin type 1 | |
| 16 | C5ar1 | 1.83 | 3.56 | 0.0298 | complement component 5a receptor 1 | A_42_P572521 |
| 129 | Ptger2 | 1.85 | 3.61 | 0.0278 | prostaglandin E receptor 2, sub- | A_43_P12508 |
| | | | | | type EP2 | |
| 77 | Il2rb | 1.86 | 3.63 | 0.0365 | interleukin 2 receptor, beta chain | A_42_P555801 |
| 53 | Fcgr3 | 1.89 | 3.71 | 0.0100 | Fc receptor, IgG, low affinity III | A_44_P168405 |
| 164 | Vav1 | 1.89 | 3.71 | 0.0229 | vav 1 oncogene | A_42_P572413 |
| 17 | Card11 | 1.93 | 3.81 | 0.0206 | caspase recruitment domain family, | A_44_P421727 |
| | | | | | member 11 | |
| 158 | Trem1 | 1.94 | 3.84 | 0.0206 | triggering receptor expressed on | A_44_P354415 |
| | | | | | myeloid cells 1 | |
| 155 | Tlr2 | 1.95 | 3.86 | 0.0315 | toll-like receptor 2 | A_43_P19763 |
| 136 | Rarres1 | 2.00 | 4.00 | 0.0345 | retinoic acid receptor responder | A_42_P528691 |
| | | | | | (tazarotene induced) 1 | |
| 89 | LOC498277 | 2.05 | 4.14 | 0.0100 | similar to Low affinity immunoglobulin | A_44_P482476 |
| | | | | | gamma Fc region receptor | |
| | | | | | III precursor (IgG Fc receptor | |
| | | | | | III) (Fc-gamma RIII) (FcRIII) | |
| 96 | LOC685157 | 2.05 | 4.14 | 0.0180 | similar to paired immunoglobin- | A_44_P745407 |
| | | | | | like type 2 receptor beta | |
| 12 | Batf | 2.06 | 4.17 | 0.0310 | basic leucine zipper transcription | A_42_P624111 |
| | | | | | factor, ATF-like | |
| 13 | Bcl2a1d | 2.08 | 4.23 | 0.0254 | B-cell leukemia/lymphoma 2 related | A_43_P13182 |
| | | | | | protein A1d | |
| 20 | Ccl3 | 2.11 | 4.32 | 0.0278 | chemokine (C-C motif) ligand 3 | A_43_P11666 |
| 163 | Tyrobp | 2.14 | 4.41 | 0.0100 | Tyro protein tyrosine kinase binding | A_42_P807697 |
| | | | | | protein | |
| 8 | Apob48r | 2.15 | 4.44 | 0.0373 | apolipoprotein B48 receptor | A_44_P194387 |
| 57 | Gpnmb | 2.19 | 4.56 | 0.0149 | glycoprotein (transmembrane) | A_42_P517381 |
| | | | | | nmb | |
| 43 | Cxcl2 | 2.21 | 4.63 | 0.0206 | chemokine (C—X—C motif) ligand 2 | A_43_P12885 |
| 103 | Msr1 | 2.22 | 4.66 | 0.0373 | macrophage scavenger receptor 1 | A_44_P928825 |
| 130 | Ptpn7 | 2.26 | 4.79 | 0.0206 | protein tyrosine phosphatase, | A_42_P653257 |
| | | | | | non-receptor type 7 | |
| 91 | LOC680910 | 2.31 | 4.96 | 0.0180 | similar to paired imnnunoglobin- | A_44_P187246 |
| | | | | | like type 2 receptor beta | |
| 162 | Trpv2 | 2.32 | 4.99 | 0.0100 | transient receptor potential cation | A_42_P816020 |
| | | | | | channel, subfamily V, member 2 | |
| 156 | Tnfaip8l2 | 2.33 | 5.03 | 0.0100 | tumor necrosis factor, alpha- | A_43_P20022 |
| | | | | | induced protein 8-like 2 | |
| 74 | Igsf6 | 2.34 | 5.06 | 0.0395 | immunoglobulin superfamily, | A_42_P588738 |
| | | | | | member 6 | |
| 140 | Rgs1 | 2.36 | 5.13 | 0.0373 | regulator of G-protein signaling 1 | A_43_P16318 |
| 107 | Ncf1 | 2.40 | 5.28 | 0.0100 | neutrophil cytosolic factor 1 | A_44_P298049 |
| 75 | Il1b | 2.43 | 5.39 | 0.0395 | interleukin 1 beta | A_43_P14911 |
| 139 | RGD1561778 | 2.55 | 5.86 | 0.0206 | similar to dendritic cell-derived | A_44_P176053 |
| | | | | | immunoglobulin(Ig)-like receptor | |
| | | | | | 1, DIgR1 - mouse | |
| 80 | Itgb2 | 2.56 | 5.90 | 0.0100 | integrin beta 2 | A_42_P591344 |
| 91 | LOC680910 | 2.59 | 6.02 | 0.0229 | similar to paired immunoglobin- | A_44_P463899 |
| | | | | | like type 2 receptor beta | |
| 72 | Igf1 | 2.61 | 6.11 | 0.0100 | insulin-like growth factor 1 | A_44_P126021 |
| 90 | LOC498277 | 2.61 | 6.11 | 0.0100 | similar to Low affinity immunoglobulin | A_43_P12955 |
| | | | | | gamma Fc region receptor | |
| | | | | | III precursor (IgG Fc receptor | |
| | | | | | III) (Fc-gamma RIII) (FcRIII) | |
| 52 | Fcgr2b | 2.62 | 6.15 | 0.0100 | Fc receptor, IgG, low affinity IIb | A_42_P735417 |
| 148 | Spic | 2.68 | 6.41 | 0.0278 | Spi-C transcription factor (Spi- | A_42_P526140 |
| | | | | | 1/PU.1 related) | |
| 109 | Nfe2 | 2.74 | 6.68 | 0.0149 | nuclear factor, erythroid derived 2 | A_42_P464736 |
| 125 | Prg4 | 2.74 | 6.68 | 0.0149 | proteoglycan 4 (megakaryocyte | A_43_P14460 |
| | | | | | stimulating factor, articular superficial | |
| | | | | | zone protein) | |
| 123 | Plscr1 | 2.83 | 7.11 | 0.0100 | phospholipid scramblase 1 | A_44_P1025102 |
| 88 | LOC100048479 | 2.97 | 7.84 | 0.0373 | one cut domain, family member 1 | A_42_P701060 |
| 62 | Gzmc | 3.11 | 8.63 | 0.0373 | granzyme C | A_42_P774527 |
| 41 | Ctss | 3.15 | 8.88 | 0.0100 | cathepsin S | A_44_P1004731 |
| 72 | Igf1 | 3.23 | 9.38 | 0.0100 | insulin-like growth factor 1 | A_44_P366723 |
| 24 | Cd36 | 3.57 | 11.88 | 0.0100 | CD36 antigen | A_43_P12588 |
| 21 | Ccl6 | 3.71 | 13.09 | 0.0100 | Rattus norvegicus chemokine (C-C | A_43_P16707 |
| | | | | | motif) ligand 6 (Ccl6), mRNA | |
| | | | | | [NM_001004202] | |
| 159 | Trem2 | 3.78 | 13.74 | 0.0100 | triggering receptor expressed on | A_42_P512838 |
| | | | | | myeloid cells 2 | |
| 63 | Hck | 3.87 | 14.62 | 0.0100 | hemopoietic cell kinase | A_43_P11749 |
| 167 | XM_226926 | 3.92 | 15.14 | 0.0149 | Rattus norvegicus similar to protein | A_44_P375194 |
| | | | | | tyrosine phosphatase, non- | |
| | | | | | receptor type substrate; brain | |
| | | | | | immunological-like with tyrosine- | |
| | | | | | based motifs (LOC310212), mRNA | |
| | | | | | [XM_226926] | |
| 22 | Ccl9 | 4.16 | 17.88 | 0.0100 | chemokine (C-C motif) ligand 9 | A_42_P560084 |
| 105 | Nat8 | 5.14 | 35.26 | 0.0100 | Rattus norvegicus endogenous | A_44_P594411 |
| | | | | | retrovirus mRNA, partial sequence | |
| | | | | | [AY212271] | |
| 54 | Fcgr3a | 5.24 | 37.79 | 0.0100 | Fc fragment of IgG, low affinity | A_42_P798429 |
| | | | | | IIIa, receptor | |
| 131 | Ptpns1l3 | 6.36 | 82.14 | 0.0100 | protein tyrosine phosphatase, | A_44_P248248 |
| | | | | | non-receptor type substrate 1-like 3 | |
| 95 | LOC685020 | 8.18 | 290.02 | 0.0100 | paired immunoglobin-like type 2 | A_44_P715240 |
| | | | | | receptor alpha | |

TABLE 8
Primer sequences used for mRNA expression analysis

| | | | Proximity | Efficiency |
|---|---|---|---|---|
| | | Amplicon | to poly-A | coefficient |
| | Primer sequence 5′-3′¹ | (bp) | (bp) | (E)² |
| RT1-A2 | F: TCCCTCCCTGCTACCCTGAG | 103 | 105 | 1.93 |
| | (SEQ ID NO: 48) | | | |
| | R: GCCATCCACACTTGGGTCAA | | | |
| | (SEQ ID NO: 49) | | | |
| RT1-DMb | F: TCAAATCTGCCTCGGGTGTTT | 80 | 53 | 1.87 |
| | (SEQ ID NO: 50) | | | |
| | R: GACAAGGTGGGGCTTTCAGG | | | |
| | (SEQ ID NO: 51) | | | |
| Psmb8 | F: CACTGCTGGGCAGACATCCT | 109 | 91 | 1.92 |
| | (SEQ ID NO: 52) | | | |
| | R: GCTTTGTCTCCAGCCCAGGT | | | |
| | (SEQ ID NO: 53) | | | |
| Ly6g6e | F: CCCAGGCAAAGGGACAGAAG | 87 | 151 | 1.97 |
| | (SEQ ID NO: 54) | | | |
| | R: TGAGACCCTCAGGCACCAAG | | | |
| | (SEQ ID NO: 55) | | | |
| Aif1 | F: TCCCCCAGCCAAGAAAGCTA | 99 | 51 | 1.86 |
| | (SEQ ID NO: 56) | | | |
| | R: TCTTTTCCCATGCTGCTGTCA | | | |
| | (SEQ ID NO: 57) | | | |
| Lst1 | F: GGGCAGGAGCTCCACTACG | 118 | 20 | 1.89 |
| | (SEQ ID NO: 58) | | | |
| | R: CGATGCAGGCATAGTCAGTGC | | | |
| | (SEQ ID NO: 59) | | | |
| RT1-CE3 | F: TGTCGTCCTTGGAGCCATCT | 62 | 106 | 1.91 |
| | (SEQ ID NO: 60) | | | |
| | R: TCCTCACAACAGGCACCAGA | | | |
| | (SEQ ID NO: 61) | | | |
| RT1-CE10 | F: ACACAGGTGGGGAAGGAGGA | 82 | 10 | 1.94 |
| | (SEQ ID NO: 62) | | | |
| | R: CAATCTGGGAGGGACACATCAG | | | |
| | (SEQ ID NO: 63) | | | |
| RT-BM1 | F: GCAGCTATGCTCATGTTCTAGGC | 62 | 7 | 1.89 |
| (RT1-S3) | (SEQ ID NO: 64) | | | |
| | R: TGCCTTCTGAGGCCAGTCAG | | | |
| | (SEQ ID NO: 65) | | | |
| Ubd | F: TGGGGTGATGAGAAGCTCAAAA | 105 | 7 | 1.92 |
| | (SEQ ID NO: 66) | | | |
| | R: CCCCACCTCAAATCTTTATTTC | | | |
| | ATTC | | | |
| | (SEQ ID NO: 67) | | | |
| Olr1 | F: GGAAGTCAGAAGAGGGCATGG | 89 | 271 | 1.90 |
| | (SEQ ID NO: 68) | | | |
| | R: TCCTGGGTTCAATTTCCAGAGT | | | |
| | (SEQ ID NO: 69) | | | |
| Ly49si1 | F: TGGCCAATCTGAATTTTCCTTG | 115 | 36 | 1.84 |
| | (SEQ ID NO: 70) | | | |
| | R: ACATGGGAAGGGGTTCATGC | | | |
| | (SEQ ID NO: 71) | | | |
| Ly49i9 | F: GGGACTTGGCAACCTCAGGA | 110 | 179 | 1.88 |
| | (SEQ ID NO: 72) | | | |
| | R: TTGGAACATCTGCACAATGGAA | | | |
| | (SEQ ID NO: 73) | | | |
| Cd3z | F: AGTGCCTGCTGGGATTTAGC | 118 | 50 | 1.93 |
| | (SEQ ID NO: 74) | | | |
| | R: CATCCATGGTCACAGGCACTT | | | |
| | (SEQ ID NO: 75) | | | |
| B2m | F: GAGCAGGTTGCTCCACAGGT | 128 | 246 | 1.94 |
| | (SEQ ID NO: 76) | | | |
| | R: CAAGCTTTGAGTGCAAGAGATTGA | | | |
| | (SEQ ID NO: 77) | | | |

¹F: forward primer, R: reverse primer
²The real-time PCR efficiency coefficient (E) of one cycle in the exponential phase was calculated according to the equation: E = 10^{[-1/slope of standard curve]}

TABLE 9

| | | | | | | Concordance | |
|---|---|---|---|---|---|---|---|
| | | | | | | rate in | |
| | | | Log2-fold | | Concordance rate | further | Log2-fold |
| | | | change (rat | | first 3 human skin | human skin | change |
| Gene | Gene description | Tested organism | microarray) | Tested organism | explants | explants | (human data) |
| Ctss | cathepsin S | Rattus novegicus | 3.15 | Homo sapiens | 3/3 | 7/9 | −1.25 |
| Pbx2 | Pre-B-cell leukemia homeobox 2 | Rattus novegicus | 0.33 | Homo sapiens | 1/3 | 7/9 | −1.5 |
| Grem1 | Gremlin-1 inhibitor in the TGF | Rattus novegicus | −1.78 | Homo sapiens | 2/3 | 6/9 | −3 |
| | beta signaling pathway | | | | | | |
| Ly6g6e | lymphocyte antigen 6 complex, | Rattus novegicus | −1.43 | Homo sapiens | 0/3 | 6/9 | −2.25 |
| | locus G6E | | | | | | |
| Spr1 | psoriasis susceptibility 1 candidate | Rattus novegicus | 1.45 | Homo sapiens | 1/3 | 5/5 | −1.25 |
| | 2 (human) | | | | | | |
| Msr1 | macrophage scavenger protein | Rattus novegicus | 2.22 | Homo sapiens | 1/3 | 4/9 | 1.5 |
| Spic | Spi-C transcription factor | Rattus novegicus | 2.68 | Homo sapiens | 0/3 | 4/9 | −2 |
| Nfe2 | nuclear factor, erythroid derived 2 | Rattus novegicus | 2.74 | Homo sapiens | 0/3 | 3/9 | −1.5 |
| Tnfaip8l2 | tumor necrosis factor, alpha- | Rattus novegicus | 2.33 | Homo sapiens | 3/3 | 3/9 | −1.5 |
| | induced protein 8-like 2 | | | | | | |
| Ier3 | Immediate early response 3 | Rattus novegicus | 0.87 | Homo sapiens | 1/3 | 2/9 | −1.5 |
| Pik3ap1 | phosphoinositide-3-kinase adaptor | Rattus novegicus | 1.21 | Homo sapiens | 3/3 | 1/9 | 1 |
| | protein 1 | | | | | | |
| Pstpip1 | proline-serine-threonine phosphatase- | Rattus novegicus | 1.07 | Homo sapiens | 3/3 | 1/9 | 2 |
| | interacting protein 1 | | | | | | |
| Ubd | ubiquitin D | Rattus novegicus | 3.19 | Homo sapiens | 3/3 | 4/9 | 1.25 |
| C2 | complement component 2 | Rattus novegicus | 1.22 | Homo sapiens | 2/3 | 1/9 | 1 |
| Lst1 | leukocyte specific transcript 1 | Rattus novegicus | 3.32 | Homo sapiens | 1/3 | 5/9 | −1.25 |
| Aif1 | allograft inflammatory factor 1 | Rattus novegicus | 2.83 | Homo sapiens | 1/3 | 3/9 | 1.25 |
| C1QTNF7 | C1q and TNF related protein 7 | Rattus novegicus | −1.08 | Homo sapiens | 0/3 | 8/9 | −2 |
| MME | Membrane metallo-endopeptidase | Rattus novegicus | −1.75 | Homo sapiens | 3/3 | 6/9 | −2 |
| | expressed by B and T cells upon | | | | | | |
| | induction of apotosis | | | | | | |
| IGFBP5 | Insulin-like growth factor-binding | Rattus novegicus | −1.36 | Homo sapiens | 1/3 | 6/9 | −2 |
| | protein 5 | | | | | | |
| CARD11 | apoptosis and scaffolding | Rattus novegicus | 1.93 | Homo sapiens | 3/3 | 6/9 | −2 |

TABLE 10

| | | Probe ID | | | | |
|---|---|---|---|---|---|---|
| | | Applied | Probe ID | | | |
| | | Biosys- | Agilent | | | |
| | | tems TLDA | microarray | | Entrez | |
| | RefSeq | card | chip | RefSeq | GeneID | Sequence |
| Gene | (human) | (human) | (rat) | (rat) | (rat) | (Agilent microarray chip, rat) |
| Ctss | NM_004079.3 | CTSS- | A_44_P1004731 | NM_ | ID: | CTGGCTTACAGCTTGTTTGTTTTATAACTT- |
| | | Hs00175403_ | | 017320 | 50654 | TACCTCTCTCTGAAAAGTCTGTAAGCAAGG |
| | | m1 | | | | (SEQ ID NO: 26) |
| Pbx2 | NM_002586.4 | PBX2- | A_42_P592157 | NM_ | ID: | AAAGCTTTCGGTTTTGTTTTTTAAACTGTT- |
| | | Hs00855025_ | | 001002828 | 406164 | TGCAGAGTGGAGAAGATCGATCAGGAAGGG |
| | | s1 | | | | (SEQ ID NO: 27) |
| Grem1 | NM_013372.5 | GREM1- | A_42_P495820 | NM_ | ID: | ATTATGCAGGCTATGACGGAACTACTACCT- |
| | | Hs00171951_ | | 019282 | 50566 | TGCTATGGATGAGGGTTGGGCAGGATTTAA |
| | | m1 | | | | (SEQ ID NO: 28) |
| Ly6g6e | NR_003673 | LY6G6E- | CUST_1_ | NM_ | ID: | GTCTCAAGAACAGAGGGCTACCTTGGGGAG- |
| | | Hs00225567_ | PI195698 | 027366 | 406866 | CCATAAAGAGTGTATTTAATAAAACGGGCT |
| | | m1 | 246 | | | (SEQ ID NO: 29) |
| Spr1 | NM_014069.2 | PSORS1C2- | A_66_P100662 | NM_ | ID: | TTTGTGGTCCCTGTTCAGTCATTATGTTGT- |
| | | Hs00204152_ | | 020576 | 57390 | CCCTTCGCTTCTCTTGATCAGCAGAAAGCA |
| | | m1 | | | | (SEQ ID NO: 31) |
| Msr1 | NM_138715.2 | MSR1- | A_44_P928825 | XM_ | ID: | GAACGTGTGCACAAAGTATCAGCAGAAATC- |
| | | Hs00234007_m1 | | 573919 | 498638 | CAGTCTGTGAAAGAAGAACAAGAGCATGTG |
| | | | | | | (SEQ ID NO: 32) |
| Spic | NM_152323.1 | SPIC- | A_42_P526140 | NM_ | ID: | CTCAGTGTCCGTGAATTGGGTATCCAAGAA- |
| | | Hs00951473_ | | 011461 | 20728 | CATCCTGAAGCCAGAATGTCTTCTCAGAAA |
| | | g1 | | | | (SEQ ID NO: 33) |
| Nfe2 | NM_001136023 | NFE2- | A_42_P464736 | NM_ | ID: | AGGCTGAGTTCTCCAGACCAAAAGACCATT- |
| | | Hs00232351_ | | 001012224 | 366998 | TGGAAGTTCAAAGATGTATTTGAGGTTTGC |
| | | m1 | | | | (SEQ ID NO: 34) |
| Tnfaip8l2 | NM_024575.3 | TNFAIP8L2- | A_43_P20022 | NM_ | ID: | AGCTCTGAGGCTCCTGAGCTCAGCACACTG- |
| | | Hs00226190_ | | 027206 | 310663 | GACTTTGGCAAAATGACTGACCGGGAAACG |
| | | m1 | | | | (SEQ ID NO: 35) |
| Ier3 | NM_003897.3 | IER3- | A_42_P515405 | NM_ | ID: | ATTTATTCTAACTTATGCAGGGGTGCGAGA- |
| | | Hs00174674_ | | 212505 | 15937 | TATGCCCCCTTGCTGTGACACAGATATTTA |
| | | m1 | | | | (SEQ ID NO: 36) |
| Pik3ap1 | NM_152309.2 | PIK3AP1- | A_43_P21121 | NM_ | ID: | ACCTGGAGACCCACTGTCACTGGTGATGGT- |
| | | Hs00381030_ | | 001106368 | 294048 | GTAGCCCTGCTGGTTTGGGTGATCCTTGAA |
| | | m1 | | | | (SEQ ID NO: 37) |
| Pstpip1 | NM_003978.3 | PSTPIP1- | A_44_P180717 | NM_ | ID: | TGGTGTGATAAAGAGGTTCTCTGGGCTGCT- |
| | | Hs00182777_ | | 011193 | 19200 | ACATGGAAGTCCCAAGACCACACCTTCTCA |
| | | m1 | | | | (SEQ ID NO: 38) |
| Ubd | NM_006398.3 | UBD- | A_42_P602724 | NM_ | ID: | GTGACTACGGGAGTGGGGTGATGAGAAGCT- |
| | | Hs00197374_ | | 053299 | 29168 | CAAAACCGACTTCCTTTAATCAATTAACCA |
| | | m1 | | | | (SEQ ID NO: 39) |
| C2 | NM_006987.2 | C2- | A_44_P332606 | NM_ | ID: | CCTGGTGAGTTGGGGTCTTTTTGACCCTTG- |
| | | Hs00163794_ | | 172222 | 12263 | TCACGGTTCCTCCAACAAAAACTTGCGCAG |
| | | m1 | | | | (SEQ ID NO: 40) |
| Lst1 | NM_001166538 | LST1- | A_43_P12274 | NM_ | ID: | AGGCAGAGGAGAAGGTGAAGGCGTAAAAGA- |
| | | Hs00394683_ | | 022634 | 64569 | AGACGCCAGCACTGACTATGCCTGCATCGT |
| | | m1 | | | | (SEQ ID NO: 41) |
| Aif1 | NM_001623.3 | AIF1- | A_44_P421534 | NM_ | ID: | TTTCTCAGAATGATGCTGGGCAAGAGATCT- |
| | | Hs00610419_ | | 019467 | 11629 | GCCATCTTGAGAATGATTCTGATGTATGAG |
| | | g1 | | | | (SEQ ID NO: 42) |
| C1QTNF7 | NM_001135170.1 | C1QTNF7- | A_44_P248172 | NM_ | ID: | GGTTTCTCCTCTATGTTGATACAGATTACC- |
| | | Hs00230467_ | | 175425 | 109323 | TGGATTCTATATCAGAAGACGATGAGTTGT |
| | | m1 | | | | (SEQ ID NO: 43) |
| MME | NM_002426.4 | MME- | A_43_P11484 | NM_ | ID: | ATCATATTGCTGAAAATCTTCAAACACAAA- |
| | | Hs00153519_ | | 012608 | 24590 | CTCTGGGGTGAGCATTACCATTGAACAGTT |
| | | m1 | | | | (SEQ ID NO: 45) |
| IGFBP5 | NM_000599.3 | IGFBP5- | A_44_P285534 | NM_ | ID: | ACCCCGGAAACGTATTCCTATTTGAAGCAA- |
| | | Hs01052296_ | | 012817 | 16011 | GTTGAACGGACAGAGAAGGGAAGAAGAGAA |
| | | m1 | | | | (SEQ ID NO: 46) |
| CARD11 | NM_032415.3 | CARD11- | A_44_P421727 | XM_ | ID: | GAGATGAGTACCTCCGGAAACAGAAGACGG- |
| | | Hs01060620_ | | 001073551 | 108723 | AGACCATCATCTACTCCCGAGAAAAGAACC |
| | | m1 | | | | (SEQ ID NO: 47) |

TABLE 11

| | | log2- | regulation in | |
|---|---|---|---|---|
| | | fold | human clinical | Gene Seq. |
| gene | p value | changes | GVHD biopsies | Ref. (human) |
| ANP32A | 0.022 | −2.03 | Down | NM_012903 |
| CARD11 | 0.0015 | 2.68 | Up | NM_032415.3 |
| C1QTNF7 | 0.0002 | −3.26 | Down | NM_001135170.1 |
| CEACAM4 | 0.003 | 4.86 | Up | NM_001817.2 |
| HCLS1 | 0.0006 | 2.53 | Up | NM_008225 |
| HTRA1 | 0.02 | −1.01 | Down | NM_031721 |
| LGALS7 | 0.0172 | −0.82 | Down | NM_022582 |
| LST1 | 0.0138 | −0.75 | Down | NM_001166538 |
| MSR1 | 0.0133 | 3.94 | Up | NM_138715.2 |
| PIK3AP1 | 0.0279 | 3.39 | Up | NM_152309.2 |
| PSTPIP1 | 0.0057 | 2.40 | Up | NM_003978.3 |
| PTGER2 | 0.0435 | 1.99 | Up | NM_031088 |
| PTPN7 | 0.0003 | 4.14 | Up | NM_177081 |
| TAP1 | 0.0174 | 3.83 | Up | NM_032055 |
| TGM2 | 0.003 | 5.12 | Up | NM_019386 |
| TREM2 | 0.001 | 4.23 | Up | NM_031254 |
| UBD | 0.0441 | 2.38 | Up | NM_006398.3 |
| CTGF | 0.036 | −1.90 | Down | NM_001901.2 |

### REFERENCES

Arora M, Weisdorf D J, Spellman S R, Haagenson M D, Klein J P, et al. (2009) HLA-identical sibling compared with 8/8 matched and mismatched unrelated donor bone marrow transplant for chronic phase chronic myeloid leukemia. J Clin Oncol 27: 1644-1652.
Yakoub-Agha I, Mesnil F, Kuentz M, Boiron J M, Ifrah N, et al. (2006) Allogeneic marrow stem-cell transplantation from human leukocyte antigen-identical siblings versus human leukocyte antigen-allelic-matched unrelated donors (10/10) in patients with standard-risk hematologic malignancy: a prospective study from the French Society of Bone Marrow Transplantation and Cell Therapy. J Clin Oncol 24: 5695-5702.
Consortium T M S (1999) Complete sequence and gene map of a human major histocompatibility complex. The MHC sequencing consortium. Nature 401: 921-923.
Petersdorf E W, Malkki M, Gooley T A, Martin P J, Guo Z (2007) MHC haplotype matching for unrelated hematopoietic cell transplantation. PLoS Med 4: e8.
Dickinson A M, Harrold J L, Cullup H (2007) Haematopoietic stem cell transplantation: can our genes predict clinical outcome? Expert Rev Mol Med 9: 1-19.
Hurt P, Walter L, Sudbrak R, Klages S, Müller I, et al. (2004) The genomic sequence and comparative analysis of the rat major histocompatibility complex. Genome Res 14: 631-639.
Naper C, Ryan J C, Kirsch R, Butcher G W, Rolstad B, et al. (1999) Genes in two major histocompatibility complex class I regions control selection, phenotype, and function of a rat Ly-49 natural killer cell subset. Eur J Immunol 29: 2046-2053.
Naper C, Dai K Z, Kveberg L, Rolstad B, Niemi E C, et al. (2005) Two structurally related rat Ly49 receptors with opposing functions (Ly49 stimulatory receptor 5 and Ly49 inhibitory receptor 5) recognize nonclassical MHC class Ib-encoded target ligands. J Immunol 174: 2702-2711.
Sviland L, Hromadnikova I, Sedlacek P, Cermakova M, Stechova K, et al. (2001) Histological correlation between different centers using the skin explant model to predict graft-versus-host disease following bone marrow transplantation. Hum Immunol 62: 1277-1281.
Dickinson A M, Wang X N, Sviland L, Vyth-Dreese F A, Jackson G H, et al. (2002) In situ dissection of the graft-versus-host activities of cytotoxic T cells specific for minor histocompatibility antigens. Nat Med 8: 410-414.
Novota P, Sviland L, Zinocker S, Stocki P, Balavarca Y, et al. (2008) Correlation of Hsp70-1 and Hsp70-2 gene expression with the degree of graft-versus-host reaction in a rat skin explant model. Transplantation 85: 1809-1816.
Thomas P D, Campbell M J, Kejariwal A, Mi H, Karlak B, et al. (2003) PANTHER: a library of protein families and subfamilies indexed by function. Genome Res 13: 2129-2141.
Pfaffl M W (2001) A new mathematical model for relative quantification in real-time RT-PCR. Nucleic Acids Res 29: e45.
Lerner K G, Kao G F, Storb R, Buckner C D, Clift R A, et al. (1974) Histopathology of graft-vs.-host reaction (GvHR) in human recipients of marrow from HL-A-matched sibling donors. Transplant Proc 6: 367-371.
Landgrebe J, Bretz F, Brunner E (2004) Efficient two-sample designs for microarray experiments with biological replications. In Silico Biol 4: 461-470.
Yang Y H, Dudoit S, Luu P, Lin D M, Peng V, et al. (2002) Normalization for cDNA microarray data: a robust composite method addressing single and multiple slide systematic variation. Nucleic Acids Res 30: e15.
Benjamini Y, Hochberg Y (1995) Controlling the false discovery rate: A practical and powerful approach to multiple testing. J Roy Statist Soc Ser B 57: 289-300.
Thomas P D, Kejariwal A, Guo N, Mi H, Campbell M J, et al. (2006) Applications for protein sequence-function evolution data: mRNA/protein expression analysis and coding SNP scoring tools. Nucleic Acids Res 34: W645-650.
Livak K J, Schmittgen T D (2001) Analysis of relative gene expression data using real-time quantitative PCR and the 2(-Delta Delta C(T)) Method. Methods 25: 402-408.
Twigger S N, Pruitt K D, Fernandez-Suarez X M, Karolchik D, Worley K C et al. (2008) What everybody should know about the rat genome and its online resources. Nat. Genet. 40: 523-527.

1-17. (canceled)

18. A method of predicting the risk of a subject to develop graft versus host reaction (GvHR) or graft versus host disease (GvHD), comprising
(a) determining the expression level of one or more prognostic RNA transcripts, or their corresponding cDNAs, or their expression products, in a sample obtained from said subject, wherein said transcript(s) or expression products is/are the transcript or expression product of one or more genes selected from the group consisting of:
(i) Msr1, Pik3ap1, Pstpip1, Ctss, Pbx2, Grem1, Ly6g6e, Olr1, Spr1, Spic, Nfe2, Tnfaip8l2, and Ier3; or
(ii) Msr1, Ctss, Pbx2, Grem1, Ly6g6e, Olr1, Spr1, Spic, and Nfe2; or
(iii) Pik3ap1, Pstpip1, Tnfaip8l2, and Ier3;
(b) comparing the expression level of said one or more prognostic RNA transcript, or its corresponding cDNA, or its expression product with a corresponding baseline value; wherein
(i) for every unit of increased expression of Olr1, Msr1, Pik3ap1, and/or Pstpip1; or the corresponding cDNA or expression product, said subject is expected to develop GvHR or GvHD; and
(ii) for every unit of decreased expression of Ctss, Pbx2, Grem1, Ly6g6e, Spr1, Spic, Nfe2, Tnfaip8l2, and/or Ier3; or the corresponding cDNA or expression product, said subject is expected to develop GvHR or GvHD.

19. The method of claim 18, wherein the expression level is determined by DNA microarray analysis or quantitative PCR and subsequent calculation of the mRNA copy number normalized to the amount of total RNA or to the expression level of one or more housekeeping genes.

20. The method of claim 18, wherein the expression level of the corresponding expression product(s) is determined by ELISA, Western blotting, protein microarray, immunohistochemistry, flow cytometry or surface plasmon resonance.

21. The method of claim 18, wherein the sample is a biopsy sample or a sample of Peripheral Blood Mononuclear Cells (PBMC).

22. The method of claim 18, wherein the subject is a mammal.

23. The method of claim 18, wherein the subject is a human.

24. The method of claim 18, wherein the baseline value is the expression level of said at least one gene in at least one healthy subject.

25. The method of claim 18, further comprising determining the prognostic transcript of one or more genes selected from the group of genes consisting of Ubd, C2, Lst1, Aif1, C1QTNF7, CEACAM4, MME, IGFBP5, Tap1, Ctgf, ANP32A, HCLS1, HTRA1, LGALS7, PTGER2, PTPN7, TGM2, TREM2 and CARD11; or their corresponding cDNAs, or their expression products, wherein
(i) for every unit of increased expression of one or more of Ubd, C2, Aif1, CEACAM4, Tap1, PTGER2, PTPN7, TGM2, TREM2, HCLS1 and/or CARD11; or the corresponding cDNA or expression product, said patient is expected to develop GvHR or GvHD; and
(ii) for every unit of decreased expression of one or more of Lst1, C1QTNF7, MME, Ctgf, ANP32A, HTRA1, LGALS7 and/or IGFBP5; or the corresponding cDNAs or expression product(s), said patient is expected to develop GvHR or GvHD.

26. A method of diagnosing graft versus host reaction (GvHR) or graft versus host disease (GvHD) in a subject following transplantation, comprising:
(a) determining the expression level of one or more prognostic RNA transcripts, or their corresponding cDNAs, or their expression products, in a sample obtained from said subject, wherein said transcript(s) or expression products is/are the transcript or expression product of one or more genes selected from the group consisting of:
(i) Msr1, Pik3ap1, Pstpip1, Ctss, Pbx2, Grem1, Ly6g6e, Olr1, Spr1, Spic, Nfe2 Tnfaip8l2, and Ier3; or
(ii) Msr1, Ctss, Pbx2, Grem1, Ly6g6e, Olr1, Spr1, Spic, and Nfe2; or
(iii) Pik3ap1, Pstpip1, Tnfaip8l2, and Ier3;
(b) comparing the expression level of said one or more prognostic RNA transcript, or its corresponding cDNA, or its expression product with a corresponding baseline value; wherein
(i) every unit of increased expression of Olr1, Msr1, Pik3ap1, and/or Pstpip1, or the corresponding cDNA or expression product, is indicative of GvHR or GvHD; and
(ii) every unit of decreased expression of Ctss, Pbx2, Grem1, Ly6g6e, Spr1, Spic, Nfe2, Tnfaip8l2, and/or Ier3, or the corresponding cDNA or expression product, is indicative of GvHR or GvHD.

27. The method of claim 26, wherein the expression level is determined by DNA microarray analysis or quantitative PCR and subsequent calculation of the mRNA copy number normalized to the amount of total RNA or to the expression level of one or more housekeeping genes.

28. The method of claim 26, wherein the expression level of the corresponding expression product(s) is determined by ELISA, Western blotting, protein microarray, immunohistochemistry, flow cytometry or surface plasmon resonance.

29. The method of claim 26, wherein the sample is a biopsy sample or a sample of Peripheral Blood Mononuclear Cells (PBMC).

30. The method of claim 26, wherein the subject is a mammal.

31. The method of claim 26, wherein the subject is a human.

32. The method of claim 26, wherein the baseline value is the expression level of said at least one gene in at least one healthy subject.

33. The method of claim 26, further comprising determining the prognostic transcript of one or more genes selected from the group of genes consisting of Ubd, C2, Lst1, Aif1, C1QTNF7, CEACAM4, MME, IGFBP5, Tap1, Ctgf, ANP32A, HCLS1, HTRA1, LGALS7, PTGER2, PTPN7, TGM2, TREM2 and CARD11; or their corresponding cDNAs, or their expression products, wherein
(i) every unit of increased expression of Ubd, C2, Aif1, CEACAM4, Tap1, PTGER2, PTPN7, TGM2, TREM2, HCLS1 and/or CARD11; or the corresponding cDNA or expression product, is indicative of GvHR or GvHD; and
(ii) every unit of decreased expression of Lst1, C1QTNF7, MME, Ctgf, ANP32A, HTRA1, LGALS7 and/or IGFBP5; or the corresponding cDNA or expression product, is indicative of GvHR or GvHD.

34. The method of claim 26, wherein the baseline value is the expression level of said at least one gene in said subject prior to said transplantation, or in at least one healthy subject, or in both.

35. A method of monitoring the efficacy of treatment of graft versus host reaction (GvHR) or graft versus host disease (GvHD) in a subject following transplantation, comprising:
(a) determining the expression level of one or more prognostic RNA transcripts, or their corresponding cDNAs, or their expression products, in a sample obtained from said subject at a first time point T1, and a later second time point T2, wherein said transcript(s) or expression products is/are the transcript or expression product of one or more genes selected from the group consisting of:
(i) Msr1, Pik3ap1, Pstpip1, Ctss, Pbx2, Grem1, Ly6g6e, Olr1d, Spr1, Spic, Nfe2, Tnfaip8l2, and Ier3; or
(ii) Msr1, Ctss, Pbx2, Grem1, Ly6g6e, Olr1, Spr1, Spic, and Nfe2; or
(iii) Pik3ap1, Pstpip1, Infaip812, and Ier3;
(b) comparing the expression level of said one or more prognostic RNA transcript, or its corresponding cDNA, or its expression product at time point T1 (Δ1) and time point T2 (Δ2) with a corresponding baseline value; wherein
(i) a decline in units of an increased expression of Oki, Msr1, Pik3ap1, and/or Pstpip1; or the corresponding cDNA or expression product at time point T2 in comparison with the increased expression of said at least one gene at the time point T1 (ΔΔ=Δ1−Δ2), is indicative of effective treatment of GvHR or GvHD; and
(ii) a decline in units of a decreased expression of Ctss, Pbx2, Grem1, Ly6g6e, Spr1, Spic, Nfe2, Tnfaip8l2, and/or Ier3; or the corresponding cDNA or expression product at time point T2 in comparison with the decreased expression of said at least one gene at the time point T1(ΔΔ=Δ1−Δ2), is indicative of effective treatment of GvHR or GvHD.

36. The method of claim 35, wherein the expression level is determined by DNA microarray analysis or quantitative PCR and subsequent calculation of the mRNA copy number normalized to the amount of total RNA or to the expression level of one or more housekeeping genes.

37. The method of claim 35, wherein the expression level of the corresponding expression product(s) is determined by ELISA, Western blotting, protein microarray, immunohistochemistry, flow cytometry or surface plasmon resonance.

38. The method of claim 35, wherein the sample is a biopsy sample or a sample of Peripheral Blood Mononuclear Cells (PBMC).

39. The method of claim 35, wherein the subject is a mammal.

40. The method of claim 35, wherein the subject is a human.

41. The method of claim 35, wherein the baseline value is the expression level of said at least one gene in said subject prior to said transplantation, or in at least one healthy subject, or in both.

42. The method of claim 35, further comprising determining the prognostic transcript of one or more genes selected from the group of genes consisting of Ubd, C2, Lst1, Aif1, C1QTNF7, CEACAM4, MME, IGFBP5, Tap1, Ctgf, ANP32A, HCLS1, HTRA1, LGALS7, PTGER2, PTPN7, TGM2, TREM2 and CARD11; or their corresponding cDNAs, or their expression products, wherein
(i) a decline in units of an increased expression of Ubd, C2, Aif1, CEACAM4, Tap1, PTGER2, PTPN7, TGM2, TREM2, HCLS1 and/or CARD11; or the corresponding cDNA or expression product at time point T2 in comparison with the increased expression of said at least one gene at the time point T1 (ΔΔ=Δ1−Δ2), is indicative of effective treatment of GvHR or GvHD; and
(ii) a decline in units of a decreased expression of Lst1, C1QTNF7, MME, Ctgf, ANP32A, HTRA1, LGALS7 and/or IGFBP5; or the corresponding cDNA or expression product at time point T2 in comparison with the decreased expression of said at least one gene at the time point T1(ΔΔ=Δ1−Δ2), is indicative of effective treatment of GvHR or GvHD.

43. A method of screening for a candidate substance for treatment of graft versus host reaction (GvHR) or graft versus host disease (GvHD), comprising:
(a) monitoring the efficacy of treatment by said candidate substance by using the method according to claim 18 in
(i) a non-human animal model which suffers from GvHR or GvHD and to which the candidate substance has been administered, or
(ii) in an ex vivo model, including but not limited to cell-based and/or tissue-based GvHR or HvHD assay such as the Skin Explant Assay, wherein said cells and/or tissue have been contacted with said candidate substance; and
(b) selecting a candidate substance which shows effective treatment of GvHR or GvHD.

44. The method of predicting the risk of developing graft versus host reaction (GvHR) or graft versus host disease (GvHD) according to claim 18, or the method of diagnosing GvHR or GvHD according to claim 26, or a method of monitoring the efficacy of treatment of GvHR or GvHD, comprising the step of using a kit, wherein the kit comprises at least one isolated polynucleotide, wherein each isolated polynucleotide independently comprises
(i) at least 20 contiguous nucleotides of the nucleotide sequence selected from SEQ ID NO: 1, 3, 5, 7, 8, 10, 12, 14, 16, 18, 20, 22, and 24; or SEQ ID NO: 26-47, or
(ii) a nucleotide sequence having at least 90% identity to (i), or
(iii) the coding region of a gene comprising a nucleotide sequence according to (i) or (ii), or
(iv) a nucleotide sequence that can specifically hybridize, under conditions of high stringency, to a polynucleotide having a nucleotide sequence according to (i), (ii) or (iii); and
wherein the kit comprises no more than 9000 isolated polynucleotides in total.

45. The method of claim 44, wherein the isolated polynucleotides comprise at least 25 contiguous nucleotides.

46. The method of claim 44, wherein the isolated polynucleotides are arranged in an array.


SEQUENCE LISTING (77 sequences)

SEQ ID NO: 1 - DNA, 4100 bp, Homo sapiens
  CDS (134)..(1129): Ctss
gtacctcatgtgacaagttccaatttcttttcaagtcaattgaactgaaatctccttgttgctttgaaatcttagaagagagcccactaattcaaggactcttactgtgggagcaactgctggttctatcacaatgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaatcggatattgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaataaaggcccagtgtctgttggtgtagatgcgcgtcatccttctttcttcctctacagaagtggtgtctactatgaaccatcctgtactcagaatgtgaatcatggtgtacttgtggttggctatggtgatcttaatgggaaagaatactggcttgtgaaaaacagctggggccacaactttggtgaagaaggatatattcggatggcaagaaataaaggaaatcattgtgggattgctagctttccctcttacccagaaatctagaggatctctcctttttataacaaatcaagaaatatgaagcactttctcttaacttaatttttcctgctgtatccagaagaaataattgtgtcatgattaatgtgtatttactgtactaattagaaaatatagtttgaggccgggcacggtggctcacgcctgtaatcccagtacttgggaggccaaggcaggcatatcaacttgaggccaggagttaaagagcagcctggctaacatggtgaaaccccatctctactaaaaatacaaaaaattagccgagcacggtggtgcatgcctgtaatcccagctacttgggaggctgaggcacgagattccttgaacccaagaggttgaggctatgttgagctgagatcacaccactgtactccagcctggatgacagagtggagactctgtttcaaaaaaacagaaaagaaaatatagtttgattcttcatttttttaaatttgcaaatctcaggataaagtttgctaagtaaattagtaatgtactatagatataactgtacaaaaattgttcaacctaaaacaatctgtaattgcttattgttttattgtatactctttgtctttttaagacccctaatagccttttgtaacttgatggcttaaaaatacttaataaatctgccatttcaaatttctatcattgccacataccattcttattcctaggcaactattaataatctatcctgagaatattaattgtggtattctggtgatggggtttagcaactttgatggaagaaaatattaggctataaatgtcctaaggactcagattgtatctttgtacagaagaggattcaaaacgccacgtgtagtggctcatgcctgtaatcccaacactttgggaggctgaagtaggaggatcgtcttgagcccaggagttcaagaccagcctggacaacatagtgagaccttgtctccacaaaaataaaaaagaaactatccaggagtggtggtgtgtgcctgtggtccctgctatgcagatgtctaagacaggaggatcacaagagcccaggaggttgagaatgcagtgagcttgtaattgcaccactgcactccagcctgggtgacagagcaagaccctgtcttaaaaaaagaggattcaacacatatttttatattatgttaaagtaaagaaatgcataaaagacaagcactttggaagaattattttaatgatcaacaatttaatgtattagtccaaattatttttacgtagtcatcaacaatttgaccagggcctttatttggcaaataactgagccaaccagaataaaataaccaatactccactgctcatatttttatctaattcagatggatcttccttacaactgctctagattagtagatgcatctaagcaggcagcaggaactttaaattttttaagttcatgtctatgacatgaacaatgtgtgggataatgtcattaatatatcctaaattaacctaaacgtatttcactaactctggctccttctccataaagcacattttaaggaacaagaattgctaaatataaaaacataaataataccataatacatggctatcatcaaaagtgtatagaatattatagtttaaaagtatttagttgattacttttcagttttgttttgttttttgagacggagtctcactctgttgcccaggctggagtgcagtggcaccatctcagttcactgcaacttctgcctcccgagttcaagcgattctcctgcctcagcctcccgagtagctggaattataggcgtgcaccaccacgcccagctaatttttgtatttttagtaaagacagggttttgccacattagccaggctggtctcaaactcctgacctcaggtgatccacccaccccagcctcccaaagtgctaagattacaggcgtgagccactgagcccagcctacttttcagtttttaacataatttttgttttatccacaacttttcaagtattgaaagtagaataaaaacatgggttcttagtctttagctatctgttaaagcctatgaatgccttcttaaaatcatgtttttaaatgcataaaatatataggattacaaaggaatctaattatatcgaaatacagttattaaaatgttaaaagataagtttgttatatattaatatgcatgcttctttataaatgcattaaataagagttaatagctatcctaaatttgaaatagtgataagcataatgaaaatagatgcaaaaaactaatgtgatatgaaaatatctgggtttttcttttgatgatgaagtattgctaatattaccgtggtttatgaactatgttcagaattgaagaaaatcctaactttcagttagaggttagtgacggggttcaggacaccctacacaaaatacagcactttgacatattgaatattttaagctgaaggcatttgaggaaattgcagaagcaggaaggtgactctgaccttctgcctgctgttctccccagaagcagccataaaacctgggaaggattttctgaccttcccctgaagtagatcataagactgtcatgtaagaggtgctctcctggcacccagagaaaaggagcatccttacctccaaaagcacagggacacaaagaggaatctaaacaaacaggcctctcagtttcccccagtttattacatttagcttgttcacactttgccctatgacatttctacatcactggctgctcttcatcaaacctactataaaaaacattcaagttcaactgtttctttgggcctttatttccttatggagcccctcgtgtcgtgtaaaacttatattaaataaatgtgcatgcttttctcttgctaatctctcttttgttatagagatctcagccctaaacctaggatggatagaaggaaacatatgttctcccctacattagtaaaaataaaaatggaattttttacccatacaaa

SEQ ID NO: 2 - protein, 331 aa, Homo sapiens
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 3 - DNA, 3231 bp, Homo sapiens
  CDS (272)..(1564): Pbx2
ctctctcacacacacccccgcttgggcctcctctctctctccggctccattttctccgccgccgggggccggggtctcctgtggggggcccagccggtatcccaggtctcccttcagtgccggggtgaacccccgggggagccgggagccgggggcagacgggcgggggttggggcggagggagcagcggccccagcgagtttggggggagaagtaaccaggcggggggaggggcggagcagggagggggcctcagggcccccccccagctatggacgaacggctactggggccgccccctccaggcgggggccgggggggcctgggattggtgagtggggagcctgggggccctggcgagcctcccggtggcggagaccccggtgggggtagcgggggggtcccgggaggccgagggaagcaagacatcggggacattctgcagcagataatgaccatcaccgaccagagcctggacgaggcccaggccaagaaacacgccctaaactgccaccgaatgaagcctgctctctttagcgtcctgtgtgaaatcaaggagaaaactggcctcagcattcggagctcccaggaggaggagccggtggacccacagctgatgcgcttggacaacatgcttctggcagagggtgtggctgggcccgagaaagggggcggctcagcagcagcagctgcagccgctgcagcctctggtggtggtgtgtcccctgacaactccatcgaacactcggactatcgcagcaaacttgcccagatccgtcacatataccactcggagctggagaagtatgagcaggcatgtaatgagttcacgacccatgtcatgaacctgctgagggagcagagccgcaccaggcccgtggcccccaaagagatggaacgcatggtgagcatcatccatcgaaagttcagcgccatccagatgcagctgaagcagagcacctgcgaggctgtgatgatcctgcgctcccgtttcctggatgccagacgaaagcgccgtaacttcagcaaacaggccactgaggtcctaaatgagtatttctactcccacctgagtaacccatatcctagtgaggaggccaaggaggagcttgccaagaagtgtggcatcaccgtgtctcaggtctccaactggtttggcaacaagaggattcgctataagaaaaacatcggaaagttccaagaggaggcaaacatctatgctgtcaagaccgccgtgtcagtcacccaggggggccacagccgcaccagctccccgacacccccttcctctgcaggctctggcggctctttcaatctctcaggatctggagacatgtttctggggatgcctgggctcaacggagattcctattctgcttcccaggtggaatcactccgacactcgatggggccagggggctatggggataacctcgggggaggccagatgtacagcccacgggaaatgagggcaaatggcagctggcaagaggctgtgaccccctcttcagtgacatccccaacggagggaccagggagtgttcactctgatacctccaactgatcttgcccctcagggtcacaggggtgggggctctcacaaggcgacttgaagaggacgcaggcttccagaggacaaaccccaatacaggagaagcacaagacagagaagggccaatggggtcatcccctccctaacgagactctctgtgctgggggtgctaattacatggcaggaagaatggggcctctaaggggagtgtggggtctgtctctcccttttttccatctttttcctctctcgctttctttcttacacagaaacatacacataccgagaaacctatttctcagacccctttttctcctctgtctttctctctccctctcccacacctcacacacacatactcccacttgcaactattctgtttctctcctgggctcccccactttcccttccccaccccacttgtatgctctggaatctgtggagacgccagccctgcccaatcagagatgccaaaaatggggacatgacttctggacagaggacatgggccacgcccccatgcatccccacccccgcccctccggacggcttacttacctcatacgcagctcatcttaaaccaatagaatcgctcggtggacgagagtgtctgactcagatatctacctcggagggagtttctgctactttagggaattattgactgggctttggggttgaacttttttttttttaaagaaagaaaaagaaaccctgggatccatctgttttttttgttgttgttgttgtttttgttgttgttggtggtggtggtggtggtggttcttaatttttaatttagtttggggaagtagcttgtttttttttttataaatatgttgatttcttgtctttttttttttttatttcttactttcccatattaggggtgatagccaaaggggttctggtaagagaaagggggacaaacagaactggtaaagaggcccccctggctccaggcctgtccatcaggaagtaaattttacagggcaccaagctttgccccctaaaatcccttaggtgttctttgttcatgcaggcaggtttctgccgcatttgatgtggaggcagtgaagggcttgccctgctggcctctcatcccccttcttcccacaacccttgggcagggctggactcagtaattttgaggaaattgaagatgccatcttcccctgtgagtgacatgtctttaattttttaaaaaactactatttgaaaattggagggggaagaatgggaagggagttattgccaaatatgttaaatatgggttggggtgcttgtatatgtatcttcctcaatttccccataaatgaggtatctttttgtcacaccaaaatcaaggggtagggagagggaggaggttgcaaaaagccagatgtgggggaaaagtaacatcaacactgtcccatcctcagccctgaactagctaccatctgatcccctcagacattctcaggattttacaagactgtcagagtggggaacccctcccattaaagatccgggcaggactggggacaggttggaagtgtgatgggtgggggggtgggaggcatgggccgggggcagttctctcctcacttgtaaacttgtgtagtttcacagaaaaaaaacaaaatgcagttttaaataaagaaatttcttttttccctgggaaaaaaaaaaaaaaaaaa

SEQ ID NO: 4 - protein, 430 aa, Homo sapiens
MDERLLGPPPPGGGRGGLGLVSGEPGGPGEPPGGGDPGGGSGGVPGGRGKQDIGDILQQIMTITDQSLDEAQAKKHALNCHRMKPALFSVLCEIKEKTGLSIRSSQEEEPVDPQLMRLDNMLLAEGVAGPEKGGGSAAAAAAAAASGGGVSPDNSIEHSDYRSKLAQIRHIYHSELEKYEQACNEFTTHVMNLLREQSRTRPVAPKEMERMVSIIHRKFSAIQMQLKQSTCEAVMILRSRFLDARRKRRNFSKQATEVLNEYFYSHLSNPYPSEEAKEELAKKCGITVSQVSNWFGNKRIRYKKNIGKFQEEANIYAVKTAVSVTQGGHSRTSSPTPPSSAGSGGSFNLSGSGDMFLGMPGLNGDSYSASQVESLRHSMGPGGYGDNLGGGQMYSPREMRANGSWQEAVTPSSVTSPTEGPGSVHSDTSN

SEQ ID NO: 5 - DNA, 4150 bp, Homo sapiens
  CDS (160)..(714): Grem1
actcggtgcgccttccgcggaccgggcgacccagtgcacggccgccgcgtcactctcggtcccgctgaccccgcgccgagccccggcggctctggccgcggccgcactcagcgccacgcgtcgaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagccccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcactccctattttgtaaacatatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgatagtgactatgtcagtctaatctcttgtttgccaaggttcctaaattaattcacttaaccatgatgcaaatgtttttcattttgtgaagaccctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacagaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttagcaagagatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcactgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcttcaagcctgaggttttatatacaaactccctgaatactctttttgccttgtatcttctcagcctcctagccaagtcctatgtaatatggaaaacaaacactgcagacttgagattcagttgccgatcaaggctctggcattcagagaacccttgcaactcgagaagctgtttttatttcgtttttgttttgatccagtgctctcccatctaacaactaaacaggagccatttcaaggcgggagatattttaaacacccaaaatgttgggtctgattttcaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccagcaaacactgaatttctcttgttgttttaactctgccacaagaatgcaatttcgttaacggagatgacttaagttggcagcagtaatcttcttttaggagcttgtaccacagtcttgcacataagtgcagatttggctcaagtaaagagaatttcctcaacactaacttcactgggataatcagcagcgtaactaccctaaaagcatatcactagccaaagagggaaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagatatttgtggtcttgatcatacctattaaaataatgccaaacaccaaatatgaattttatgatgtacactttgtgcttggcattaaaagaaaaaaacacacatcctggaagtctgtaagttgttttttgttactgtaggtcttcaaagttaagagtgtaagtgaaaaatctggaggagaggataatttccactgtgtggaatgtgaatagttaaatgaaaagttatggttatttaatgtaattattacttcaaatcctttggtcactgtgatttcaagcatgttttctttttctcctttatatgactttctctgagttgggcaaagaagaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaaagagcaactaataaattaaacctattctttctgtgaaaaaaaaaaaa

SEQ ID NO: 6 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 7 - DNA, 652 bp, Homo sapiens
  misc_feature: Ly6g6e
agagacaacatgctgagagagcagaggagaccctccaggcaagggcagactccttgcaggggcaggctgggggcccccgctgcctgctgggtcaggctggtgaatctggtcatggttccgccccccagattcactccctaggtgtgtttgtttactggttcctcactgtcttgctcaaatgctccaactctacaaatcccgggatctcggggtgcagatcacctctcccagattcctgagcctgtgtctggccatgggcacctccagcatcttcctctgcgtgctgttcctctgtggggcactgggtctcaccatgtcccctgcccggggaaggctccgctgctacatctgtggcttcaccaaaccctgccaccctgttcccaccgagtgtcgggacgatgaagcttgtggcatcagtattggcacttcagaccagagtgagatcactgagtgaaaaagctgcctctcaagggcccagtgccctctgccaggctatgccacctactggctgcactcctacactctgtggcaccactgctgcgagcaggacctgtgcaacatagccgcttccccacagcagctcaccagcctcctcgcctccctgcccctctttgtggccagcttcgctgggagaggacacctcctccactag

SEQ ID NO: 8 - DNA, 2533 bp, Homo sapiens
  CDS (115)..(936): Olr1
attcttctattagataacagtagctatttaaatacttctgcagaagctcacatatttttagtttgttgaagttcgtgactgcttcactctctcattcttagcttgaatttggaaatgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcagctccttgtccgcaagactggatctggcatggagaaaactgttacctattttcctcgggctcatttaactgggaaaagagccaagagaagtgcttgtctttggatgccaagttgctgaaaattaatagcacagctgatctggacttcatccagcaagcaatttcctattccagttttccattctggatggggctgtctcggaggaaccccagctacccatggctctgggaggacggttctcctttgatgccccacttatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtgaatttgaaggctctggaagaaaagaaaaaagtctttgagttttattctggaatttaagctattctttgtcacttgggtgccaaacatgagagcccagaaaactgtcatttagctggctgcagaactcctttgcagaaactggggttccaggtgcctggcacctttatgtcaacatttttgattctagctacctgtattatttcacctagcttgtcccaagcttccctgccagcctgaagtccattttcccctttttattttaaaatttgactcctcttcaagcttgaaaaccctctgaactcagtcttctttacctcattatcaccttcccctcacactcctaaaattgcatgaaagacagaacatggagaacttgctcaagtgcaggcagagagcaaaaaggggaaatatgtctgggaaaaagtgcacgtgaagaaacaaagaaggacagaggccattccgaaatcaagaaactcatgttcttaactttaaaaaaggtatcaatccttggtttttaaactgtggtccatctccagactctaccacttacggacagacagacagacagacacacacacacacacacacacacattttgggacaagtggggagcccaagaaagtaattagtaagtgagtggtcttttctgtaagctaatccacaacctgttaccacttcctgaatcagttattatttcttcatttttttttctaccagaggacagattaatagatttaacccttcacaacagttcttgttagaatcatgggatgtgtggcccagaggtaagaatagaatttctttccctaaagaacataccttttgtagatgaactcttctcaactctgttttgctatgctataattccgaaacatacaagacaaaaaaaatgaagacactcaatctagaacaaactaagccaggtatgcaaatatcgctgaatagaaacagatggaattagaaatatatcttctatttttaggcttctatttcctttccacccactcttcacaggctattctactttaaaggaagcctttttattttgctgcacacaatctagcaggaatctttttttttttttaagagctgtgtcatccttatgtaggcaagagatgtttgcttttgttaaaagctttattgagatataattaacataaaataaactgaacatatttaaagtgtactatttgataagttttcacaccttgtggagaacatgcatactacaattaagagagtgaacatatccatcatccctcaaagtgtcacaatgctcctcctgatgactcctccccagaaaaccaccaatcggctttcattttgcattttgtagttttatgtgaatggaatcatatagtatgtcttttttttttgtctggcttctttcactttgcataattattttgagattcatatgtctccatcttgatgctcgtatgaattcattcttttaaatgttgaatattcccttgtatggatataccacaattcatttacccatttacttgttgatgacatttgggttgttttagttttgggatattacaaataaagctgctgtgaacatttgtgtacaagaaaaaaaaaaaaaaaaa

SEQ ID NO: 9 - protein, 273 aa, Homo sapiens
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 10 - DNA, 1185 bp, Homo sapiens
  CDS (325)..(735): Spr1
gtgattacggagatgccaagtgggtattgactgctccaggatgtggatggagggtgtgaaaaccagggtggggtgacgcaggctctgggtcatgatagggagagcaggcagctgggtcctgggctggaggactaaaataagggacgccaccttcaggggtgacacatcagcccaggccttcccaacgggtttgaccagttctgttctgatggtattcctgtgccactgggctggtccctcctccactcctcccctataaagcctcttggggttcccaggcacccagactcagcccaccccagctttgggggccagtacatagccatgatcctcaactggaagctcctggggatcctggtcctttgcctgcacaccagaggcatctcaggcagcgagggccacccctctcacccacccgcagaggaccgagaggaggcaggctccccaacattgcctcagggccccccagtccccggtgacccttggccaggggcaccccctctctttgaagatcctccgcctacccgccccagtcgtccctggagagacctgcctgaaactggagtctggctccctgaaccgcctagaacggatcctcctcaacctccccggcctgacgacccttggccggcaggaccccagcccccagaaaacccctggcctcctgcccctgaggtggacaaccgacctcaggaggagccagacctagacccaccccgggaagagtacagataatggagtcccctcagccgttctgttcccaggcatctccaggcacccacgccctctccaccctctgattccccgtgaattcttcccaatttagcctgtctccttaaacctcttcctcattccctcggttttattctgaacccgtaaggtggtgttctcaatatttcctgtcccctcctgagatccatacttagtcctcacatcgcccgttttttcctctgacagcctaagcctactctcctacctcgcctccaggcctcggccgcacctacctcccacccggtcttcctgcccgcgcgatcgctggggcagggctatggtactgtgttcccttctgccacctggtggccggcggcaggaactatcagtagacagctgctgcttccatgaaacggaaaaataaaaatcatgttttcttaaaaaaaaaaaaaaaaaaaaaaaaaaaaaaaaaaa

SEQ ID NO: 11 - protein, 136 aa, Homo sapiens
MILNWKLLGILVLCLHTRGISGSEGHPSHPPAEDREEAGSPTLPQGPPVPGDPWPGAPPLFEDPPPTRPSRPWRDLPETGVWLPEPPRTDPPQPPRPDDPWPAGPQPPENPWPPAPEVDNRPQEEPDLDPPREEYR

SEQ ID NO: 12 - DNA, 3761 bp, Homo sapiens
  CDS (199)..(1554): Msr1
aaatttagattttgcaaacctgtgcattgatgagagtgctattgaaacacattaagaaagattttcaacgcaggaatgtgtcatttcctttcttcatgtaccagatgctgaaatactatgagataaagattttaggtttcaattgtaaagagagagaagtggataaatcagtgctgctttctttaggacgaaagaagtatggagcagtgggatcactttcacaatcaacaggaggacactgatagctgctccgaatctgtgaaatttgatgctcgctcaatgacagctttgcttcctccgaatcctaaaaacagcccttcccttcaagagaaactgaagtccttcaaagctgcactgattgccctttacctcctcgtgtttgcagttctcatccctctcattggaatagtggcagctcaactcctgaagtgggaaacgaagaattgctcagttagttcaactaatgcaaatgatataactcaaagtctcacgggaaaaggaaatgacagcgaagaggaaatgagatttcaagaagtctttatggaacacatgagcaacatggagaagagaatccagcatattttagacatggaagccaacctcatggacacagagcatttccaaaatttcagcatgacaactgatcaaagatttaatgacattcttctgcagctaagtaccttgttttcctcagtccagggacatgggaatgcaatagatgaaatctccaagtccttaataagtttgaataccacattgcttgatttgcagctcaacatagaaaatctgaatggcaaaatccaagagaataccttcaaacaacaagaggaaatcagtaaattagaggagcgtgtttacaatgtatcagcagaaattatggctatgaaagaagaacaagtgcatttggaacaggaaataaaaggagaagtgaaagtactgaataacatcactaatgatctcagactgaaagattgggaacattctcagaccttgagaaatatcactttaattcaaggtcctcctggacccccgggtgaaaaaggagatcgaggtcccactggagaaagtggtccacgaggatttccaggtccaataggtcctccgggtcttaaaggtgatcggggagcaattggctttcctggaagtcgaggactcccaggatatgccggaaggccaggaaattctggaccaaaaggccagaaaggggaaaaggggagtggaaacacattaactccatttacgaaagttcgactggtcggtgggagcggccctcacgaggggagggtggagatactccacagcggccagtggggtacaatttgtgacgatcgctgggaagtgcgcgttggacaggtcgtctgtaggagcttgggatacccaggtgttcaagccgtgcacaaggcagctcactttggacaaggtactggtccaatatggctgaatgaagtgttttgttttgggagagaatcatctattgaagaatgtaaaattcggcaatgggggacaagagcctgttcacattctgaagatgctggagtcacttgcactttataatgcatcatattttcattcacaactatgaaatcgctgctcaaaaatgattttattaccttgttcctgtaaaatccatttaatcaatatttaagagattaagaatattgcccaaataatattttagattacaggattaatatattgaacaccttcatgcttactattttatgtctatatttaaatcattttaacttctataggtttttaaatggaattttctaatataatgacttatatgctgaattgaacattttgaagtttatagcttccagattacaaaggccaagggtaatagaaatgcataccagtaattggctccaattcataatatgttcaccaggagattacaattttttgctcttcttgtctttgtaatctatttagttgattttaattactttctgaataacggaagggatcagaagatatcttttgtgcctagattgcaaaatctccaatccacacatattgttttaaaataagaatgttatccaactattaagatatctcaatgtgcaataacttgtgtattagatatcaatgttaatgatatgtcttggccactatggaccagggagcttatttttcttgtcatgtactgacaactgtttaattgaatcatgaagtaaattgaaagcaggacatatgagaaaactgaccatcagtatatttgtccagataattggtggatcaaaaatgccacttaacaggaagtttagtttgttatgcactttaaatggaataattagcttgttacaattctaggacatggtgtttaaaatttaaatctgattaatccattttaacaaacaatgcaaacatcttcagtgcagaaggaagagtggtttcaactgtttggagtcttttatgaagtcagtcaacatttacaaccaaagggcgggggggggggtggggggtgcgtctttagtcctaaagggacaataactctgagcatgccccaaaaaagtagtttagcaaccttttgttggtagtcaacccatccccagggccatagtgtagagtgtgaaaagctaccctgaaacccagtaattctaccctgaaagtgactgcctgcagaaagaccagcagttgatattaaagcgcaaatgaattcaacctcagccctgaaaataacagaattctgaagtttcctatgactaattcacaaaaaaagtaattgtaaactagtactattatggaattactctactgttctttctttaatagtggcaaatgaaagcataagcttaagcattttttcatattctgaagtctcaccacacataataaccaagtggtagactcacagccgtccaacttaaaaaggcaaaaccttaccttggaattggaattactgtaaacagcctactgaaaatgcatttttatcatgtaacattcttctacttgtttaacattgctgattttctctggcagcataattttgtggttaagagaatgaattctgaatgtacactttctgtctcaaaccctggctgtaatttcagctagttaataattctttgtgttcagttccactatctaggtattttcttcaaaaggtaaatacaatggtttctgaaagaatcatttgcattatcagcctgtttgggatgtctgagatcagtgcctctgggttgttaatactgtattgctgtatggtatatgtatgctgatttactacttatgcgtaagtggtatgcatgggatgtctgaaatcagtgcctatgggttgtcaatagtattaactattagtgttaactgttagtattaactattagtattattaacactaataatagtactattactattactatttttattttaaaataaaatttacctttaaaataataatagtactattgctagtactagtactattgctattactagtactattactagtactagtactatgacactgttaatagtactattaacaacccataggcacttgggatgtctgagatcagtgcctatgggttgttaatactatattgctgtatggtatatgcatgctgatttaccacttatgcatagatatatctttaataagtaatctaaaaatcctttttgtatttgagagaatctactaagttcagtccagtcaagaaaagaacctaatagcaccaatacaaattgaggacttaatttactttggaatgttgaattgcatttgttccattaaaaaaaacagaaatttgcga

SEQ ID NO: 13 - protein, 451 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 14 - DNA, 1116 bp, Homo sapiens
  CDS (27)..(773): Spic
cagaattgtcaatttattaatgaaatatgacgtgtgttgaacaagacaagctgggtcaagcatttgaagatgcttttgaggttctgaggcaacattcaactggagatcttcagtactcgccagattacagaaattacctggctttaatcaaccatcgtcctcatgtcaaaggaaattccagctgctatggagtgttgcctacagaggagcctgtctataattggagaacggtaattaacagtgctgcggacttctattttgaaggaaatattcatcaatctctgcagaacataactgaaaaccagctggtacaacccactcttctccagcaaaaggggggaaaaggcaggaagaagctccgactgtttgaataccttcacgaatccctgtataatccggagatggcatcttgtattcagtgggtagataaaaccaaaggcatctttcagtttgtatcaaaaaacaaagaaaaacttgccgagctttgggggaaaagaaaaggcaacaggaagaccatgacttaccagaaaatggccagggcactcagaaattacggaagaagtggggaaattaccaaaatccggaggaagctgacttaccagttcagtgaggccattctccaaagactctctccatcctatttcctggggaaagagatcttctattcacagtgtgttcaacctgatcaagaatatctcagtttaaataactggaatgcaaattataattatacatatgccaattaccatgagctaaatcaccatgattgctaaatatactttcatatttcatggtttactggcatcggaaatctctacaagttttaatgatttctccctccctctctttttttcctcctctgaagaaatttaggatttttctcttaaagcaaatactaaagaggaaaaaaaattaactttattgttgcttttatcaaagagtatgtaatctatactaacttgttgggaaattctgccaatgaacaacttttttataataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - protein, 248 aa, Homo sapiens
MTCVEQDKLGQAFEDAFEVLRQHSTGDLQYSPDYRNYLALINHRPHVKGNSSCYGVLPTEEPVYNWRTVINSAADFYFEGNIHQSLQNITENQLVQPTLLQQKGGKGRKKLRLFEYLHESLYNPEMASCIQWVDKTKGIFQFVSKNKEKLAELWGKRKGNRKTMTYQKMARALRNYGRSGEITKIRRKLTYQFSEAILQRLSPSYFLGKEIFYSQCVQPDQEYLSLNNWNANYNYTYANYHELNHHDC

SEQ ID NO: 16 - DNA, 1663 bp, Homo sapiens
  CDS (264)..(1385): Nfe2
gtgcgcctgcttggggctcctgtgctcagctcagcctgagcttccacactcagcgctcagcaatggcccgggggcggggcgcggtcctcgcagattctcaaaggtagccgggatcctcgtccagcagtgtcagctcaggctcagcctccccagagacaacaccgggagcctcatctctctcctcaccctgctgtgactccaccacaggtttctagagccatctgggctttccgggaacctggaccagactctggcccagtaggatgtccccgtgtcctccccagcagagcaggaacagggtgatacagctgtccacttcagagctaggagagatggaactgacttggcaggagatcatgtccatcaccgagctgcagggtctgaatgctccaagtgagccatcatttgagccccaagccccagctccataccttggacctccaccacccacaacttactgcccctgctcaatccacccagattctggcttcccacttcctccaccaccttatgagctcccagcatccacatcccatgtcccagatcccccatactcctatggcaacatggccataccagtctccaagccactgagcctctcaggcctgctcagtgagccgctccaagaccccttagccctcctggacattgggctgccagcagggccacctaagccccaagaagacccagaatccgactcaggattatccctcaactatagcgatgctgaatctcttgagctggaggggacagaggctggtcggcggcgcagcgaatatgtagagatgtacccagtggagtacccctactcactcatgcccaactccttggcccactccaactataccttgccagctgctgagacccccttggccttagagccctcctcaggccctgtgcgggctaagcccactgcacggggggaggcagggagtcgggatgaacgtcgggccttggccatgaagattccttttcctacggacaagattgtcaacttgccggtagatgactttaatgagctattggcaaggtacccgctgacagagagccagctagcgctagtccgggacatccgacgacggggcaaaaacaaggtggcagcccagaactgccgcaagaggaagctggaaaccattgtgcagctggagcgggagctggagcggctgaccaatgaacgggagcggcttctcagggcccgcggggaggcagaccggaccctggaggtcatgcgccaacagctgacagagctgtaccgtgacattttccagcaccttcgggatgaatcaggcaacagctactctcctgaagagtacgcgctgcaacaggctgccgatgggaccatcttccttgtgccccgggggaccaagatggaggccacagactgagctggcccagaggggtggaactgctgatgggatttccttcattcccttctgataaaggtactccccaaccctgagtcccagaaggagctgagttctctagaccagaagaggatgacaatggcaacaagtgtttggaagttccaaggtgtgttcaaagaggcttgccttgagggagggctggaatctgtcttccctgactcggctcctcaggtctttagcctccaccttgtctaagctttggtctataaagtgcgctacagaaaaaaaaaaaaaaaaaa

SEQ ID NO: 17 - protein, 373 aa, Homo sapiens
MSPCPPQQSRNRVIQLSTSELGEMELTWQEIMSITELQGLNAPSEPSFEPQAPAPYLGPPPPTTYCPCSIHPDSGFPLPPPPYELPASTSHVPDPPYSYGNMAIPVSKPLSLSGLLSEPLQDPLALLDIGLPAGPPKPQEDPESDSGLSLNYSDAESLELEGTEAGRRRSEYVEMYPVEYPYSLMPNSLAHSNYTLPAAETPLALEPSSGPVRAKPTARGEAGSRDERRALAMKIPFPTDKIVNLPVDDFNELLARYPLTESQLALVRDIRRRGKNKVAAQNCRKRKLETIVQLERELERLTNERERLLRARGEADRTLEVMRQQLTELYRDIFQHLRDESGNSYSPEEYALQQAADGTIFLVPRGTKMEATD

SEQ ID NO: 18 - DNA, 1248 bp, Homo sapiens
  CDS (127)..(681): Tnfaip8l2
ggccaagccaaagggctctcacactaagtgaagcttctccattctgtaagctttccgggaacatccaaggcaagactggcacccagcacagcagtgactgaccacataccccactctccaggacccatggagtccttcagctcaaagagcctggcactgcaagcagagaagaagctactgagtaagatggcgggtcgctctgtggctcatctcttcatagatgagacaagcagtgaggtgctagatgagctctaccgtgtgtccaaggagtacacgcacagccggccccaggcccagcgcgtgatcaaggacctgatcaaagtggccatcaaggtggctgtgctgcaccgcaatggctcctttggccccagtgagctggccctggctacccgctttcgccagaagctgcggcagggtgccatgacggcacttagctttggtgaggtagacttcaccttcgaggctgctgttctggctggcctgctgaccgagtgccgggatgtgctgctagagttggtggaacaccacctcacgcccaagtcacatggccgcatccgccacgtgtttgatcacttctctgacccaggtctgctcacggccctctatgggcctgacttcactcagcaccttggcaagatctgtgacggactcaggaagctgctagacgaagggaagctctgagagccctgagcctagcacattccaccttgacaaaatggttgactgagaaaacacagataatgggcttcctaaccctgctcacctggcactaacacttttcaatcttcaggcttcattccttcccaagagtgcttttgactctgagaccagcccacccccaaacagctagtggagaaggagcaatgctgaggggtgaggcctctctcccactccagccccaggacaggaaacagaactgcctgaaaaaggtgaagtgaaacttggatctctatttctcccataagggacttctgaaacagggaagccccctcccatgtgaaccaaggaaaggaggcacagcccagagaacccctttggggatactaaagacagaagaggggaaggtggcccttagagacagagcttggacagatgccagaggctctgttccagagtgcaggaagaaggggctagggcaggggagattctcataggggaaataaaactactaaaatatgaaaaaaaaaaaaaaaaaaaaaaaaaaaaaaaaaaaaaaaaaaaaaaaaaaaaaaaaaaaaaaaaaaaaaa

SEQ ID NO: 19 - protein, 184 aa, Homo sapiens
MESFSSKSLALQAEKKLLSKMAGRSVAHLFIDETSSEVLDELYRVSKEYTHSRPQAQRVIKDLIKVAIKVAVLHRNGSFGPSELALATRFRQKLRQGAMTALSFGEVDFTFEAAVLAGLLTECRDVLLELVEHHLTPKSHGRIRHVFDHFSDPGLLTALYGPDFTQHLGKICDGLRKLLDEGKL

SEQ ID NO: 20 - DNA, 1254 bp, Homo sapiens
  CDS (33)..(503): Ier3
ctcacttggccttacactccgctcggctcaccatgtgtcactctcgcagctgccacccgaccatgaccatcctgcaggccccgaccccggccccctccaccatcccgggaccccggcggggctccggtcctgagatcttcaccttcgaccctctcccggagcccgcagcggcccctgccgggcgccccagcgcctctcgcgggcaccgaaagcgcagccgcagggttctctaccctcgagtggtccggcgccagctgccagtcgaggaaccgaacccagccaaaaggcttctctttctgctgctcaccatcgtcttctgccagatcctgatggctgaagagggtgtgccggcgcccctgcctccagaggacgcccctaacgccgcatccctggcgcccacccctgtgtccgccgtcctcgagccctttaatctgacttcggagccctcggactacgctctggacctcagcactttcctccagcaacacccggccgccttctaactgtgactccccgcactccccaaaaagaatccgaaaaaccacaaagaaacaccaggcgtacctggtgcgcgagagcgtatccccaactgggacttccgaggcaacttgaactcagaacactacagcggagacgccacccggtgcttgaggcgggaccgaggcgcacagagaccgaggcgcatagagaccgaggcacagcccagctggggctaggcccggtgggaaggagagcgtcgttaatttatttcttattgctcctaattaatatttatatgtatttatgtacgtcctcctaggtgatggagatgtgtacgtaatatttattttaacttatgcaagggtgtgagatgttccccctgctgtaaatgcaggtctcttggtatttattgagctttgtgggactggtggaagcaggacacctggaactgcggcaaagtaggagaagaaatggggaggactcgggtgggggaggacgtcccggctgggatgaagtctggtggtgggtcgtaagtttaggaggtgactgcatcctccagcatctcaactccgtctgtctactgtgtgagacttcggcggaccattaggaatgagatccgtgagatccttccatcttcttgaagtcgcctttagggtggctgcgaggtagagggttgggggttggtgggctgtcacggagcgactgtcgagatcgcctagtatgttctgtgaacacaaataaaattgatttactgtctgcaaaaaaaaaaaaaaa

SEQ ID NO: 21 - protein, 156 aa, Homo sapiens
MCHSRSCHPTMTILQAPTPAPSTIPGPRRGSGPEIFTFDPLPEPAAAPAGRPSASRGHRKRSRRVLYPRVVRRQLPVEEPNPAKRLLFLLLTIVFCQILMAEEGVPAPLPPEDAPNAASLAPTPVSAVLEPFNLTSEPSDYALDLSTFLQQHPAAF

SEQ ID NO: 22 - DNA, 4817 bp, Homo sapiens
  CDS (129)..(2546): Pik3ap1
gaagcgccagagcggcggccggtcccgcgcggagcccggcgcccctccagcccgagccaggacgccgccggccccggtcccggccccgggcacgcagcgagccagggatgtgagcggcgccccgcggcatggcagcctcaggggtgcccagaggatgcgacatcctcatcgtctacagcccggatgccgaggaatggtgccagtacctgcagaccctgttcctgtccagtcggcaggtccgcagccagaagatactgactcacaggctgggccccgaggcctccttctcggcagaggacctaagccttttcctcagcacccgctgtgtcgtggtgctgctgtccgcggagctggtgcagcacttccacaagcccgccttgctgcccctgctgcagagagctttccatcctccgcaccgcgtggtcaggctgctctgcggcgtgcgggacagcgaggagttcctagacttctttccagattgggcccattggcaggagctcacctgtgacgatgagccagagacctacgtggcagctgtgaaaaaagccatttccgaagattctggctgtgactcagtcactgacactgagcctgaggacgagaaggttgtttcctactcgaagcagcagaacctgccgacggtgacttcacctgggaacctgatggtggtgcagccggaccgcattcgctgtggggcagaaaccactgtctatgttattgtgagatgtaagctggatgacagggtggcgacagaagcagagttttctcctgaggattctccctctgtaaggatggaagccaaggtggagaatgagtacaccatttcagtgaaggctcccaacctttcatctgggaacgtttctctgaagatatattctggagacttagtggtgtgtgaaaccgttatcagctattatactgacatggaagaaattgggaatttattgtccaatgccgcgaatcctgtggaattcatgtgtcaggcctttaaaattgtgccctacaacacagagacccttgataaactgctaaccgaatccctgaagaacaatatccctgcaagcggactgcacctctttggaatcaaccagctggaagaagaagatatgatgacaaatcagagggatgaagagctgcccaccctgttgcattttgctgcgaagtatggactgaagaacctcactgccttgttgctcacctgcccaggagccctgcaggcgtacagcgtggccaacaagcatggccactaccccaacaccatcgctgagaaacacggcttcagggacctgcggcagttcatcgacgagtatgtggaaacggtggacatgctcaagagtcacattaaagaggaactgatgcacggggaggaggctgatgctgtgtacgagtccatggcccacctttccacagacctgcttatgaaatgctcgctcaaccccggctgtgacgaggatctctatgagtccatggctgcctttgtcccagctgccactgaagacctctatgttgaaatgcttcaggccagtacatctaacccaatccctggagatggtttctctcgggccactaaggactctatgatccgcaagtttttagaaggcaacagcatgggaatgaccaatctggagagagatcagtgccatcttggtcaggaagaagatgtttatcacacggtggatgacgatgaggccttttctgtggacctggccagcaggccccctgtcccagtgcccagaccagagaccactgctcctggtgctcaccagctgcctgacaacgaaccatacatttttaaagtttttgcagaaaaaagtcaagagcggcctgggaatttctacgtttcctcagagagcatcaggaaagggccgcccgtcagaccatggagggacaggccccagtcgagtatatatgacccttttgcgggaatgaaaacgccaggccagcggcagcttatcaccctccaggagcaggtgaagctgggcattgtcaacgtggatgaggctgtgctccacttcaaagagtggcagctcaaccagaagaaacgatcggagtcctttcgtttccagcaggaaaatcttaaacggctaagagacagcatcacccgaagacagagagagaagcaaaaatcaggaaagcagacagacttggagatcacggtcccaattcggcactcacagcacctgcctgcaaaagtggagtttggagtctatgagagtggccccaggaaaagtgtcattccccctaggacggagctgagacgaggagactggaaaacagacagcacctccagcacagcaagtagcacaagtaaccgctccagcacccggagcctcctcagtgtgagcagcgggatggaaggggacaacgaggataatgaagtccctgaggttaccagaagtcgcagtccaggccccccacaagtggatgggacacccaccatgtccctcgagagaccccccagggtgcctccgagagctgcctcacagaggcctccgaccagggagaccttccatcctcctccacctgttccacccagaggacgctgattccacctcctaaaacctgcctacttcaggactttaagactcacagtcttcagcctgttaatgatgtcttcatgttgagttttatagcatgactgttgaccttaagatccattctcattgctgataatgctgcagccctgctggtttgggcttgcctcgaagattttattaaggcacgaagaagtgaaaaactaagggcttcattcaccatcaccaagtatatcgaaccatatacttgtttgccaaaaggatgaagacttaatcgaaatacttacctctaatttgccatatcagaagcctaaaaagaatgatcataaatgtacttcaccagtgattttactgaaatgcacttatattagtctttatgtatttgctagttcagcctgatttctagaagaggttatagtgtgagacttgtagtattcaagtaagataagtgacctaattttaaaataattcttctacttttctgtatattcagcagggtatttaagtgctagggctggtcacacacaaccaactgaaaaagactagagggattagtacaaactcctcttatacagaaggcaaatctgaggttccacagaagtctggaaccaagactattcagttggttaaataaagaggttagtctagactgggcctgctcattctaggtcaccacattttccatctccaaatagccaggccctctctccctcaagaaatgcccagatgtagaaattcatcagtgcctattggtcttccagaattttccatcttccgtatctcccaggcatgagactaccaagtttgtttgttttctttccaatttgggaatttatacttcagtatggtttcaacgcagttatgtttccagagaacatctagaagtggctggaaaccagaagctggggattccagggaccccacttagtgctctatttcctttataggttttatttctggtcatagagagagaaggacctttgactttttcttcgttgaggcttctgaggaggaaaaacaaacctaaaatagaaatacagtcagcctttcaaatccatgggttctgtgtccgtggattcaaccaaccttggatcaaaaatatttgaaaaaaaatctacaaagtttcaaaaagcaaaacttgaatttgctgcatgccaagaagtatgttgaattcatgtaaatgaagtgatgtgtaggcattgtattagatattataagaaatctagaaatgatttaaagcatacaggaggatgtgcataggttatatgcaaatactatgctattttatatatgggacttgagcatttgtggattttgatactgggggatcctggaaccaatcccccatggataccaaagtacgactgtagttatctattttttacatacttattattaccaccatgctcagtaagtccatttttgcatggaatatggagccttaaaacatgtcatgaatttggagtccctggcacataaatctaccttcaaatcagaggtccttaatgatgcctaaacatacagtaaaattagaatcagaaatacttctttaaaaaatattcaaaatgtgtttgtttcccatgggattattctctatcccacacgaatgtaaaaaaatccacattaatgatccatttaagtatagttttattgggtccttttctaatgattaaaggttctttctcaatttcattcctcagtcctgcaagtaaggactcatactgaagagtactgaaacaaggacttcttgtcagaaacagcttctggaatcttgggttttgtttttgttttttgacaaaatacactattggccatgtccatcacgagagtgtttgtagtaattaattaccttgtacaggacctggcacttagtagcattcttcaaatgttccctcagtgatccttttactctccttgtcacttatttgggagaaataggggcacgtgagataagaagaagaataattttgatgttggtatgcttgccctgttacttatagacagtctttgtcataggcaaacttgaatttgatttaaaatagggctgggaaaaatattcaataactgtaagcccccttttggatgccaaacttagaattttgtacattctctgatgaacaagcatttagatcgtaacatggtaaagcctattaccagccaatgttgttagcatctttgtatgcacatcactgtttgtgcaatatatgaatattttgttgcattgtattcttatataaaaataagtagaaaacataaaattgaaattgctgatgagaaaaaaaa

SEQ ID NO: 23 - protein, 805 aa, Homo sapiens
MAASGVPRGCDILIVYSPDAEEWCQYLQTLFLSSRQVRSQKILTHRLGPEASFSAEDLSLFLSTRCVVVLLSAELVQHFHKPALLPLLQRAFHPPHRVVRLLCGVRDSEEFLDFFPDWAHWQELTCDDEPETYVAAVKKAISEDSGCDSVTDTEPEDEKVVSYSKQQNLPTVTSPGNLMVVQPDRIRCGAETTVYVIVRCKLDDRVATEAEFSPEDSPSVRMEAKVENEYTISVKAPNLSSGNVSLKIYSGDLVVCETVISYYTDMEEIGNLLSNAANPVEFMCQAFKIVPYNTETLDKLLTESLKNNIPASGLHLFGINQLEEEDMMTNQRDEELPTLLHFAAKYGLKNLTALLLTCPGALQAYSVANKHGHYPNTIAEKHGFRDLRQFIDEYVETVDMLKSHIKEELMHGEEADAVYESMAHLSTDLLMKCSLNPGCDEDLYESMAAFVPAATEDLYVEMLQASTSNPIPGDGFSRATKDSMIRKFLEGNSMGMTNLERDQCHLGQEEDVYHTVDDDEAFSVDLASRPPVPVPRPETTAPGAHQLPDNEPYIFKVFAEKSQERPGNFYVSSESIRKGPPVRPWRDRPQSSIYDPFAGMKTPGQRQLITLQEQVKLGIVNVDEAVLHFKEWQLNQKKRSESFRFQQENLKRLRDSITRRQREKQKSGKQTDLEITVPIRHSQHLPAKVEFGVYESGPRKSVIPPRTELRRGDWKTDSTSSTASSTSNRSSTRSLLSVSSGMEGDNEDNEVPEVTRSRSPGPPQVDGTPTMSLERPPRVPPRAASQRPPTRETFHPPPPVPPRGR

SEQ ID NO: 24 - DNA, 1870 bp, Homo sapiens
  CDS (451)..(1701): Pstpip1
acaaaccttcctgcgcaggcctcgggctgcctgcctgcctgcctgcctggcccggcccgagctccagcctgcctcttccactggccactgcctcccacccagggctggcatccctgctccctgccctgggtcccagactgtgtcctccatcaccgcagggtcggtgaggggctgggctggacaccagggcccgccctcccatcactgagctccactccttcctcattttgctgctgattctagccccaaacaaaacaggttgagctttttcctcccctcagaagctcctctctggctcgtggctgccttctgagtgttgcagacggcgccggccgggaaggggggcctgggccagccctgccaggactgggacgctgctgctggcgcctggccctccatcaggccagcctgtggcaggagagtgagctttgccgcggcagacgcctgaggatgatgccccagctgcagttcaaagatgccttttggtgcagggacttcacagcccacacgggctacgaggtgctgctgcagcggcttctggatggcaggaagatgtgcaaagacatggaggagctactgaggcagagggcccaggcggaggagcggtacgggaaggagctggtgcagatcgcacggaaggcaggtggccagacggagatcaactccctgagggcctcctttgactccttgaagcagcaaatggagaatgtgggcagctcacacatccagctggccctgaccctgcgtgaggagctgcggagtctcgaggagtttcgtgagaggcagaaggagcagaggaagaagtatgaggccgtcatggaccgggtccagaagagcaagctgtcgctctacaagaaggccatggagtccaagaagacatacgagcagaagtgccgggacgcggacgacgcggagcaggccttcgagcgcattagcgccaacggccaccagaagcaggtggagaagagtcagaacaaagccaggcagtgcaaggactcggccaccgaggcagagcgggtatacaggcagagcattgcgcagctggagaaggtccgggctgagtgggagcaggagcaccggaccacctgtgaggcctttcagctgcaagagtttgaccggctgaccattctccgcaacgccctgtgggtgcacagcaaccagctctccatgcagtgtgtcaaggatgatgagctctacgaggaagtgcggctgacgctggaaggctgcagcatagacgccgacatcgacagtttcatccaggccaagagcacgggcacagagccccccgctccggtgccctaccagaactattacgatcgggaggtcaccccgctgaccagcagccctggcatacagccgtcctgcggcatgataaagaggttctctggactgctgcacggaagtcccaagaccacttcgttggcagcttctgctgcgtccacagagaccctgacccccacccccgagcggaatgagggtgtctacacagccatcgcagtgcaggagatacagggaaacccggcctcaccagcccaggagtaccgggcgctctacgattatacagcgcagaacccagatgagctggacctgtccgcgggagacatcctggaggtgatcctggaaggggaggatggctggtggactgtggagaggaacgggcagcgtggcttcgtccctggttcctacctggagaagctttgaggaagggccaggagccccttcggacctgccctgccagtggagccagcagtgcccccagcactgtccccaccttgctagggcccagaaccaagcgtcccccagccccgagagggagcctgtcgtctcccagggaataaaggagtgcgttctgttcaaaaaaaaaaaaaaa

SEQ ID NO: 25 - protein, 416 aa, Homo sapiens
MMPQLQFKDAFWCRDFTAHTGYEVLLQRLLDGRKMCKDMEELLRQRAQAEERYGKELVQIARKAGGQTEINSLRASFDSLKQQMENVGSSHIQLALTLREELRSLEEFRERQKEQRKKYEAVMDRVQKSKLSLYKKAMESKKTYEQKCRDADDAEQAFERISANGHQKQVEKSQNKARQCKDSATEAERVYRQSIAQLEKVRAEWEQEHRTTCEAFQLQEFDRLTILRNALWVHSNQLSMQCVKDDELYEEVRLTLEGCSIDADIDSFIQAKSTGTEPPAPVPYQNYYDREVTPLTSSPGIQPSCGMIKRFSGLLHGSPKTTSLAASAASTETLTPTPERNEGVYTAIAVQEIQGNPASPAQEYRALYDYTAQNPDELDLSAGDILEVILEGEDGWWTVERNGQRGFVPGSYLEKL

SEQ ID NO: 26 - DNA, 60 bp, Artificial
  Probe A_44_P1004731
ctggcttacagcttgtttgttttataactttacctctctctgaaaagtctgtaagcaagg

SEQ ID NO: 27 - DNA, 60 bp, Artificial
  Probe A_42_P592157
aaagctttcggttttgttttttaaactgtttgcagagtggagaagatcgatcaggaaggg

SEQ ID NO: 28 - DNA, 60 bp, Artificial
  Probe A_42_P495820
attatgcaggctatgacggaactactaccttgctatggatgagggttgggcaggatttaa

SEQ ID NO: 29 - DNA, 60 bp, Artificial
  Probe CUST_1_PI195698246
gtctcaagaacagagggctaccttggggagccataaagagtgtatttaataaaacgggct

SEQ ID NO: 30 - DNA, 60 bp, Artificial
  Probe A_44_P377266
aatgaaataactaagcatatcttttgagaattttattttcttacattttaaatctgaagg

SEQ ID NO: 31 - DNA, 60 bp, Artificial
  Probe A_66_P100662
tttgtggtccctgttcagtcattatgttgtcccttcgcttctcttgatcagcagaaagca

SEQ ID NO: 32 - DNA, 60 bp, Artificial
  Probe A_44_P928825
gaacgtgtgcacaaagtatcagcagaaatccagtctgtgaaagaagaacaagagcatgtg

SEQ ID NO: 33 - DNA, 60 bp, Artificial
  Probe A_42_P526140
ctcagtgtccgtgaattgggtatccaagaacatcctgaagccagaatgtcttctcagaaa

SEQ ID NO: 34 - DNA, 60 bp, Artificial
  Probe A_42_P464736
aggctgagttctccagaccaaaagaccatttggaagttcaaagatgtatttgaggtttgc

SEQ ID NO: 35 - DNA, 60 bp, Artificial
  Probe A_43_P20022
agctctgaggctcctgagctcagcacactggactttggcaaaatgactgaccgggaaacg

SEQ ID NO: 36 - DNA, 60 bp, Artificial
  Probe A_42_P515405
atttattctaacttatgcaggggtgcgagatatgcccccttgctgtgacacagatattta

SEQ ID NO: 37 - DNA, 60 bp, Artificial
  Probe A_43_P21121
acctggagacccactgtcactggtgatggtgtagccctgctggtttgggtgatccttgaa

SEQ ID NO: 38 - DNA, 60 bp, Artificial
  Probe A_44_P180717
tggtgtgataaagaggttctctgggctgctacatggaagtcccaagaccacaccttctca

SEQ ID NO: 39 - DNA, 60 bp, Artificial
  Probe A_42_P602724
gtgactacgggagtggggtgatgagaagctcaaaaccgacttcctttaatcaattaacca

SEQ ID NO: 40 - DNA, 60 bp, Artificial
  Probe A_44_P332606
cctggtgagttggggtctttttgacccttgtcacggttcctccaacaaaaacttgcgcag

SEQ ID NO: 41 - DNA, 60 bp, Artificial
  Probe A_43_P12274
aggcagaggagaaggtgaaggcgtaaaagaagacgccagcactgactatgcctgcatcgt

SEQ ID NO: 42 - DNA, 60 bp, Artificial
  Probe A_44_P421534
tttctcagaatgatgctgggcaagagatctgccatcttgagaatgattctgatgtatgag

SEQ ID NO: 43 - DNA, 60 bp, Artificial
  Probe A_44_P248172
ggtttctcctctatgttgatacagattacctggattctatatcagaagacgatgagttgt

SEQ ID NO: 44 - DNA, 60 bp, Artificial
  Probe A_43_P13426
ttctcctttgattgctcttcctaactgggaccaagttctgggaaagaaggctgatgttca

SEQ ID NO: 45 - DNA, 60 bp, Artificial
  Probe A_43_P11484
atcatattgctgaaaatcttcaaacacaaactctggggtgagcattaccattgaacagtt

SEQ ID NO: 46 - DNA, 60 bp, Artificial
  Probe A_44_P285534
accccggaaacgtattcctatttgaagcaagttgaacggacagagaagggaagaagagaa

SEQ ID NO: 47 - DNA, 60 bp, Artificial
  Probe A_44_P421727
gagatgagtacctccggaaacagaagacggagaccatcatctactcccgagaaaagaacc

SEQ ID NO: 48 - DNA, 20 bp, Artificial
  Primer RT1-A2 F
tccctccctgctaccctgag

SEQ ID NO: 49 - DNA, 20 bp, Artificial
  Primer RT1-A2 R
gccatccacacttgggtcaa

SEQ ID NO: 50 - DNA, 21 bp, Artificial
  Primer RT1-DMb F
tcaaatctgcctcgggtgttt

SEQ ID NO: 51 - DNA, 20 bp, Artificial
  Primer RT1-DMb R
gacaaggtggggctttcagg

SEQ ID NO: 52 - DNA, 20 bp, Artificial
  Primer Psmb8 F
cactgctgggcagacatcct

SEQ ID NO: 53 - DNA, 20 bp, Artificial
  Primer Psmb8 R
gctttgtctccagcccaggt

SEQ ID NO: 54 - DNA, 20 bp, Artificial
  Primer Ly6g6e F
cccaggcaaagggacagaag

SEQ ID NO: 55 - DNA, 20 bp, Artificial
  Primer Ly6g6e R
tgagaccctcaggcaccaag

SEQ ID NO: 56 - DNA, 20 bp, Artificial
  Primer Aif1 F
tcccccagccaagaaagcta

SEQ ID NO: 57 - DNA, 21 bp, Artificial
  Primer Aif1 R
tcttttcccatgctgctgtca

SEQ ID NO: 58 - DNA, 19 bp, Artificial
  Primer Lst1 F
gggcaggagctccactacg

SEQ ID NO: 59 - DNA, 21 bp, Artificial
  Primer Lst1 R
cgatgcaggcatagtcagtgc

SEQ ID NO: 60 - DNA, 20 bp, Artificial
  Primer RT1-CE3 F
tgtcgtccttggagccatct

SEQ ID NO: 61 - DNA, 20 bp, Artificial
  Primer RT1-CE3 R
tcctcacaacaggcaccaga

SEQ ID NO: 62 - DNA, 20 bp, Artificial
  Primer RT1-CE10 F
acacaggtggggaaggagga

SEQ ID NO: 63 - DNA, 22 bp, Artificial
  Primer RT1-CE10 R
caatctgggagggacacatcag

SEQ ID NO: 64 - DNA, 23 bp, Artificial
  Primer RT-BM1(RT1-S3) F
gcagctatgctcatgttctaggc

SEQ ID NO: 65 - DNA, 20 bp, Artificial
  Primer RT-BM1(RT1-S3) R
tgccttctgaggccagtcag

SEQ ID NO: 66 - DNA, 22 bp, Artificial
  Primer Ubd F
tggggtgatgagaagctcaaaa

SEQ ID NO: 67 - DNA, 26 bp, Artificial
  Primer Ubd R
ccccacctcaaatctttatttcattc

SEQ ID NO: 68 - DNA, 21 bp, Artificial
  Primer Olr1 F
ggaagtcagaagagggcatgg

SEQ ID NO: 69 - DNA, 22 bp, Artificial
  Primer Olr1 R
tcctgggttcaatttccagagt

SEQ ID NO: 70 - DNA, 22 bp, Artificial
  Primer Ly49si1 F
tggccaatctgaattttccttg

SEQ ID NO: 71 - DNA, 20 bp, Artificial
  Primer Ly49si1 R
acatgggaaggggttcatgc

SEQ ID NO: 72 - DNA, 20 bp, Artificial
  Primer Ly49i9 F
gggacttggcaacctcagga

SEQ ID NO: 73 - DNA, 22 bp, Artificial
  Primer Ly49i9 R
ttggaacatctgcacaatggaa

SEQ ID NO: 74 - DNA, 20 bp, Artificial
  Primer Cd3z F
agtgcctgctgggatttagc

SEQ ID NO: 75 - DNA, 21 bp, Artificial
  Primer Cd3z R
catccatggtcacaggcactt

SEQ ID NO: 76 - DNA, 20 bp, Artificial
  Primer B2m F
gagcaggttgctccacaggt

SEQ ID NO: 77 - DNA, 24 bp, Artificial
  Primer B2m R
caagctttgagtgcaagagattga